(19) United States
(12) Patent Application Publication
(10) Pub. No.: US 2009/0233930 A9
(43) Pub. Date: Sep. 17, 2009

(54) Novel 2-Amino-Imidazole-4-One Compounds and Their Use in the Manufacture of a Medicament to Be Used in the Treatment of Cognitive Impairment, Alzheimer's Disease, Neurodegeneration and Dementia

(75) Inventors: Stefan Berg, Sodertalje (SE); Jeremy Burrows, Sodertalje (SE); Gianni Chessari, Cambridge (GB); Miles Stuart Congreve, Cambridge (GB); Johan Hedstrom, Sodertalje (SE); Sven Hellberg, Sodertalje (SE); Katharina Hogdin, Sodertalje (SE); Jacob Kihlstrom, Sodertalje (SE); Karin Kolmodin, Sodertalje (SE); Johan Lindstrom, Sodertalje (SE); Christopher Murray, Cambridge (GB); Sahil Patel, Cambridge (GB)

Correspondence Address: Pepper Hamilton LLP, 400 Berwyn Park, 899 Cassatt Road, Berwyn, PA, 19312-1183, US

(73) Assignees: ASTRAZENECA AB, Sodertalje (SE); ASTEX THERAPEUTICS LIMITED, Cambridge (GB)

(21) Appl. No.: 12/094,276

(22) PCT Filed: Nov. 20, 2006
(86) PCT No.: PCT/SE2006/001317

§ 371 Date: May 20, 2008

Related U.S. Application Data
(60) Provisional application No. 60/738,650, filed on Nov. 21, 2005.
(60) Provisional application No. 60/815,746, filed on Jun. 22, 2006.

(51) Int. Cl. A61K 31/5377; C07D 401/10; A61K 31/4725; C07D 339/08; A61P 25/28; C07F 7/08; C12N 9/99; C07D 413/12

(52) U.S. Cl. 514/235.8; 546/146; 514/307; 544/139; 435/184; 549/4; 556/436

(57) ABSTRACT

This invention relates to novel compounds having the structural formula I below: and to their pharmaceutically acceptable salt, compositions and methods of use. These novel compounds provide a treatment or prophylaxis of cognitive impairment, Alzheimer Disease, neurodegeneration and dementia.

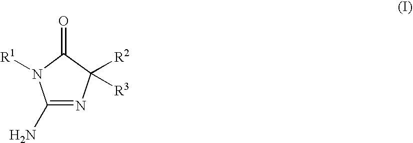

The present invention relates to novel compounds, their pharmaceutical compositions. In addition, the present invention relates to therapeutic methods for the treatment and/or prevention of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

### BACKGROUND OF THE INVENTION

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, EP871720, U.S. Pat. Nos. 5,942,400 and 5,744,346, EP855444, U.S. Pat. No. 6,319,689, WO99/64587, WO99/31236, EP1037977, WO00/17369, WO01/23533, WO0047618, WO00/58479, WO00/69262, WO01/00663, WO01/00665, U.S. Pat. No. 6,313,268.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999)) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class I transmembrane protein called APP, or amyloid precursor protein. Aβ-secretase activity cleaves this protein between residues Met671 and Asp672 (numbering of 770aa isoform of APP) to form the N-terminus of Aβ. A second cleavage of the peptide is associated with γ-secretase to form the C-terminus of the Aβ peptide.

Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much greater chance of developing AD, and also of developing it at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to acquire Alzheimer's disease at an early age, with almost all those over 40 years of age showing Alzheimer's-type pathology (Oyama et al., 1994). This is thought to be due to the extra copy of the APP gene found in these patients, which leads to overexpression of APP and therefore to increased levels of APPβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533 A2, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO 05/097767, US2005/0282826).

The compounds of the present invention show improved properties compared to the potential inhibitors known in the art, e.g. improved hERG selectivity.

### DISCLOSURE OF THE INVENTION

Provided herein are novel compounds of structural formula I:

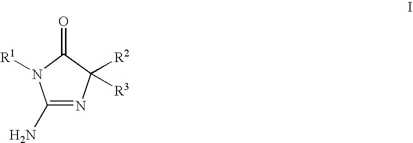

wherein
R¹ is selected from hydrogen, C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl, wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one, two or three A;
R² is selected from hydrogen, nitro, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl, wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl is optionally substituted by one, two or three R⁷;
-Q- is a direct bond, —CONH—, —CO—, —CON(C₁₋₆alkyl)-, —CON(C₃₋₆cycloalkyl)-, —SO—, —SO₂—, —SO₂NH—, —SO₂N(C₁₋₆alkyl)-, —SO₂N(C₃₋₆cycloalkyl)-, —NHSO₂—, —N(C₁₋₆alkyl)SO₂—, —NHCO—, —N(C₁₋₆alkyl)CO—, —N(C₃₋₆cycloalkyl)CO— or —N(C₃₋₆cycloalkyl)SO₂—;
R³ is (C(R⁴)(R⁵))ₙR⁶, C₂₋₄alkenylR⁶, C₂₋₄alkynylR⁶, C₅₋₇cycloalkenylR⁶, nitro or cyano and if n>1 then each C(R⁴)(R⁵) is independent of the others;
R⁴ and R⁵ are independently selected from hydrogen, C₁₋₆alkyl, cyano, halo or nitro; or R⁴ and R⁵ together form oxo, C₃₋₆cycloalkyl or heterocyclyl;
R⁶ is selected from methyl, C₃₋₆cycloalkyl, heterocyclyl, aryl or heteroaryl wherein each of the said methyl, C₃₋₆cycloalkyl, heterocyclyl, aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four A with the proviso that the bicyclic ring is not an indane, benzo[1,3]dioxole or 2,3-dihydrobenzo[1,4]-dioxine ring system;
R⁷ is selected from halogen, nitro, CHO, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸(CO)R⁹, O(CO)NR⁸R⁹, NR⁸(CO)OR⁹, NR⁸(CO)NR⁸R⁹, O(CO)OR⁸, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₁₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl, wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four A with the proviso that said bicyclic ring system is not an indane, benzo[1,3]dioxole or 2,3-dihydrobenzo[1,4]-dioxine ring system;
R¹⁴ is selected from halogen, nitro, CHO, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸(CO)R⁹, O(CO)NR⁸R⁹, NR⁸(CO)OR⁹, NR⁸(CO)NR⁸R⁹, OR⁸, O(CO)OR⁸, O(CO)R⁸, C₀₋₆alkylCOR⁸, OC₁₋₆alkylCOR⁸, NR⁸(CO)(CO)R¹, NR⁸(CO)(CO)NR⁸R⁹, C₀₋₆alkylSR⁸, C₀₋₆alkyl(SO₂)NR⁸R⁹, OC₂₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, OSO₂R⁸R⁹, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl may be optionally substituted by between one and four A;
R⁸ and R⁹ are independently selected from hydrogen, C₁₋₆alkyl, C₂₋₆alkenyl, C₂₋₆alkynyl, fluoromethyl, difluoromethyl, trifluoromethyl, fluoromethoxy, difluoromethoxy, trifluoromethoxy, C₀₋₆alkylC₃₋₆cycloalkyl, C₀₋₆alkylaryl, C₀₋₆alkylheteroaryl, C₀₋₆alkylheterocyclyl and C₀₋₆alkylNR¹⁰R¹¹, wherein the C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl or C₀₋₆alkylheterocyclyl are optionally substituted by A; or
R⁸ and R⁹ may together form a 4 to 6 membered heterocyclic ring containing one or more heteroatoms selected from N, O or S that is optionally substituted by A; whenever two R⁸ groups occur in the structure then they may optionally together form a 5 or 6 membered heterocyclic ring containing one or more heteroatoms selected from N, O or S, that is optionally substituted by A;
R¹⁰ and R¹¹ are independently selected from hydrogen, C₁₋₆alkyl, C₃₋₆alkenyl, C₃₋₆alkynyl, C₀₋₆alkylC₃₋₆cycloalkyl, C₀₋₆alkylaryl, C₀₋₆alkylheterocyclyl and C₀₋₆alkylheteroaryl, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R¹⁰ and R¹¹ may together form a 4 to 6 membered heterocyclic ring containing one or more heteroatoms selected from N, O or S optionally substituted by A;
n is 0, 1, 2 or 3
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, OC₂₋₆alkylNR¹²R¹³, NR¹²R¹³, CONR¹²R¹³, NR¹²(CO)R¹³, O(CO)C₁₋₆alkyl, (CO)OC₁₋₆alkyl, COR¹², (SO₂)NR¹²R¹³, NSO₂R¹², SO₂R¹², SOR¹², (CO)C₁₋₆alkylNR¹²R¹³, (SO₂)C₁₋₆alkylNR¹²R¹³, OSO₂R¹², SO₃R¹² wherein the C₁₋₆alkyl, C₂₋₆alkenyl, C₂₋₆alkynyl, C₀₋₆alkylaryl, C₀₋₆alkylheteroaryl, C₀₋₆alkylheterocyclyl and C₀₋₆alkylC₃₋₆cycloalkyl groups may be optionally substituted with halo, OSO₂R¹², SO₃R¹², nitro, cyano, OR¹², C₁₋₆alkyl, fluoromethyl, difluoromethyl, trifluoromethyl, fluoromethoxy, difluoromethoxy and trifluoromethoxy;
R¹² and R¹³ are independently selected from hydrogen, C₁₋₆alkyl, C₃₋₆cycloalkyl, aryl, heteroaryl or heterocyclyl wherein said C₁₋₆alkyl, C₃₋₆cycloalkyl, aryl, heteroaryl or heterocyclyl is optionally substituted by one, two or three hydroxy, cyano, halo or C₁₋₃alkyloxy; or
R¹² and R¹³ may together form a 4 to 6 membered heterocyclic ring containing one or more heteroatoms selected from N, O or S optionally substituted by hydroxy, C₁₋₃alkyloxy, cyano or halo;
provided that either any of the aryl or heteroaryl groups in R¹, R² or R³ is substituted with a OSO₂R⁸, SO₃R⁸, OSO₂R¹² or SO₃R¹² group;
or
provided that any of the individual aryl or heteroaryl groups in R¹, R² or R³ are fused with a 4, 5, 6 or 7 membered cycloalkyl, cycloalkenyl or heterocyclyl group to form a bicyclic ring system where the bicyclic ring system is optionally substituted with between one and four A with the proviso that the bicyclic ring is not an indane, benzo[1,3]dioxole or 2,3-dihydrobenzo[1,4]-dioxine ring system;
or
provided that R¹ is C₃₋₆alkynyl or C₅₋₇cycloalkenyl optionally substituted with one, two or three A;
or
provided that Q is selected from —NHSO₂—, —N(C₁₋₆alkyl)SO₂—, —SO₂NH—, —SO₂N(C₁₋₆alkyl)- or —SO₂N(C₃₋₆cycloalkyl)-, —SO—, —SO₂— or —N(C₃₋₆cycloalkyl)SO₂—;
or
provided that R³ is selected from C₂₋₄alkenylR⁶, C₂₋₄alkynylR⁶, C₅₋₇cycloalkenylR⁶, nitro or cyano;
or
provided that R² is selected from nitro, cyano, C₂₋₆alkynyl, C₅₋₇cycloalkenyl or C₂₋₆alkenyl group where the C₂₋₆alkynyl, C₅₋₇cycloalkenyl or C₂₋₆alkenyl group is optionally substituted by one, two or three R⁷;
as a free base or a pharmaceutically acceptable salt, solvate or solvate of a salt thereof.

The present invention further provides compositions comprising a compound of formula I, and at least one pharmaceutically acceptable carrier, diluent or excipient.

The present invention further provides methods of modulating activity of BACE comprising contacting the BACE with a compound of formula I.

The present invention further provides methods of treating or preventing an Aβ-related pathology in a patient, comprising administering to the patient a therapeutically effective amount of a compound of formula I.

The present invention further provides a compound described herein for use as a medicament.

The present invention further provides a compound described herein for the manufacture of a medicament.

In one aspect of the invention, there is provided a compound of formula I, wherein

R¹ is selected from C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one or two A;
R² is selected from hydrogen, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl is optionally substituted by one, two or three R⁷;
-Q- is a direct bond, —CONH—, —CO—, —CON(C₁₋₆alkyl)-, —CON(C₃₋₆cycloalkyl)-, —NHCO—, —N(C₁₋₆alkyl)CO— or —N(C₃₋₆cycloalkyl)CO—;
R³ is (C(R⁴)(R⁵))ₙR⁶, C₂₋₄alkenylR⁶, C₂₋₄alkynylR⁶ or C₅₋₇cycloalkenylR⁶ and if n>1 then each C(R⁴)(R⁵) is independent of the others;
R⁴ and R⁵ are independently selected from hydrogen, C₁₋₆alkyl, cyano, or halo; or R⁴ and R⁵ together form oxo, C₃₋₆cycloalkyl or heterocyclyl;
R⁶ is selected from methyl, C₃₋₆cycloalkyl, heterocyclyl, aryl or heteroaryl wherein each of the said methyl, C₃₋₆cycloalkyl, heterocyclyl, aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that the bicyclic ring is not an indane, benzo[1,3]dioxole or 2,3-dihydrobenzo[1,4]-dioxine ring system;
R⁷ is selected from halogen, nitro, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸ (CO)R⁹, O(CO)NR⁸R⁹, NR⁸(CO)OR⁹, NR⁸(CO)NR⁸R⁹, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₁₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that said bicyclic ring system is not an indane, benzo[1,3]dioxole or 2,3-dihydrobenzo[1,4]-dioxine ring system;
R¹⁴ is selected from halogen, nitro, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸(CO)R⁹, O(CO)NR⁸R⁹, NR⁸(CO)OR⁹, NR⁸(CO)NR⁸R⁹, OR⁸, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₂₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, OSO₂R⁸R⁹, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl may be optionally substituted by between one and three A;
R⁸ and R⁹ are independently selected from hydrogen, C₁₋₆alkyl, C₃₋₆alkenyl, C₃₋₆alkynyl, fluoromethyl, difluoromethyl, trifluoromethyl, C₀₋₆alkylC₃₋₆cycloalkyl, C₀₋₆alkylaryl, C₀₋₆alkylheteroaryl, C₀₋₆alkylheterocyclyl, C₀₋₆alkylNR¹⁰R¹¹ and C₁₋₆alkylNR¹⁰R¹¹, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R⁸ and R⁹ may together form a 4 to 6 membered heterocyclic ring containing one or more heteroatoms selected from N, O or S that is optionally substituted by A; whenever two R⁸ groups occur in the structure then they may together form a 5 or 6 membered heterocyclic ring containing one or more heteroatoms selected from N, O or S, that is optionally substituted by A;
R¹⁰ and R¹¹ are independently selected from hydrogen, C₁₋₆alkyl, C₃₋₆alkenyl, C₃₋₆alkynyl, C₀₋₆alkylC₃₋₆cycloalkyl, C₀₋₆alkylaryl, C₀₋₆alkylheterocyclyl and C₀₋₆alkylheteroaryl, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl or C₀₋₆alkylheterocyclyl are optionally substituted by A; or
R¹⁰ and R¹¹ may together form a 4 to 6 membered heterocyclic ring containing one or more heteroatoms selected from N, O or S optionally substituted by A;
n is 0, 1, or 2
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, OC₂₋₆alkylNR¹²R¹³, NR¹²R¹³, CONR¹²R¹³, NR¹²(CO)R¹³, O(CO)C₁₋₆alkyl, (CO)OC₁₋₆alkyl, COR¹², (SO₂)NR¹²R¹³, NSO₂R¹², SO₂R¹², SOR¹², (CO)C₁₋₆alkylNR¹²R¹³, (SO₂)C₁₋₆alkylNR¹²R¹³, OSO₂R¹², SO₃R¹² wherein the C₁₋₆alkyl, C₂₋₆alkenyl, C₂₋₆alkynyl, C₀₋₆alkylaryl, C₀₋₆alkylheteroaryl, C₀₋₆alkylheterocyclyl and C₀₋₆alkylC₃₋₆cycloalkyl groups may be optionally substituted with halo, OSO₂R¹², SO₃R¹², nitro, cyano, OR¹², C₁₋₆alkyl, fluoromethyl, difluoromethyl, trifluoromethyl, fluoromethoxy, difluoromethoxy and trifluoromethoxy;
R¹² and R¹³ are independently selected from hydrogen, C₁₋₆alkyl, C₃₋₆cycloalkyl, aryl, heteroaryl or heterocyclyl wherein said C₁₋₆alkyl, C₃₋₆cycloalkyl, aryl, heteroaryl or heterocyclyl is optionally substituted by one or two hydroxy, cyano, halo or C₁₋₃alkyloxy; or
R¹² and R¹³ may together form a 4 to 6 membered heterocyclic ring containing one or more heteroatoms selected from N, O or S optionally substituted by hydroxy, cyano, C₁₋₃alkyloxy or halo;
as a free base or a pharmaceutically acceptable salt, solvate or solvate of a salt thereof.

In another aspect of the invention, there is provided a compound of formula I, wherein

R¹ is selected from C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one or two A;
R² is selected from -Q-aryl and -Q-heteroaryl, wherein said -Q-aryl or -Q-heteroaryl is optionally substituted by one, two or three R⁷;
-Q- is a direct bond, —CONH—, —CO—, —CON(C₁₋₆alkyl)-, —CON(C₃₋₆cycloalkyl)-, —NHCO—, —N(C₁₋₆alkyl)CO— or —N(C₃₋₆cycloalkyl)CO—;
R³ is (C(R⁴)(R⁵))ₙR⁶;
R⁶ is selected aryl or heteroaryl wherein each of the said aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that the bicyclic ring is not an indane, benzo[1,3]dioxole or 2,3-dihydrobenzo[1,4]-dioxine ring system;
R⁷ is selected from halogen, nitro, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, OSO₂R⁸, C₀₋₆alkylaryl and C₀₋₆alkylheteroaryl, wherein any C₀₋₆alkylaryl or C₀₋₆alkylheteroaryl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that said bicyclic ring system is not an indane, benzo[1,3]dioxole or 2,3-dihydrobenzo[1,4]-dioxine ring system;
R¹⁴ is selected from halogen, nitro, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylCO₂R⁸, C₀₋₆alkylNR⁸(CO)R⁹, OR⁸, O(CO)R⁸, C₀₋₆alkylCOR⁸, OSO₂R⁸, OSO₂R⁸R⁹C₁₋₆alkyl;
R⁸ and R⁹ are independently selected from hydrogen, C₁₋₆alkyl, C₃₋₆alkenyl, C₃₋₆alkynyl, fluoromethyl, difluoromethyl, trifluoromethyl, C₀₋₆alkylC₃₋₆cycloalkyl, C₀₋₆alkylaryl, C₀₋₆alkylheteroaryl, C₀₋₆alkylheterocyclyl, C₀₋₆alkylNR¹⁰R¹¹ and C₁₋₆alkylNR¹⁰R¹¹, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R⁸ and R⁹ may together form a 4 to 6 membered heterocyclic ring containing one or more heteroatoms selected from N, O or S that is optionally substituted by A; whenever two R⁸ is groups occur in the structure then they may together form a 5 or 6 membered heterocyclic ring containing one or more heteroatoms selected from N, O or S, that is optionally substituted by A;
R¹⁰ and R¹¹ are independently selected from hydrogen or C₁₋₆alkyl; or
R¹⁰ and R¹¹ may together form a 4 to 6 membered heterocyclic ring containing one or more heteroatoms selected from N, O or S optionally substituted by A;
n is 0;
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy and COR¹²;
R¹² and R¹³ are independently selected from hydrogen and C₁₋₆alkyl;
as a free base or a pharmaceutically acceptable salt, solvate or solvate of a salt thereof.

In another aspect of the invention, there is provided a compound of formula I, wherein R¹ is C₁₋₆alkyl.

A compound according to any one of claims 1 to 4, wherein -Q- in R² represents a direct bond.

In another aspect of the invention, there is provided a compound of formula I, wherein R² is -Q-aryl, said aryl, optionally is substituted with R⁷.

In another aspect of the invention, there is provided a compound of formula I, wherein R² is -Q-heteroaryl, said heteroaryl, being optionally substituted by one, two or three R⁷.

In another aspect of the invention, there is provided a compound of formula I, wherein R⁷ is selected from OSO₂R⁸ and C₀₋₆alkylaryl and wherein said C₀₋₆alkylaryl may be optionally substituted by one or more R¹⁴, and wherein any of the individual aryl groups may be optionally fused with a 4, 5, 6 or 7 membered cycloalkyl, a 5, 6 or 7 membered cycloalkenyl or heterocyclyl group to form a bicyclic ring system where the bicyclic ring system is optionally substituted with between one and three A.

In another aspect of the invention, there is provided a compound of formula I, wherein R⁷ is selected from halogen, nitro, C₀₋₆alkylCN, OC₂₋₆alkylOR⁸, trifluoromethyl, fluoromethoxy, trifluoromethoxy, OSO₂R⁸, C₀₋₆alkylaryl and C₀₋₆alkylheteroaryl, wherein any C₀₋₆alkylaryl or C₀₋₆alkylheteroaryl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A. In one embodiment of this aspect, there is provided a compound of formula I, wherein said C₀₋₆alkylaryl represents phenyl.

In another aspect of the invention, there is provided a compound of formula I, wherein said R¹⁴ represents C₀₋₆alkylOR⁸ or OSO₂R⁸.

In another aspect of the invention, there is provided a compound of formula I, wherein said R¹⁴ is selected from halogen, nitro, C₀₋₆alkylCN, C₀₋₆alkylOR⁸, fluoromethyl, difluoromethyl, trifluoromethyl, fluoromethoxy, difluoromethoxy, trifluoromethoxy, C₀₋₆alkylCO₂R⁸, C₀₋₆alkylNR⁸(CO)R⁹, OR⁸, O(CO)R⁸, C₀₋₆alkylCOR⁸, OSO₂R⁸ and OSO₂R⁸R⁹C₁₋₆alkyl.

In another aspect of the invention, there is provided a compound of formula I, wherein said R⁸ represents C₁₋₆alkyl or trifluoromethyl.

In another aspect of the invention, there is provided a compound of formula I, wherein said R⁸ and R⁹ are independently selected from hydrogen, C₁₋₆alkyl, trifluoromethyl, C₀₋₆alkylaryl and C₀₋₆alkylNR¹⁰R¹¹, wherein the C₁₋₆alkyl, or C₀₋₆alkylaryl, C₀₋₆alkylheteroaryl, C₀₋₆alkylheterocyclyl are optionally substituted by A; or

R⁸ and R⁹ may together form a 4 to 6 membered heterocyclic ring containing one or more heteroatoms selected from N, O or S.

In another aspect of the invention, there is provided a compound of formula I, wherein n is 0.

In another aspect of the invention, there is provided a compound of formula I, wherein A is selected from OR¹², C₁₋₆alkyl and COR¹²; and R¹² is C₁₋₆alkyl.

In another aspect of the invention, there is provided a compound of formula I, wherein R⁶ is aryl, wherein said aryl is optionally substituted with between one and four R⁷, and wherein said aryl may be optionally fused with a 4, 5, 6 or 7 membered cycloalkyl, a 5, 6 or 7 membered cycloalkenyl or heterocyclyl group to form a bicyclic ring system where the bicyclic ring system is optionally substituted with between one and three A.

In another aspect of the invention, there is provided a compound of formula I, wherein R⁷ is selected from OSO₂R⁸ and C₀₋₆alkylaryl and wherein said C₀₋₆alkylaryl may be optionally substituted by one or more R¹⁴.

In another aspect of the invention, there is provided a compound of formula I, wherein said R¹⁴ represents C₀₋₆alkylOR⁸ or OSO₂R⁸.

In another aspect of the invention, there is provided a compound of formula I, wherein said aryl may be optionally fused with a 5 or 6 membered heterocyclyl group to form a bicyclic ring system where the bicyclic ring system is optionally substituted with between one and three A.

In another aspect of the invention, there is provided a compound of formula I, wherein said A is COR¹² and R¹² is C₁₋₆alkyl.

In another aspect of the invention, there is provided a compound of formula I, wherein R⁶ is selected from aryl or heteroaryl wherein each of the said aryl or heteroaryl is optionally substituted with between one and four R⁷, and wherein any of the individual aryl or heteroaryl groups may be optionally fused with a 4, 5, 6 or 7 membered cycloalkyl or heterocyclyl group to form a bicyclic ring system where the bicyclic ring system is optionally substituted with between one and three A.

In another aspect of the invention, there is provided a compound of formula I, wherein R⁷ is selected from halogen, nitro, C₀₋₆alkylCN, OC₂₋₆alkylOR⁸, trifluoromethyl, fluoromethoxy, trifluoromethoxy, OSO₂R⁸, C₀₋₆alkylaryl and C₀₋₆alkylheteroaryl, wherein any C₀₋₆alkylaryl or C₀₋₆alkylheteroaryl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A.

In another aspect of the invention, there is provided a compound of formula I, wherein R¹⁴ is selected from halogen, nitro, C₀₋₆alkylCN, C₀₋₆alkylOR⁸, fluoromethyl, difluoromethyl, trifluoromethyl, fluoromethoxy, difluoromethoxy, trifluoromethoxy, C₀₋₆alkylCO₂R⁸, C₀₋₆alkylNR⁸ (CO)R⁹, OR⁸, C₀₋₆alkylCOR⁸, OSO₂R⁸ and OSO₂R⁸R⁹C₁₋₆alkyl.

In another aspect of the invention, there is provided a compound of formula I, wherein R⁸ and R⁹ are independently selected from hydrogen, C₁₋₆alkyl, trifluoromethyl, C₀₋₆alkylaryl, C₀₋₆alkylNR¹⁰R¹¹, wherein the C₁₋₆alkyl or C₀₋₆alkylaryl, are optionally substituted by A;

or
R⁸ and R⁹ may together form a 4 to 6 membered heterocyclic ring containing one or more heteroatoms selected from N, O or S.

In another aspect of the invention, there is provided a compound of formula I, wherein R¹⁰ and R¹¹ are independently selected from hydrogen or C₁₋₆alkyl; or

R¹⁰ and R¹¹ may together form a 4 to 6 membered heterocyclic ring containing one or more heteroatoms selected from N, O or S.

In another aspect of the invention, there is provided a compound of formula I, wherein A is selected from OR¹², C₁₋₆alkyl, and COR¹².

In another aspect of the invention, there is provided a compound of formula I, wherein R¹² is C₁₋₆alkyl.

Another embodiment of the present invention provides compounds of formula I comprising the following:
2-Amino-5-(3-bromophenyl)-3-methyl-5-phenyl-3,5-dihydro-imidazol-4-one;
5-[3-(2-Acetyl-1,2,3,4-tetrahydroisoquinolin-7-yl)phenyl]-2-amino-3-methyl-5-phenyl-3,5-dihydro-4H-imidazol-4-one;
5-[3-(2-Acetyl-1,2,3,4-tetrahydroisoquinolin-7-yl)phenyl]-2-amino-3-methyl-5-phenyl-3,5-dihydro-4H-imidazol-4-one hydrochloride;
5-[3-(1-Acetyl-2,3-dihydro-1H-indol-5-yl)phenyl]-2-amino-3-methyl-5-phenyl-3,5-dihydro-4H-imidazol-4-one;
5-[3-(1-Acetyl-2,3-dihydro-1H-indol-5-yl)phenyl]-2-amino-3-methyl-5-phenyl-3,5-dihydro-4H-imidazol-4-one hydrochloride;
2-Amino-5-[3-(2,3-dihydro-1-benzofuran-6-yl)phenyl]-3-methyl-5-phenyl-3,5-dihydro-4H-imidazol-4-one;
2-Amino-5-[3-(2,3-dihydro-1-benzofuran-6-yl)phenyl]-3-methyl-5-phenyl-3,5-dihydro-4H-imidazol-4-one hydrochloride;
4-[2-Amino-4-(3′-methoxybiphenyl-3-yl)-1-methyl-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl methanesulfonate hydrochloride;
4-[2-Amino-4-(3′-methoxybiphenyl-3-yl)-1-methyl-5-oxo-4,5-dihydro-1H-imidazol-4-yl]phenyl trifluoromethanesulfonate;
4-[2-Amino-4-(3′-methoxybiphenyl-3-yl)-1-methyl-5-oxo-4,5-dihydro-1H-imidazol-4-yl]phenyl trifluoromethanesulfonate hydrochloride;
2-Amino-5-(3′-hydroxybiphenyl-3-yl)-3-methyl-5-phenyl-3,5-dihydro-4H-imidazol-4-one;
3′-(2-Amino-1-methyl-5-oxo-4-phenyl-4,5-dihydro-1H-imidazol-4-yl)biphenyl-3-yl trifluoromethanesulfonate hydrochloride;
2-Amino-5-(2′-hydroxy-5′-methoxybiphenyl-3-yl)-3-methyl-5-phenyl-3,5-dihydro-4H-imidazol-4-one;
3′-(2-Amino-1-methyl-5-oxo-4-phenyl-4,5-dihydro-1H-imidazol-4-yl)-5-methoxybiphenyl-2-yl trifluoromethanesulfonate hydrochloride;
3-(2-Amino-1-methyl-5-oxo-4-phenyl-4,5-dihydro-1H-imidazol-4-yl)phenyl methanesulfonate hydrochloride;
2-Amino-5-(3-hydroxyphenyl)-3-methyl-5-phenyl-3,5-dihydro-4H-imidazol-4-one;
3-(2-Amino-1-methyl-5-oxo-4-phenyl-4,5-dihydro-1H-imidazol-4-yl)phenyl trifluoromethanesulfonate hydrochloride;
2-Amino-5-(3-bromo-4-hydroxyphenyl)-3-methyl-5-phenyl-3,5-dihydro-4H-imidazol-4-one;
2-Amino-5-(6-hydroxy-3′-methoxybiphenyl-3-yl)-3-methyl-5-phenyl-3,5-dihydro-4H-imidazol-4-one;
5-(2-Amino-1-methyl-5-oxo-4-phenyl-4,5-dihydro-1H-imidazol-4-yl)-3′-methoxybiphenyl-2-yl trifluoromethanesulfonate;
3-[2-Amino-4-(3′-methoxybiphenyl-3-yl)-1-methyl-5-oxo-4,5-dihydro-1H-imidazol-4-yl]phenyl trifluoromethanesulfonate hydrochloride;
3-[2-Amino-4-(3′-methoxybiphenyl-3-yl)-1-methyl-5-oxo-4,5-dihydro-1H-imidazol-4-yl]phenyl methanesulfonate hydrochloride;
2-Amino-5-[3-(2,3-dihydro-1-benzofuran-5-yl)phenyl]-3-methyl-5-phenyl-3,5-dihydro-4H-imidazol-4-one;
2-Amino-5-[3-(2,3-dihydro-1-benzofuran-5-yl)phenyl]-3-methyl-5-phenyl-3,5-dihydro-4H-imidazol-4-one hydrochloride;
2-Amino-5-(2,3-dihydro-1-benzofuran-5-yl)-3-methyl-5-phenyl-3,5-dihydro-4H-imidazol-4-one;
2-Amino-5-(2,3-dihydro-1-benzofuran-5-yl)-3-methyl-5-phenyl-3,5-dihydro-4H-imidazol-4-one hydrochloride;
(R)-4-[2-amino-4-(3′-methoxybiphenyl-3-yl)-1-methyl-5-oxo-4,5-dihydro-1H-imidazol-4-yl]phenyl methanesulfonate hydrochloride;
(S)-4-[2-amino-4-(3′-methoxybiphenyl-3-yl)-1-methyl-5-oxo-4,5-dihydro-1H-imidazol-4-yl]phenyl methanesulfonate hydrochloride;
2-Amino-3-methyl-5-[3-(4-methyl-3,4-dihydro-2H-1,4-benzoxazin-7-yl)phenyl]-5-phenyl-3,5-dihydro-4H-imidazol-4-one;
2-Amino-3-methyl-5-[3-(4-methyl-3,4-dihydro-2H-1,4-benzoxazin-7-yl)phenyl]-5-phenyl-3,5-dihydro-4H-imidazol-4-one hydrochloride;
5-[3-(2-Acetyl-1,2,3,4-tetrahydroisoquinolin-5-yl)phenyl]-2-amino-3-methyl-5-phenyl-3,5-dihydro-4H-imidazol-4-one;
5-[3-(2-Acetyl-1,2,3,4-tetrahydroisoquinolin-5-yl)phenyl]-2-amino-3-methyl-5-phenyl-3,5-dihydro-4H-imidazol-4-one hydrochloride;
(R)-4-[2-amino-4-(3′-methoxybiphenyl-3-yl)-1-methyl-5-oxo-4,5-dihydro-1H-imidazol-4-yl]phenyl trifluoromethanesulfonate hydrochloride;
(S)-4-[2-amino-4-(3′-methoxybiphenyl-3-yl)-1-methyl-5-oxo-4,5-dihydro-1H-imidazol-4-yl]phenyl trifluoromethanesulfonate hydrochloride;
2-Amino-3-methyl-5-(4-methyl-3,4-dihydro-2H-1,4-benzoxazin-7-yl)-5-phenyl-3,5-dihydro-4H-imidazol-4-one acetic acid salt;
2-Amino-3-methyl-5-phenyl-5-(5,6,7,8-tetrahydronaphthalen-2-yl)-3,5-dihydro-4H-imidazol-4-one hydrochloride;
5-(1-Acetyl-2,3-dihydro-1H-indol-5-yl)-2-amino-3-methyl-5-phenyl-3,5-dihydro-4H-imidazol-4-one hydrochloride;
5-[3-(1-Acetyl-2,3-dihydro-1H-indol-4-yl)phenyl]-2-amino-3-methyl-5-phenyl-3,5-dihydro-4H-imidazol-4-one hydrochloride;
2-Amino-5-[3-(3,4-dihydro-2H-chromen-8-yl)phenyl]-3-methyl-5-phenyl-3,5-dihydro-4H-imidazol-4-one acetic acid salt;
3′-(2-Amino-1-methyl-5-oxo-4-pyridin-4-yl-4,5-dihydro-1H-imidazol-4-yl)-5-methoxybiphenyl-3-yl methanesulfonate;
3′-(2-Amino-1-methyl-5-oxo-4-pyridin-2-yl-4,5-dihydro-1H-imidazol-4-yl)-5-methoxybiphenyl-3-yl methanesulfonate;
3′-[2-Amino-1-methyl-5-oxo-4-(1,3-thiazol-5-yl)-4,5-dihydro-1H-imidazol-4-yl]-5-methoxybiphenyl-3-yl methanesulfonate;
3′-[2-Amino-1-methyl-5-oxo-4-(1,3-thiazol-4-yl)-4,5-dihydro-1H-imidazol-4-yl]-5-methoxybiphenyl-3-yl methanesulfonate;
4-[2-Amino-4-(3-bromophenyl)-1-methyl-5-oxo-4,5-dihydro-1H-imidazol-4-yl]phenyl methanesulfonate;
4-[2-Amino-1-methyl-5-oxo-4-(3-pyridin-3-ylphenyl)-4,5-dihydro-1H-imidazol-4-yl]phenyl methanesulfonate 0.25 acetate;
4-[2-Amino-4-(3-bromophenyl)-1-methyl-5-oxo-4,5-dihydro-1H-imidazol-4-yl]phenyl 2-methoxyethanesulfonate;
4-[2-Amino-4-(3-bromophenyl)-1-methyl-5-oxo-4,5-dihydro-1H-imidazol-4-yl]phenyl propane-1-sulfonate;
4-[2-Amino-4-(5′-chloro-2′-methoxybiphenyl-3-yl)-1-methyl-5-oxo-4,5-dihydro-1H-imidazol-4-yl]phenyl methanesulfonate 0.25 acetate;
4-[2-Amino-4-(5′-fluoro-2′-methylbiphenyl-3-yl)-1-methyl-5-oxo-4,5-dihydro-1H-imidazol-4-yl]phenyl methanesulfonate;
Methyl 3′-(2-amino-1-methyl-4-{4-[(methylsulfonyl)oxy]phenyl}-5-oxo-4,5-dihydro-1H-imidazol-4-yl)biphenyl-3-carboxylate;
4-{2-Amino-4-[3-(1,3-benzodioxol-5-yl)phenyl]-1-methyl-5-oxo-4,5-dihydro-1H-imidazol-4-yl}phenyl methanesulfonate;
4-{2-Amino-4-[3-(1H-indol-5-yl)phenyl]-1-methyl-5-oxo-4,5-dihydro-1H-imidazol-4-yl}phenyl methanesulfonate 0.25 acetate;
4-[2-Amino-4-(3′-cyanobiphenyl-3-yl)-1-methyl-5-oxo-4,5-dihydro-1H-imidazol-4-yl]phenyl methanesulfonate;
4-{2-Amino-1-methyl-5-oxo-4-[3′-(trifluoromethoxy)biphenyl-3-yl]-4,5-dihydro-1H-imidazol-4-yl}phenyl methanesulfonate;
4-{2-Amino-4-[3-(2-formyl-3-thienyl)phenyl]-1-methyl-5-oxo-4,5-dihydro-1H-imidazol-4-yl}phenyl methanesulfonate;
4-{2-Amino-4-[3-(5-formyl-2-thienyl)phenyl]-1-methyl-5-oxo-4,5-dihydro-1H-imidazol-4-yl}phenyl methanesulfonate;
4-{2-Amino-4-[3-(2-chloropyridin-4-yl)phenyl]-1-methyl-5-oxo-4,5-dihydro-1H-imidazol-4-yl}phenyl methanesulfonate;
4-{4-[3′-(Acetylamino)biphenyl-3-yl]-2-amino-1-methyl-5-oxo-4,5-dihydro-1H-imidazol-4-yl}phenyl methanesulfonate 0.25 acetate;
4-[2-Amino-1-methyl-4-(3′-nitrobiphenyl-3-yl)-5-oxo-4,5-dihydro-1H-imidazol-4-yl]phenyl propane-1-sulfonate;
4-[2-Amino-4-(3′-cyanobiphenyl-3-yl)-1-methyl-5-oxo-4,5-dihydro-1H-imidazol-4-yl]phenyl propane-1-sulfonate;
4-[2-Amino-4-(2′,5′-dimethoxybiphenyl-3-yl)-1-methyl-5-oxo-4,5-dihydro-1H-imidazol-4-yl]phenyl propane-1-sulfonate;
4-[2-Amino-4-(3′-ethoxybiphenyl-3-yl)-1-methyl-5-oxo-4,5-dihydro-1H-imidazol-4-yl]phenyl propane-1-sulfonate;
4-[2-Amino-4-(2′-fluoro-3′-methoxybiphenyl-3-yl)-1-methyl-5-oxo-4,5-dihydro-1H-imidazol-4-yl]phenyl propane-1-sulfonate;
4-[2-Amino-4-(2′-fluoro-5′-methoxybiphenyl-3-yl)-1-methyl-5-oxo-4,5-dihydro-1H-imidazol-4-yl]phenyl propane-1-sulfonate;
4-[2-Amino-4-(2′,6′-difluoro-3′-methoxybiphenyl-3-yl)-1-methyl-5-oxo-4,5-dihydro-1H-imidazol-4-yl]phenyl propane-1-sulfonate;
4-[2-Amino-4-(3′-cyano-4′-fluorobiphenyl-3-yl)-1-methyl-5-oxo-4,5-dihydro-1H-imidazol-4-yl]phenyl propane-1-sulfonate;
4-[2-Amino-4-(5′-cyano-2′-fluorobiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyridin-3-ylphenyl)-4,5-dihydro-1H-imidazol-4-yl]phenyl propane-1-sulfonate;
4-{2-Amino-4-[3-(2-fluoropyridin-3-yl)phenyl]-1-methyl-5-oxo-4,5-dihydro-1H-imidazol-4-yl}phenyl propane-1-sulfonate;
4-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
4-{2-Amino-4-[3-(6-fluoropyridin-3-yl)phenyl]-1-methyl-5-oxo-4,5-dihydro-1H-imidazol-4-yl}phenyl propane-1-sulfonate;
4-[2-Amino-1-methyl-5-oxo-4-(3-pyrimidin-5-ylphenyl)-4,5-dihydro-1H-imidazol-4-yl]phenyl propane-1-sulfonate;
4-{2-Amino-4-[3-(2-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-3-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-5-fluoropyridin-3-yl)phenyl]-1-methyl-5-oxo-4,5-dihydro-1H-imidazol-4-yl}phenyl propane-1-sulfonate;
4-{2-Amino-4-[3-(2,6-difluoropyridin-3-yl)phenyl]-1-methyl-5-oxo-4,5-dihydro-1H-imidazol-4-yl}phenyl propane-1-sulfonate;
4-[2-Amino-1-methyl-4-(3′-nitrobiphenyl-3-yl)-5-oxo-4,5-dihydro-1H-imidazol-4-yl]phenyl 2-methoxyethanesulfonate;
4-[2-Amino-4-(3′-cyanobiphenyl-3-yl)-1-methyl-5-oxo-4,5-dihydro-1H-imidazol-4-yl]phenyl 2-methoxyethanesulfonate;
4-[2-Amino-4-(2′,5′-dimethoxybiphenyl-3-yl)-1-methyl-5-oxo-4,5-dihydro-1H-imidazol-4-yl]phenyl 2-methoxyethanesulfonate;
4-[2-Amino-4-(3′-ethoxybiphenyl-3-yl)-1-methyl-5-oxo-4,5-dihydro-1H-imidazol-4-yl]phenyl 2-methoxyethanesulfonate;
4-[2-Amino-4-(2′-fluoro-3′-methoxybiphenyl-3-yl)-1-methyl-5-oxo-4,5-dihydro-1H-imidazol-4-yl]phenyl 2-methoxyethanesulfonate;
4-[2-Amino-4-(2′-fluoro-5′-methoxybiphenyl-3-yl)-1-methyl-5-oxo-4,5-dihydro-1H-imidazol-4-yl]phenyl 2-methoxyethanesulfonate;
4-[2-Amino-4-(2′,6′-difluoro-3′-methoxybiphenyl-3-yl)-1-methyl-5-oxo-4,5-dihydro-1H-imidazol-4-yl]phenyl 2-methoxyethanesulfonate;
4-[2-Amino-4-(3′-cyano-4′-fluorobiphenyl-3-yl)-1-methyl-5-oxo-4,5-dihydro-1H-imidazol-4-yl]phenyl 2-methoxyethanesulfonate;
4-[2-Amino-4-(5′-cyano-2′-fluorobiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyridin-3-ylphenyl)-4,5-dihydro-1H-imidazol-4-yl]phenyl 2-methoxyethanesulfonate;
4-{2-Amino-4-[3-(2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(6-fluoropyridin-3-yl)phenyl]-1-methyl-5-oxo-4,5-dihydro-1H-imidazol-4-yl}phenyl 2-methoxyethanesulfonate;
4-[2-Amino-1-methyl-5-oxo-4-(3-pyrimidin-5-ylphenyl)-4,5-dihydro-1H-imidazol-4-yl]phenyl 2-methoxyethanesulfonate;
4-{2-Amino-4-[3-(2-chloro-3-fluoropyridin-4-yl)phenyl]-1-methyl-5-oxo-4,5-dihydro-1H-imidazol-4-yl}phenyl 2-methoxyethanesulfonate;
4-{2-Amino-4-[3-(2-chloro-5-fluoropyridin-3-yl)phenyl]-1-methyl-5-oxo-4,5-dihydro-1H-imidazol-4-yl}phenyl 2-methoxyethanesulfonate;
4-{2-Amino-4-[3-(2,6-difluoropyridin-3-yl)phenyl]-1-methyl-5-oxo-4,5-dihydro-1H-imidazol-4-yl}phenyl 2-methoxyethanesulfonate;
5-(4-Hydroxyphenyl)-3-methyl-5-(3-phenoxyphenyl)-2-thioxoimidazolidin-4-one;
4-(2-Amino-4-{3′-methoxy-5′-[(methylsulfonyl)oxy]biphenyl-3-yl}-1-methyl-5-oxo-4,5-dihydro-1H-imidazol-4-yl)phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl propane-1-sulfonate;
4-[2-Amino-4-(4′-fluoro-3′-methoxybiphenyl-3-yl)-1-methyl-5-oxo-4,5-dihydro-1H-imidazol-4-yl]phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl 2-methoxyethanesulfonate;
4-[2-Amino-4-(4′-fluoro-3′-methoxybiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyrazin-2-ylphenyl)-4,5-dihydro-1H-imidazol-4-yl]phenyl 2-methoxyethanesulfonate 0.25 acetate;
4-[2-Amino-1′-methyl-5-oxo-4-(3-pyrazin-2-ylphenyl)-4,5-dihydro-1H-imidazol-4-yl]phenyl propane-1-sulfonate 0.25 acetate;
(R)-4-[2-Amino-1′-methyl-5-oxo-4-(3-pyridin-3-ylphenyl)-4,5-dihydro-1H-imidazol-4-yl]phenyl methanesulfonate;
(S)-4-[2-Amino-1′-methyl-5-oxo-4-(3-pyridin-3-ylphenyl)-4,5-dihydro-1H-imidazol-4-yl]phenyl methanesulfonate;
3′-(2-Amino-1-methyl-5-oxo-4-phenyl-4,5-dihydro-1H-imidazol-4-yl)-5-methoxybiphenyl-3-yl methanesulfonate hydrochloride;
3-[2-Amino-4-(3-bromophenyl)-1′-methyl-5-oxo-4,5-dihydro-1H-imidazol-4-yl]phenyl methanesulfonate;
3-[2-Amino-4-(3-bromophenyl)-1′-methyl-5-oxo-4,5-dihydro-1H-imidazol-4-yl]phenyl 2-methoxyethanesulfonate;
3-[4-(3-Bromophenyl)-1′-methyl-5-oxo-2-thioxoimidazolidin-4-yl]phenyl propane-1-sulfonate;
3-[2-Amino-4-(3-bromophenyl)-1′-methyl-5-oxo-4,5-dihydro-1H-imidazol-4-yl]phenyl propane-1-sulfonate;
3-[4-(3-Bromophenyl)-1′-methyl-5-oxo-2-thioxoimidazolidin-4-yl]phenyl propane-2-sulfonate;
3-[2-Amino-4-(3-bromophenyl)-1′-methyl-5-oxo-4,5-dihydro-1H-imidazol-4-yl]phenyl propane-2-sulfonate;
3-[4-(3-Bromophenyl)-1′-methyl-5-oxo-2-thioxoimidazolidin-4-yl]phenyl cyclopropanesulfonate;
3-[2-Amino-4-(3-bromophenyl)-1′-methyl-5-oxo-4,5-dihydro-1H-imidazol-4-yl]phenyl cyclopropanesulfonate;
3-[4-(3-Bromophenyl)-1′-methyl-5-oxo-2-thioxoimidazolidin-4-yl]phenyl dimethylsulfamate;
3-[2-Amino-4-(3-bromophenyl)-1′-methyl-5-oxo-4,5-dihydro-1H-imidazol-4-yl]phenyl dimethylsulfamate;
3-[4-(3-Bromophenyl)-1′-methyl-5-oxo-2-thioxoimidazolidin-4-yl]phenyl morpholine-4-sulfonate;
3-[2-Amino-4-(3-bromophenyl)-1′-methyl-5-oxo-4,5-dihydro-1H-imidazol-4-yl]phenyl morpholine-4-sulfonate;
3-[4-(3-Bromophenyl)-1-methyl-5-oxo-2-thioxoimidazolidin-4-yl]phenyl ethanesulfonate;
3-[2-Amino-4-(3-bromophenyl)-1-methyl-5-oxo-4,5-dihydro-1H-imidazol-4-yl]phenyl ethanesulfonate;
2-Amino-5-(3-bromophenyl)-5-(3-hydroxyphenyl)-3-methyl-3,5-dihydro-4H-imidazol-4-one;
3-{2-Amino-4-[3-(5-methoxypyridin-3-yl)phenyl]-1-methyl-5-oxo-4,5-dihydro-1H-imidazol-4-yl}phenyl ethanesulfonate 0.75 acetate;
4-{2-Amino-4-[3-(5-methoxypyridin-3-yl)phenyl]-1-methyl-5-oxo-4,5-dihydro-1H-imidazol-4-yl}phenyl methanesulfonate acetic acid;
2-Amino-5-[3-(2-fluoropyridin-3-yl)phenyl]-5-(3-hydroxyphenyl)-3-methyl-3,5-dihydro-4H-imidazol-4-one;
2-Amino-5-(3-hydroxyphenyl)-3-methyl-5-(3-pyrimidin-5-ylphenyl)-3,5-dihydro-4H-imidazol-4-one;
2-Amino-5-(3-hydroxyphenyl)-3-methyl-5-(3-pyridin-3-ylphenyl)-3,5-dihydro-4H-imidazol-4-one;
2-Amino-5-(3-hydroxyphenyl)-5-[3-(5-methoxypyridin-3-yl)phenyl]-3-methyl-3,5-dihydro-4H-imidazol-4-one;
3-{2-Amino-4-[3-(2-fluoropyridin-3-yl)phenyl]-1-methyl-5-oxo-4,5-dihydro-1H-imidazol-4-yl}phenyl trifluoromethanesulfonate hydrochloride;
3-[2-Amino-1-methyl-5-oxo-4-(3-pyrimidin-5-ylphenyl)-4,5-dihydro-1H-imidazol-4-yl]phenyl trifluoromethanesulfonate hydrochloride;
3-[2-Amino-1-methyl-5-oxo-4-(3-pyridin-3-ylphenyl)-4,5-dihydro-1H-imidazol-4-yl]phenyl trifluoromethanesulfonate hydrochloride;
3-{2-Amino-4-[3-(5-methoxypyridin-3-yl)phenyl]-1-methyl-5-oxo-4,5-dihydro-1H-imidazol-4-yl}phenyl trifluoromethanesulfonate hydrochloride;
(R)-3′-(2-amino-1-methyl-5-oxo-4-phenyl-4,5-dihydro-1H-imidazol-4-yl)-5-methoxybiphenyl-3-yl methanesulfonate hydrochloride;
(S)-3′-(2-amino-1-methyl-5-oxo-4-phenyl-4,5-dihydro-1H-imidazol-4-yl)-5-methoxybiphenyl-3-yl methanesulfonate hydrochloride;
4-[2-Amino-4-(3′,5′-dichlorobiphenyl-3-yl)-1-methyl-5-oxo-4,5-dihydro-1H-imidazol-4-yl]phenyl methanesulfonate hydrochloride;
3-[2-Amino-1-methyl-5-oxo-4-(3-pyridin-3-ylphenyl)-4,5-dihydro-1H-imidazol-4-yl]phenyl methanesulfonate;
3-{2-Amino-4-[3-(6-fluoropyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl methanesulfonate;
3-{2-Amino-4-[3-(5-chloro-2-fluoropyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-5-ylphenyl)-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl propane-1-sulfonate;
3-{2-Amino-4-[3-(6-fluoropyridin-3-yl)phenyl]-1-methyl-5-oxo-4,5-dihydro-1H-imidazol-4-yl}phenyl propane-1-sulfonate;
3-{2-Amino-4-[3-(2-fluoropyridin-3-yl)phenyl]-1-methyl-5-oxo-4,5-dihydro-1H-imidazol-4-yl}phenyl propane-1-sulfonate;
3-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-5-ylphenyl)-4,5-dihydro-1H-imidazol-4-yl]phenyl propane-1-sulfonate;
3-[2-Amino-1-methyl-5-oxo-4-(3-pyridin-3-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl propane-2-sulfonate;
3-{2-Amino-4-[3-(2-fluoropyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-5-ylphenyl)-4,5-dihydro-1H-imidazol-4-yl]phenyl propane-2-sulfonate;
3-[2-Amino-1-methyl-5-oxo-4-(3-pyridin-3-ylphenyl)-4,5-dihydro-1H-imidazol-4-yl]phenyl 2-methoxyethanesulfonate;
3-{2-Amino-4-[3-(6-fluoropyridin-3-yl)phenyl]-1-methyl-5-oxo-4,5-dihydro-1H-imidazol-4-yl}phenyl 2-methoxyethanesulfonate;
3-{2-Amino-4-[3-(2-fluoropyridin-3-yl)phenyl]-1-methyl-5-oxo-4,5-dihydro-1H-imidazol-4-yl}phenyl 2-methoxyethanesulfonate;
3-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate acetate;
3-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
3-[2-Amino-1-methyl-5-oxo-4-(3-pyridin-3-ylphenyl)-4,5-dihydro-1H-imidazol-4-yl]phenyl dimethylsulfamate;
3-{2-Amino-4-[3-(6-fluoropyridin-3-yl)phenyl]-1-methyl-5-oxo-4,5-dihydro-1H-imidazol-4-yl}phenyl dimethylsulfamate;
3-{2-Amino-4-[3-(5-chloro-2-fluoropyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-5-ylphenyl)-4,5-dihydro-1H-imidazol-4-yl]phenyl dimethylsulfamate;
3-[2-Amino-1-methyl-5-oxo-4-(3-pyridin-3-ylphenyl)-4,5-dihydro-1H-imidazol-4-yl]phenyl cyclopropanesulfonate;
3-{2-Amino-4-[3-(6-fluoropyridin-3-yl)phenyl]-1-methyl-5-oxo-4,5-dihydro-1H-imidazol-4-yl}phenyl cyclopropanesulfonate;
-{2-Amino-4-[3-(2-fluoropyridin-3-yl)phenyl]-1-methyl-5-oxo-4,5-dihydro-1H-imidazol-4-yl}phenyl cyclopropanesulfonate;
3-{2-Amino-4-[3-(5-chloro-2-fluoropyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-5-ylphenyl)-4,5-dihydro-1H-imidazol-4-yl]phenyl 2-methoxyethanesulfonate acetate;
3-[2-Amino-1-methyl-5-oxo-4-(3-pyridin-3-ylphenyl)-4,5-dihydro-1H-imidazol-4-yl]phenyl morpholine-4-sulfonate;
3-{2-Amino-4-[3-(6-fluoropyridin-3-yl)phenyl]-1-methyl-5-oxo-4,5-dihydro-1H-imidazol-4-yl}phenyl morpholine-4-sulfonate;
3-{2-Amino-4-[3-(2-fluoropyridin-3-yl)phenyl]-1-methyl-5-oxo-4,5-dihydro-1H-imidazol-4-yl}phenyl morpholine-4-sulfonate;
3-[2-Amino-1-methyl-5-oxo-4-(3-pyrimidin-5-ylphenyl)-4,5-dihydro-1H-imidazol-4-yl]phenyl morpholine-4-sulfonate;
3-{2-Amino-4-[3-(5-methoxypyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-2-ylphenyl)-4,5-dihydro-1H-imidazol-4-yl]phenyl methanesulfonate;
3-{2-Amino-4-[3-(5-methoxypyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-2-ylphenyl)-4,5-dihydro-1H-imidazol-4-yl]phenyl propane-1-sulfonate;
3-{2-Amino-4-[3-(5-methoxypyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-2-ylphenyl)-4,5-dihydro-1H-imidazol-4-yl]phenyl propane-2-sulfonate;
3-{2-Amino-4-[3-(5-methoxypyridin-3-yl)phenyl]-1-methyl-5-oxo-4,5-dihydro-1H-imidazol-4-yl}phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-2-ylphenyl)-4,5-dihydro-1H-imidazol-4-yl]phenyl dimethylsulfamate;
3-{2-Amino-4-[3-(5-methoxypyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-2-ylphenyl)-4,5-dihydro-1H-imidazol-4-yl]phenyl cyclopropanesulfonate;
3-[2-Amino-4-(4-methoxyphenyl)-1-methyl-5-oxo-4,5-dihydro-1H-imidazol-4-yl]phenyl 3-methoxypropane-1-sulfonate hydrochloride;
3-[2-Amino-4-(4-methoxyphenyl)-1-methyl-5-oxo-4,5-dihydro-1H-imidazol-4-yl]phenyl methanesulfonate hydrochloride;
3-[2-Amino-4-(4-methoxyphenyl)-1-methyl-5-oxo-4,5-dihydro-1H-imidazol-4-yl]phenyl 2-ethoxyethanesulfonate hydrochloride;
3-[2-Amino-4-(4-methoxyphenyl)-1-methyl-5-oxo-4,5-dihydro-1H-imidazol-4-yl]phenyl 2-methoxyethanesulfonate hydrochloride;
4-[2-Amino-1-methyl-5-oxo-4-(3-phenoxyphenyl)-4,5-dihydro-1H-imidazol-4-yl]phenyl methanesulfonate hydrochloride;
4-[2-Amino-1-methyl-5-oxo-4-(3-phenoxyphenyl)-4,5-dihydro-1H-imidazol-4-yl]phenyl propane-1-sulfonate hydrochloride;
4-{2-Amino-4-[3-(3-methoxyphenoxy)phenyl]-1-methyl-5-oxo-4,5-dihydro-1H-imidazol-4-yl}phenyl methanesulfonate hydrochloride;
3-[2-Amino-4-(3′-methoxybiphenyl-3-yl)-1-methyl-5-oxo-4,5-dihydro-1H-imidazol-4-yl]phenyl propane-2-sulfonate hydrochloride;
2-Amino-5-(3′-hydroxy-5′-methoxybiphenyl-3-yl)-3-methyl-5-phenyl-3,5-dihydro-4H-imidazol-4-one;
3′-(2-Amino-1-methyl-5-oxo-4-phenyl-4,5-dihydro-1H-imidazol-4-yl)-5-methoxybiphenyl-3-yl trifluoromethanesulfonate hydrochloride;
4-{2-Amino-4-[3-(5-methoxypyridin-3-yl)phenyl]-1-methyl-5-oxo-4,5-dihydro-1H-imidazol-4-yl}phenyl propane-1-sulfonate hydrochloride;
2-Amino-5-(3-bromophenyl)-5-(6-methoxy-5,6,7,8-tetrahydronaphthalen-2-yl)-3-methyl-3,5-dihydro-4H-imidazol-4-one;
5-(1-Acetyl-1,2,3,4-tetrahydroquinolin-6-yl)-2-amino-5-(3-bromophenyl)-3-methyl-3,5-dihydro-4H-imidazol-4-one;
2-Amino-5-(3-bromophenyl)-3-methyl-5-(5,6,7,8-tetrahydronaphthalen-2-yl)imidazolidin-4-one;
2-Amino-5-(3-bromophenyl)-5-(2,3-dihydro-1H-inden-5-yl)-3-methylimidazolidin-4-one;
2-Amino-5-(3′-methoxybiphenyl-3-yl)-5-(6-methoxy-5,6,7,8-tetrahydronaphthalen-2-yl)-3-methyl-3,5-dihydro-4H-imidazol-4-one 0.25 acetate;
2-Amino-5-(2′-fluoro-5′-methoxybiphenyl-3-yl)-5-(6-methoxy-5,6,7,8-tetrahydronaphthalen-2-yl)-3-methyl-3,5-dihydro-4H-imidazol-4-one;
5-(1-Acetyl-1,2,3,4-tetrahydroquinolin-6-yl)-2-amino-5-(3′-methoxybiphenyl-3-yl)-3-methyl-3,5-dihydro-4H-imidazol-4-one;
5-(1-Acetyl-1,2,3,4-tetrahydroquinolin-6-yl)-2-amino-5-(2′,5′-dimethoxybiphenyl-3-yl)-3-methyl-3,5-dihydro-4H-imidazol-4-one 0.25 acetate;
3′-[4-(1-Acetyl-1,2,3,4-tetrahydroquinolin-6-yl)-2-amino-1-methyl-5-oxo-4,5-dihydro-1H-imidazol-4-yl]biphenyl-3-carbonitrile;
3′-[4-(1-Acetyl-1,2,3,4-tetrahydroquinolin-6-yl)-2-amino-1-methyl-5-oxo-4,5-dihydro-1H-imidazol-4-yl]-4-fluorobiphenyl-3-carbonitrile;
5-(1-Acetyl-1,2,3,4-tetrahydroquinolin-6-yl)-2-amino-5-(2′-fluoro-5′-methoxybiphenyl-3-yl)-3-methyl-3,5-dihydro-4H-imidazol-4-one 0.25 acetate;
5-(1-Acetyl-1,2,3,4-tetrahydroquinolin-6-yl)-2-amino-3-methyl-5-(3′-nitrobiphenyl-3-yl)-3,5-dihydro-4H-imidazol-4-one 0.25 acetate;
5-(1-Acetyl-1,2,3,4-tetrahydroquinolin-6-yl)-2-amino-5-(2′-fluoro-3′-methoxybiphenyl-3-yl)-3-methyl-3,5-dihydro-4H-imidazol-4-one;
5-(1-Acetyl-1,2,3,4-tetrahydroquinolin-6-yl)-2-amino-5-[3-(3-furyl)phenyl]-3-methyl-3,5-dihydro-4H-imidazol-4-one;
2-Amino-5-(3′-methoxybiphenyl-3-yl)-3-methyl-5-(5,6,7,8-tetrahydronaphthalen-2-yl)-3,5-dihydro-4H-imidazol-4-one;
3′-[2-Amino-1-methyl-5-oxo-4-(5,6,7,8-tetrahydronaphthalen-2-yl)-4,5-dihydro-1H-imidazol-4-yl]-6-fluorobiphenyl-3-carbonitrile;
2-Amino-5-(2′,5′-dimethoxybiphenyl-3-yl)-3-methyl-5-(5,6,7,8-tetrahydronaphthalen-2-yl)-3,5-dihydro-4H-imidazol-4-one;
2-Amino-5-[3-(1,3-benzodioxol-5-yl)phenyl]-3-methyl-5-(5,6,7,8-tetrahydronaphthalen-2-yl)-3,5-dihydro-4H-imidazol-4-one 0.25 acetate;
2-Amino-5-(3′-ethoxybiphenyl-3-yl)-3-methyl-5-(5,6,7,8-tetrahydronaphthalen-2-yl)-3,5-dihydro-4H-imidazol-4-one;
3′-[2-Amino-1-methyl-5-oxo-4-(5,6,7,8-tetrahydronaphthalen-2-yl)-4,5-dihydro-1H-imidazol-4-yl]biphenyl-3-carbonitrile;
3′-[2-Amino-1-methyl-5-oxo-4-(5,6,7,8-tetrahydronaphthalen-2-yl)-4,5-dihydro-1H-imidazol-4-yl]-4-fluorobiphenyl-3-carbonitrile;
2-Amino-5-(2′-fluoro-5′-methoxybiphenyl-3-yl)-3-methyl-5-(5,6,7,8-tetrahydronaphthalen-2-yl)-3,5-dihydro-4H-imidazol-4-one;
2-Amino-3-methyl-5-(3′-nitrobiphenyl-3-yl)-5-(5,6,7,8-tetrahydronaphthalen-2-yl)-3,5-dihydro-4H-imidazol-4-one;
2-Amino-5-(2′-fluoro-3′-methoxybiphenyl-3-yl)-3-methyl-5-(5,6,7,8-tetrahydronaphthalen-2-yl)-3,5-dihydro-4H-imidazol-4-one;
2-Amino-5-[3-(3-furyl)phenyl]-3-methyl-5-(5,6,7,8-tetrahydronaphthalen-2-yl)-3,5-dihydro-4H-imidazol-4-one;
2-Amino-5-(2,3-dihydro-1H-inden-5-yl)-5-(3′-methoxybiphenyl-3-yl)-3-methyl-3,5-dihydro-4H-imidazol-4-one 0.25 acetate;
3′-[2-Amino-4-(6-methoxy-5,6,7,8-tetrahydronaphthalen-2-yl)-1-methyl-5-oxo-4,5-dihydro-1H-imidazol-4-yl]-5-methoxybiphenyl-3-yl methanesulfonate;
3′-[4-(1-Acetyl-1,2,3,4-tetrahydroquinolin-6-yl)-2-amino-1-methyl-5-oxo-4,5-dihydro-1H-imidazol-4-yl]-5-methoxybiphenyl-3-yl methanesulfonate;
3′-[2-Amino-1-methyl-5-oxo-4-(5,6,7,8-tetrahydronaphthalen-2-yl)-4,5-dihydro-1H-imidazol-4-yl]-5-methoxybiphenyl-3-yl methanesulfonate;
3′-[2-Amino-4-(2,3-dihydro-1H-inden-5-yl)-1-methyl-5-oxo-4,5-dihydro-1H-imidazol-4-yl]-5-methoxybiphenyl-3-yl methanesulfonate;
2-Amino-5-(3-bromophenyl)-5-(2,3-dihydro-1-benzofuran-5-yl)-3-methyl-3,5-dihydro-4H-imidazol-4-one;
2-Amino-5-(3-bromophenyl)-5-(3,4-dihydro-2H-chromen-6-yl)-3-methyl-3,5-dihydro-4H-imidazol-4-one;
2-Amino-5-(2,3-dihydro-1-benzofuran-5-yl)-5-(3′-methoxybiphenyl-3-yl)-3-methyl-3,5-dihydro-4H-imidazol-4-one hydrochloride;
5-[3-(2-Acetyl-2,3-dihydro-1H-isoindol-4-yl)phenyl]-2-amino-3-methyl-5-phenyl-3,5-dihydro-4H-imidazol-4-one acetate;
2-Amino-5-(3-bromophenyl)-5-(3,4-dihydro-1H-isochromen-7-yl)-3-methyl-3,5-dihydro-4H-imidazol-4-one;
2-Amino-5-(2,3-dihydro-1-benzofuran-5-yl)-5-(3′-ethoxybiphenyl-3-yl)-3-methyl-3,5-dihydro-4H-imidazol-4-one 0.25 acetate;
2-Amino-5-(2,3-dihydro-1-benzofuran-5-yl)-5-(2′,5′-dimethoxybiphenyl-3-yl)-3-methyl-3,5-dihydro-4H-imidazol-4-one 0.25 acetate;
2-Amino-5-[3-(1,3-benzodioxol-5-yl)phenyl]-5-(2,3-dihydro-1-benzofuran-5-yl)-3-methyl-3,5-dihydro-4H-imidazol-4-one 0.25 acetate;
2-Amino-5-(2,3-dihydro-1-benzofuran-5-yl)-5-(2′-fluoro-5′-methoxybiphenyl-3-yl)-3-methyl-3,5-dihydro-4H-imidazol-4-one 0.25 acetate;
2-Amino-5-(2,3-dihydro-1-benzofuran-5-yl)-5-(2′-fluoro-3′-methoxybiphenyl-3-yl)-3-methyl-3,5-dihydro-4H-imidazol-4-one 0.25 acetate;
3′-[2-Amino-4-(2,3-dihydro-1-benzofuran-5-yl)-1-methyl-5-oxo-4,5-dihydro-1H-imidazol-4-yl]-6-fluorobiphenyl-3-carbonitrile 0.25 acetate;
2-Amino-5-(2,3-dihydro-1-benzofuran-5-yl)-3-methyl-5-(3′-nitrobiphenyl-3-yl)-3,5-dihydro-4H-imidazol-4-one 0.25 acetate;
2-Amino-5-(2,3-dihydro-1-benzofuran-5-yl)-5-[3-(3-furyl)phenyl]-3-methyl-3,5-dihydro-4H-imidazol-4-one 0.25 acetate;
2-Amino-5-[3-(1-benzofuran-2-yl)phenyl]-5-(2,3-dihydro-1-benzofuran-5-yl)-3-methyl-3,5-dihydro-4H-imidazol-4-one 0.25 acetate;
2-Amino-5-(2,3-dihydro-1-benzofuran-5-yl)-3-methyl-5-[3′-(trifluoromethoxy)biphenyl-3-yl]-3,5-dihydro-4H-imidazol-4-one 0.25 acetate;
2-Amino-5-(3′-chlorobiphenyl-3-yl)-5-(2,3-dihydro-1-benzofuran-5-yl)-3-methyl-3,5-dihydro-4H-imidazol-4-one x 0.25 acetate;
2-Amino-5-(3,4-dihydro-2H-chromen-6-yl)-5-(3′-methoxybiphenyl-3-yl)-3-methyl-3,5-dihydro-4H-imidazol-4-one 0.25 acetate;
2-Amino-5-(3,4-dihydro-2H-chromen-6-yl)-5-(3′-ethoxybiphenyl-3-yl)-3-methyl-3,5-dihydro-4H-imidazol-4-one;
2-Amino-5-(3,4-dihydro-2H-chromen-6-yl)-5-(2′,5′-dimethoxybiphenyl-3-yl)-3-methyl-3,5-dihydro-4H-imidazol-4-one 0.25 acetate;
2-Amino-5-[3-(1,3-benzodioxol-5-yl)phenyl]-5-(3,4-dihydro-2H-chromen-6-yl)-3-methyl-3,5-dihydro-4H-imidazol-4-one 0.25 acetate;
2-Amino-5-(3,4-dihydro-2H-chromen-6-yl)-5-(2′-fluoro-5′-methoxybiphenyl-3-yl)-3-methyl-3,5-dihydro-4H-imidazol-4-one;
2-Amino-5-(3,4-dihydro-2H-chromen-6-yl)-5-(2′-fluoro-3′-methoxybiphenyl-3-yl)-3-methyl-3,5-dihydro-4H-imidazol-4-one;
3′-[2-Amino-4-(3,4-dihydro-2H-chromen-6-yl)-1-methyl-5-oxo-4,5-dihydro-1H-imidazol-4-yl]-4-fluorobiphenyl-3-carbonitrile;
3′-[2-Amino-4-(3,4-dihydro-2H-chromen-6-yl)-1-methyl-5-oxo-4,5-dihydro-1H-imidazol-4-yl]-6-fluorobiphenyl-3-carbonitrile 0.25 acetate;
2-Amino-5-(3,4-dihydro-2H-chromen-6-yl)-3-methyl-5-(3′-nitrobiphenyl-3-yl)-3,5-dihydro-4H-imidazol-4-one 0.25 acetate;
2-Amino-5-(3,4-dihydro-2H-chromen-6-yl)-5-[3-(3-furyl)phenyl]-3-methyl-3,5-dihydro-4H-imidazol-4-one 0.25 acetate;
2-Amino-5-[3-(1-benzofuran-2-yl)phenyl]-5-(3,4-dihydro-2H-chromen-6-yl)-3-methyl-3,5-dihydro-4H-imidazol-4-one;
2-Amino-5-(3,4-dihydro-2H-chromen-6-yl)-3-methyl-5-[3′-(trifluoromethoxy)biphenyl-3-yl]-3,5-dihydro-4H-imidazol-4-one 0.25 acetate;
2-Amino-5-(3′-chlorobiphenyl-3-yl)-5-(3,4-dihydro-2H-chromen-6-yl)-3-methyl-3,5-dihydro-4H-imidazol-4-one;
2-Amino-5-(3,4-dihydro-1H-isochromen-7-yl)-5-(2′-fluoro-5′-methoxybiphenyl-3-yl)-3-methyl-3,5-dihydro-4H-imidazol-4-one;
3′-[2-Amino-4-(3,4-dihydro-1H-isochromen-7-yl)-1-methyl-5-oxo-4,5-dihydro-1H-imidazol-4-yl]-6-fluorobiphenyl-3-carbonitrile 0.25 acetate;
2-Amino-5-(3,4-dihydro-1H-isochromen-7-yl)-5-[3-(3-furyl)phenyl]-3-methyl-3,5-dihydro-4H-imidazol-4-one 0.25 acetate;
3′-[2-Amino-4-(2,3-dihydro-1-benzofuran-5-yl)-1-methyl-5-oxo-4,5-dihydro-1H-imidazol-4-yl]-5-methoxybiphenyl-3-yl methanesulfonate 0.25 acetate;
3′-[2-Amino-4-(3,4-dihydro-2H-chromen-6-yl)-1-methyl-5-oxo-4,5-dihydro-1H-imidazol-4-yl]-5-methoxybiphenyl-3-yl methanesulfonate 0.25 acetate;
3′-[2-Amino-4-(3,4-dihydro-1H-isochromen-7-yl)-1-methyl-5-oxo-4,5-dihydro-1H-imidazol-4-yl]-5-methoxybiphenyl-3-yl methanesulfonate;
4-[2-Amino-4-(3-bromo-4-fluorophenyl)-1-methyl-5-oxo-4,5-dihydro-1H-imidazol-4-yl]phenyl methanesulfonate;
4-[2-Amino-4-(3′-cyano-6-fluorobiphenyl-3-yl)-1-methyl-5-oxo-4,5-dihydro-1H-imidazol-4-yl]phenyl methanesulfonate;
4-[2-Amino-4-(3′-cyano-6-fluorobiphenyl-3-yl)-5-oxo-4,5-dihydro-1H-imidazol-4-yl]phenyl methanesulfonate;
4-[2-Amino-4-(2′,6-difluoro-3′-methoxybiphenyl-3-yl)-5-oxo-4,5-dihydro-1H-imidazol-4-yl]phenyl methanesulfonate;
4-[2-Amino-4-(2′,6-difluoro-5′-methoxybiphenyl-3-yl)-5-oxo-4,5-dihydro-1H-imidazol-4-yl]phenyl methanesulfonate;
4-[2-Amino-4-(3′-cyano-4′,6-difluorobiphenyl-3-yl)-5-oxo-4,5-dihydro-1H-imidazol-4-yl]phenyl methanesulfonate;
4-[2-Amino-4-(5′-cyano-2′,6-difluorobiphenyl-3-yl)-5-oxo-4,5-dihydro-1H-imidazol-4-yl]phenyl methanesulfonate;
4-[2-Amino-4-(4-fluoro-3-pyridin-3-ylphenyl)-5-oxo-4,5-dihydro-1H-imidazol-4-yl]phenyl methanesulfonate;
4-{2-Amino-4-[4-fluoro-3-(2-fluoropyridin-3-yl)phenyl]-5-oxo-4,5-dihydro-1H-imidazol-4-yl}phenyl methanesulfonate;
4-{2-Amino-4-[3-(5-chloro-2-fluoropyridin-3-yl)-4-fluorophenyl]-5-oxo-4,5-dihydro-1H-imidazol-4-yl}phenyl methanesulfonate;
4-{2-Amino-4-[4-fluoro-3-(6-fluoropyridin-3-yl)phenyl]-5-oxo-4,5-dihydro-1H-imidazol-4-yl}phenyl methanesulfonate;
4-{2-Amino-4-[4-fluoro-3-(2-fluoropyridin-4-yl)phenyl]-5-oxo-4,5-dihydro-1H-imidazol-4-yl}phenyl methanesulfonate;
4-{2-Amino-4-[3-(2-chloro-3-fluoropyridin-4-yl)-4-fluorophenyl]-5-oxo-4,5-dihydro-1H-imidazol-4-yl}phenyl methanesulfonate;
4-{2-Amino-4-[3-(2-chloro-5-fluoropyridin-3-yl)-4-fluorophenyl]-5-oxo-4,5-dihydro-1H-imidazol-4-yl}phenyl methanesulfonate;
4-{2-Amino-4-[3-(2,6-difluoropyridin-3-yl)-4-fluorophenyl]-5-oxo-4,5-dihydro-1H-imidazol-4-yl}phenyl methanesulfonate;
4-{2-Amino-4-[6-fluoro-3′-(trifluoromethoxy)biphenyl-3-yl]-5-oxo-4,5-dihydro-1H-imidazol-4-yl}phenyl methanesulfonate;
4-[2-Amino-4-(3′-chloro-6-fluorobiphenyl-3-yl)-5-oxo-4,5-dihydro-1H-imidazol-4-yl]phenyl methanesulfonate;
4-{2-Amino-4-[3-(1,3-benzodioxol-5-yl)-4-fluorophenyl]-5-oxo-4,5-dihydro-1H-imidazol-4-yl}phenyl methanesulfonate;
4-[2-Amino-4-(4-fluoro-3-pyridin-4-ylphenyl)-5-oxo-4,5-dihydro-1H-imidazol-4-yl]phenyl methanesulfonate;
4-[2-Amino-4-(4-fluoro-3-pyrimidin-5-ylphenyl)-5-oxo-4,5-dihydro-1H-imidazol-4-yl]phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-(2-Amino-4-{6-fluoro-3′-methoxy-5′-[(methylsulfonyl)oxy]biphenyl-3-yl}-5-oxo-4,5-dihydro-1H-imidazol-4-yl)phenyl methanesulfonate;
4-{2-Amino-4-[4-fluoro-3-(5-fluoropyridin-3-yl)phenyl]-5-oxo-4,5-dihydro-1H-imidazol-4-yl}phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
2-Amino-5-(4-hydroxyphenyl)-5-(3′-methoxybiphenyl-3-yl)-3-methyl-3,5-dihydro-4H-imidazol-4-one;
4-[2-Amino-4-(3′-methoxybiphenyl-3-yl)-1-methyl-5-oxo-4,5-dihydro-1H-imidazol-4-yl]phenyl propane-1-sulfonate hydrochloride;
4-[2-Amino-4-(3′-methoxybiphenyl-3-yl)-1-methyl-5-oxo-4,5-dihydro-1H-imidazol-4-yl]phenyl propane-2-sulfonate hydrochloride;
4-[2-Amino-4-(3′-methoxybiphenyl-3-yl)-1-methyl-5-oxo-4,5-dihydro-1H-imidazol-4-yl]phenyl dimethylsulfamate hydrochloride;
4-[2-Amino-4-(3′-methoxybiphenyl-3-yl)-1-methyl-5-oxo-4,5-dihydro-1H-imidazol-4-yl]phenyl morpholine-4-sulfonate; and
4-[2-Amino-4-(3′-methoxybiphenyl-3-yl)-1-methyl-5-oxo-4,5-dihydro-1H-imidazol-4-yl]phenyl 2-methoxyethanesulfonate hydrochloride;
   as a free base or a pharmaceutically acceptable salt, solvate or solvate of a salt thereof.

Some compounds of formula I may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula I as hereinbefore defined as well as to the salts thereof. Salts for use in pharmaceutical compositions will be pharmaceutically acceptable salts, but other salts may be useful in the production of the compounds of formula I.

It is to be understood that the present invention relates to any and all tautomeric forms of the compounds of formula I.

Compounds of the invention can be used as medicaments. In some embodiments, the present invention provides compounds of formula I, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

In a further embodiment, the compounds of the present invention are represented by formula (I) or a pharmaceutically acceptable salt thereof as recited herein for use as a medicament.

In a further embodiment, the compounds of the present invention are represented by formula (I) or a pharmaceutically acceptable salt thereof as recited herein in the manufacture of a medicament for the treatment or prophylaxis of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia.

In a further embodiment, the compounds of the present invention are represented by a method for the treatment of Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined herein.

In a further embodiment, the compounds of the present invention are represented by a method for the prophylaxis of Aβ-related pathologies such as Downs syndrome and β-amyloidangiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia comprising administering to a human a therapeutically effective amount of a compound of formula (I) or a pharmaceutically acceptable salt, thereof as defined herein.

In a further embodiment, the compounds of the present invention are represented by a method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer Disease, neurodegeneration associated with diseases such as Alzheimer Disease or dementia including dementia of mixed vascular and degenerative origin, pre-senile dementia, senile dementia and dementia associated with Parkinson's Disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined herein and a cognitive and/or memory enhancing agent.

In a further embodiment, the compounds of the present invention are represented by a method of treating Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, Parkinson's Disease, Frontotemporal dementia Parkinson's Type, Parkinson dementia complex of Guam, HIV dementia, diseases with associated neurofibrillar tangle pathologies, dementia pugilistica, amyotrophic lateral sclerosis, corticobasal degeneration, Down syndrome, Huntington's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, anxiety, schizophrenia, cognitive disorders, hair loss, contraceptive medication, predemented states, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, vascular dementia, dementia with Lewy bodies, Frontotemporal dementia and androgenetic alopecia by administering to a human a compound of formula (I) or a pharmaceutically acceptable salt, thereof as defined herein and a choline esterase inhibitor or anti-inflammatory agent. In a further embodiment, the present invention provides a method of treating or preventing Aβ-related pathologies such as Downs syndrome and β-amyloid angiopathy, such as but not limited to cerebral amyloid angiopathy, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, or any other disease, disorder, or condition described herein, by administering to a mammal (including human) a compound of the present invention and an atypical antipsychotic agent. Atypical antipsychotic agents includes, but not limited to, Olanzapine (marketed as Zyprexa), Aripiprazole (marketed as Abilify), Risperidone (marketed as Risperdal), Quetiapine (marketed as Seroquel), Clozapine (marketed as Clozaril), Ziprasidone (marketed as Geodon) and Olanzapine/Fluoxetine (marketed as Symbyax).

In a further embodiment, the present invention provides that the mammal or human being treated with a compound of the invention has been diagnosed with a particular disease or disorder, such as those described herein. In these cases, the mammal or human being treated is in need of such treatment. Diagnosis, however, need not be previously performed.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Cognitive enhancing agents memory enhancing agents and choline esterase inhibitors includes, but not limited to, onepezil (Aricept), galantamine (Reminyl or Razadyne), rivastigmine (Exelon), tacrine (Cognex) and memantine (Namenda, Axura or Ebixa).

Schizophrenia and other Psychotic Disorder(s) includes but is not limited to Psychotic Disorder(s), Schizophreniform Disorder(s), Schizoaeffective Disorder(s), Delusional Disorder(s), Brief Psychotic Disorder(s), Shared Psychotic Disorder(s), and Psychotic Disorder(s) Due to a General Medical Condition. 2) Dementia and other Cognitive Disorder(s). 3) Anxiety Disorder(s) including but not limited to Panic Disorder(s) Without Agoraphobia, Panic Disorder(s) With Agoraphobia, Agoraphobia Without History of Panic Disorder(s), Specific Phobia, Social Phobia, Obsessive-Compulsive Disorder(s), Stress related Disorder(s), Posttraumatic Stress Disorder(s), Acute Stress Disorder(s), Generalized Anxiety Disorder(s) and Generalized Anxiety Disorder(s) Due to a General Medical Condition. 4) Mood Disorder(s) including but not limited to a) Depressive Disorder(s), including but not limited to Major Depressive Disorder(s) and Dysthymic Disorder(s) and b) Bipolar Depression and/or Bipolar mania including but not limited to Bipolar I, including but not limited to those with manic, depressive or mixed episodes, and Bipolar II, c) Cyclothymiac's Disorder(s), d) Mood Disorder(s) Due to a General Medical Condition. 5) Sleep Disorder(s). 6) Disorder(s) Usually First Diagnosed in Infancy, Childhood, or Adolescence including but not limited to Mental Retardation, Downs Syndrome, Learning Disorder(s), Motor Skills Disorder(s), Communication Disorders(s), Pervasive Developmental Disorder(s), Attention-Deficit and Disruptive Behavior Disorder(s), Feeding and Eating Disorder(s) of Infancy or Early Childhood, Tic Disorder(s), and Elimination Disorder(s). 7) Substance-Related Disorder(s) including but not limited to Substance Dependence, Substance Abuse, Substance Intoxication, Substance Withdrawal, Alcohol-Related Disorder(s), Amphetamines (or Amphetamine-Like)-Related Disorder(s), Caffeine-Related Disorder(s), Cannabis-Related Disorder(s), Cocaine-Related Disorder(s), Hallucinogen-Related Disorder(s), Inhalant-Related Disorder(s), Nicotine-Related Disorder(s)s, Opioid-Related Disorder(s)s, Phencyclidine (or Phencyclidine-Like)-Related Disorder(s), and Sedative-, Hypnotic- or Anxiolytic-Related Disorder(s). 8) Attention-Deficit and Disruptive Behavior Disorder(s). 9) Eating Disorder(s). 10) Personality Disorder(s) including but not limited to Obsessive-Compulsive Personality Disorder(s). 11) Impulse-Control Disorder(s).

Neurodegenerative Disorder(s) includes, but is not limited to, Alzheimer's Disease, Mild Cognitive Impairment, Dementia, Age-Associated Memory Impairment, Age-Related Cognitive Decline, Disorder(s) associated with neurofibrillar tangle pathologies, Dementia due to Alzheimer's Disease, Dementia due to Schizophrenia, Dementia due to Parkinson's Disease, Dementia due to Creutzfeld-Jacob Disease, Dementia due to Huntington's Disease, Dementia due to Pick's Disease, Stroke, Head Trauma, Spinal Injury, Multiple Sclerosis, Migraine, Pain, Systemic Pain, Localized Pain, Nociceptive Pain, Neuropathic Pain, Urinary Incontinence, Sexual Dysfunction, Premature Ejaculation, Motor Disorder(s), Endocrine Disorder(s), Gastrointestinal Disorder(s), and Vasospasm.

Many of the above conditions and disorder(s) are defined for example in the American Psychiatric Association Diagnostic and Statistical Manual of Mental Disorders, Fourth Edition, Text Revision, Washington, D.C., American Psychiatric Association, 2000.

The present invention also includes pharmaceutical compositions that contain, as the active ingredient, one or more of the compounds of the invention herein together with at least one pharmaceutically acceptable carrier, diluent or excipient.

The definitions set forth in this application are intended to clarify terms used throughout this application. The term “herein” means the entire application.

As used in this application, the term “optionally substituted,” as used herein, means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example when a substituent is methyl (i.e., CH₃), then 3 hydrogens on the carbon atom can be replaced. Examples of such substituents include, but are not limited to: halogen, CN, NH₂, OH, SO, SO₂, COOH, OC₁₋₆alkyl, CH₂OH, SO₂H, C₁₋₆alkyl, OC₁₋₆alkyl, C(═O)C₁₋₆alkyl, C(═O)OC₁₋₆alkyl, C(═O)NH₂, C(═O)NHC₁₋₆alkyl, C(═O)N(C₁₋₆alkyl)₂, SO₂C₁₋₆alkyl, SO₂NHC₁₋₆alkyl, SO₂N(C₁₋₆alkyl)₂, NH(C₁₋₆alkyl), N(C₁₋₆alkyl)₂, NHC(═O)C₁₋₆alkyl, NC(═O)(C₁₋₆alkyl)₂, C₅₋₆aryl, OC₅₋₆aryl, C(═O)C₅₋₆aryl, C(═O)OC₅₋₆aryl, C(═O)NHC₅₋₆aryl, C(═O)N(C₅₋₆aryl)₂, SO₂C₅₋₆aryl, SO₂NHC₅₋₆aryl, SO₂N(C₅₋₆aryl)₂, NH(C₅₋₆aryl), N(C₅₋₆aryl)₂, NC(═O)C₅₋₆aryl, NC(═O)(C₅₋₆aryl)₂, C₅₋₆heterocyclyl, OC₅₋₆heterocyclyl, C(═O)C₅₋₆heterocyclyl, C(═O)OC₅₋₆heterocyclyl, C(═O)NHC₅₋₆heterocyclyl, C(═O)N(C₅₋₆heterocyclyl)₂, SO₂C₅₋₆heterocyclyl, SO₂NHC₅₋₆heterocyclyl, SO₂N(C₅₋₆heterocyclyl)₂, NH(C₅₋₆heterocyclyl), N(C₅₋₆heterocyclyl)₂, NC(═O)C₅₋₆heterocyclyl, NC(═O)(C₅₋₆heterocyclyl)₂.

A variety of compounds in the present invention may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl”, “alkylenyl” or “alkylene” used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. As used herein, “C₁₋₃ alkyl”, whether a terminal substituent or an alkylene (or alkylenyl) group linking two substituents, is understood to specifically include both branched and straight chain methyl, ethyl, and propyl.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used alone or as a suffix or prefix is intended to include both branched and straight-chain alkyne containing aliphatic hydrocarbon groups having from 2 to 12 carbon atoms or if a specified number of carbon atoms is provided then that specific number would be intended. For example “C₂₋₆alkynyl” denotes alkynyl having 2, 3, 4, 5 or 6 carbon atoms. Examples of alkynyl include, but are not limited to, ethynyl, 1-propynyl, 2-propynyl, 3-butynyl, -pentynyl, hexynyl and 1-methylpent-2-ynyl.

As used herein, “aromatic” refers to hydrocarbonyl groups having one or more unsaturated carbon ring(s) having aromatic characters, (e.g. 4n+2 delocalized electrons) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “cycloalkenyl” refers to ring-containing hydrocarbyl groups having at least one carbon-carbon double bond in the ring, and having from 4 to 12 carbons atoms.

As used herein, “cycloalkynyl” refers to ring-containing hydrocarbyl groups having at least one carbon-carbon triple bond in the ring, and having from 7 to 12 carbons atoms.

As used herein, “halo” or “halogen” refers to fluoro, chloro, bromo, and iodo. “Counterion” is used to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 4 to about 14, 3 to about 7, or 5 to 6 ring-forming atoms. In some embodiments, the heteroaryl group has 1 to about 4, 1 to about 3, or 1 to 2 heteroatoms. In some embodiments, the heteroaryl group has 1 heteroatom.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

As used herein, “in vivo hydrolysable precursors” means an in vivo hydroysable (or cleavable) ester of a compound of formula Ia or formula Ib that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled compound” is a compound that has incorporated at least one radionuclide. In some embodiments the radionuclide is selected from the group consisting of ³H, ¹⁴C, ¹²⁵I, ³⁵S and ⁸²Br.

The anti-dementia treatment defined herein may be applied as a sole therapy or may involve, in addition to the compound of the invention, conventional chemotherapy. Such chemotherapy may include one or more of the following categories of agents: acetyl cholinesterase inhibitors, anti-inflammatory agents, cognitive and/or memory enhancing agents or atypical antipsychotic agents.

Such conjoint treatment may be achieved by way of the simultaneous, sequential or separate dosing of the individual components of the treatment. Such combination products employ the compounds of this invention.

Compounds of the present invention may be administered orally, parenteral, buccal, vaginal, rectal, inhalation, insufflation, sublingually, intramuscularly, subcutaneously, topically, intranasally, intraperitoneally, intrathoracially, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

An effective amount of a compound of the present invention for use in therapy of dementia is an amount sufficient to symptomatically relieve in a warm-blooded animal, particularly a human the symptoms of dementia, to slow the progression of dementia, or to reduce in patients with symptoms of dementia the risk of getting worse.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents; it can also be an encapsulating material.

In powders, the carrier is a finely divided solid, which is in a mixture with the finely divided active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Some of the compounds of the present invention are capable of forming salts with various inorganic and organic acids and bases and such salts are also within the scope of this invention. For example, such conventional non-toxic salts include those derived from inorganic acids such as hydrochloric, phosphoric, and the like; and the salts prepared from organic acids such as lactic, maleic, citric, benzoic, methanesulfonic, trifluoroacetate and the like.

In some embodiments, the present invention provides a compound of formula Ia or formula Ib or a pharmaceutically acceptable salt thereof for the therapeutic treatment (including prophylactic treatment) of mammals including humans, it is normally formulated in accordance with standard pharmaceutical practice as a pharmaceutical composition.

In addition to the compounds of the present invention, the pharmaceutical composition of this invention may also contain, or be co-administered (simultaneously or sequentially) with, one or more pharmacological agents of value in treating one or more disease conditions referred to herein.

The term composition is intended to include the formulation of the active component or a pharmaceutically acceptable salt with a pharmaceutically acceptable carrier. For example this invention may be formulated by means known in the art into the form of, for example, tablets, capsules, aqueous or oily solutions, suspensions, emulsions, creams, ointments, gels, nasal sprays, suppositories, finely divided powders or aerosols or nebulisers for inhalation, and for parenteral use (including intravenous, intramuscular or infusion) sterile aqueous or oily solutions or suspensions or sterile emulsions.

Liquid form compositions include solutions, suspensions, and emulsions. Sterile water or water-propylene glycol solutions of the active compounds may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The compounds of the invention may be derivatised in various ways. As used herein “derivatives” of the compounds includes salts (e.g. pharmaceutically acceptable salts), any complexes (e.g. inclusion complexes or clathrates with compounds such as cyclodextrins, or coordination complexes with metal ions such as Mn²⁺ and Zn²⁺), esters such as in vivo hydrolysable esters, free acids or bases, polymorphic forms of the compounds, solvates (e.g. hydrates), prodrugs or lipids, coupling partners and protecting groups. By “prodrugs” is meant for example any compound that is converted in vivo into a biologically active compound.

Salts of the compounds of the invention are preferably physiologically well tolerated and non toxic. Many examples of salts are known to those skilled in the art. All such salts are within the scope of this invention, and references to compounds include the salt forms of the compounds.

Compounds having acidic groups, such as carboxylate, phosphates or sulfates, can form salts with alkaline or alkaline earth metals such as Na, K, Mg and Ca, and with organic amines such as triethylamine and Tris(2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Acid addition salts may be formed with a wide variety of acids, both inorganic and organic. Examples of acid addition salts include salts formed with hydrochloric, hydriodic, phosphoric, nitric, sulphuric, citric, lactic, succinic, maleic, malic, isethionic, fumaric, benzenesulphonic, toluenesulphonic, methanesulphonic, ethanesulphonic, naphthalenesulphonic, valeric, acetic, propanoic, butanoic, malonic, glucuronic and lactobionic acids.

If the compound is anionic, or has a functional group that may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Esters can be formed between hydroxyl or carboxylic acid groups present in the compound and an appropriate carboxylic acid or alcohol reaction partner, using techniques well known in the art. Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Derivatives that are prodrugs of the compounds are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., -Me, -Et, -nPr, -iPr, -nBu, -sBu, -iBu, -tBu); C₁₋₇aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; pivaloyloxymethyl; acetoxymethyl; 1-acetoxyethyl; 1-(1-methoxy-1-methyl)ethyl-carbonyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl;(4-tetrahydropyranyl)carbonyloxymethyl; and 1-(4-tetrahydropyranyl)carbonyloxyethyl).

Also, some prodrugs are activated enzymatically to yield the active compound, or a compound that, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug, an antibody or an inhibitor. Coupling partners can be covalently linked to compounds of the invention via an appropriate functional group on the compound such as a hydroxyl group, a carboxyl group or an amino group. Other derivatives include formulating the compounds with liposomes.

Where the compounds contain chiral centres, all individual optical forms such as enantiomers, epimers and diastereoisomers, as well as racemic mixtures of the compounds are within the scope of the invention.

Compounds may exist in a number of different geometric isomeric, and tautomeric forms and references to compounds include all such forms. For the avoidance of doubt, where a compound can exist in one of several geometric isomeric or tautomeric forms and only one is specifically described or shown, all others are nevertheless embraced by the scope of this invention.

The quantity of the compound to be administered will vary for the patient being treated and will vary from about 100 ng/kg of body weight to 100 mg/kg of body weight per day and preferably will be from 10 ng/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Compounds of the present invention have been shown to inhibit beta secretase (including BACE) activity in vitro. Inhibitors of beta secretase have been shown to be useful in blocking formation or aggregation of Aβ peptide and therefore have a beneficial effect in treatment of Alzheimer's Disease and other neurodegenerative diseases associated with elevated levels and/or deposition of Aβ peptide. Therefore it is believed that the compounds of the present invention may be used for the treatment of Alzheimer disease and disease associated with dementia Hence compounds of the present invention and their salts are expected to be active against age-related diseases such as Alzheimer, as well as other Aβ related pathologies such as Downs syndrome and β-amyloid angiopathy. It is expected that the compounds of the present invention would most likely be used in combination with a broad range of cognition deficit enhancement agents but could also be used as a single agent.

A number of compounds of the present invention are useful as intermediates in the manufacture of compounds of formula I. These compounds include but are not limited to:
2-Acetyl-7-bromo-1,2,3,4-tetrahydroisoquinoline;
2-Acetyl-7-(4,4,5,5-tetramethyl-1,3,2-dioxaborolan-2-yl)-1,2,3,4-tetrahydroisoquinoline;
5-(3-Bromo-phenyl)-3-methyl-5-phenyl-2-thioxo-imidazolidin-4-one;
1-Acetyl-5-(4,4,5,5-tetramethyl-1,3,2-dioxaborolan-2-yl)indoline;
6-(4,4,5,5-Tetramethyl-1,3,2-dioxaborolan-2-yl)-2,3-dihydro-1-benzofuran;
(4-{[tert-Butyl(diphenyl)silyl]oxy}phenyl)[2-(3′-methoxybiphenyl-3-yl)-1,3-dithian-2-yl]methanol;
1-(4-{[tert-Butyl(diphenyl)silyl]oxy}phenyl)-2-(3′-methoxybiphenyl-3-yl)ethane-1,2-dione;
5-(4-Hydroxyphenyl)-5-(3′-methoxybiphenyl-3-yl)-3-methyl-2-thioxoimidazolidin-4-one;
4-[4-(3′-Methoxybiphenyl-3-yl)-1-methyl-5-oxo-2-thioxoimidazolidin-4-yl]phenyl methanesulfonate;
4-[4-(3′-Methoxybiphenyl-3-yl)-1-methyl-5-oxo-2-thioxoimidazolidin-4-yl]phenyl trifluoromethanesulfonate;
4-Methoxy-2-(4,4,5,5-tetramethyl-1,3,2-dioxaborolan-2-yl)phenol;
1-(3-Hydroxyphenyl)-2-phenylethane-1,2-dione;
5-(3-Hydroxyphenyl)-3-methyl-5-phenyl-2-thioxoimidazolidin-4-one;
Methyl-5-oxo-4-phenyl-2-thioxoimidazolidin-4-yl)phenyl methanesulfonate;
3-Bromo-4-{[tert-butyl(diphenyl)silyl]oxy}benzaldehyde;
3-Bromo-4-{[tert-butyl(diphenyl)silyl]oxy}phenyl)(2-phenyl-1,3-dithian-2-yl)methanol;
1-(3-Bromo-4-{[tert-butyl(diphenyl)silyl]oxy}phenyl)-2-phenylethane-1,2-dione;
5-(3-Bromo-4-hydroxyphenyl)-3-methyl-5-phenyl-2-thioxoimidazolidin-4-one;
2-(3′-Methoxybiphenyl-3-yl)-1,3-dithiane;
(3-{[tert-Butyl(diphenyl)silyl]oxy}phenyl)[2-(3′-methoxybiphenyl-3-yl)-1,3-dithian-2-yl]methanol;
1-(3-{[tert-Butyl(diphenyl)silyl]oxy}phenyl)-2-(3′-methoxybiphenyl-3-yl)ethane-1,2-dione;
5-(3-Hydroxyphenyl)-5-(3′-methoxybiphenyl-3-yl)-3-methyl-2-thioxoimidazolidin-4-one;
3-[4-(3′-Methoxybiphenyl-3-yl)-1-methyl-5-oxo-2-thioxoimidazolidin-4-yl]phenyl trifluoromethanesulfonate;
3-[4-(3′-Methoxybiphenyl-3-yl)-1-methyl-5-oxo-2-thioxoimidazolidin-4-yl]phenyl methanesulfonate;
5-(4,4,5,5-Tetramethyl-1,3,2-dioxaborolan-2-yl)-2,3-dihydro-1-benzofuran;
2,3-Dihydro-1-benzofuran-5-yl(2-phenyl-1,3-dithian-2-yl)methanol;
1-(2,3-Dihydro-1-benzofuran-5-yl)-2-phenylethane-1,2-dione;
5-(2,3-Dihydro-1-benzofuran-5-yl)-3-methyl-5-phenyl-2-thioxoimidazolidin-4-one;
2-Acetyl-5-chloro-1,2,3,4-tetrahydroisoquinoline;
2-Acetyl-5-(4,4,5,5-tetramethyl-1,3,2-dioxaborolan-2-yl)-1,2,3,4-tetrahydroisoquinoline;
(4-Methyl-3,4-dihydro-2H-1,4-benzoxazin-7-yl)(2-phenyl-1,3-dithian-2-yl)methanol
1-(4-Methyl-3,4-dihydro-2H-1,4-benzoxazin-7-yl)-2-phenylethane-1,2-dione;
3-Methyl-5-(4-methyl-3,4-dihydro-2H-1,4-benzoxazin-7-yl)-5-phenyl-2-thioxoimidazolidin-4-one;
6-Iodo-1,2,3,4-tetrahydronaphthalene;
6-(Phenylethynyl)-1,2,3,4-tetrahydronaphthalene;
1-Phenyl-2-(5,6,7,8-tetrahydronaphthalen-2-yl)ethane-1,2-dione;
3-Methyl-5-phenyl-5-(5,6,7,8-tetrahydronaphthalen-2-yl)-2-thioxoimidazolidin-4-one;
1-Acetyl-5-iodoindoline;
1-Acetyl-5-(phenylethynyl)indoline;
1-(1-Acetyl-2,3-dihydro-1H-indol-5-yl)-2-phenylethane-1,2-dione;
5-(1-Acetyl-2,3-dihydro-1H-indol-5-yl)-3-methyl-5-phenyl-2-thioxoimidazolidin-4-one;
1-Acetyl-4-(4,4,5,5-tetramethyl-1,3,2-dioxaborolan-2-yl)indoline;
8-(4,4,5,5-Tetramethyl-1,3,2-dioxaborolan-2-yl)chromane;
3-Methoxy-5-(4,4,5,5-tetramethyl-1,3,2-dioxaborolan-2-yl)phenol;
3′-Hydroxy-5′-methoxybiphenyl-3-carbaldehyde;
3′-{[tert-Butyl(diphenyl)silyl]oxy}-5′-methoxybiphenyl-3-carbaldehyde;
tert-Butyl{[3′-(1,3-dithian-2-yl)-5-methoxybiphenyl-3-yl]oxy}diphenylsilane;
[2-(3′-{[tert-Butyl(diphenyl)silyl]oxy}-5′-methoxybiphenyl-3-yl)-1,3-dithian-2-yl](pyridin-4-yl)methanol;
1-(3′-{[tert-Butyl(diphenyl)silyl]oxy}-5′-methoxybiphenyl-3-yl)-2-pyridin-4-ylethane-1,2-dione;
5-(3′-Hydroxy-5′-methoxybiphenyl-3-yl)-3-methyl-5-pyridin-4-yl-2-thioxoimidazolidin-4-one;
5-Methoxy-3′-(1-methyl-5-oxo-4-pyridin-4-yl-2-thioxoimidazolidin-4-yl)biphenyl-3-yl methanesulfonate;
[2-(3′-{[tert-Butyl(diphenyl)silyl]oxy}-5′-methoxybiphenyl-3-yl)-1,3-dithian-2-yl](pyridin-2-yl)methanol;
1-(3′-{[tert-Butyl(diphenyl)silyl]oxy}-5′-methoxybiphenyl-3-yl)-2-pyridin-2-ylethane-1,2-dione;
5-(3′-Hydroxy-5′-methoxybiphenyl-3-yl)-3-methyl-5-pyridin-2-yl-2-thioxoimidazolidin-4-one;
5-Methoxy-3′-(1-methyl-5-oxo-4-pyridin-2-yl-2-thioxoimidazolidin-4-yl)biphenyl-3-yl methanesulfonate;
[2-(3′-{[tert-Butyl(diphenyl)silyl]oxy}-5′-methoxybiphenyl-3-yl)-1,3-dithian-2-yl](3-furyl)methanol;
1-(3′-{[tert-Butyl(diphenyl)silyl]oxy}-5′-methoxybiphenyl-3-yl)-2-(3-furyl)ethane-1,2-dione;
5-(3-Furyl)-5-(3′-hydroxy-5′-methoxybiphenyl-3-yl)-3-methyl-2-thioxoimidazolidin-4-one;
3′-[4-(3-Furyl)-1-methyl-5-oxo-2-thioxoimidazolidin-4-yl]-5-methoxybiphenyl-3-yl methanesulfonate;
3′-[2-Amino-4-(3-furyl)-1-methyl-5-oxo-4,5-dihydro-1H-imidazol-4-yl]-5-methoxybiphenyl-3-yl methanesulfonate;
[2-(3′-{[tert-Butyl(diphenyl)silyl]oxy}-5′-methoxybiphenyl-3-yl)-1,3-dithian-2-yl](1,3-thiazol-5-yl)methanol;
1-(3′-{[tert-Butyl(diphenyl)silyl]oxy}-5′-methoxybiphenyl-3-yl)-2-(1,3-thiazol-5-yl)ethane-1,2-dione;
5-(3′-Hydroxy-5′-methoxybiphenyl-3-yl)-3-methyl-5-(1,3-thiazol-5-yl)-2-thioxoimidazolidin-4-one;
5-Methoxy-3′-[1-methyl-5-oxo-4-(1,3-thiazol-5-yl)-2-thioxoimidazolidin-4-yl]biphenyl-3-yl methanesulfonate;
[2-(3′-{[tert-Butyl(diphenyl)silyl]oxy}-5′-methoxybiphenyl-3-yl)-1,3-dithian-2-yl](1,3-thiazol-4-yl)methanol;
1-(3′-{[tert-Butyl(diphenyl)silyl]oxy}-5′-methoxybiphenyl-3-yl)-2-(1,3-thiazol-4-yl)ethane-1,2-dione;
5-(3′-Hydroxy-5′-methoxybiphenyl-3-yl)-3-methyl-5-(1,3-thiazol-4-yl)-2-thioxoimidazolidin-4-one;
5-Methoxy-3′-[1-methyl-5-oxo-4-(1,3-thiazol-4-yl)-2-thioxoimidazolidin-4-yl]biphenyl-3-yl methanesulfonate;
4-Bromo-1-fluoro-2-methoxybenzene;
2-(4-Fluoro-3-methoxyphenyl)-4,4,5,5-tetramethyl-1,3,2-dioxaborolane
4-[(3-Bromophenyl)ethynyl]phenol;
1-(3-Bromophenyl)-2-(4-hydroxyphenyl)ethane-1,2-dione;
5-(3-Bromophenyl)-5-(4-hydroxyphenyl)-3-methyl-2-thioxoimidazolidin-4-one;
4-[4-(3-Bromophenyl)-1-methyl-5-oxo-2-thioxoimidazolidin-4-yl]phenyl methanesulfonate;
4-[4-(3-Bromophenyl)-1-methyl-5-oxo-2-thioxoimidazolidin-4-yl]phenyl 2-methoxyethanesulfonate;
4-[4-(3-Bromophenyl)-1-methyl-5-oxo-2-thioxoimidazolidin-4-yl]phenyl propane-1-sulfonate;
3-Methoxy-5-(4,4,5,5-tetramethyl-1,3,2-dioxaborolan-2-yl)phenyl methanesulfonate;
3-[(3-Bromophenyl)ethynyl]phenol;
1-(3-Bromophenyl)-2-(3-hydroxyphenyl)ethane-1,2-dione;
5-(3-Bromophenyl)-5-(3-hydroxyphenyl)-3-methyl-2-thioxoimidazolidin-4-one;
3-[4-(3-Bromophenyl)-1-methyl-5-oxo-2-thioxoimidazolidin-4-yl]phenyl methanesulfonate;
3-[4-(3-Bromophenyl)-1-methyl-5-oxo-2-thioxoimidazolidin-4-yl]phenyl 2-methoxyethanesulfonate;
2-(4,4,5,5-Tetramethyl-1,3,2-dioxaborolan-2-yl)pyrimidine;
1-Ethynyl-3-(3-methoxyphenoxy)benzene;
3-[(4-Methoxyphenyl)ethynyl]phenol;
4-{[3-(3-Methoxyphenoxy)phenyl]ethynyl}phenol;
1-(3-Hydroxyphenyl)-2-(4-methoxyphenyl)ethane-1,2-dione;
5-(3-Hydroxyphenyl)-5-(4-methoxyphenyl)-3-methyl-2-thioxoimidazolidin-4-one;
1-(4-Hydroxyphenyl)-2-[3-(3-methoxyphenoxy)phenyl]ethane-1,2-dione;
5-(4-Hydroxyphenyl)-5-[3-(3-methoxyphenoxy)phenyl]-3-methyl-2-thioxoimidazolidin-4-one;
1-(4-Methoxyphenyl)-2-(3-phenoxyphenyl)ethane-1,2-dione;
5-(4-Methoxyphenyl)-3-methyl-5-(3-phenoxyphenyl)-2-thioxoimidazolidin-4-one;
3-Methoxypropane-1-sulfonyl chloride;
3-[4-(3′-Methoxybiphenyl-3-yl)-1-methyl-5-oxo-2-thioxoimidazolidin-4-yl]phenyl propane-2-sulfonate;
3-[4-(3′-Methoxybiphenyl-3-yl)-1-methyl-5-oxo-2-thioxoimidazolidin-4-yl]phenyl morpholine-4-sulfonate;
3-[2-Amino-4-(3′-methoxybiphenyl-3-yl)-1-methyl-5-oxo-4,5-dihydro-1H-imidazol-4-yl]phenyl morpholine-4-sulfonate hydrochloride;
6-Iodo-1,2,3,4-tetrahydronaphthalene;
6-[(3-Bromophenyl)ethynyl]-1,2,3,4-tetrahydronaphthalene;
1-(3-Bromophenyl)-2-(5,6,7,8-tetrahydronaphthalen-2-yl)ethane-1,2-dione;
5-(3-Bromophenyl)-3-methyl-5-(5,6,7,8-tetrahydronaphthalen-2-yl)-2-thioxoimidazolidin-4-one;
6-Bromo-1,2,3,4-tetrahydronaphthalen-2-yl methyl ether;
[(6-Methoxy-5,6,7,8-tetrahydronaphthalen-2-yl)ethynyl](trimethyl)silane;
6-Ethynyl-1,2,3,4-tetrahydronaphthalen-2-yl methyl ether;
6-[(3-Bromophenyl)ethynyl]-1,2,3,4-tetrahydronaphthalen-2-yl methyl ether;
1-(3-Bromophenyl)-2-(6-methoxy-5,6,7,8-tetrahydronaphthalen-2-yl)ethane-1,2-dione;
5-(3-Bromophenyl)-5-(6-methoxy-5,6,7,8-tetrahydronaphthalen-2-yl)-3-methyl-2-thioxoimidazolidin-4-one;
1-Acetyl-6-iodo-1,2,3,4-tetrahydroquinoline;
1-Acetyl-6-[(3-bromophenyl)ethynyl]-1,2,3,4-tetrahydroquinoline;
1-(1-Acetyl-1,2,3,4-tetrahydroquinolin-6-yl)-2-(3-bromophenyl)ethane-1,2-dione;
5-(1-Acetyl-1,2,3,4-tetrahydroquinolin-6-yl)-5-(3-bromophenyl)-3-methyl-2-thioxoimidazolidin-4-one;
5-[(3-Bromophenyl)ethynyl]indane;
1-(3-Bromophenyl)-2-(2,3-dihydro-1H-inden-5-yl)ethane-1,2-dione;
5-(3-Bromophenyl)-5-(2,3-dihydro-1H-inden-5-yl)-3-methyl-2-thioxoimidazolidin-4-one;
2,3-Dihydro-1-benzofuran-5-yl[2-(3′-methoxybiphenyl-3-yl)-1,3-dithian-2-yl]methanol;
1-(2,3-Dihydro-1-benzofuran-5-yl)-2-(3′-methoxybiphenyl-3-yl)ethane-1,2-dione;
5-(2,3-Dihydro-1-benzofuran-5-yl)-5-(3′-methoxybiphenyl-3-yl)-3-methyl-2-thioxoimidazolidin-4-one;
2-Acetyl-4-chloroisoindoline;
5-[(3-Bromophenyl)ethynyl]-2,3-dihydro-1-benzofuran;
1-(3-Bromophenyl)-2-(2,3-dihydro-1-benzofuran-5-yl)ethane-1,2-dione;
5-(3-Bromophenyl)-5-(2,3-dihydro-1-benzofuran-5-yl)-3-methyl-2-thioxoimidazolidin-4-one;
6-[(3-Bromophenyl)ethynyl]chromane;
1-(3-Bromophenyl)-2-(3,4-dihydro-2H-chromen-6-yl)ethane-1,2-dione;
5-(3-Bromophenyl)-5-(3,4-dihydro-2H-chromen-6-yl)-3-methyl-2-thioxoimidazolidin-4-one;
(3,4-Dihydro-1H-isochromen-7-ylethynyl)(trimethyl)silane;
7-Ethynyl-3,4-dihydro-1H-isochromene;
7-[(3-Bromophenyl)ethynyl]-3,4-dihydro-1H-isochromene;
1-(3-Bromophenyl)-2-(3,4-dihydro-1H-isochromen-7-yl)ethane-1,2-dione;
5-(3-Bromophenyl)-5-(3,4-dihydro-1H-isochromen-7-yl)-3-methyl-2-thioxoimidazolidin-4-one;
4-{[tert-Butyl(diphenyl)silyl]oxy}benzaldehyde;
tert-Butyl[4-(1,3-dithian-2-yl)phenoxy]diphenylsilane;
(3-Bromo-4-fluorophenyl)[2-(4-{[tert-butyl(diphenyl)silyl]oxy}phenyl)-1,3-dithian-2-yl]methanol;
1-(3-Bromo-4-fluorophenyl)-2-(4-{[tert-butyl(diphenyl)silyl]oxy}phenyl)ethane-1,2-dione;
5-(3-Bromo-4-fluorophenyl)-5-(4-hydroxyphenyl)-3-methyl-2-thioxoimidazolidin-4-one; and
4-[4-(3-Bromo-4-fluorophenyl)-1-methyl-5-oxo-2-thioxoimidazolidin-4-yl]phenyl methanesulfonate.

### Methods of Preparation

The present invention also relates to processes for preparing the compound of formula I as a free base or a pharmaceutically acceptable salt thereof. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in “Protective Groups in Organic Synthesis”, T. W. Greene, P. G. M Wutz, Wiley-Interscience, New York, 1999. It is understood that microwaves can be used for the heating of reaction mixtures.

### Preparation of Intermediates

The process, wherein R¹, R², R³, R⁷, R⁸, and R¹⁴, unless otherwise specified, are as defined hereinbefore, comprises,

(i) acylation of a compound of formula II to obtain a compound of formula III, wherein P represents a 4, 5, 6 or 7 membered nitrogen containing heterocyclyl group, and Halo represents a halogen such as iodine, bromine or chlorine, and R¹⁵ is a group such as alkyl, cycloalky, heterocyclyl, aryl or heteroaryl.

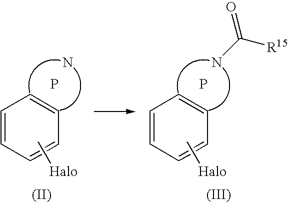

may be carried out by reaction with a suitable acylating reagent such as an anhydride e.g. acetic anhydride, or an acyl chloride e.g. acetyl chloride, in a suitable solvent such as dichloromethane, chloroform, toluene or acetonitrile at a temperature between −20° C. and reflux. The reaction is advantageously effected by the presence of a base. A suitable base may be an organic amine base such as pyridine, 2,6-lutidine, collidine, triethylamine, morpholine, N-methylmorpholine, diazabicyclo[5.4.0]undec-7-ene or tetramethylguanidine or an alkali metal or an alkaline earth metal carbonate or hydroxide such as sodium carbonate, potassium carbonate, calcium carbonate, sodium hydroxide or potassium hydroxide. The reaction may be aided by the presence of 4-dimethylaminopyridine. In addition standard amide coupling conditions can be employed using the activated ester of a carboxylic acid.
(ii) silylation of a compound of formula IV to obtain a compound of formula V, wherein Halo represents halogen e.g. iodine, bromine or chlorine and R¹⁶ is alkyl or aryl,

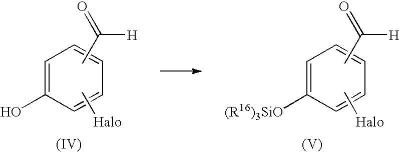

may be carried out with a suitable reagent such as a trialkylsilyl chloride such as t-butyldiphenylsilyl chloride, trimethylsilyl chloride or triisopropyl silylchloride, or a trialkylsilyltrifluoromethane sulfonate such as triethylsilyltrifluoromethane sulfonate, in the presence of a suitable base such as an organic amine base such as imidazole, pyridine, 2,6-lutidine, collidine, 4-dimethylaminopyridine, triethylamine, morpholine, N-methylmorpholine, diazabicyclo[5.4.0]undec-7-ene, tetramethylguanidine or an alkali metal hydride such as sodium hydride, in a suitable solvent such as dichloromethane, tetrahydrofuran, dimethylformamide at a temperature between 0° C. and 100° C. The reaction may be aided by 4-dimethylaminopyridine.
(iii) conversion of a compound of formula VIa or VIb to obtain a compound of formula VIIIa or VIb, wherein R¹⁷ and R¹⁸ are either exactly as defined hereinbefore for R² and R³ respectively or a protected variant,

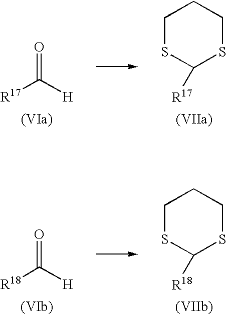

may be carried out by reaction with a suitable reagent such as 1,3-propanedithiol in the presence of an acid such as hydrochloric acid or p-toluenesulfonic acid, or a Lewis acid such as boron trifluoride or titanium tetrachloride, in a suitable solvent such as dichloromethane, acetonitrile, chloroform, toluene or diethylether, at a temperature between −78° C. and reflux.
(iv) reaction between a compound of formula VIa or VIb with a compound of formula VIIa or VIIb to obtain a compound of formula VIIIa or VIIIb, wherein R¹⁷ and R¹⁸ are defined as in (iii) above,

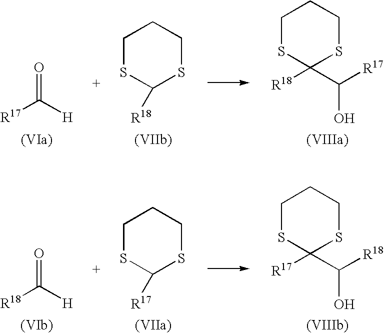

may be carried out by treating VII with a suitable base such as an alkyllithium reagent e.g. n-butyl lithium or t-butyl lithium before addition of VI. The reaction may be carried out in a solvent such as tetrahydrofuran or diethylether, or a mixture of tetrahydrofuran or diethylether with hexane, at a temperature between −100° C. and 0° C. The reaction may be aided by the presence of reagents such as hexamethylphosphoric triamide or N,N,N,N-tetramethyl-1,2-ethanediamine.
(v) oxidative deprotection of a compound of formula VIIIa or VIIIb to obtain a compound of formula IX, wherein R¹⁷ and R¹⁸ are defined as in (iii) above,

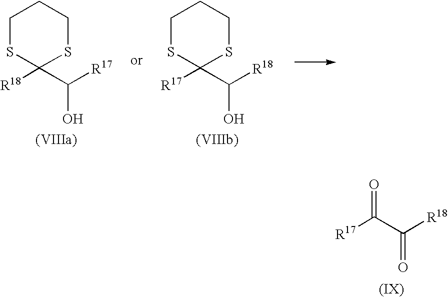

may be carried out by:
a) reaction with a suitable reagent such as 1,1,1-tris(acetyloxy)-1,1-dihydro-1,2-benziodoxol-3(1H)-one, bis(trifluoroacetoxy)iodobenzene, N-bromosuccinimide, or a mixture of trifluoroacetic acid with either sodium nitrite or formaldehyde, in a suitable solvent such as dichloromethane, acetonitrile, chloroform, acetone or water or a mixture thereof, between −5° C. to 40° C. The reaction may be aided by the presence of an alcohol such as t-butanol.
or,
b) hydrolysis by treatment with a suitable reagent or reagent combination such as N-chlorosuccinimide and silver nitrate, N-iodosuccinimide, 3-chloroperoxybenzoic acid, ammonium cerium(IV) nitrate, thallium(III) nitrate, mercury(II) chloride and calcium carbonate, or mercery(II) acetate, in a suitable solvent such as water, acetonitrile, methanol, acetone or diethyl ether or mixtures thereof, between −50° C. and 50° C., followed or preceded by, oxidation with a reagent such as 1,1,1-tris(acetyloxy)-1,1-dihydro-1,2-benziodoxol-3(1H)-one, manganese dioxide, hydrogen peroxide, potassium permanganate, pyridinium chlorochromate, copper sulfate or bromine, in a suitable solvent such as dichloromethane, water, acetonitrile, chloroform or dimethyl formamide, at between 0° C. and reflux.
(vi) oxidation of a compound of formula X to obtain a compound of formula IX, wherein R¹⁷ and R¹⁸ are defined as in (iii) above,

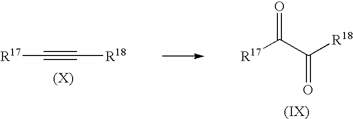

may be performed by reaction with a suitable reagent or mixture of reagents, such as sodium periodate and ruthenium dioxide, iodine and dimethyl sulfoxide, palladium chloride and dimethyl sulfoxide, oxone, hydrogen peroxide, oxygen, potassium permanganate, ruthenium tetroxide, or selenium dioxide, in a suitable solvent such as dimethyl sulfoxide, dichloromethane, acetonitrile, water, acetone, chloroform or carbon tetrachloride at a temperature between −78° C. and 150° C. The reaction may be aided by the presence of a catalyst such as ruthenium(III) chloride or iron(III) chloride.
(vii) conversion of a compound of formula IX to obtain a compound of formula XI, wherein R¹⁷ and R¹⁸ are as defined in (iii) above and R¹⁹ is either exactly as defined hereinbefore for R¹ or a protected variant,

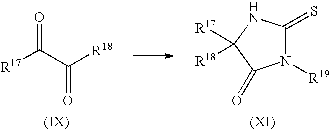

may be carried out by reaction with an appropriately N-substituted thiourea, such as N-methyl thiourea, in the presence of a suitable base such as potassium hydroxide or sodium hydroxide in a suitable solvent such as water, dimethyl sulfoxide, ethanol or methanol or mixtures thereof, between 20° C. and reflux.
(viii) conversion of a compound of formula XI to obtain a compound of formula XII, wherein R¹⁷, R¹⁸ and R¹⁹ are defined as in (iii) and (vii) above, with the proviso that XII is not a final compound,

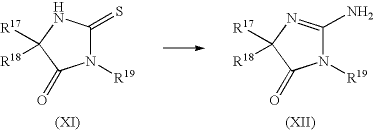

may be carried out by reaction with ammonia, or an ammonia equivalent, together with an alkylhydroperoxide such as t-butylhydroperoxide in a solvent such as ethanol, methanol or water, or a mixture thereof, at 0° C. to 50° C.
(ix) borylation of a compound of formula XIII to obtain a compound of formula XIV, wherein Halo represents halogen such as iodine, bromine or chlorine, R²⁰ may be a group outlined in Scheme I, wherein R²¹ and R²² are groups such as OH, C₁₋₆alkylO or C₂₋₃alkylO fused together to form a 5 or 6 membered boron containing heterocycle and the

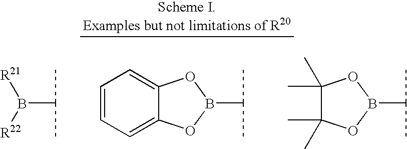

alkyl, cycloalkyl or aryl moieties may be optionally substituted; R²³ includes hydrogen or those definitions covered by R⁷ hereinbefore, provided that the substitutent is compatible with the cross-coupling chemistry. Alternative optionally substituted aromatic and heteroaromatic ring systems in addition to phenyl are also covered within this process.

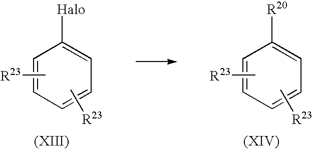

may be carried out by a reaction with:
a) an alkyllithium such as butyllithium, or magnesium, and a suitable boron compound such as trimethyl borate or triisopropyl borate. The reaction may be performed in a suitable solvent such as tetrahydrofuran, hexane or dichloromethane in a temperature range between −78° C. and +20° C.;
or,
b) a suitable boron species such as biscatecholatodiboron, bispinacolatodiboron or pinacolborane in the presence of a suitable palladium catalyst such as palladium(0) tetrakistriphenylphosphine, palladium diphenylphosphineferrocene dichloride or palladium acetate, with or without a suitable ligand such as 2-(dicyclohexylphosphino)biphenyl, and a suitable base, such as a tertiary amine, such as trietylamine or diisopropylethylamine, or potassium acetate may be used. The reaction may be performed in a solvent such as dioxane, toluene, acetonitrile, water, ethanol or 1,2-dimethoxyethane, or mixtures thereof, at temperatures between 20° C. and +160° C.
(x) conversion of a compound of formula XV to a compound of formula XVI, wherein Halo represents halogen such as chlorine, bromine or iodine, R¹⁹ is as defined in (vii) above, R²⁴ is either exactly as described for R² or R³ or a protected variant thereof, and R²⁵ is as defined for a compound of formula XIV in (ix) above, but with the point of attachment in place of R²⁰, with the proviso that the resultant compound with formula XVI is not a final compound,

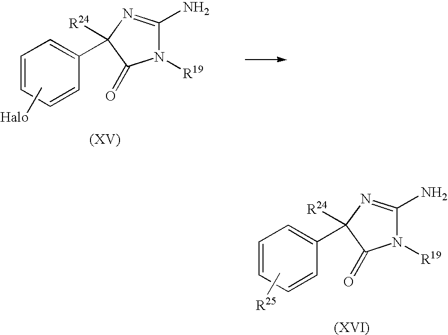

may be carried out by a de-halogen coupling with a suitable compound of formula XIV. The reaction may be carried out by coupling of a compound of formula XV with an appropriate aryl boronic acid or boronic ester of formula XIV. The reaction may be carried out using a suitable palladium catalyst such as tetrakis(triphenylphosphine)palladium(0), palladium diphenylphosphineferrocene dichloride or palladium acetate, together with, or without, a suitable ligand such as tri-tert-butylphosphine or 2-(dicyclohexylphosphino)biphenyl, or using a nickel catalyst such as nickel on charcoal or 1,2-Bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a temperature range between +20° C. and +160° C., in a suitable solvent such as toluene, tetrahydrofuran, dioxane, dimethoxyethane, water, ethanol or N,N-dimethylformamide, or mixtures thereof.
(xi) conversion of a compound of formula XVII to obtain a compound of formula XVIII, wherein R¹⁹ and R²⁴ are as defined above and R²⁶ is either covered by R⁸ or R¹²,

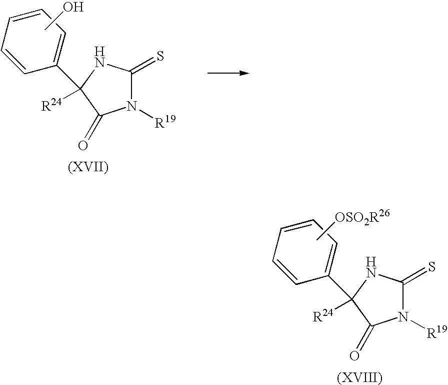

may be carried out by reaction with a suitable reagent such as an alkyl sulfonyl chloride e.g. methane sulfonyl chloride, an alkyl sulfonic anhydride, e.g. trifluoromethanesulfonic anhydride, or a sulfonamide e.g. N-phenyl-bis(trifluoromethanesulfonimide, in the presence of a suitable base such as an organic amine base such as pyridine, 2,6-lutidine, s-collidine, triethylamine, diispropyl ethylamine, morpholine, N-methylmorpholine, diazabicyclo[5.4.0]undec-7-ene or tetramethylguanidine, or an alkali metal or an alkaline earth metal carbonate such as sodium carbonate, potassium carbonate or calcium carbonate, or potassium phosphate, in a suitable solvent such as dichloromethane, tetrahydrofuran, chloroform, toluene, dimethyl formamide or pyridine at a temperature of −78° C. to 120° C. 4-Dimethylaminopyridine may aid the reaction.

### Methods of Preparation of End Products

Another object of the invention are processes a, b or c for the preparation of compounds of general formula I, wherein R¹, R² and R³, unless otherwise specified, are defined as hereinbefore, and salts thereof. When it is desired to obtain the acid salt, the free base may be treated with an acid such as a hydrogen halide such as hydrogen chloride, sulphuric acid, a sulphonic acid such as methane sulphonic acid or a carboxylic acid such as acetic or citric acid in a suitable solvent such as tetrahydrofuran, diethyl ether, methanol, ethanol, chloroform or dichloromethane or mixtures thereof, the reaction may occur between −30° C. to +50° C.

These processes comprise;

(a) conversion of a compound of formula XIX to obtain a compound of formula I.

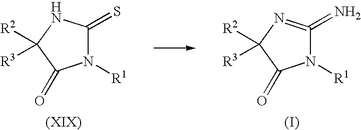

The reaction of process (a) may be carried out by reaction with ammonia, or an ammonia equivalent, together with an alkylhydroperoxide such as t-butylhydroperoxide in a solvent such as ethanol, methanol or water or a mixture thereof at a temperature of 0° C. to 50° C.

(b) conversion of a compound of formula XX to obtain a compound of formula I′ (where I′ is covered within the general definitions of a compound of formula I), wherein Halo represents halogen such as chlorine, bromine or iodine, ring B is defined such that once Halo has been substituted by R⁷ then the resultant final product I′ is covered by a compound of formula I,

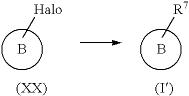

The reaction of process (b) may be carried out by a de-halogen coupling with a suitable compound of formula XIV.

The reaction may be carried out by coupling of a compound of formula XX with an appropriate aryl boronic acid or a boronic ester of formula XIV. The reaction may be carried out using a suitable palladium catalyst such as tetrakis(triphenylphosphine)palladium(0), palladium diphenylphosphineferrocene dichloride or palladium(II) acetate, together with, or without, a suitable ligand such as tri-tert-butylphosphine or 2-(dicyclohexylphosphino)biphenyl, or using a nickel catalyst such as nickel on charcoal or 1,2-Bis(diphenylphosphino)ethanenickel dichloride together with zinc and sodium triphenylphosphinetrimetasulfonate. A suitable base such as cesium fluoride, an alkyl amine such as triethyl amine, or an alkali metal or alkaline earth metal carbonate or hydroxide such as potassium carbonate, sodium carbonate, cesium carbonate, or sodium hydroxide may be used in the reaction, which may be performed in a temperature range between +20° C. and +160° C., in a suitable solvent such as toluene, tetrahydrofuran, dioxane, dimethoxyethane, water, ethanol or N,N-dimethylformamide, or mixtures thereof.

(c) conversion of a compound of formula XXI to obtain a compound of formula I″ (where I″ is covered within the general definitions of a compound of formula I), wherein ring C is defined such that when the —OH is replaced by —OSO₂R⁸ or —OSO₂R¹² then the resultant final product I″ is covered by a compound of formula I,

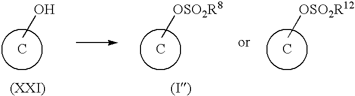

The reaction of process (c) may be carried out by reaction with a suitable reagent such as an alkyl sulfonyl chloride e.g. methane sulfonyl chloride, an alkyl sulfonic anhydride e.g. trifluoromethanesulfonic anhydride, or a sulfonimide e.g. N-phenyl-bis(trifluoromethanesulfonimide, in the presence of a suitable base such as an organic amine base such as pyridine, 2,6-lutidine, s-collidine, 4-dimethylaminopyridine, triethylamine, diispropyl ethylamine, morpholine, N-methylmorpholine, diazabicyclo[5.4.0]undec-7-ene or tetramethylguanidine, or an alkali metal or an alkaline earth metal carbonate or hydroxide such as sodium hydroxide, sodium carbonate, potassium carbonate or calcium carbonate, or potassium phosphate, in a suitable solvent such as dichloromethane, tetrahydrofuran, chloroform, toluene, dimethyl formamide or pyridine at a temperature of-78° C. to 120° C. 4-Dimethylaminopyridine may aid the reaction.

### EXAMPLES

Below follows a number of non-limiting examples of compounds of the invention.

### General Methods

Starting materials used were available from commercial sources, or prepared according to literature procedures.

Microwave heating was performed in a Creator, Initiator or Smith Synthesizer Single-mode microwave cavity producing continuous irradiation at 2450 MHz.

¹H NMR spectra were recorded in the indicated deuterated solvent at 400 MHz, unless stated otherwise, using a Bruker av400 NMR spectrometer equipped with a 3 mm flow injection SEI ¹H/D-¹³C probe head with Z-gradients, using a BEST 215 liquid handler for sample injection, or using a Bruker DPX400 NMR spectrometer equipped with a 4-nucleus probehead with Z-gradients. 600 MHz ¹H NMR were recorded using a Bruker DRX600 NMR spectrometer equipped with a 5 mm TXI probehead with Z-gradients. Chemical shifts are given in ppm down- and upfield from TMS. Resonance multiplicities are denoted s, d, t, q, m and br for singlet, doublet, triplet, quartet, multiplet, and broad respectively. LC-MS analyses were recorded on a Waters LCMS equipped with a Waters X-Terra MS, C8-column, (3.5 μm, 100 mm×3.0 mm i.d.). The mobile phase system consisted of A: 10 mM ammonium acetate in water/acetonitrile (95:5) and B: acetonitrile. A linear gradient was applied running from 0% to 100% B in 4-5 minutes with a flow rate of 1.0 mL/min. The mass spectrometer was equipped with an electrospray ion source (ESI) operated in a positive or negative ion mode. The capillary voltage was 3 kV and the mass spectrometer was typically scanned between m/z 100-700.

GC-MS analyses were performed on a Agilent 6890N GC equipped with a Chrompack CP-Sil 5CB column (25 m×0.25 mm i.d. df=0.25)), coupled to an Agilent 5973 Mass Selective Detector operating in a chemical ionization (CI) mode and the MS was scanned between m/z 50-500.

HPLC assays were performed using an Agilent HP1100 Series system equipped with a Waters X-Terra MS, C₈ column (3.0×100 mm, 3.5 μm). The column temperature was set to 40° C. and the flow rate to 1.0 mL/min. The Diode Array Detector was scanned from 200-300 nm. A linear gradient was applied, run from 0% to 100% B in 4 min. Mobile phase A: 10 mM ammonium acetate in water/acetonitrile (95:5), mobile phase B: acetonitrile.

Preparative HPLC was performed on a Waters Auto purification HPLC-UV system with a diode array detector using a Waters XTerra MS C₈ column (19×300 mm, 7 μm) and a linear gradient of mobile phase B was applied. Mobile phase A: 0.1 M ammonium acetate in water/acetonitrile (95:5) and mobile phase B: acetonitrile. Flow rate: 20 mL/min. Or preparative chromatography was run on a Waters FractionLynx system with a Autosampler combined Automated Fraction Collector (Waters 2767), Gradient Pump (Waters 2525), Regeneration Pump (Waters 600), Make Up Pump (Waters 515), Waters Active Splitter, Column Switch (Waters CFO), PDA (Waters 2996) and Waters ZQ mass spectrometer. Column; XBridge™ Prep C8 5 μm OBD™ 19×100 mm, with guard column; XTerra® Prep MS C8 10 μm 19×10 mm Cartridge. A gradient from 100% A (95% 0.1M NH₄OAc in MilliQ water and 5% MeCN) to 100% B (100% MeCN) was applied for LC-separation at flow rate 25 ml/min. The PDA was scanned from 210-350 nm. The ZQ mass spectrometer was run with ESI in positive mode. The Capillary Voltage was 3 kV and the Cone Voltage was 30V. Mixed triggering, UV and MS signal, determined the fraction collection.

Thin layer chromatography (TLC) was performed on Merch TLC-plates (Silica gel 60 F₂₅₄) and spots were UV visualized. Flash chromatography was performed using Merck Silica gel 60 (0.040-0.063 mm), or employing a Combi Flash® Companion™ system using RediSep™ normal-phase flash columns.

Compounds have been named using ACD/Name, version 8.08, software from Advanced Chemistry Development, Inc. (ACD/Labs), Toronto ON, Canada, www.acdlabs.com, 2004.

### Example 1

### 2-Acetyl-7-bromo-1,2,3,4-tetrahydroisoquinoline

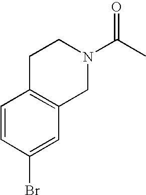

7-Bromo-1,2,3,4-tetrahydroisoquinoline (550 mg, 2.21 mmol), triethylamine (0.68 mL, 4.87 mmol) and N,N-dimethylpyridin-4-amine (27 mg, 0.22 mmol) were dissolved in dry dichloromethane (10 mL) and cooled to 0° C. under argon. Acetic anhydride (0.22 mL, 2.32 mmol) was added dropwise and the resulting mixture was stirred under argon at ambient temperature for 15 h. The mixture was then poured into aqueous hydrochloric acid (1M, 10 mL) and the organic phase was separated, washed with aqueous hydrochloric acid (1M, 10 mL) and saturated aqueous sodium bicarbonate (10 mL), dried over magnesium sulfate and concentrated in vacuo to yield the title compound, 556 mg (99% yield). ¹H-NMR (DMSO-d₆): δ 7.44-7.41, (m, 1H), 7.37-7.33 (m, 1H), 7.14 (d, J=8.0 Hz, 1H), 4.64 (s, 1H), 4.58 (s, 1H), 3.63 (t, J=6.0 Hz, 2H), 2.81 (t, J=5.9 Hz, 1H), 2.70 (t, J=5.9 Hz, 1H), 2.07 (s, 3H); MS (ESI) m/z 254 and 256 [M+1]⁺.

### Example 2

### 2-Acetyl-7-(4,4,5,5-tetramethyl-1,3,2-dioxaborolan-2-yl)-1,2,3,4-tetrahydroisoquinoline

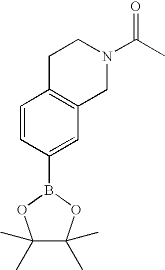

2-Acetyl-7-bromo-1,2,3,4-tetrahydroisoquinoline (150 mg, 0.59 mmol), 4,4,4′,4′,5,5,5′,5′-octamethyl-2,2′-bi-1,3,2-dioxaborolane (180 mg, 0.71 mmol), [1,1′-bis(diphenylphosphino)ferrocene]palladium(II) chloride dichloromethane adduct (15 mg, 0.018 mmol), potassium acetate (174 mg, 1.77 mmol) and 1,2-dimethoxyethan (4 mL) was irradiated in a microwave at 150° C. for 15 min. When cooled to ambient temperature the mixture was diluted with water (4 mL) and extracted with diethyl ether (3×5 mL). The combined organic extracts were passed through a silica pad. The product fractions were collected and concentrated to yield the title compound, 125 mg (70% yield). ¹H-NMR (DMSO-d₆): δ 7.53-7.44 (m, 2H), 7.21-7.16 (m, 1H), 4.65 (s, 1H), 4.58 (s, 1H), 3.64 (t, J=5.8 Hz, 2H), 2.87 (t, J=5.8 Hz, 1H), 2.77 (t, J=5.9 Hz, 1H), 2.07 (s, 3H), 1.28 (s, 6H), 1.16 (s, 6H); MS (ESI) m/z 302 [M+1]⁺.

### Example 3

### 5-(3-Bromo-phenyl)-3-methyl-5-phenyl-2-thioxo-imidazolidin-4-one

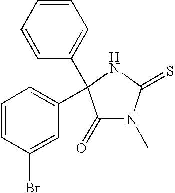

m-Bromobenzil (10.99 g, 38 mmol, described in Christy, M. E. et al. J. Med. Chem. 1977, 20, 421.) was dissolved in dimethyl sulfoxide (65 mL). N-Methylthiourea (6.85 g, 76 mmol) was added, and the solution was heated to 100° C. An aqueous solution of potassium hydroxide (1.5 M, 26 mL, 38 mmol) was added and the resulting solution was stirred at this temperature for 3 min, allowed to cool, and then poured into water (300 mL). The resulting slurry was vigorously stirred and the pH was adjusted to below 7 with aqueous hydrochloric acid (12 M, ca 4 mL). Stirring was continued for 20 min., and the precipitate was collected by filtration. The filter cake was washed with water (150 mL) and then dried in vacuo to yield 13.98 g (100% yield) of the title compound. ¹H-NMR (DMSO-d₆): δ 11.61 (s, 1H), 7.57 (d, J=8 Hz, 1H), 7.48 (s, 1H), 7.35-7.40 (m, 5H), 7.26 (d, J=8 Hz, 2H), 3.14 (s, 3H); MS (ESI) m/z 359 and 361 [M+1]⁺.

### Example 4

### 2-Amino-5-(3-bromophenyl)-3-methyl-5-phenyl-3,5-dihydro-imidazol-4-one

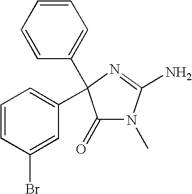

5-(3-Bromo-phenyl)-3-methyl-5-phenyl-2-thioxo-imidazolidin-4-one (2.53 g, 7 mmol) was added to a mixture of methanol (30 mL) and aqueous ammonia (25%, 10 mL). Aqueous t-butylhydroperoxide (70%, 12.5 mL, 105 mmol) was added, and the resulting mixture was stirred at 35° C. for 2 h. The mixture was then poured into water (300 mL) and extracted with dichloromethane (3×30 mL). The combined organic phases were washed with water (200 mL), dried over magnesium sulfate and concentrated in vacuo. The residue was dissolved in dichloromethane:methanol 90:10 (20 mL), suction filtered through a silica pad and concentrated in vacuo. Recrystallization from chloroform gave 1.48 g (68% yield) of the title compound. ¹H-NMR (DMSO-d₆): δ 7.61 (s, 1H), 7.40-7.50 (m, 4H), 7.22-7.32 (m, 4H), 6.72 (s, 2H), 2.98 (s, 3H); MS (ESI) m/z 344 and 346 [M+1]⁺

### Example 5

### 5-[3-(2-Acetyl-1,2,3,4-tetrahydroisoquinolin-7-yl)phenyl]-2-amino-3-methyl-5-phenyl-3,5-dihydro-4H-imidazol-4-one

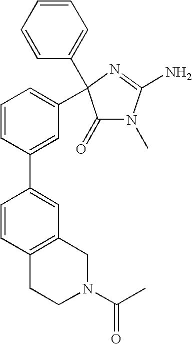

2-Acetyl-7-(4,4,5,5-tetramethyl-1,3,2-dioxaborolan-2-yl)-1,2,3,4-tetrahydroisoquinoline (125 mg, 0.42 mmol), 2-amino-5-(3-bromo-phenyl)-3-methyl-5-phenyl-3,5-dihydro-imidazol-4-one (143 mg, 0.42 mmol), [1,1′-bis(diphenylphosphino)ferrocene]palladium(II) chloride dichloromethane adduct (17 mg, 0.021 mmol), cesium carbonate (406 mg, 1.25 mmol) and solvent (4 mL of 1,2-dimethoxyethan:water:ethanol 6:3:1) was irradiated in a microwave at 150° C. for 15 min. When cooled to ambient temperature the mixture was diluted with water (5 mL) and extracted with diethyl ether (3×5 mL). The combined organic extracts were concentrated and purified by preparative HPLC to yield 14 mg (8% yield) of the title compound. MS (ESI) m/z 439 [M+1]⁺.

### Example 6

### 5-[3-(2-Acetyl-1,2,3,4-tetrahydroisoquinolin-7-yl)phenyl]-2-amino-3-methyl-5-phenyl-3,5-dihydro-4H-imidazol-4-one hydrochloride

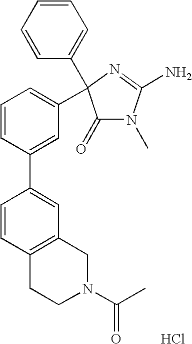

5-[3-(2-Acetyl-1,2,3,4-tetrahydroisoquinolin-7-yl)phenyl]-2-amino-3-methyl-5-phenyl-3,5-dihydro-4H-imidazol-4-one (14 mg, 0.032 mmol) was dissolved in dichloromethane (1 mL) and hydrochloric acid (1.0 M in diethyl ether, 33 μL, 0.033 mmol) was added. The resulting mixture was concentrated to dryness in vacuo to give the title compound, 12 mg (79% yield). ¹H-NMR (DMSO-d₆): δ 9.60 (s, 2H), 7.73-7.63 (m, 2H), 7.58-7.50 (m, 1H), 7.49-7.36 (m, 8H), 7.30-7.26 (m, 1H), 4.70 (s, 1H), 4.65 (s, 1H), 3.67 (t, J=5.9 Hz, 2H), 3.21 (s, 3H), 2.89 (t, J=5.9 Hz, 1H), 2.78 (t, J=5.9 Hz, 1H), 2.09 (s, 3H); MS (ESI) m/z 439 [M+1]⁺.

### Example 7

### 1-Acetyl-5-(4,4,5,5-tetramethyl-1,3,2-dioxaborolan-2-yl)indoline

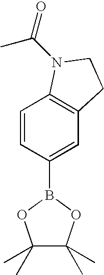

The title compound was synthesized as described for Example 2 in 85% yield starting from 1′-acetyl-5-bromoindoline. ¹H-NMR (DMSO-d₆): δ 8.04-8.00 (m, 1H), 7.51-7.45 (m, 2H), 4.09 (t, J=8.5 Hz, 2H), 3.12 (t, J=8.5 Hz, 2H), 2.16 (s, 3H), 1.27 (s, 6H), 1.16 (s, 6H); MS (CI) m/z 288 [M+1]⁺.

### Example 8

### 5-[3-(1-Acetyl-2,3-dihydro-1H-indol-5-yl)phenyl]-2-amino-3-methyl-5-phenyl-3,5-dihydro-4H-imidazol-4-one

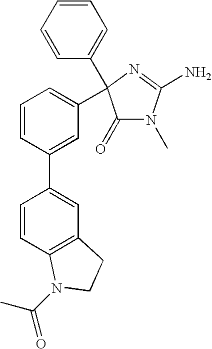

The title compound was synthesized as described for Example 5 in 15% yield starting from 2-amino-5-(3-bromo-phenyl)-3-methyl-5-phenyl-3,5-dihydro-imidazol-4-one and 1-acetyl-5-(4,4,5,5-tetramethyl-1,3,2-dioxaborolan-2-yl)indoline. MS (ESI) m/z 425 [M+1]⁺.

### Example 9

### 5-[3-(1-Acetyl-2,3-dihydro-1H-indol-5-yl)phenyl]-2-amino-3-methyl-5-phenyl-3,5-dihydro-4H-imidazol-4-one hydrochloride

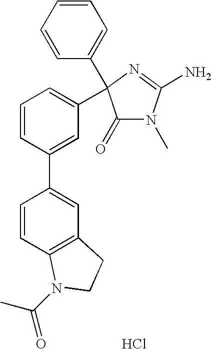

The title compound was synthesized as described for Example 6 in 93% yield starting from 5-[3-(1-acetyl-2,3-dihydro-1H-indol-5-yl)phenyl]-2-amino-3-methyl-5-phenyl-3,5-dihydro-4H-imidazol-4-one. ¹H-NMR (DMSO-d₆): δ 11.66 (s, 1H), 9.62 (s, 2H), 8.11-8.07 (m, 1H), 7.75-7.63 (m, 2H), 7.46-7.37 (m, 9H), 4.13 (t, J=8.5 Hz, 2H), 3.42-3.30 (m, 2H, obscured by water signal), 3.21 (s, 3H), 2.17 (s, 3H); MS (ESI) m/z 425 [M+1]⁺.

### Example 10

### 6-(4,4,5,5-Tetramethyl-1,3,2-dioxaborolan-2-yl)-2,3-dihydro-1-benzofuran

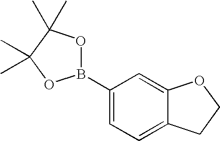

The title compound was synthesized as described for Example 2 starting from 6-bromo-2,3-dihydro-1-benzofuran (described in Song, Z. et al. Org. Lett. 2001, 3, 3357.). The crude product was subjected to flash chromatography using a gradient of dichloromethane and methanol with 1% aqueous ammonia to give the title compound in 97% yield. ¹H NMR (DMSO-d₆) δ 7.24 (m, 1H), 7.16 (dd, J=7.3, 0.8 Hz, 1H), 6.95 (s, 1H), 4.49 (t, J=8.8 Hz, 2H), 3.18 (t, J=8.7 Hz, 2H), 1.27 (s, 12H); MS (CI) m/z 247 [M+1]⁺.

### Example 11

### 2-Amino-5-[3-(2,3-dihydro-1-benzofuran-6-yl)phenyl]-3-methyl-5-phenyl-3,5-dihydro-4H-imidazol-4-one

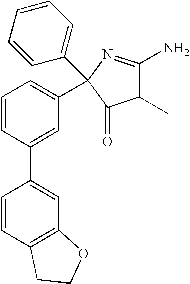

The title compound was synthesized as described for Example 5 starting from 2-amino-5-(3-bromophenyl)-3-methyl-5-phenyl-3,5-dihydro-4H-imidazol-4-one and 6-(4,4,5,5-tetramethyl-1,3,2-dioxaborolan-2-yl)-2,3-dihydro-1-benzofuran. The crude product was subjected to flash chromatography using a gradient of dichloromethane and methanol with 1% aqueous ammonia, followed by preparative HPLC giving the title compound in 61% yield. ¹H NMR (DMSO-d₆) δ 7.68 (s, 1H), 7.51-7.42 (m, 4H), 7.38-7.27 (m, 4H), 7.24-7.18 (m, 1H), 6.98 (dd, J=7.7, 1.6 Hz, 1H), 6.89 (d, J=1.3 Hz, 1H), 6.68 (br s, 2H), 4.56 (t, J=8.7 Hz, 2H), 3.19 (t, J=8.6 Hz, 2H), 2.99 (s, 3H); MS (ESI) m/z 384 [M+1]⁺.

### Example 12

### 2-Amino-5-[3-(2,3-dihydro-1-benzofuran-6-yl)phenyl]-3-methyl-5-phenyl-3,5-dihydro-4H-imidazol-4-one hydrochloride

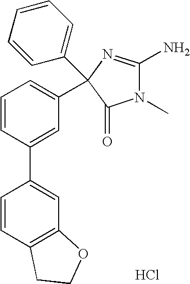

The title compound was synthesized as described for Example 6 in 96% yield starting from 2-amino-5-[3-(2,3-dihydro-1-benzofuran-6-yl)phenyl]-3-methyl-5-phenyl-3,5-dihydro-4H-imidazol-4-one. The crude product was subjected to flash chromatography using a gradient of dichloromethane and methanol with 1% aqueous ammonia, followed by preparative HPLC giving the title compound in 61% yield. ¹H NMR (DMSO-d₆) δ 7.68 (s, 1H), 7.51-7.42 (m, 4H), 7.38-7.27 (m, 4H), 7.24-7.18 (m, 1H), 6.98 (dd, J=7.7, 1.6 Hz, 1H), 6.89 (d, J=1.3 Hz, 1H), 6.68 (br s, 2H), 4.56 (t, J=8.7 Hz, 2H), 3.19 (t, J=8.6 Hz, 2H), 2.99 (s, 3H); MS (ESI) m/z 384 [M+1]⁺.

### Example 13

### (4-{[tert-Butyl(diphenyl)silyl]oxy}phenyl)[2-(3′-methoxybiphenyl-3-yl)-1,3-dithian-2-yl]methanol

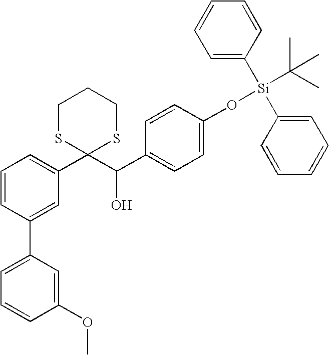

2-(3′-Methoxybiphenyl-3-yl)-1,3-dithiane (799 mg, 2.64 mmol) was dissolved in anhydrous tetrahydrofuran (10 mL) and the resulting mixture was cooled to −78° C. n-Butyllithium (1.6 M in hexanes, 1.82 mL, 2.91 mmol) was added, and the resulting solution was stirred for 2 h before 4-{[tert-butyl(diphenyl)silyl]oxy}benzaldehyde (1.00 g, 2.77 mmol, described in Mullen, D. G.; Barany, G., J. Org. Chem. 53, 1988, 5240.) dissolved in tetrahydrofuran (1.5 mL) was added. The resulting mixture was allowed to reach ambient temperature during 4 h. Saturated aqueous ammonium chloride (10 mL) was added and the phases were separated. The aqueous layer was extracted three times with dichloromethane and the combined organic extracts were dried over magnesium sulfate, filtered and concentrated in vacuo to give 1.80 g (72% yield) of the title product which was used without further purification. MS (ESI) m/z 645 [M-water]⁺.

### Example 14

### 1-(4-{[tert-Butyl(diphenyl)silyl]oxy}phenyl)-2-(3′-methoxybiphenyl-3-yl)ethane-1,2-dione

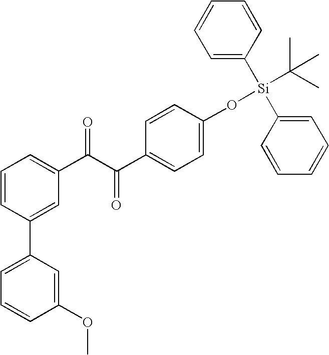

(4-{[tert-Butyl(diphenyl)silyl]oxy}phenyl)[2-(3′-methoxybiphenyl-3-yl)-1,3-dithian-2-yl]methanol (1.60 g, 2.41 mmol) and t-butanol (630 mg, 8.44 mmol) were dissolved in dichloromethane (20 mL). 1,1,1-Tris(acetyloxy)-1λ⁵,2-benziodoxol-3(1H)-one (2.56 g, 6.03 mmol) was added and the resulting mixture was stirred for 18 h. Sodium thiosulfate (2.50 g) in saturated aqueous sodium hydrogencarbonate (40 mL) followed by dichloromethane (20 mL) were added and the phases separated. The aqueous layer was extracted three times with dichloromethane and the combined organic extracts were dried over magnesium sulfate, filtered and concentrated in vacuo. Purification by flash chromatography (heptane:ethyl acetate gradient elution) yielded 1.27 g (74% yield) of the title compound. ¹H NMR (CDCl₃) δ 8.16 (t, J=1.8 Hz, 1H), 7.92-7.87 (m, 1H), 7.87-7.83 (m, 1H), 7.80-7.76 (m, 2H), 7.72-7.67 (m, 4H), 7.55 (t, J=7.7 Hz, 1H), 7.48-7.42 (m, 2H), 7.42-7.36 (m, 5H), 7.19-7.14 (m, 1H), 7.12-7.09 (m, 1H), 6.96-6.92 (m, 1H), 6.86-6.82 (m, 2H), 3.87 (s, 3H), 1.11 (s, 9H); MS (ESI) m/z 571 [M+1]⁺.

### Example 15

### 5-(4-Hydroxyphenyl)-5-(3′-methoxybiphenyl-3-yl)-3-methyl-2-thioxoimidazolidin-4-one

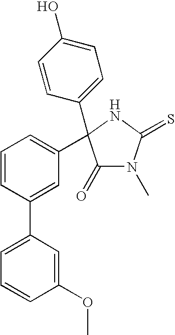

1-(4-{[tert-Butyl(diphenyl)silyl]oxy}phenyl)-2-(3′-methoxybiphenyl-3-yl)ethane-1,2-dione (1.21 g, 2.12 mmol) and N-methylthiourea (382 mg, 4.24 mmol) were dissolved in dimethyl sulfoxide (6 mL). Aqueous potassium hydroxide (1.2 M, 3.53 mL, 4.24 mmol) was added and the resulting mixture was heated to 100° C. for 2 min. by microwave irradiation. The mixture was diluted with chloroform and water after cooling, and the pH of the aqueous phase was adjusted to 4 by careful addition of aqueous hydrochloric acid (2 M). The phases were separated and the aqueous layer was extracted three times with chloroform. The combined organic extracts were washed three times with water and once with brine, dried over magnesium sulfate, filtered and concentrated in vacuo. Purification by flash chromatography (heptane:ethyl acetate gradient elution) yielded 525 mg (55% yield) of the title compound. ¹H NMR (CDCl₃) δ 8.11 (s, 1H), 7.60-7.52 (m, 2H), 7.44 (t, J=7.8 Hz, 1H), 7.36-7.31 (m, 2H), 7.20-7.15 (m, 2H), 7.12-7.08 (m, 1H), 7.07-7.04 (m, 1H), 6.92-6.88 (m, 1H), 6.82-6.77 (m, 2H), 3.84 (s, 3H), 3.34 (s, 3H); MS (ESI) m/z 405 [M+1]⁺.

### Example 16

### 4-[4-(3′-Methoxybiphenyl-3-yl)-1-methyl-5-oxo-2-thioxoimidazolidin-4-yl]phenyl methanesulfonate

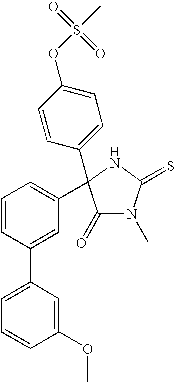

5-(4-Hydroxyphenyl)-5-(3′-methoxybiphenyl-3-yl)-3-methyl-2-thioxoimidazolidin-4-one (150 mg, 0.37 mmol) was dissolved in dichloromethane (3 mL) and cooled to 0° C. Triethylamine (52 μL, 0.37 mmol) followed by methanesulfonyl chloride (32 μL, 0.41 mmol) were added, and after 5 min the mixture was allowed to reach ambient temperature. After 2.5 h the mixture was cooled to 0° C. and additional triethylamine (10 μL, 0.07 mmol) followed by methanesulfonyl chloride (7 μL, 0.09 mmol) were added. The mixture was warmed to ambient temperature and stirred for 2.5 h. It was then diluted with dichloromethane and washed with aqueous hydrochloric acid (1.2 M). The aqueous layer was re-extracted with dichloromethane and the combined organic extracts were dried over magnesium sulfate, filtered and concentrated in vacuo. Purification by flash chromatography (heptane:ethyl acetate gradient elution) gave 141 mg (79% yield) of the title compound. ¹H NMR (CDCl₃) δ 8.23 (s, 1H), 7.62-7.58 (m, 1H), 7.51 (t, J=1.77 Hz, 1H), 7.49-7.43 (m, 3H), 7.37-7.28 (m, 4H), 7.12-7.08 (m, 1H), 7.06-7.03 (m, 1H), 6.93-6.88 (m, 1H), 3.84 (s, 3H), 3.34 (m, 3H), 3.17 (s, 3H); MS (ESI) m/z 483 [M+1]⁺.

### Example 17

### 4-[2-Amino-4-(3′-methoxybiphenyl-3-yl)-1-methyl-5-oxo-4,5-dihydro-1H-imidazol-4-yl]phenyl methanesulfonate

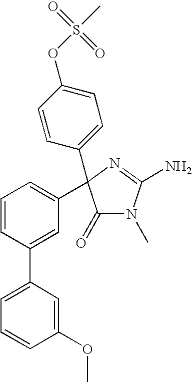

The title compound was synthesized from 4-[4-(3′-methoxybiphenyl-3-yl)-1-methyl-5-oxo-2-thioxoimidazolidin-4-yl]phenyl methanesulfonate as described for Example 4 except that purification was done by flash chromatography (heptane:ethyl acetate gradient elution) to give 87 mg (64% yield) of the title compound. ¹H NMR (CDCl₃) δ 7.70 (t, J=1.8 Hz, 1H), 7.61-7.55 (m, 2H), 7.51-7.47 (m, 1H), 7.46-7.42 (m, 1H), 7.39-7.29 (m, 2H), 7.22-7.16 (m, 2H), 7.15-7.11 (m, 1H), 7.10-7.06 (m, 1H), 6.90-6.85 (m, 1H), 5.56 (br s, 2H), 3.82 (s, 3H), 3.08 (s, 3H), 3.06 (s, 3H); MS (ESI) m/z 466 [M+1]⁺.

### Example 18

### 4-[2-Amino-4-(3′-methoxybiphenyl-3-yl)-1-methyl-5-oxo-4,5-dihydro-1H-imidazol-4-yl]phenyl methanesulfonate hydrochloride

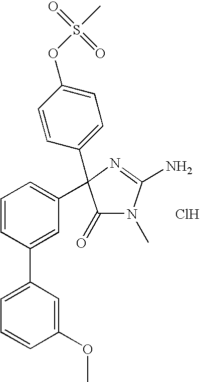

The title compound was synthesized in 88% yield from 4-[2-amino-4-(3′-methoxybiphenyl-3-yl)-1-methyl-5-oxo-4,5-dihydro-1H-imidazol-4-yl]phenyl methanesulfonate as described in Example 6. MS (ESI) m/z 466 [M+1]⁺.

### Example 19

### 4-[4-(3′-Methoxybiphenyl-3-yl)-1-methyl-5-oxo-2-thioxoimidazolidin-4-yl]phenyl trifluoromethanesulfonate

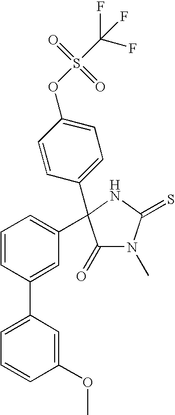

5-(4-Hydroxyphenyl)-5-(3′-methoxybiphenyl-3-yl)-3-methyl-2-thioxoimidazolidin-4-one (150 mg, 0.37 mmol) was dissolved in dichloromethane (3 mL) and cooled to 0° C. Triethylamine (57 μL, 0.41 mmol) followed by trifluoromethanesulfonic anhydride (69 μL, 0.41 mmol) were added, and after 5 minutes the mixture was allowed to reach ambient temperature. After 2.5 h the mixture was diluted with dichloromethane and washed with aqueous hydrochloric acid (1.2 M). The aqueous layer was re-extracted with dichloromethane and the combined organic extracts were dried over magnesium sulfate, filtered and concentrated in vacuo. Purification by flash chromatography (heptane:ethyl acetate gradient elution) gave 101 mg (51% yield) of the title compound. ¹H NMR (CDCl₃) δ 8.25 (s, 1H), 7.63-7.58 (m, 1H), 7.54-7.45 (m, 4H), 7.38-7.27 (m, 4H), 7.12-7.06 (m, 1H), 7.05-7.02 (m, 1H), 6.93-6.88 (m, 1H), 3.84 (s, 3H), 3.35 (s, 3H); MS (ESI) m/z 537 [M+1]⁺.

### Example 20

### 4-[2-Amino-4-(3′-methoxybiphenyl-3-yl)-1-methyl-5-oxo-4,5-dihydro-1H-imidazol-4-yl]phenyl trifluoromethanesulfonate

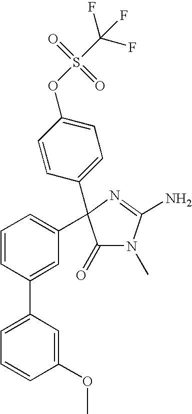

The title compound was synthesized from 4-[4-(3′-methoxybiphenyl-3-yl)-1-methyl-5-oxo-2-thioxoimidazolidin-4-yl]phenyl trifluoromethanesulfonate as described for Example 4, except that purification was done by flash chromatography (heptane:ethyl acetate gradient elution) to give 23 mg (24% yield) of the title compound. ¹H NMR (CDCl₃) δ 7.70-7.64 (m, 3H), 7.53-7.48 (m, 1H), 7.46-7.41 (m, 1H), 7.41-7.30 (m, 2H), 7.23-7.18 (m, 2H), 7.15-7.10 (m, 1H), 7.09-7.06 (m, 1H), 6.91-6.86 (m, 1H), 5.08 (br s, 2H), 3.83 (s, 3H), 3.11 (s, 3H); MS (ESI) m/z 520 [M+1]⁺.

### Example 21

### 4-[2-Amino-4-(3′-methoxybiphenyl-3-yl)-1-methyl-5-oxo-4,5-dihydro-1H-imidazol-4-yl]phenyl trifluoromethanesulfonate hydrochloride

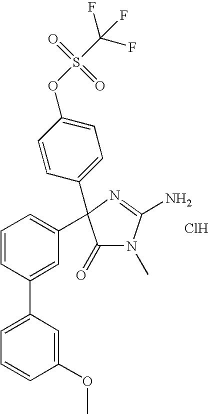

The title compound was synthesized in 86% yield as described for Example 6 starting from 4-[2-amino-4-(3′-methoxybiphenyl-3-yl)-1-methyl-5-oxo-4,5-dihydro-1H-imidazol-4-yl]phenyl trifluoromethanesulfonate. MS (ESI) m/z 520 [M+1]⁺.

### Example 22

### 2-Amino-5-(3′-hydroxybiphenyl-3-yl)-3-methyl-5-phenyl-3,5-dihydro-4H-imidazol-4-one

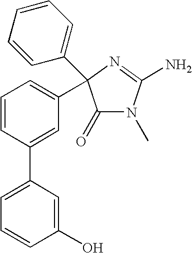

The title compound was synthesized as described for Example 5 starting from 2-amino-5-(3-bromo-phenyl)-3-methyl-5-phenyl-3,5-dihydro-imidazol-4-one and 3-(4,4,5,5-tetramethyl-1,3,2-dioxaborolan-2-yl)phenol. After the reaction the solvents were evaporated and the residue was purified by flash chromatography using a gradient of dichloromethane/methanol as eluent, to give 19% yield of the title compound. MS (ESI) m/z 357 [M+1]⁺.

### Example 23

### 3′-(2-Amino-1-methyl-5-oxo-4-phenyl-4,5-dihydro-1H-imidazol-4-yl)biphenyl-3-yl trifluoromethanesulfonate hydrochloride

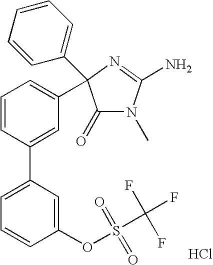

2-Amino-5-(3′-hydroxybiphenyl-3-yl)-3-methyl-5-phenyl-3,5-dihydro-4H-imidazol-4-one (76 mg, 0.21 mmol), N-phenyl-bis(trifluoromethanesulfonimide) (76 mg, 0.21 mmol), anhydrous potassium carbonate (178 mg, 1.29 mmol) and tetrahydrofuran (1 mL) were mixed and microwave irradiated to 120° C. for 6 min. The mixture was filtrated, the solvent was evaporated and the residue was purified by flash chromatography using a gradient of dichloromethane/methanol as eluent, to give 80 mg of the base. The base was dissolved in dichloromethane (3 mL) and hydrochloric acid (1M in diethyl ether, 0.36 mL). The mixture was stirred at room temperature for 5 min and the solvent was evaporated to give 86 mg (77% yield) of the title compound. ¹H NMR (DMSO-d₆) δ 11.62 (br s, 1H), 9.59 (br s, 2H), 7.78 (m, 2H), 7.74-7.67 (m, 3H), 7.59 (t, J=7.8 Hz, 1H), 7.53 (m, 1H), 7.48-7.41 (m, 3H), 7.41-7.37 (m, 3H), 3.21 (s, 3H); MS (ESI) m/z 490 [M+1]⁺.

### Example 24

### 4-Methoxy-2-(4,4,5,5-tetramethyl-1,3,2-dioxaborolan-2-yl)phenol

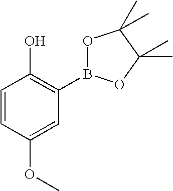

The title compound was synthesized as described for Example 2 using 2-bromo-4-methoxyphenol as starting material. The product was used in the next step without purification.

### Example 25

### 2-Amino-5-(2′-hydroxy-5′-methoxybiphenyl-3-yl)-3-methyl-5-phenyl-3,5-dihydro-4H-imidazol-4-one

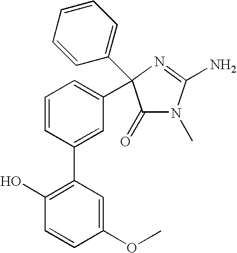

Synthesized as described for Example 5, using 4-methoxy-2-(4,4,5,5-tetramethyl-1,3,2-dioxaborolan-2-yl)phenol as the starting material, to give 43% yield of the title compound. MS (ES) m/z 388 [M+1]⁺.

### Example 26

### 3′-(2-Amino-1-methyl-5-oxo-4-phenyl-4,5-dihydro-1H-imidazol-4-yl)-5-methoxybiphenyl-2-yl trifluoromethanesulfonate hydrochloride

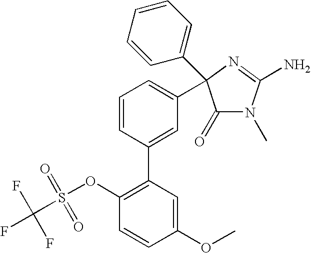

Synthesized as described for Example 23 using 2-amino-5-(2′-hydroxy-5′-methoxybiphenyl-3-yl)-3-methyl-5-phenyl-3,5-dihydro-4H-imidazol-4-one as the starting material to give 47% yield of the title compound. ¹H NMR (DMSO-d₆) δ 11.44 (br s, 1H), 9.55 (br s, 2H), 7.63-7.54 (m, 2H), 7.51-7.46 (m, 3H), 7.46-7.40 (m, 3H), 7.38-7.32 (m, 2H), 7.12 (dd, J=9.3, 3.0 Hz, 1H), 7.05 (d, J=3.3 Hz, 1H), 3.84 (s, 3H), 3.18 (s, 3H); MS (ESI) m/z 520 [M+1]⁺.

### Example 27

### 1-(3-Hydroxyphenyl)-2-phenylethane-1,2-dione

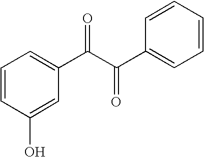

A solution of 3-(phenylethynyl)phenol (1.80 g, 9.27 mmol, described in Mohamed Ahmed, M. S.; Mori, A. Tetrahedron 2004, 60, 9977.) and palladium(II) chloride (0.176 g, 0.992 mmol) in dimethyl sulfoxide (25 mL) was heated at 140° C. for 5 h. The solvent was evaporated and the residue was purified by flash chromatography using a gradient of heptane/ethyl acetate as eluent, to give 1.057 g (50% yield) of the title compound. ¹H NMR (DMSO-d₆) δ 10.08 (br s, 1H), 7.90 (d, J=7.3 Hz, 2H), 7.80 (m, 1H), 7.63 (m, 2H), 7.43 (m, 1H), 7.30 (m, 2H), 7.17 (m, 1H); MS (ESI) m/z 225 [M−1]⁻.

### Example 28

### 5-(3-Hydroxyphenyl)-3-methyl-5-phenyl-2-thioxoimidazolidin-4-one

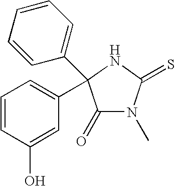

Synthesized as described for Example 3 using 1-(3-hydroxyphenyl)-2-phenylethane-1,2-dione as starting material, to give 99% yield of the title compound. ¹H NMR (DMSO-d₆) δ 11.56 (br s, 1H), 9.60 (br s, 1H), 7.45-7.36 (m, 3H), 7.34 (m, 2H), 7.20 (m, 1H), 6.73 (m, 3H), 3.17 (s, 3H); MS (ESI) m/z 297 [M−1]⁻.

### Example 29

### 3-(1-Methyl-5-oxo-4-phenyl-2-thioxoimidazolidin-4-yl)phenyl methanesulfonate

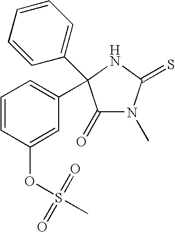

Triethylamine (0.38 mL, 2.73 mmol) was added to a cooled (0° C.) solution of 5-(3-hydroxyphenyl)-3-methyl-5-phenyl-2-thioxoimidazolidin-4-one (0.165 g, 0.55 mmol) in dichloromethane (4 mL), followed by addition of methanesulfonyl chloride (0.050 mL, 0.65 mmol). The resulting mixture was stirred at 0° C. for 1.5 h and was the allowed to warm up to ambient temperature. Dichloromethane was added and the organic phase was washed with aqueous hydrochloric acid (2M) and water, dried over magnesium sulfate and concentrated in vacuo to give the title compound that was used in the next without purification. MS (ESI) m/z 375 [M−1]⁻.

### Example 30

### 3-(2-Amino-1-methyl-5-oxo-4-phenyl-4,5-dihydro-1H-imidazol-4-yl)phenyl methanesulfonate hydrochloride

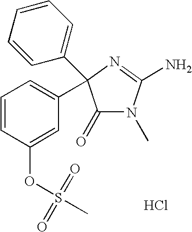

Synthesized as described for Example 4 using 3-(1-methyl-5-oxo-4-phenyl-2-thioxoimidazolidin-4-yl)phenyl methanesulfonate as the starting material, to give 79 mg, 40% yield of the base. The base was dissolved in dichloromethane, hydrochloric acid (1M in diethyl ether, 0.44 mL) was added, and the resulting mixture was stirred at ambient temperature for 10 min. The solvents were evaporated to give 87 mg (100% yield) of the title compound. ¹H NMR (DMSO-d₆) δ 11.73 (br s, 1H), 9.69 (br s, 2H), 7.59 (t, J=8.0 Hz, 1H), 7.50-7.40 (m, 5H), 7.39-7.32 (m, 3H), 3.41 (s, 3H), 3.20 (s, 3H); MS (ESI) m/z 360.0 [M+1]⁺.

### Example 31

### 2-Amino-5-(3-hydroxyphenyl)-3-methyl-5-phenyl-3,5-dihydro-4H-imidazol-4-one

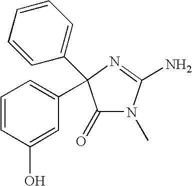

Synthesized as described for Example 4 using 5-(3-hydroxyphenyl)-3-methyl-5-phenyl-2-thioxoimidazolidin-4-one as starting material to give 80% yield of the title compound. ¹H NMR (DMSO-d₆) δ 10.73 (br s, 1H), 9.29 (br s, 2H), 7.42 (m, 2H), 7.28 (m, 2H), 7.22 (m, 1H), 7.06 (m, 1H), 6.85 (m, 2H), 6.59 (m, 1H), 2.97 (s, 3H); MS (ESI) m/z 282 [M+1]⁺.

### Example 32

### 3-(2-Amino-1-methyl-5-oxo-4-phenyl-4,5-dihydro-1H-imidazol-4-yl)phenyl trifluoromethanesulfonate hydrochloride

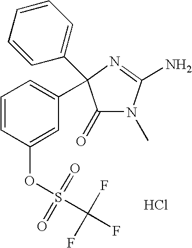

Synthesized as described for Example 23 using 2-amino-5-(3-hydroxyphenyl)-3-methyl-5-phenyl-3,5-dihydro-4H-imidazol-4-one as the starting material to give 35% yield of the title compound. ¹H NMR (DMSO-d₆) δ 11.70 (br s, 1H), 9.72 (br s, 2H), 7.69 (t, J=8.0 Hz, 1H), 7.61 (m, 1H), 7.56 (m, 1H), 7.50-7.42 (m, 4H), 7.34 (m, 2H), 3.20 (s, 3H); MS (ESI) m/z 414 [M+1]⁺.

### Example 33

### 3-Bromo-4-{[tert-butyl(diphenyl)silyl]oxy}benzaldehyde

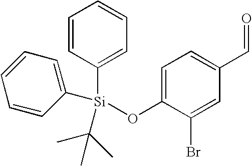

3-Bromo-4-hydroxybenzaldehyde (505 mg, 2.51 mmol) and imidazole (342 mg, 5.02 mmol) were dissolved in dimethyl formamide (15 mL) under a nitrogen atmosphere. tert-Butyl(chloro)diphenylsilane (898 mg, 3.27 mmol) was added. The resulting reaction mixture was stirred at ambient temperature for 3 h. Dichloromethane and water was added and the phases separated. The organic phase was dried over sodium sulfate and concentrated in vacuo. The crude product was purified by flash chromatography (heptane:ethyl acetate gradient elution) to yield 501 mg (45% yield) of the title compound. ¹H NMR (CDCl₃) 8 ppm 9.76 (s, 1H), 8.11 (d, J=2.0 Hz, 1H), 7.73 (m, 4H), 7.51-7.37 (m, 7H), 6.54 (d, J=8.3 Hz, 1H), 1.17 (s, 9H); MS (ESI) m/z 439 and 441 [M+1]⁺.

### Example 34

### 3-Bromo-4-{[tert-butyl(diphenyl)silyl]oxy}phenyl)(2-phenyl-1,3-dithian-2-yl)methanol

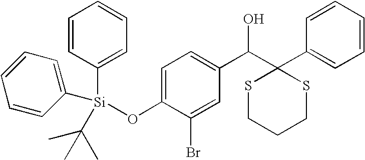

The title compound was synthesized as described for Example 13 starting from 2-phenyl-1,3-dithiane (213 mg, 1.08 mmol) and 3-bromo-4-{[tert-butyl(diphenyl)silyl]oxy}benzaldehyde (500 mg, 1.14 mmol). The crude product was purified by flash chromatography (heptane:ethyl acetate gradient elution) to yield 633 mg (92% yield) of the title compound. ¹H NMR (DMSO-d₆) 8 ppm 7.65 (m, 4H), 7.54-7.42 (m, 8H), 7.25-7.21 (m, 3H), 6.84 (d, J=2.0 Hz, 1H), 6.40 (dd, J=8.4, 2.1 Hz, 1H), 6.15 (d, J=8.3 Hz, 1H), 5.94 (br s, 1H), 4.77 (s, 1H), 2.70 (m, 2H), 2.47-2.31 (m, 2H), 1.82 (m, 1H), 1.67 (m, 1H), 1.06 (s, 9H); MS (ESI) m/z 617 and 619 [M+1-water]⁺.

### Example 35

### 1-(3-Bromo-4-{[tert-butyl(diphenyl)silyl]oxy}phenyl)-2-phenylethane-1,2-dione

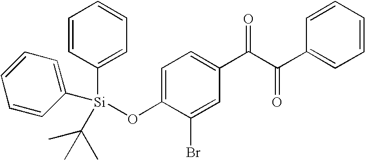

The title compound was synthesized as described for Example 14 starting from 3-bromo-4-{[tert-butyl(diphenyl)silyl]oxy}phenyl)(2-phenyl-1,3-dithian-2-yl)methanol. The crude product was purified by flash chromatography (heptane:ethyl acetate gradient elution) to yield 80% of the title compound. ¹H NMR (DMSO-d₆) δ 8.19 (d, J=2.3 Hz, 1H), 7.92-7.86 (m, 2H), 7.80-7.73 (m, 1H), 7.71-7.66 (m, 4H), 7.64-7.56 (m, 3H), 7.55-7.44 (m, 5H), 7.41-7.36 (m, 1H), 6.55 (d, J=8.5 Hz, 1H), 1.09 (s, 9H); MS (ESI) m/z 543 and 545 [M+1]⁺.

### Example 36

### 5-(3-Bromo-4-hydroxyphenyl)-3-methyl-5-phenyl-2-thioxoimidazolidin-4-one

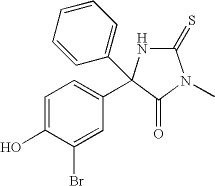

The title compound was synthesized as described for Example 3 starting from 1-(3-bromo-4-{[tert-butyl(diphenyl)silyl]oxy}phenyl)-2-phenylethane-1,2-dione, except that the product was extracted with dichloromethane from the water phase after the acidification, dried over magnesium sulfate, concentrated in vacuo and then purified by flash chromatography (heptane: ethyl acetate gradient elution) to yield 67% of the title compound. ¹H NMR (DMSO-d₆) δ 11.54 (s, 1H), 10.58 (br s, 1H), 7.44-7.35 (m, 4H), 7.29 (m, 2H), 7.16 (dd, J=8.5, 2.3 Hz, 1H), 6.98 (d, J=8.8 Hz, 1H), 3.17 (s, 3H); MS (ESI) m/z 377 and 379 [M+1]⁺.

### Example 37

### 2-Amino-5-(3-bromo-4-hydroxyphenyl)-3-methyl-5-phenyl-3,5-dihydro-4H-imidazol-4-one

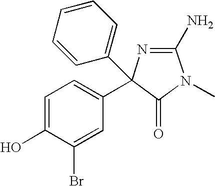

The title compound was synthesized as described for Example 4 starting from 5-(3-bromo-4-hydroxyphenyl)-3-methyl-5-phenyl-2-thioxoimidazolidin-4-one, except that the reaction mixture was stirred at 35° C. until judged complete by HPLC. The crude product was purified by flash chromatography (gradient elution dichloromethane: methanol with 1% aqueous ammonia) to yield 80% of the title compound. ¹H NMR (DMSO-d₆) δ 7.50 (d, J=1.5 Hz, 1H), 7.38 (d, J=7.5 Hz, 2H), 7.31-7.19 (m, 4H), 6.87 (d, J=8.5 Hz, 1H), 2.97 (s, 3H); MS (ESI) m/z 360 and 362 [M+1]⁺.

### Example 38

### 2-Amino-5-(6-hydroxy-3′-methoxybiphenyl-3-yl)-3-methyl-5-phenyl-3,5-dihydro-4H-imidazol-4-one

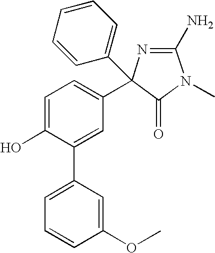

The title compound was synthesized as described for Example 5 starting from 2-amino-5-(3-bromo-4-hydroxyphenyl)-3-methyl-5-phenyl-3,5-dihydro-4H-imidazol-4-one and (3-methoxyphenyl)boronic acid. The crude product was purified by flash chromatography (gradient elution dichloromethane:methanol with 1% aqueous ammonia) to yield 27% of the title compound. MS (ESI) m/z 388 [M+1]⁺.

### Example 39

### 5-(2-Amino-1-methyl-5-oxo-4-phenyl-4,5-dihydro-1H-imidazol-4-yl)-3′-methoxybiphenyl-2-yl trifluoromethanesulfonate

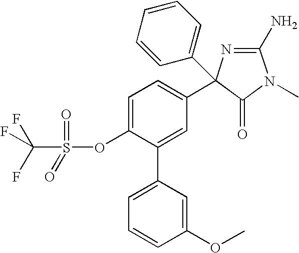

2-amino-5-(6-hydroxy-3′-methoxybiphenyl-3-yl)-3-methyl-5-phenyl-3,5-dihydro-4H-imidazol-4-one (14 mg, 0.036 mmol), N-phenyl-bis(trifluoromethanesulfonimide) (20 mg, 0.054 mmol), anhydrous potassium carbonate (30 mg, 0.216 mmol) and tetrahydrofuran (1.5 mL) were mixed and microwave irradiated to 120° C. for 18 min. Another portion of N-phenyl-bis(trifluoromethanesulfonimide) (40 mg, 0.018 mmol) was added and the reaction mixture was microwave irradiated to 120° C. for 18 min to ensure complete conversion. Dichloromethane was added and the mixture was filtrated. The filtrate was concentrated in vacuo and the residue was purified by flash chromatography (gradient elution dichloromethane:methanol with 1% aqueous ammonia) followed by preparative HPLC to yield 4 mg (21% yield) of the title compound. ¹H NMR (CDCl₃) δ 7.66 (d, J=2.3 Hz, 1H), 7.58 (dd, J=8.7, 2.4 Hz, 1H), 7.50-7.45 (m, 2H), 7.37-7.29 (m, 5H), 7.03 (m, 1H), 6.99 (m, 1H), 6.94 (ddd, J=8.3, 2.6, 1.0 Hz, 1H), 3.84 (s, 3H), 3.13 (s, 3H); MS (ESI) m/z 520 [M+1]⁺.

### Example 40

### 2-(3′-Methoxybiphenyl-3-yl)-1,3-dithiane

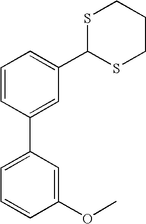

Boron trifluoride-diethyl etherate (14 mL, 110 mmol) was added dropwise to a cooled (0° C.) solution of 3′-methoxybiphenyl-3-carbaldehyde (5.95 g, 28.0 mmol, described in Mor, M. et al. J Med. Chem. 2004, 47, 4998.), and 1,3-propanedithiol (2.8 mL, 28 mmol) in dichloromethane (80 mL). The resulting mixture was stirred at 0° C. for 1 h. Saturated aqueous sodium bicarbonate was added and the aqueous phase was extracted with dichloromethane. The combined organic phases were washed with water, aqueous potassium hydroxide (10%), and water, dried over magnesium sulfate, and concentrated in vacuo to give 8.36 g (99% yield) of the title compound. ¹H NMR (DMSO-d₆) δ 7.68 (s, 1H), 7.62 (m, 1H), 7.49-7.35 (m, 3H), 7.19 (t, J=7.8 Hz, 1H), 7.15 (m, 1H), 6.96 (m, 1H), 5.47 (s, 1H), 3.83 (s, 3H), 3.10 (m, 2H), 2.91 (m, 2H), 2.14 (m, 1H), 1.75 (m, 1H); MS (ESI) m/z 303 [M+1]⁺.

### Example 41

### (3-{[tert-Butyl(diphenyl)silyl]oxy}phenyl)[2-(3′-methoxybiphenyl-3-yl)-1,3-dithian-2-yl]methanol

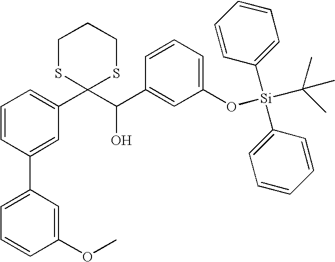

The title compound was synthesized as described for Example 13 starting from 2-(3′-methoxybiphenyl-3-yl)-1,3-dithiane and 3-{[tert-butyl(diphenyl)silyl]oxy}benzaldehyde (described in: Mäkelä, T. et al. Tetrahedron 2000, 56, 1873.) The crude product was purified by flash chromatography using cyclohexane/ethyl acetate, (20:1) as eluent, to give 39% yield of the title compound. ¹H NMR (DMSO-d₆) δ 7.73 (br s, 1H), 7.68 (d, J=7.8 Hz, 1H), 7.62-7.56 (m, 4H), 7.51 (d, J=7.8 Hz, 1H), 7.47-7.29 (m, 8H), 7.02 (d, J=7.8 Hz, 1H), 6.95-6.90 (m, 2H), 6.81 (t, J=7.8 Hz, 1H), 6.45-6.39 (m, 2H), 6.34 (d, J=7.8 Hz, 1H), 5.93 (d, J=4.3 Hz, 1H), 4.78 (d, J=3.8 Hz, 1H), 3.75 (s, 3H), 2.76-2.62 (m, 2H), 2.49-2.35 (m, 2H), 1.88-1.79 (m, 1H), 1.73-1.60 (m, 1H), 0.96 (s, 9H).

### Example 42

### 1-(3-{[tert-Butyl(diphenyl)silyl]oxy}phenyl)-2-(3′-methoxybiphenyl-3-yl)ethane-1,2-dione

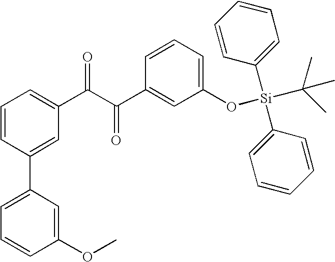

The title compound was synthesized as described for Example 14 starting from (3-{[tert-butyl(diphenyl)silyl]oxy}phenyl)[2-(3′-methoxybiphenyl-3-yl)-1,3-dithian-2-yl]methanol. The crude product was purified by flash chromatography with diisopropylether as eluent, to give 1g (73% yield) of the title compound. ¹H NMR (DMSO-d₆) δ 8.11-8.06 (m, 1H), 7.98-7.94 (m, 1H), 7.73-7.64 (m, 2H), 7.60 (dd, J=7.8, 1.5 Hz, 4H), 7.50-7.33 (m, 9H), 7.25-7.18 (m, 3H), 7.17-7.15 (m, 1H), 7.02 (dd, J=8.0, 2.3 Hz, 1H), 3.84 (s, 3H), 1.04 (s, 9H).

### Example 43

### 5-(3-Hydroxyphenyl)-5-(3′-methoxybiphenyl-3-yl)-3-methyl-2-thioxoimidazolidin-4-one

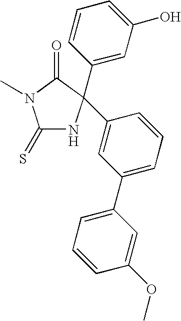

The title compound was synthesized as described for Example 15 starting from 1-(3-{[tert-butyl(diphenyl)silyl]oxy}phenyl)-2-(3′-methoxybiphenyl-3-yl)ethane-1,2-dione, except that the product was used without purification. The product was dried at 35° C. in a vacuum-cabinet over night, to afford 77% yield of the title compound. ¹H NMR (DMSO-d₆) δ 11.63 (s, 1H), 9.61 (s, 1H), 7.71-7.66 (m, 1H), 7.64-7.61 (m, 1H), 7.52 (t, J=7.8 Hz, 1H), 7.43-7.36 (m, 2H), 7.24-7.12 (m, 3H), 6.97 (dd, J=7.9, 2.1 Hz, 1H), 6.77-6.72 (m, 3H), 3.81 (s, 3H), 3.19 (s, 3H); MS (ESI) m/z 403 [M−1]⁻.

### Example 44

### 3-[4-(3′-Methoxybiphenyl-3-yl)-1-methyl-5-oxo-2-thioxoimidazolidin-4-yl]phenyl trifluoromethanesulfonate

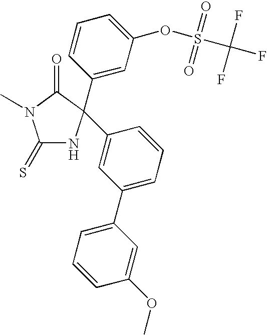

A mixture of 5-(3-hydroxyphenyl)-5-(3′-methoxybiphenyl-3-yl)-3-methyl-2-thioxoimidazolidin-4-one (0.1 g, 0.24 mmol), 1,1,1-trifluoro-N-phenyl-N-[(trifluoromethyl)sulfonyl]methanesulfonamide (0.088 g, 0.24 mmol), aqueous potassium carbonate (0.21 g, 1.50 mmol) in anhydrous tetrahydrofuran (3 mL) was heated in a microwave reactor at 120° C. for 6 minutes. The mixture was evaporated, ethyl acetate was added and undissolved material was removed by filtration. The solution was evaporated to give 125 mg (yield 97%) of the title compound. ¹H NMR (CDCl₃, 600 MHz) δ 8.07 (s, 1H), 7.61 (d, J=7.7 Hz, 1H), 7.53-7.44 (m, 4H), 7.37-7.30 (m, 3H), 7.28-7.25 (m, 1H), 7.09 (d, J=7.7 Hz, 1H), 7.05-7.02 (m, 1H), 6.91 (dd, J=8.3, 2.6 Hz, 1H), 3.85 (s, 3H), 3.36 (s, 3H); MS (ESI) m/z 535.0 [M−1]⁻.

### Example 45

### 3-[2-Amino-4-(3′-methoxybiphenyl-3-yl)-1-methyl-5-oxo-4,5-dihydro-1H-imidazol-4-yl]phenyl trifluoromethanesulfonate hydrochloride

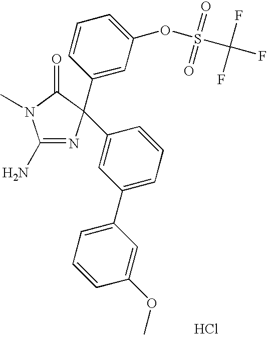

t-Butylhydroperoxide (70% in water, 0.40 mL) was added to a solution of 3-[4-(3′-methoxybiphenyl-3-yl)-1-methyl-5-oxo-2-thioxoimidazolidin-4-yl]phenyl trifluoromethanesulfonate (160 mg, 0.30 mmol) in ethanol (5 mL) and aqueous ammonia (25%, 2.5 mL). The resulting mixture was stirred at 35° C. over night and then poured into water (5 mL) and extracted with dichloromethane. The combined organic extracts were dried over magnesium sulfate, and the solvents were removed in vacuo. The residue was purified by flash chromatography with acetonitril/triethylamine (90:10), as eluent to give 35 mg of the base. The base was dissolved in dichloromethane and treated with hydrochloric acid in diethyl ether (4 M). The mixture was concentrated in vacuo and the residue was dried at 45° C. in a vacuum-cabinet over night to afford 45 mg (27% yield) of the title compound. ¹H NMR (DMSO-d₆) δ 11.79 (br s, 1H), 9.73 (br s, 2H), 7.74 (d, J=8.0 Hz, 1H), 7.68 (d, J=8.3 Hz, 1H), 7.63-7.58 (m, 3H), 7.56-7.52 (m, 2H), 7.42-7.34 (m, 2H), 7.19-7.13 (m, 2H), 6.97 (dd, J=8.2, 1.9 Hz, 1H), 3.81 (s, 3H), 3.20 (s, 3H); MS (ESI) m/z 518 [M−1]⁻.

### Example 46

### 3-[4-(3′-Methoxybiphenyl-3-yl)-1-methyl-5-oxo-2-thioxoimidazolidin-4-yl]phenyl methanesulfonate

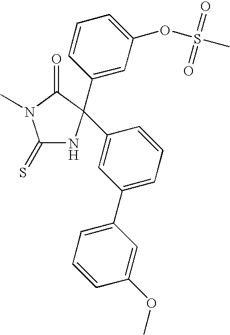

The title compound was synthesized in 95% yield as described for Example 29 starting from 5-(3-hydroxyphenyl)-5-(3′-methoxybiphenyl-3-yl)-3-methyl-2-thioxoimidazolidin-4-one. ¹H NMR (CDCl₃, 600 MHz) δ 7.96 (br s, 1H), 7.60 (d, J=7.7 Hz, 1H), 7.49-7.45 (m, 3H), 7.40 (d, J=8.3 Hz, 1H), 7.37-7.33 (m, 2H), 7.32-7.30 (m, 1H), 7.30-7.27 (m, 1H), 7.10 (d, J=7.7 Hz, 1H), 7.06-7.03 (m, 1H), 6.91 (dd, J=8.0, 2.0 Hz, 1H), 3.85 (s, 3H), 3.36 (s, 3H), 3.15 (s, 3H); MS (ESI) m/z 481 [M−1]⁻.

### Example 47

### 3-[2-Amino-4-(3′-methoxybiphenyl-3-yl)-1-methyl-5-oxo-4,5-dihydro-1H-imidazol-4-yl]phenyl methanesulfonate hydrochloride

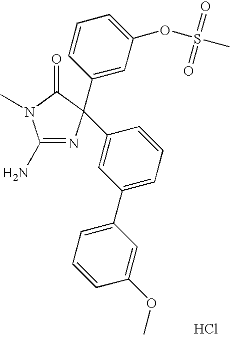

The title compound was synthesized as described in Example 45 starting from 3-[4-(3′-methoxybiphenyl-3-yl)-1-methyl-5-oxo-2-thioxoimidazolidin-4-yl]phenyl methanesulfonate, except that the formed hydrochloride salt was precipitated by addition of diethyl ether and the product was collected by filtration, yield 35%. ¹H NMR (DMSO-d₆) δ 11.71 (br s, 1H), 9.67 (br s, 2H), 7.73 (d, J=7.8 Hz, 1H), 7.65-7.57 (m, 2H), 7.54 (t, J=7.9 Hz, 1H), 7.48-7.42 (m, 2H), 7.42-7.35 (m, 3H), 7.20-7.14 (m, 2H), 6.97 (dd, J=8.0, 2.3 Hz, 1H), 3.81 (s, 3H), 3.41 (s, 3H), 3.20 (s, 3H); MS (ESI) m/z 464.0 [M−1]⁻.

### Example 48

### 5-(4,4,5,5-Tetramethyl-1,3,2-dioxaborolan-2-yl)-2,3-dihydro-1-benzofuran

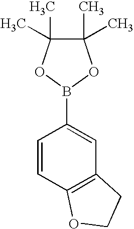

The title compound was synthesized as described for Example 2 in 99% yield starting from 5-bromo-2,3-dihydro-1-benzofuran. ¹H-NMR (DMSO-d₆): δ 7.52 (s, 1H), 7.40-7.45 (m, 1H), 6.75 (d, J=7.8 Hz, 1H), 4.53 (t, J=8.8 Hz, 2H), 3.16 (t, J=8.8 Hz, 2H), 1.26 (s, 6H), 1.16 (s, 6H); MS (CI) m/z 247 [M+1]⁺.

### Example 49

### 2-Amino-5-[3-(2,3-dihydro-1-benzofuran-5-yl)phenyl]-3-methyl-5-phenyl-3,5-dihydro-4H-imidazol-4-one

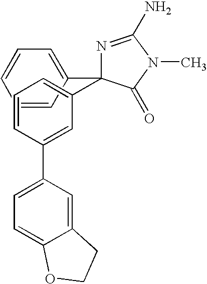

The title compound was synthesized as described for Example 5 in 20% yield starting from 2-amino-5-(3-bromo-phenyl)-3-methyl-5-phenyl-3,5-dihydro-imidazol-4-one and 5-(4,4,5,5-tetramethyl-1,3,2-dioxaborolan-2-yl)-2,3-dihydro-1-benzofuran. MS (ESI) m/z 384 [M+1]⁺.

### Example 50

### 2-Amino-5-[3-(2,3-dihydro-1-benzofuran-5-yl)phenyl]-3-methyl-5-phenyl-3,5-dihydro-4H-imidazol-4-one hydrochloride

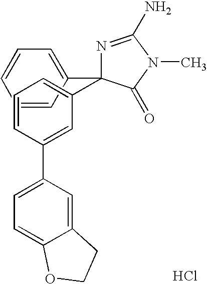

The title compound was synthesized as described for Example 6 in 99% yield starting from 2-Amino-5-[3-(2,3-dihydro-1-benzofuran-5-yl)phenyl]-3-methyl-5-phenyl-3,5-dihydro-4H-imidazol-4-one. ¹H NMR (DMSO-d₆) δ 11.61 (s, 1H), 9.61 (s, 2H), 7.27-7.66 (m, 11H), 6.85 (d, J=8.3 Hz, 1H), 4.56 (t, J=8.8 Hz, 2H), 3.23 (t, J=8.8 Hz, 2H, obscured by signal at 3.21), 3.21 (s, 3H); MS (ESI) m/z 384 [M+1]⁺.

### Example 51

### 2,3-Dihydro-1-benzofuran-5-yl(2-phenyl-1,3-dithian-2-yl)methanol

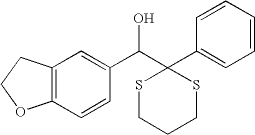

The title compound was synthesized as described for Example 13 starting from 2-Phenyl-[1,3]-dithiane and 2,3-dihydro-benzofuran-5-carbaldehyde. The product was purified by flash chromatography (n-heptane/ethyl acetate). This gave 82% yield of the title compound. ¹H NMR (CDCl₃) δ 7.73 (ddd, J=6.38, 1.71, 1.52 Hz, 2H), 7.28-7.36 (m, 3H), 6.71 (s, 1H), 6.51-6.60 (m, 2H), 4.94 (s, 1H), 4.53 (t, J=8.84 Hz, 2H), 3.08 (t, J=8.72 Hz, 2H), 2.65-2.77 (m, 4H), 1.89-2.00 (m, 2H).

### Example 52

### 1-(2,3-Dihydro-1-benzofuran-5-yl)-2-phenylethane-1,2-dione

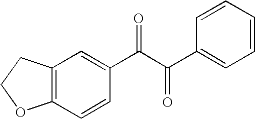

The title compound was synthesized in 65% yield as described for Example 14 starting from (2,3-Dihydro-benzofuran-5-yl)-(2-phenyl-[1,3]dithian-2-yl)-methanol. ¹H NMR (CDCl₃) δ 7.96-8.02 (m, 2H), 7.87 (d, J=1.52 Hz, 1H), 7.81 (dd, J=8.46, 1.89 Hz, 1H), 7.62-7.69 (m, 1H), 7.48-7.55 (m, 2H), 6.86 (d, J=8.34 Hz, 1H), 4.70 (t, J=8.84 Hz, 2H), 3.27 (t, J=8.72 Hz, 2H); MS (CI) m/z 253 [M+1]⁺.

### Example 53

### 5-(2,3-Dihydro-1-benzofuran-5-yl)-3-methyl-5-phenyl-2-thioxoimidazolidin-4-one

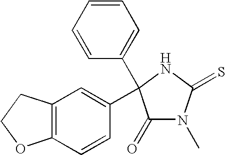

The title compound was synthesized as described in Example 3 starting from 1-(2,3-dihydro-1-benzofuran-5-yl)-2-phenylethane-1,2-dione, except that the workup was different: Water (30 mL) was added and pH adjusted to 5 with concentrated hydrochloric acid. The product was extracted with dichloromethane (3×30 mL). The combined organic phases were concentrated in vacuo and the product was purified by flash chromatography (n-heptane/ethyl acetate) to give 71% yield of the title compound. ¹H NMR (CDCl₃) δ 7.46 (s, 1H), 7.33-7.42 (m, 5H), 7.11 (s, 1H), 7.02 (dd, J=8.34, 2.27 Hz, 1H), 6.76 (d, J=8.59 Hz, 1H), 4.60 (t, J=8.72 Hz, 2H), 3.34 (s, 3H), 3.19 (t, J=8.72 Hz, 2H); MS (ESI) m/z 323 [M−1]⁻.

### Example 54

### 2-Amino-5-(2,3-dihydro-1-benzofuran-5-yl)-3-methyl-5-phenyl-3,5-dihydro-4H-imidazol-4-one

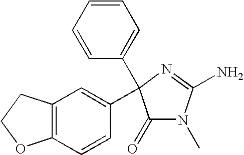

The title compound was synthesized as described for Example 4 starting from 5-(2,3-dihydro-1-benzofuran-5-yl)-3-methyl-5-phenyl-2-thioxoimidazolidin-4-one, except that the reaction was run for 3 h. The product was purified by flash chromatography (n-heptane/ethyl acetate). This gave 84% yield of the title compound. ¹H NMR (CDCl₃) δ ppm 7.43-7.49 (m, 2H), 7.28-7.36 (m, 3H), 7.24 (s, 1H), 7.18 (dd, J=8.34, 2.02 Hz, 1H), 6.72 (d, J=8.59 Hz, 1H), 4.55 (t, J=8.72 Hz, 2H), 3.16 (t, 2H), 3.14 (s, 3H); MS (ESI) m/z 308 [M+1]⁺.

### Example 55

### 2-Amino-5-(2,3-dihydro-1-benzofuran-5-yl)-3-methyl-5-phenyl-3,5-dihydro-4H-imidazol-4-one hydrochloride

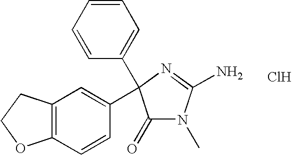

The title compound was synthesized as described for Example 6 starting from 2-amino-5-(2,3-dihydro-1-benzofuran-5-yl)-3-methyl-5-phenyl-3,5-dihydro-4H-imidazol-4-one

### Examples 56 and 57

### (R) and (S) 4-[2-amino-4-(3′-methoxybiphenyl-3-yl)-1-methyl-5-oxo-4,5-dihydro-1H-imidazol-4-yl]phenyl methanesulfonate hydrochloride

Chromatographic separation of the enantiomers of 4-[2-amino-4-(3′-methoxybiphenyl-3-yl)-1-methyl-5-oxo-4,5-dihydro-1H-imidazol-4-yl]phenyl methanesulfonate hydrochloride

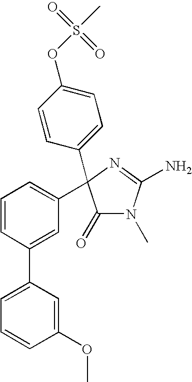

4-[2-amino-4-(3′-methoxybiphenyl-3-yl)-1-methyl-5-oxo-4,5-dihydro-1H-imidazol-4-yl]phenyl methanesulfonate hydrochloride (50 mg) was dissolved in ethanol (7 mL) and the resulting solution was divided into two equal portions. Chiral HPLC separation was carried out on a Chiralpak AD column (21.2×250 mm), using heptane:ethanol (70:30) as eluent at a flow rate of 12 mL/min. Detection was monitored at 254 nm and the two isomers were collected and concentrated in vacuo.

Isomer 1, Example 56, the first enantiomer to elute (23 mg, 50 μmol), was dissolved in chloroform (1 mL) and treated with hydrochloric acid (1.0 M in diethyl ether, 50 μL, 50 μmol) to generate the hydrochloric salt. The resulting mixture was concentrated to dryness to give 23 mg of (S) 4-[2-amino-4-(3′-methoxybiphenyl-3-yl)-1-methyl-5-oxo-4,5-dihydro-1H-imidazol-4-yl]phenyl methanesulfonate hydrochloride. ¹H NMR (CDCl₃) δ 7.71 (t, J=1.8 Hz, 1H), 7.64-7.61 (m, 2H), 7.53-7.45 (m, 2H), 7.42-7.32 (m, 2H), 7.27-7.23 (m, 2H), 7.16-7.13 (m, 1H), 7.10-7.08 (m, 1H), 6.91-6.88 (m, 1H), 3.86 (s, 3H), 3.15 (s, 3H), 3.12 (s, 3H); MS (ESI) m/z 466 [M+1]⁺.

Isomer 2, Example 57, the last enantiomer to elute, (25 mg, 55 μmol) was treated in the same manner as isomer 1 to generate 25 mg of (R) 4-[2-amino-4-(3′-methoxybiphenyl-3-yl)-1-methyl-5-oxo-4,5-dihydro-1H-imidazol-4-yl]phenyl methanesulfonate hydrochloride. ¹H NMR (CDCl₃) δ 7.72 (t, J=1.6 Hz, 1H), 7.65-7.60 (m, 2H), 7.54-7.45 (m, 2H), 7.42-7.31 (m, 2H), 7.27-7.23 (m, 2H), 7.17-7.13 (m, 1H), 7.10-7.08 (m, 1H), 6.92-6.87 (m, 1H), 3.86 (s, 3H), 3.14 (s, 3H), 3.12 (s, 3H); MS (ESI) m/z 466 [M+1]⁺.

### Example 58

### 2-Amino-3-methyl-5-[3-(4-methyl-3,4-dihydro-2H-1,4-benzoxazin-7-yl)phenyl]-5-phenyl-3,5-dihydro-4H-imidazol-4-one

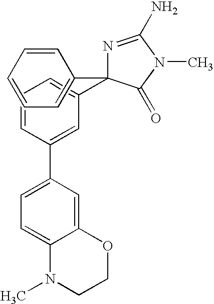

The title compound was synthesized as described for Example 5 starting from 2-amino-5-(3-bromophenyl)-3-methyl-5-phenyl-3,5-dihydro-4H-imidazol-4-one and 4-methyl-7-(4,4,5,5-tetramethyl-1,3,2-dioxaborolan-2-yl)-3,4-dihydro-2H-1,4-benzoxazine. The crude product was subjected to flash chromatography using a gradient of dichloromethane and methanol with 1% aqueous ammonia giving the title compound in 19% yield. ¹H NMR (DMSO-d₆) δ 7.62 (s, 1H), 7.48-7.19 (m, 8H), 7.00-6.95 (m, 1H), 6.85-6.83 (m, 1H), 6.75 (d, J=8.28 Hz, 1H), 4.28-4.22 (m, 2H), 3.27-3.23 (m, 2H), 2.99 (s, 3H), 2.85 (s, 3H). MS (ESI) m/z 413 [M+1]⁺.

### Example 59

### 2-Amino-3-methyl-5-[3-(4-methyl-3,4-dihydro-2H-1,4-benzoxazin-7-yl)phenyl]-5-phenyl-3,5-dihydro-4H-imidazol-4-one hydrochloride

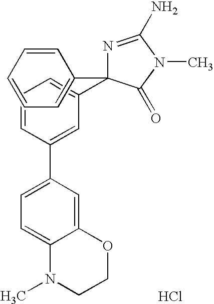

The title compound was synthesized as described for Example 6 in 99% yield starting from 2-amino-3-methyl-5-[3-(4-methyl-3,4-dihydro-2H-1,4-benzoxazin-7-yl)phenyl]-5-phenyl-3,5-dihydro-4H-imidazol-4-one. ¹H NMR (DMSO-d₆) δ ppm 11.64 (s, 1H), 9.63 (s, 2H), 7.60 (d, J=7.78 Hz, 1H), 7.55-7.51 (m, 1H), 7.48-7.36 (m, 6H), 7.25 (d, J=8.78 Hz, 1H), 7.06 (dd, J=8.28, 2.26 Hz, 1H), 6.95 (d, J=2.26 Hz, 1H), 6.76 (d, J=8.28 Hz, 1H), 4.27-4.20 (m, 2H), 3.29-3.24 (m, 2H), 3.21 (s, 3H), 2.86 (s, 3H). MS (ESI) m/z 413 [M+1]⁺.

### Example 60

### 2-Acetyl-5-chloro-1,2,3,4-tetrahydroisoquinoline

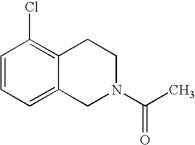

The title compound was synthesized as described for Example 1 in 100% yield starting from 5-chloro-1,2,3,4-tetrahydroisoquinoline. ¹H NMR (DMSO-d₆) δ ppm 7.32 (d, J=7.03 Hz, 1H), 7.26-7.17 (m, 2H), 4.67-4.61 (m, 2H), 3.70 (td, J=6.02, 2.51 Hz, 2H), 2.86 (t, J=6.02 Hz, 1H), 2.74 (t, J=6.02 Hz, 1H), 2.10-2.07 (m, 3H). MS (ESI) m/z 210 [M+1]⁺.

### Example 61

### 2-Acetyl-5-(4,4,5,5-tetramethyl-1,3,2-dioxaborolan-2-yl)-1,2,3,4-tetrahydroisoquinoline

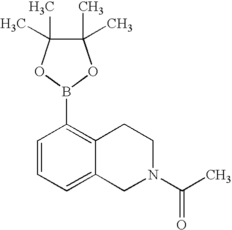

Tris(dibenzylideneacetone)dipalladium(0) chloroform adduct (21 mg, 0.02 mmol) and tricyclohexylphosphine (26 mg, 0.09 mmol) was dissolved in 1,2-dimethoxyethan (4 mL) and stirred at ambient temperature for 30 min. 2-acetyl-5-chloro-1,2,3,4-tetrahydroisoquinoline (140 mg, 0.67 mmol), 4,4,4′,4′,5,5,5′,5′-octamethyl-2,2′-bi-1,3,2-dioxaborolane (188 mg, 0.74 mmol) and potassium acetate (99 mg, 1.01 mmol) was added and the resulting mixture was irradiated in a microwave at 150° C. for 2 h. When cooled to ambient temperature the mixture was diluted with water (4 mL) and extracted with diethyl ether (3×5 mL). The combined organic extracts were passed through a silica pad. The product fractions were collected and concentrated to yield the title compound, 96 mg (32% yield). MS (ESI) m/z 302 [M+1]⁺.

### Example 62

### 5-[3-(2-Acetyl-1,2,3,4-tetrahydroisoquinolin-5-yl)phenyl]-2-amino-3-methyl-5-phenyl-3,5-dihydro-4H-imidazol-4-one

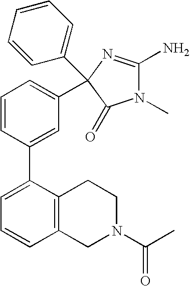

The title compound was synthesized as described for Example 5 in 23% yield starting from 2-amino-5-(3-bromophenyl)-3-methyl-5-phenyl-3,5-dihydro-4H-imidazol-4-one and 2-acetyl-5-(4,4,5,5-tetramethyl-1,3,2-dioxaborolan-2-yl)-1,2,3,4-tetrahydroisoquinoline. ¹H NMR (CDCl₃) δ ppm 7.54-7.07 (m, 12H), 4.77-4.59 (m, 2H), 3.76-3.43 (m, 2H), 3.16-3.06 (m, 3H), 2.82-2.60 (m, 2H), 2.22-2.08 (m, 3H). MS (ESI) m/z 439 [M+1]⁺.

### Example 63

### 5-[3-(2-Acetyl-1,2,3,4-tetrahydroisoquinolin-5-yl)phenyl]-2-amino-3-methyl-5-phenyl-3,5-dihydro-4H-imidazol-4-one hydrochloride

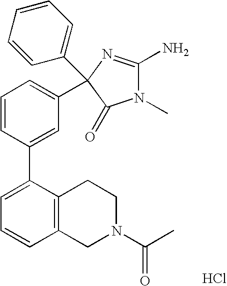

The title compound was synthesized as described for Example 6 in 89% yield starting from 5-[3-(2-acetyl-1,2,3,4-tetrahydroisoquinolin-5-yl)phenyl]-2-amino-3-methyl-5-phenyl-3,5-dihydro-4H-imidazol-4-one. MS (ESI) m/z 439 [M+1]⁺.

### Examples 64 and 65

### (R) and (S) 4-[2-amino-4-(3′-methoxybiphenyl-3-yl)-1-methyl-5-oxo-4,5-dihydro-1H-imidazol-4-yl]phenyl trifluoromethanesulfonate hydrochloride

Chromatographic separation of the enantiomers of 4-[2-amino-4-(3′-methoxybiphenyl-3-yl)-1-methyl-5-oxo-4,5-dihydro-1H-imidazol-4-yl]phenyl trifluoromethanesulfonate hydrochloride

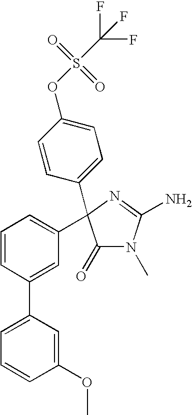

4-[2-amino-4-(3′-methoxybiphenyl-3-yl)-1-methyl-5-oxo-4,5-dihydro-1H-imidazol-4-yl]phenyl trifluoromethanesulfonate hydrochloride (16 mg) was dissolved in a mixture of isopropyl alcohol (2 mL) and heptane (2 mL). Chiral HPLC separation was carried out on a Chiralpak AD column (21.2×250 mm), using heptane:isopropyl alcohol (90:10) as eluent at a flow rate of 12 mL/min. Detection was monitored at 254 nm and the two isomers were collected and concentrated in vacuo.

Isomer 1, Example 64, the first enantiomer to elute: 4.4 mg. ¹H NMR (CDCl₃) δ 7.70-7.64 (m, 3H), 7.53-7.49 (m, 1H), 7.46-7.36 (m, 2H), 7.36-7.31 (m, 1H), 7.25-7.20 (m, 2H), 7.15-7.10 (m, 1H), 7.09-7.06 (m, 1H), 6.91-6.86 (m, 1H), 3.84 (s, 3H), 3.15 (s, 3H); MS (ESI) m/z 520 [M+1]⁺.

Isomer 2, Example 65, the last enantiomer to elute: 4.5 mg. ¹H NMR (CDCl₃) δ 7.71-7.64 (m, 3H), 7.53-7.48 (m, 1H), 7.45-7.38 (m, 2H), 7.37-7.31 (m, 1H), 7.25-7.19 (m, 2H), 7.15-7.10 (m, 1H), 7.09-7.05 (m, 1H), 6.92-6.86 (m, 1H), 3.84 (s, 3H), 3.13 (s, 3H); MS (ESI) nm/z 520 [M+1]⁺.

### Example 66

### (4-Methyl-3,4-dihydro-2H-1,4-benzoxazin-7-yl)(2-phenyl-1,3-dithian-2-yl)methanol

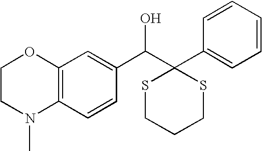

2-Phenyl-[1,3]-dithiane (527 mg, 2.69 mmol) was dissolved in 15 mL dry tetrahydrofuran under nitrogen and cooled to −78° C. n-Butyllithium (1.18 mL, 2.5 M) was added dropwise via syringe. The solution was stirred at −78° C. for 20 min and was then treated with 4-methyl-3,4-dihydro-2H-1,4-benzoxazine-7-carbaldehyde (500 mg, 2.82 mmol) via syringe. The reaction was stirred for another 20 min and was then allowed to reach room temperature. After one hour 20 mL ammonium chloride (saturated) was added to the reaction and it was then extracted with 2×20 mL dichloromethane. The combined organic phases were concentrated in vacuo and the product was isolated using flash chromatography (ethyl acetate/n-Heptane). This gave 290 mg of the title which was used in the next step without further purification. Yield: 14%; MS (ESI): m/z 374 [M+1]⁺.

### Example 67

### 1-(4-Methyl-3,4-dihydro-2H-1,4-benzoxazin-7-yl)-2-phenylethane-1,2-dione

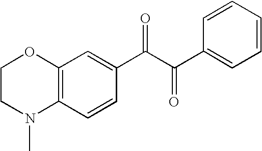

(4-Methyl-3,4-dihydro-2H-1,4-benzoxazin-7-yl)(2-phenyl-1,3-dithian-2-yl)methanol (590 mg, 0.79 mmol) from the step above was dissolved in 20 mL dichloromethane together with tert-butanol (204 mg, 2.76 mmol) under nitrogen. 1,1,1-Tris(acetyloxy)-1λ⁵,2-benziodoxol-3(1H)-one (755 mg, 1.78 mmol) was added to the solution and the reaction was stirred over night. The reaction was quenched with 10 mL aqueous sodium thiosulphate (1M) and 10 mL sodium bicarbonate (saturated) and diluted with dichloromethane. The layers were separated and the organic phase concentrated in vacuo. Purification using flash chromatography (ethyl acetate/n-Heptane) gave 150 mg of the title compound. Yield: 34%; MS (CI): m/z 282 [M+1]⁺.

### Example 68

### 3-Methyl-5-(4-methyl-3,4-dihydro-2H-1,4-benzoxazin-7-yl)-5-phenyl-2-thioxoimidazolidin-4-one

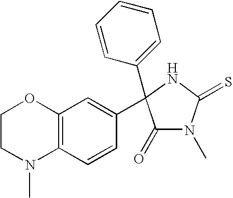

1-(4-Methyl-3,4-dihydro-2H-1,4-benzoxazin-7-yl)-2-phenylethane-1,2-dione (150 mg, 0.267 mmol) and N-methylthiourea (48.1 mg, 0.533 mmol) were dissolved in 7 mL dimethylsulphoxide and heated to 100° C. Aqueous potassium hydroxide (455 μl, 1.2 M) was added slowly to the solution via syringe. The reaction was stirred at 100° C. for 5 minutes and was then allowed to cool to ambient temperature. The solution was diluted with 30 mL water and carefully acidified to pH 5 with concentrated hydrochloric acid and was then extracted 3×30 mL dichloromethane. The combined organic phases were concentrated in vacuo and the product was isolated using preparative HPLC. The collected fractions were made basic using 10 mL 2M sodium hydroxide and extracted with 20 mL dichloromethane. This gave 33 mg of the title compound. Yield: 24%; MS (ESI): m/z 354 [M+1]⁺.

### Example 69

### 2-Amino-3-methyl-5-(4-methyl-3,4-dihydro-2H-1,4-benzoxazin-7-yl)-5-phenyl-3,5-dihydro-4H-imidazol-4-one acetic acid salt

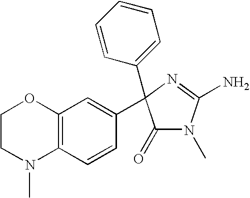

3-Methyl-5-(4-methyl-3,4-dihydro-2H-1,4-benzoxazin-7-yl)-5-phenyl-2-thioxoimidazolidin-4-one (22.1 mg, 0.0623 mmol) was diluted in 40 mL of 3:1 methanol/ammonium hydroxide and was then treated with tert-butyl hydroperoxide (84.7 mg, 0.94 mmol). The mixture was stirred at room temperature over night and was then placed in vacuo and concentrated until approximately 50% of the volume remained. The mixture was diluted with 30 mL water and 40 mL dichloromethane. The layers were separated and the organic phase was concentrated in vacuo and the product isolated using preparative HPLC. This gave 4 mg of the title product as the acetic acid salt. Yield: 16%; MS (ESI): m/z 337 [M+1]⁺; ¹H NMR (CDCl₃) δ ppm 7.54-7.45 (m, 2H), 7.36-7.28 (m, 3H), 6.86-6.80 (m, 2H), 6.59 (d, J=8.34 Hz, 1H), 4.27-4.17 (m, 2H), 3.28-3.19 (m, 2H), 3.15 (s, 3H), 2.85 (s, 3H).

### Example 70

### 6-Iodo-1,2,3,4-tetrahydronaphthalene

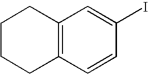

1,2,3,4-Tetrahydronaphthalene (500 mg, 3.79 mmol) and iodine (961 mg, 3.79 mmol) was dissolved in 50 mL dichloromethane and was cooled to 0° C. Silver nitrate (644 mg, 3.79 mmol) was added in portions to the solution while stirring. The reaction was allowed to reach room temperature and was stirred for 3 days. A yellow precipitate was filtered off and the filtrate was washed with 3×30 mL sodium bicarbonate (saturated). The organic phase was concentrated in vacuo and the remaining liquid was purified using kugelrohr distillation to give 538 mg of the title compound. Yield: 33%; MS (CI): m/z 258 [M+1]⁺; ¹H NMR (CDCl₃) δ ppm 7.41 (s, 1H), 7.39-7.37 (d, 1H), 6.81-6.78 (d, 1H), 2.76-2.64 (m, 4H), 1.78-1.72 (m, 4H).

### Example 71

### 6-(Phenylethynyl)-1,2,3,4-tetrahydronaphthalene

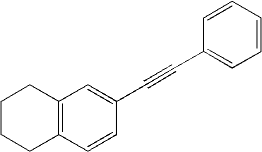

6-Iodo-1,2,3,4-tetrahydronaphthalene (1.2 g, 2.79 mmol), ethynylbenzene (475 mg, 4.65 mg), bis(triphenylphosphine)palladium(II) dichloride (16 mg, 0.023 mmol) and copper (I) iodide (4.4 mg, 0.023 mmol) were dissolved in 15 mL dry tetrahydrofuran and 15 mL dry triethylamine and flushed with nitrogen. The reaction was stirred at room temperature over night under a nitrogen atmosphere. The reaction was quenched by addition of 30 mL 2M hydrochloric acid and 30 mL dichloromethane and the layers were separated—The organic phase was concentrated in vacuo, diluted with dimethylsulphoxide and split into 7 fractions of 1750 μl which were purified using preparative HPLC to give 540 mg of the title compound. Yield 68%; MS (CI): m/z 233 [M+1]⁺.

### Example 72

### 1-Phenyl-2-(5,6,7,8-tetrahydronaphthalen-2-yl)ethane-1,2-dione

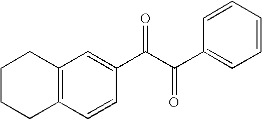

6-(Phenylethynyl)-1,2,3,4-tetrahydronaphthalene (540 mg, 2.32 mmol) and palladium (II) dichloride (41 mg, 0.23 mmol) were dissolved in 20 mL dimethylsulphoxide, heated to 140° C. and stirred over night. The reaction was allowed to cool to room temperature and was quenched with 50 mL water and 30 mL dichloromethane. The layers were separated and the organic phase was washed with 2×50 mL water and then concentrated in vacuo. The product was isolated using flash chromatography (ethyl acetate/n-Heptane) to give 337 mg of the title compound. Yield: 55%; MS (CI): m/z 263 [M−1]⁻; 1H NMR (CDCl₃) δ ppm 8.03-7.95 (d, 2H), 7.72-7.63 (m, 3H), 7.51 (t, J=7.83 Hz, 2H), 7.20 (d, J=7.83 Hz, 1H), 2.88-2.78 (m, 4H), 1.82 (dt, J=6.57, 3.28 Hz, 4H).

### Example 73

### 3-Methyl-5-phenyl-5-(5,6,7,8-tetrahydronaphthalen-2-yl)-2-thioxoimidazolidin-4-one

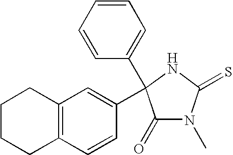

1-Phenyl-2-(5,6,7,8-tetrahydronaphthalen-2-yl)ethane-1,2-dione (337 mg, 1.27 mmol) and N-methylthiourea (230 mg, 2.55 mmol) were dissolved in 10 mL dimethylsulphoxide and heated to 100° C. Aqueous potassium hydroxide (2.18 mL, 1.2 M) was added slowly to the solution drop wise via syringe. The reaction was stirred at 100° C. for 5 min and was then allowed to cool to room temperature. The solution was diluted with 30 mL water and carefully acidified to pH 5 with concentrated hydrochloric acid and was then extracted with 3×30 mL dichloromethane and washed with 2×20 mL water. The organic phase was concentrated in vacuo. This gave 496 mg of the title compound. MS (ESI): m/z 337 [M+1]⁺; ¹H NMR (CDCl₃) δ ppm 7.40-7.32 (m, 5H), 7.07-7.05 (d, 1H), 7.01-6.97 (m, 2H), 3.33 (s, 4H), 2.74 (dd, J=6.44, 3.66 Hz, 4H), 1.79 (ddd, J=6.44, 3.41, 3.28 Hz, 4H)

### Example 74

### 2-Amino-3-methyl-5-phenyl-5-(5,6,7,8-tetrahydronaphthalen-2-yl)-3,5-dihydro-4H-imidazol-4-one hydrochloride

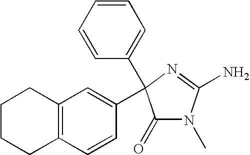

3-Methyl-5-phenyl-5-(5,6,7,8-tetrahydronaphthalen-2-yl)-2-thioxoimidazolidin-4-one (250 mg, 0.743 mmol) was diluted in 40 mL of 3:1 methanol/ammonium hydroxide and was then treated with tert-butyl hydroperoxide (1 g, 11.15 mmol). The mixture was stirred at room temperature over night and was then placed in vacuo and concentrated until approximately 50% of the volume remained. The mixture was diluted with 30 mL water and 40 mL dichloromethane. The layers were separated and the organic phase was concentrated in vacuo and the product isolated using flash chromatography (1:9 33% ammoniumhydroxide/methanol). This gave 208 mg of a solid. The product was further purified using preparative HPLC to give 90 mg of the product which was dissolved in 263 μl 1M HCl in ether to finally give 93 mg as the hydrochloride. Yield: 35%; MS (ESI): m/z 320 [M+1]⁺; ¹H NMR (DMSO-d₆) δ ppm 7.46-7.35 (d, 2H), 7.28 (t, J=7.45 Hz, 2H), 7.21 (d, J=7.07 Hz, 1H), 7.16-7.09 (m, 2H), 6.95 (d, J=8.59 Hz, 1H), 2.96 (s, 3H), 2.64 (m, 4H), 1.75-1.65 (m, 4H).

### Example 75

### 1-Acetyl-5-iodoindoline

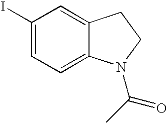

1-Acetyl-5-bromoindoline (250 mg, 1.04 mmol), sodium iodide (624 mg, 4.16 mmol) and copper (I) iodide (40 mg, 0.208 mmol) were dissolved in 30 mL dry dioxane and 500 μl triethylamine. N,N′-Dimethylethane-1,2-diamine (37 mg, 0.416 mmol) was added and the reaction was refluxed for 3 days. The solution was filtered through a short silica-plug and concentrated. The crude was diluted with 30 mL dichloromethane and 30 mL water and the layers were separated. The organic phase was concentrated in vacuo and this gave 247 mg product. Yield: 62%; MS (CI): m/z 288 [M+1]⁺.

### Example 76

### 1-Acetyl-5-(phenylethynyl)indoline

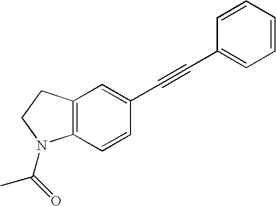

The title compound was synthesized as described for Example 71 starting from 1-acetyl-5-iodoindoline, with the exception that the product was purified using flash chromatography (ethyl acetate/n-Heptane) instead of preparative HPLC. Yield: 70%; MS (ESI): m/z 262 [M+1]⁺.

### Example 77

### 1-(1-Acetyl-2,3-dihydro-1H-indol-5-yl)-2-phenylethane-1,2-dione

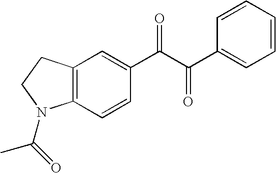

The title compound was synthesized as described for Example 72 starting from 1-acetyl-5-(phenylethynyl)indoline. This gave a product which was used in the next step without further purification. MS (ESI): m/z 293 [M+1]⁺.

### Example 78

### 5-(1-Acetyl-2,3-dihydro-1H-indol-5-yl)-3-methyl-5-phenyl-2-thioxoimidazolidin -4-one

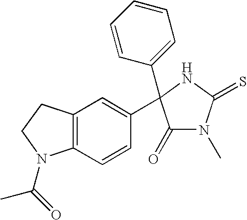

The title compound was synthesized as described for Example 73 starting from 1-(1-acetyl-2,3-dihydro-1H-indol-5-yl)-2-phenylethane-1,2-dione. This gave 80 mg of the product. Yield: 55%; MS (ESI): m/z 366 [M+1]⁺.

### Example 79

### 5-(1-Acetyl-2,3-dihydro-1H-indol-5-yl)-2-amino-3-methyl-5-phenyl-3,5-dihydro-4H-imidazol-4-one hydrochloride

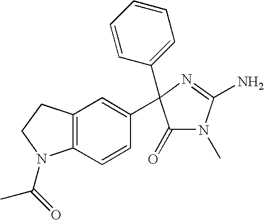

The title compound was synthesised as described for Example 74 starting from 5-(1-acetyl-2,3-dihydro-1H-indol-5-yl)-3-methyl-5-phenyl-2-thioxoimidazolidin-4-one. This gave 23 mg of the product as the hydrochloride salt. Yield: 29% MS (ESI): m/z 349 [M+1]⁺; ¹H NMR (CDCl₃) δ ppm 8.11 (d, 1H), 7.46-7.40 (d, 2H), 7.33-7.22 (m, 5H), 4.02 (t, J=8.59 Hz, 2H), 3.14-3.09 (m, 5H), 2.20 (s, 3H)

### Example 80

### 1-Acetyl-4-(4,4,5,5-tetramethyl-1,3,2-dioxaborolan-2-yl)indoline

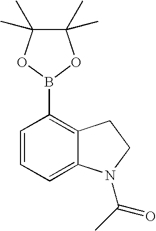

The title compound was synthesized as described for Example 2 in 70% yield starting from 1-acetyl-4-bromoindoline (described in Berrier, C. et al. New Journal of Chemistry 1987, 11(8-9), 605-9). Purified on silica gel column using acetonitrile as the eluent. ¹H-NMR (DMSO-d₆): δ 8.16 (d, J=7.8 Hz, 1H), 7.27 (dd, J=7.5, 1.0 Hz, 1H), 7.14 (t, J=7.6 Hz, 1H), 4.05 (t, J=8.5 Hz, 2H), 3.25 (t, J=8.5 Hz, 2H), 2.15 (s, 3H), 1.29 (s, 12H); MS (ESI) m/z 288 [M+1]⁺.

### Example 81

### 5-[3-(1-Acetyl-2,3-dihydro-1H-indol-4-yl)phenyl]-2-amino-3-methyl-5-phenyl-3,5-dihydro-4H-imidazol-4-one hydrochloride

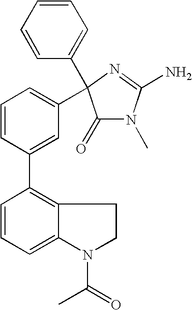

The title compound was synthesized as described for Example 5 and the salt of the base was prepared as described in Example 6 in 40% yield starting from 2-amino-5-(3-bromo-phenyl)-3-methyl-5-phenyl-3,5-dihydro-imidazol-4-one and 1-acetyl-4-(4,4,5,5-tetramethyl-1,3,2-dioxaborolan-2-yl)indoline. The crude product was purified on flash chromatography using acetonitrile/triethylamine, (95/5), as eluent. The formed hydrochloride salt precipitated after addition of diethyl ether. ¹H-NMR (DMSO-d₆): δ 11.62 (br s, 1H), 9.58 (br s, 2H), 8.10 (d, J=8.0 Hz, 1H), 7.56-7.52 (m, 2H), 7.50-7.36 (m, 7H), 7.26 (t, J=7.9 Hz, 1H), 7.00 (d, J=7.5 Hz, 1H), 4.06 (t, J=8.3 Hz, 2H), 3.20 (s, 3H), 3.14-3.06 (m, 2H), 2.17 (s, 3H); MS (ESI) m/z 425 [M+1]⁺.

### Example 82

### 8-(4,4,5,5-Tetramethyl-1,3,2-dioxaborolan-2-yl)chromane

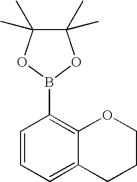

The title compound was synthesized as described for Example 2 in 56% yield starting from 8-bromochromane (described in Gerard H. Thomas et al. Tetrahedron Letters, 1998, 39, 2219-2222). Purification was on a silica gel column using dichloromethane/methanol, (95/5), as the eluent. ¹H-NMR (DMSO-d₆): δ 7.32 (dd, J=7.3, 1.5 Hz, 1H), 7.12 (dd, J=7.4, 1.6 Hz, 1H), 6.76 (t, J=7.4 Hz, 1H), 4.12 (t, J=5.0 Hz, 2H), 2.71 (t, J=6.5 Hz, 2H), 1.92-1.84 (m, 2H), 1.25 (s, 12H).

### Example 83

### 2-Amino-5-[3-(3,4-dihydro-2H-chromen-8-yl)phenyl]-3-methyl-5-phenyl-3,5-dihydro-4H-imidazol-4-one acetic acid salt

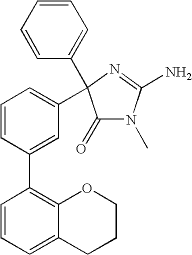

The title compound was synthesized as described for Example 5 starting from 2-amino-5-(3-bromo-phenyl)-3-methyl-5-phenyl-3,5-dihydro-imidazol-4-one and 8-(4,4,5,5-tetramethyl-1,3,2-dioxaborolan-2-yl)chromane. The crude product was subjected to acid/base-extraction followed by flash chromatography using acetonitrile/triethylamine, (95/5), as eluent, and then by preparative HPLC giving the title compound in 8% yield after freeze drying. ¹H-NMR (600 MHz, DMSO-d₆): δ 7.54 (s, 1H), 7.49 (d, J=7.4 Hz, 2H), 7.37 (d, J=7.7 Hz, 1H), 7.33-7.26 (m, 4H), 7.24-7.20 (m, 1H), 7.03 (d, J=7.4 Hz, 1H), 6.96 (d, J=6.6 Hz, 1H), 6.86 (t, J=7.5 Hz, 1H), 4.06 (t, J=5.0 Hz, 2H), 2.98 (s, 3H), 2.78 (t, J=6.4 Hz, 2H), 1.93-1.88 (m, 4H); MS (ESI) m/z 398 [M+1]⁺.

### Example 84

### 3-Methoxy-5-(4,4,5,5-tetramethyl-1,3,2-dioxaborolan-2-yl)phenol

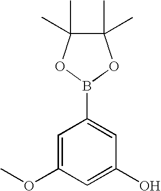

3-Chloro-5-methoxyphenol (1.59 g, 10.0 mmol), bis(pinacolato)diboron (2.79 g, 11.0 mmol), tris(dibenzylideneacetone)dipalladium (0.28 g, 0.30 mmol), potassium acetate (1.47 g, 15.0 mmol), tricyclohexylphosphine (0.33 g, 1.2 mmol) and 1,2-dimethoxyethane (12 mL) was irradiated in a microwave at 150° C. for 2 h. The reaction was performed three times, the combined reaction mixtures was poured into water and extracted with diethyl ether, the organic phase was dried over magnesium sulfate and concentrated. Purification by column chromatography using a gradient of methanol in chloroform (0-3%) gave 6.61 g (88% yield) of the title compound. ¹H NMR (CDCl₃) δ 6.85 (d, J=2.02 Hz, 1H), 6.78 (d, J=2.27 Hz, 1H), 6.45 (t, J=2.40 Hz, 1H), 4.59 (br s, 1H), 3.73 (s, 3H), 1.27 (s, 12H); MS (ES) m/z 251 [M+1]⁺.

### Example 85

### 3′-Hydroxy-5′-methoxybiphenyl-3-carbaldehyde

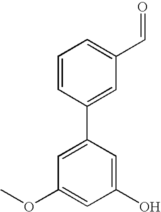

3-Bromobenzaldehyde (1.22 g, 6.6 mmol), 3-methoxy-5-(4,4,5,5-tetramethyl-1,3,2-dioxaborolan-2-yl)phenol (1.50 g, 6.0 mmol), [1,1′-bis(diphenylphosphino)ferrocene]-palladium(II) chloride dichloromethane adduct (0.25 g, 0.30 mmol), cesium carbonate (3.91 g, 12.0 mmol), 1,2-dimethoxyethane (9 mL), water (4.5 mL) and ethanol (1.5 mL) was irradiated in a microwave at 150° C. for 40 min. The reaction was performed three times, to the combined reaction mixtures was saturated ammonium chloride solution and water added. The mixture was extracted with dichloromethane, the organic phase was dried over magnesium sulfate and concentrated. Purification by column chromatography using heptane/ethyl acetate 3/1 afforded 2.47 g (60% yield) of the title compound. ¹H NMR (CDCl₃) δ 10.01 (s, 1H), 8.00 (t, J=1.64 Hz, 1H), 7.72-7.83 (m, 2H), 7.53 (t, J=7.58 Hz, 1H), 6.66-6.69 (m, 1H), 6.61-6.64 (m, 1H), 6.38 (t, J=2.27 Hz, 1H), 4.81 (br s, 1H), 3.78 (s, 3H); MS (ES) m/z 227 [M−1]⁻.

### Example 86

### 3′-{[tert-Butyl(diphenyl)silyl]oxy}-5′-methoxybiphenyl-3-carbaldehyde

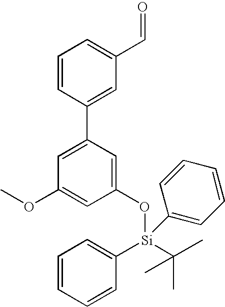

A solution of 3′-hydroxy-5′-methoxybiphenyl-3-carbaldehyde (2.46 g, 10.8 mmol), triethylamine (1.7 mL, 11.9 mmol), 4-dimethylaminopyridine (0.066 g, 0.54 mmol) and tert-butyl(chloro)diphenylsilane (3.1 mL, 11.9 mmol) in dichloromethane (50 mL) was stirred overnight. Dichloromethane was added and the organic phase was washed with water, dried over magnesium sulfate and concentrated. The residue was purified by column chromatography using a stepwise gradient of heptane/ethyl acetate 9/1-6/1 to give 4.44 g (88% yield) of the title compound. ¹H NMR (CDCl₃) δ 10.02 (s, 1H), 7.73-7.84 (m, 6H), 7.58-7.63 (m, 1H), 7.39-7.54 (m, 7H), 6.69-6.72 (m, 1H), 6.57-6.60 (m, 1H), 6.39 (t, J=2.15 Hz, 1H), 3.68 (s, 3H), 1.17 (s, 9H); MS (ES) m/z 467 [M+1]⁺.

### Example 87

### tert-Butyl{[3′-(1,3-dithian-2-yl)-5-methoxybiphenyl-3-yl]oxy}diphenylsilane

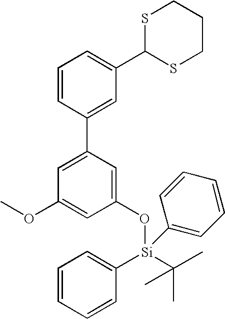

Boron trifluoride-diethyl etherate (4.8 mL, 38.0 mmol) was added dropwise to a mixture of 3′-{[tert-butyl(diphenyl)silyl]oxy}-5′-methoxybiphenyl-3-carbaldehyde (4.44 g, 9.5 mmol), 1,3-propanedithiol (0.91 mL, 9.0 mmol), 4 Å molecular sieves (8 g) in dichloromethane (50 mL) at 0° C. After stirring the reaction for 1.5 h at 0° C. a saturated sodium bicarbonate solution was added, the mixture was extracted with dichloromethane and the organic phase was washed consecutively with water, 10% potassium hydroxide solution and water, dried over magnesium sulfate and concentrated. Purification by column chromatography using heptane/ethyl acetate 6/1 afforded 4.52 g (85% yield) of the title compound. ¹H NMR (CDCl₃) δ 7.64-7.71 (m, 4H), 7.28-7.41 (m, 8H), 7.23 (t, J=7.58 Hz, 1H), 7.14-7.19 (m, 1H), 6.56-6.60 (m, 1H), 6.51-6.54 (m, 1H), 6.20 (t, J=2.27 Hz, 1H), 5.09 (s, 1H), 3.54 (s, 3H), 2.95-3.06 (m, 2H), 2.83-2.89 (m, 2H), 2.08-2.16 (m, 1H), 1.82-1.95 (m, 1H), 1.06 (s, 9H); MS (ES) m/z 557 [M+1]⁺.

### Example 88

### [2-(3′-{[tert-Butyl(diphenyl)silyl]oxy}-5′-methoxybiphenyl-3-yl)-1,3-dithian-2-yl](pyridin-4-yl)methanol

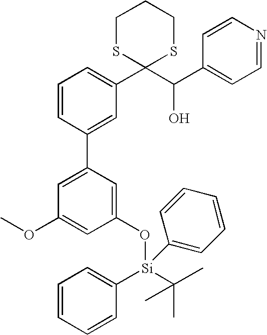

n-Butyllithium (1.6 M in hexane, 3.3 mL, 5.3 mmol) was added to a −78° C. solution of tert-butyl{[3′-(1,3-dithian-2-yl)-5-methoxybiphenyl-3-yl]oxy}diphenylsilane (2.68 g, 4.8 mmol) in tetrahydrofuran. After 40 min a solution of 4-pyridinecarboxaldehyde (0.51 mL, 5.3 mmol) in tetrahydrofuran (5 mL) was added and the reaction was stirred at −78° C. for 40 min and at room temperature for 1 h. The reaction was partitioned between water and dichloromethane, the organic phase was dried over magnesium sulfate and concentrated. Purification by column chromatography using chloroform gave 1.09 g (34% yield) of the title compound. ¹H NMR (CDCl₃) δ 8.11-8.18 (m, J=6.57 Hz, 2H), 7.58-7.63 (m, 1H), 7.50-7.58 (m, J=6.57 Hz, 5H), 7.11-7.28 (m, 9H), 6.97-7.02 (m, 1H), 6.36-6.41 (m, 1H), 6.28-6.32 (m, 1H), 6.19 (t, J=2.15 Hz, 1H), 4.86 (s, 1H), 3.46 (s, 3H), 2.39-2.65 (m, 4H), 1.68-1.78 (m, 2H), 0.92 (s, 9H); MS (ES) m/z 664 [M+1]⁺.

### Example 89

### 1-(3′-{[tert-Butyl(diphenyl)silyl]oxy}-5′-methoxybiphenyl-3-yl)-2-pyridin-4-ylethane-1,2-dione

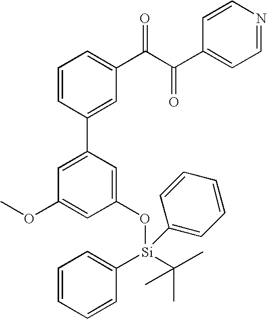

A mixture of [2-(3′-{[tert-butyl(diphenyl)silyl]oxy}-5′-methoxybiphenyl-3-yl)-1,3-dithian-2-yl](pyridin-4-yl)methanol (1.09 g, 1.6 mmol), tert-butanol (0.39 mL, 4.1 mmol), Dess-Martin periodinane (1.73 g, 4.1 mmol) was stirred overnight, saturated solutions of sodium bicarbonate and sodium thiosulfate was added. After 0.5 h the mixture was extracted with chloroform, the organic phase was dried over magnesium sulfate and concentrated. Purification by column chromatography using chloroform/methanol 99/1 afforded 0.27 g (29% yield) of the title compound. ¹H NMR (CDCl₃) δ 8.86-8.94 (m, 2H), 7.36-8.05 (m, 16H), 6.64-6.68 (m, 1H), 6.56-6.59 (m, 1H), 6.37 (t, J=2.27 Hz, 1H), 3.65 (s, 3H), 1.16 (s, 9H); MS (ES) m/z 572 [M+1]⁺.

### Example 90

### 5-(3′-Hydroxy-5′-methoxybiphenyl-3-yl)-3-methyl-5-pyridin-4-yl-2-thioxoimidazolidin-4-one

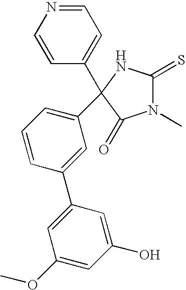

1-(3′-{[tert-Butyl(diphenyl)silyl]oxy}-5′-methoxybiphenyl-3-yl)-2-pyridin-4-ylethane-1,2-dione (0.27 g, 0.47 mmol), N-methylthiourea (0.085 g, 0.94 mmol) and dimethyl sulfoxide (2 mL) was kept at 100° C. for 5 min, a solution of potassium hydroxide (1 M, 0.99 mL) was added and the mixture was kept at 100° C. for an additional 5 minutes. Water was added followed by neutralization with hydrochloric acid (1 M). The mixture was extracted with dichloromethane, the organic phase was dried over magnesium sulfate and the solvent was evaporated. Purification by column chromatography using chloroform/methanol 98/2 afforded 0.16 g (85% yield) of the title compound. ¹H NMR (CDCl₃) δ 8.81 (br s, 1H), 8.50-8.55 (m, 2H), 7.44-7.49 (m, 1H), 7.33-7.37 (m, 2H), 7.28-7.31 (m, 2H), 7.20-7.24 (m, 1H), 6.48-6.52 (m, 1H), 6.45-6.47 (m, 1H), 6.32 (t, J=2.15 Hz, 1H), 3.70 (s, 3H), 3.24 (s, 3H); MS (ES) m/z 406 [M+1]⁺.

### Example 91

### 5-Methoxy-3′-(1-methyl-5-oxo-4-pyridin-4-yl-2-thioxoimidazolidin-4-yl)biphenyl-3-yl methanesulfonate

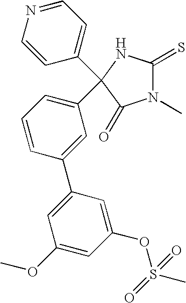

Methanesulfonyl chloride (0.034 mL, 0.44 mmol) was added to a 0° C. solution of 5-(3′-hydroxy-5′-methoxybiphenyl-3-yl)-3-methyl-5-pyridin-4-yl-2-thioxoimidazolidin-4-one (0.16 g, 0.40 mmol) and triethylamine (0.073 mL, 0.52 mmol) in dichloromethane (3 mL). After 2 h at 0° C., water was added and the mixture was extracted with dichloromethane, the organic phase was dried over magnesium sulfate and concentrated. This gave the title compound which was used without further purification. MS (ES) m/z 484 [M+1]⁺.

### Example 92

### 3′-(2-Amino-1-methyl-5-oxo-4-pyridin-4-yl-4,5-dihydro-1H-imidazol-4-yl)-5-methoxybiphenyl-3-yl methanesulfonate

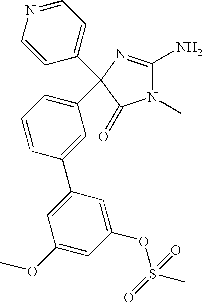

A mixture of 5-methoxy-3′-(1-methyl-5-oxo-4-pyridin-4-yl-2-thioxoimidazolidin-4-yl)biphenyl-3-yl methanesulfonate, methanol (3 mL), ammonium hydroxide (25%, 0.4 mL) and tert-butyl hydroperoxide (70% in water, 0.58 mL, 6.0 mmol) was stirred overnight, water (5 mL) was added and the mixture was extracted with dichloromethane, dried over magnesium sulfate and evaporated. Purification by column chromatography using a stepwise gradient of chloroform/methanol 95/5-85/15 gave 0.069 g (37% yield) of the title compound. ¹H NMR (CDCl₃) δ 8.50-8.54 (m, 2H), 7.70 (t, J=1.64 Hz, 1H), 7.53-7.59 (m, 1H), 7.45-7.51 (m, 3H), 7.39 (t, J=7.71 Hz, 1H), 7.01-7.07 (m, 2H), 6.78 (t, J=2.15 Hz, 1H), 5.43 (br s, 2H), 3.82 (s, 3H), 3.13 (s, 3H), 3.12 (s, 3H); MS (ES) m/z 467 [M+1]⁺.

### Example 93

### [2-(3′-{[tert-Butyl(diphenyl)silyl]oxy}-5′-methoxybiphenyl-3-yl)-1,3-dithian-2-yl](pyridin-2-yl)methanol

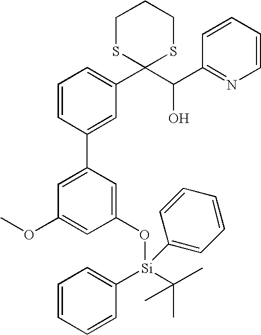

The title compound was synthesized as described for Example 88 in 27% yield using pyridine-2-carboxaldehyde as the starting material. ¹H NMR (CDCl₃) δ 8.37-8.44 (m, 1H), 7.70-7.81 (m, 5H), 7.58-7.63 (m, 1H), 7.36-7.51 (m, 7H), 7.27-7.35 (m, 1H), 7.20-7.25 (m, 1H), 7.11-7.17 (m, 1H), 6.76-6.83 (m, 1H), 6.49-6.52 (m, 1H), 6.42-6.47 (m, 1H), 6.31 (t, J=2.15 Hz, 1H), 5.15 (br s, 1H), 3.63 (s, 3H), 2.55-2.89 (m, 4H), 1.85-2.04 (m, 2H), 1.15 (s, 9H); MS (ES) m/z 664 [M+1]⁺.

### Example 94

### 1-(3′-{[tert-Butyl(diphenyl)silyl]oxy}-5′-methoxybiphenyl-3-yl)-2-pyridin-2-ylethane-1,2-dione

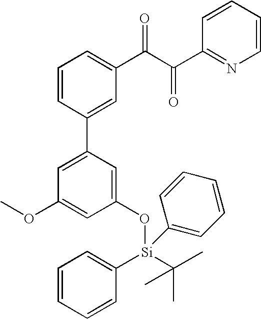

The title compound was synthesized as described for Example 89 in 57% yield starting from [2-(3′-{[tert-butyl(diphenyl)silyl]oxy}-5′-methoxybiphenyl-3-yl)-1,3-dithian-2-yl](pyridin-2-yl)methanol. MS (ES) m/z 572 [M+1]⁺.

### Example 95

### 5-(3′-Hydroxy-5′-methoxybiphenyl-3-yl)-3-methyl-5-pyridin-2-yl-2-thioxoimidazolidin-4-one

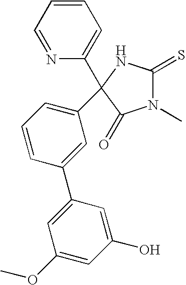

The title compound was synthesized as described for Example 90 in 76% yield starting from 1-(3′-{[tert-butyl(diphenyl)silyl]oxy}-5′-methoxybiphenyl-3-yl)-2-pyridin-2-ylethane-1,2-dione. MS (ES) m/z 406 [M+1]⁺.

### Example 96

### 5-Methoxy-3′-(1-methyl-5-oxo-4-pyridin-2-yl-2-thioxoimidazolidin-4-yl)biphenyl-3-yl methanesulfonate

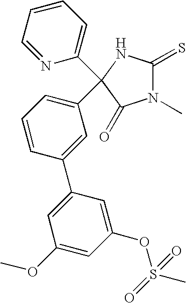

The title compound was synthesized as described for Example 91 starting from 5-(3′-hydroxy-5′-methoxybiphenyl-3-yl)-3-methyl-5-pyridin-2-yl-2-thioxoimidazolidin-4-one. MS (ES) m/z 484 [M+1]⁺.

### Example 97

### 3′-(2-Amino-1-methyl-5-oxo-4-pyridin-2-yl-4,5-dihydro-1H-imidazol-4-yl)-5-methoxybiphenyl-3-yl methanesulfonate

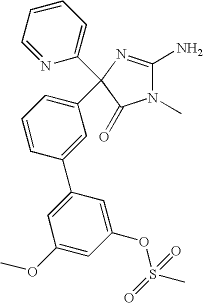

The title compound was synthesized as described for Example 92 in 6% yield starting from 5-methoxy-3′-(1-methyl-5-oxo-4-pyridin-2-yl-2-thioxoimidazolidin-4-yl)biphenyl-3-yl methanesulfonate. ¹H NMR (CDCl₃) δ 8.44-8.51 (m, 1H), 7.69-7.76 (m, 1H), 7.49-7.65 (m, 3H), 7.37-7.43 (m, 1H), 7.33 (t, J=7.71 Hz, 1H), 7.11-7.17 (m, 1H), 6.92-6.98 (m, 2H), 6.74 (t, J=2.15 Hz, 1H), 3.77 (s, 3H), 3.08 (s, 3H), 3.07 (s, 3H); MS (ES) m/z 467 [M+1]⁺.

### Example 98

### [2-(3′-{[tert-Butyl(diphenyl)silyl]oxy}-5′-methoxybiphenyl-3-yl)-1,3-dithian-2-yl](3-furyl)methanol

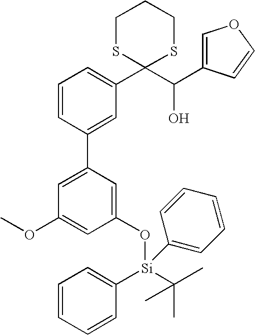

The title compound was synthesized as described for Example 88 in 84% yield using 3-furaldehyde as the starting material. ¹H NMR (CDCl₃) δ 7.64-7.74 (m, 6H), 7.28-7.40 (m, 6H), 7.24 (t, J=7.71 Hz, 1H), 7.14-7.19 (m, 1H), 7.04 (t, J=1.64 Hz, 1H), 6.98-7.01 (m, 1H), 6.49-6.52 (m, 1H), 6.44-6.46 (m, 1H), 6.24 (t, J=2.15 Hz, 1H), 5.72-5.75 (m, 1H), 4.85 (d, J=3.79 Hz, 1H), 3.56 (s, 3H), 2.53-2.71 (m, 4H), 1.80-1.88 (m, 2H), 1.06 (s, 9H); MS (ES) m/z 653 [M+1]⁺.

### Example 99

### 1-(3′-{[tert-Butyl(diphenyl)silyl]oxy}-5′-methoxybiphenyl-3-yl)-2-(3-furyl)ethane-1,2-dione

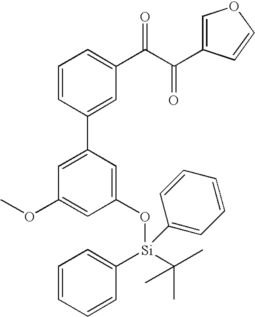

The title compound was synthesized as described for Example 89 in 67% yield starting from [2-(3′-{[tert-butyl(diphenyl)silyl]oxy}-5′-methoxybiphenyl-3-yl)-1,3-dithian-2-yl](3-furyl)methanol. ¹H NMR (CDCl₃) δ 8.21-8.24 (m, 1H), 8.07 (t, J=1.52 Hz, 1H), 7.93-7.98 (m, 1H), 7.73-7.80 (m, 4H), 7.53-7.63 (m, 2H), 7.37-7.52 (m, 7H), 6.96-6.99 (m, 1H), 6.66-6.70 (m, 1H), 6.58-6.62 (m, 1H), 6.35 (t, J=2.15 Hz, 1H), 3.65 (s, 3H), 1.16 (s, 9H); MS (ES) m/z 561 [M+1]⁺.

### Example 100

### 5-(3-Furyl)-5-(3′-hydroxy-5′-methoxybiphenyl-3-yl)-3-methyl-2-thioxoimidazolidin-4-one

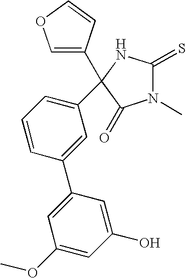

The title compound was synthesized as described for Example 90 in 84% yield starting from 1-(3′-{[tert-butyl(diphenyl)silyl]oxy}-5′-methoxybiphenyl-3-yl)-2-(3-furyl)ethane-1,2-dione. ¹H NMR (CDCl₃) δ 7.55-7.61 (m, 2H), 7.44-7.52 (m, 3H), 7.39-7.43 (m, 1H), 6.65-6.69 (m, 1H), 6.58-6.61 (m, 1H), 6.44 (t, J=2.15 Hz, 1H), 6.36 (dd, J=1.89, 0.88 Hz, 1H), 4.81 (s, 1H), 3.85 (s, 3H), 3.36 (s, 3H); MS (ES) m/z 393 [M−1]⁻.

### Example 101

### 3′-[4-(3-Furyl)-1-methyl-5-oxo-2-thioxoimidazolidin-4-yl]-5-methoxybiphenyl-3-yl methanesulfonate

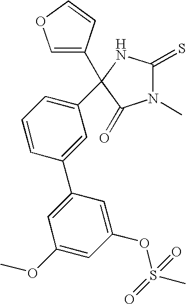

The title compound was synthesized as described for Example 91 starting from 5-(3-furyl)-5-(3′-hydroxy-5′-methoxybiphenyl-3-yl)-3-methyl-2-thioxoimidazolidin-4-one.

MS (ES) m/z 471 [M−1]⁻.

### Example 102

### 3′-[2-Amino-4-(3-furyl)-1-methyl-5-oxo-4,5-dihydro-1H-imidazol-4-yl]-5-methoxybiphenyl-3-yl methanesulfonate

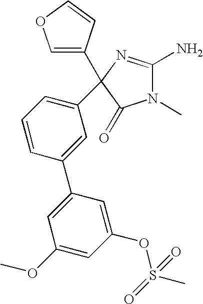

The title compound was synthesized as described for Example 92 in 59% yield starting from 3′-[4-(3-furyl)-1-methyl-5-oxo-2-thioxoimidazolidin-4-yl]-5-methoxybiphenyl-3-yl methanesulfonate. ¹H NMR (CDCl₃) δ 7.76 (t, J=1.64 Hz, 1H), 7.58-7.64 (m, 1H), 7.44-7.52 (m, 2H), 7.34-7.42 (m, 2H), 7.04 (d, J=2.02 Hz, 2H), 6.78 (t, J=2.15 Hz, 1H), 6.41 (dd, J=1.77, 0.76 Hz, 1H), 5.83 (br s, 2H), 3.82 (s, 3H), 3.12 (s, 3H), 3.09 (s, 3H); MS (ES) m/z 456 [M+1]⁺.

### Example 103

### [2-(3′-{[tert-Butyl(diphenyl)silyl]oxy}-5′-methoxybiphenyl-3-yl)-1,3-dithian-2-yl](1,3-thiazol-5-yl)methanol

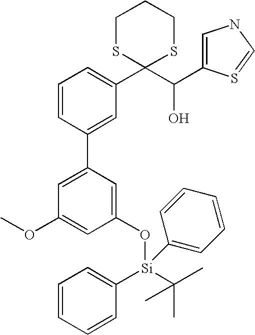

The title compound was synthesized as described for Example 88 in 54% yield using thiazole-5-carboxaldehyde as the starting material. ¹H NMR (CDCl₃) δ 9.05 (s, 1H), 7.71-7.82 (m, 5H), 7.30-7.55 (m, 10H), 6.62-6.65 (m, 1H), 6.48-6.51 (m, 1H), 6.41 (t, J=2.15 Hz, 1H), 5.36 (s, 1H), 3.70 (s, 3H), 2.66-2.95 (m, 4H), 1.94-2.06 (m, 2H), 1.15 (s, 9H); MS (ES) m/z 670 [M+1]⁺.

### Example 104

### 1-(3′-{[tert-Butyl(diphenyl)silyl]oxy}-5′-methoxybiphenyl-3-yl)-2-(1,3-thiazol-5-yl)ethane-1,2-dione

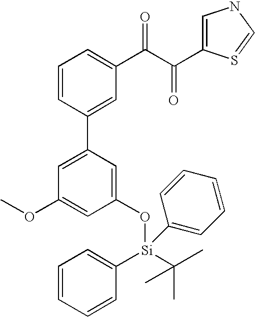

The title compound was synthesized as described for Example 89 in 58% yield starting from [2-(3′-{[tert-butyl(diphenyl)silyl]oxy}-5′-methoxybiphenyl-3-yl)-1,3-dithian-2-yl](1,3-thiazol-5-yl)methanol. ¹H NMR (CDCl₃) δ 9.17 (s, 1H), 8.64 (s, 1H), 8.09 (t, J=1.77 Hz, 1H), 7.96-8.02 (m, 1H), 7.74-7.80 (m, 4H), 7.60-7.66 (m, 1H), 7.38-7.54 (m, 7H), 6.66-6.70 (m, 1H), 6.58-6.62 (m, 1H), 6.36 (t, J=2.15 Hz, 1H), 3.65 (s, 3H), 1.16 (s, 9H); MS (ES) m/z 578 [M+1]⁺.

### Example 105

### 5-(3′-Hydroxy-5′-methoxybiphenyl-3-yl)-3-methyl-5-(1,3-thiazol-5-yl)-2-thioxoimidazolidin-4-one

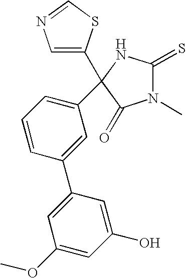

The title compound was synthesized as described for Example 90 in 65% yield starting from 1-(3′-{[tert-butyl(diphenyl)silyl]oxy}-5′-methoxybiphenyl-3-yl)-2-(1,3-thiazol-5-yl)ethane-1,2-dione. MS (ES) m/z 412 [M+1]⁺.

### Example 106

### 5-Methoxy-3′-[1-methyl-5-oxo-4-(1,3-thiazol-5-yl)-2-thioxoimidazolidin-4-yl]biphenyl-3-yl methanesulfonate

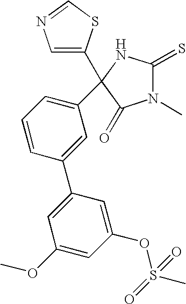

The title compound was synthesized as described for Example 91 starting from 5-(3′-hydroxy-5′-methoxybiphenyl-3-yl)-3-methyl-5-(1,3-thiazol-5-yl)-2-thioxoimidazolidin-4-one. MS (ES) m/z 490 [M+1]⁺.

### Example 107

### 3′-[2-Amino-1-methyl-5-oxo-4-(1,3-thiazol-5-yl)-4,5-dihydro-1H-imidazol-4-yl]-5-methoxybiphenyl-3-yl methanesulfonate

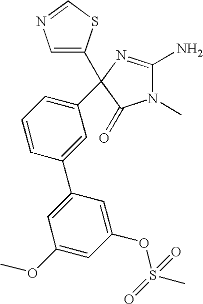

The title compound was synthesized as described for Example 92 in 22% yield starting from 5-methoxy-3′-[1-methyl-5-oxo-4-(1,3-thiazol-5-yl)-2-thioxoimidazolidin-4-yl]biphenyl-3-yl methanesulfonate. ¹H NMR (DMSO-d₆) δ 8.98 (s, 1H), 7.89 (s, 1H), 7.75-7.81 (m, 1H), 7.57-7.65 (m, 2H), 7.46 (t, J=7.71 Hz, 1H), 7.05-7.13 (m, 2H), 6.98 (t, J=2.15 Hz, 1H), 6.94 (s, 2H), 3.86 (s, 3H), 3.44 (s, 3H), 3.00 (s, 3H); MS (ES) m/z 473 [M+1]⁺.

### Example 108

### [2-(3′-{[tert-Butyl(diphenyl)silyl]oxy}-5′-methoxybiphenyl-3-yl)-1,3-dithian-2-yl](1,3-thiazol-4-yl)methanol

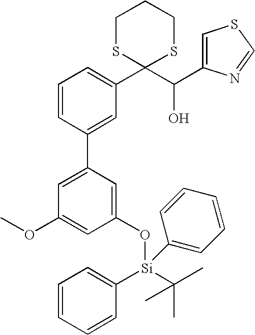

The title compound was synthesized as described for Example 88 in 66% yield using thiazole-4-carboxaldehyde as the starting material. MS (ES) m/z 670 [M+1]⁺.

### Example 109

### 1-(3′-{[tert-Butyl(diphenyl)silyl]oxy}-5′-methoxybiphenyl-3-yl)-2-(1,3-thiazol-4-yl)ethane-1,2-dione

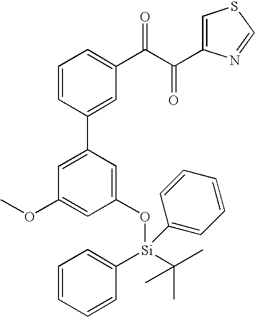

The title compound was synthesized as described for Example 89 in 63% yield starting from [2-(3′-{[tert-butyl(diphenyl)silyl]oxy}-5′-methoxybiphenyl-3-yl)-1,3-dithian-2-yl](1,3-thiazol-4-yl)methanol. MS (ES) m/z 578 [M+1]⁺.

### Example 110

### 5-(3′-Hydroxy-5′-methoxybiphenyl-3-yl)-3-methyl-5-(1,3-thiazol-4-yl)-2-thioxoimidazolidin-4-one

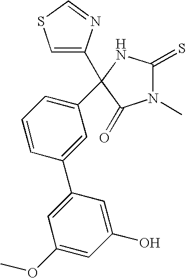

The title compound was synthesized as described for Example 90 in 93% yield starting from 1-(3′-{[tert-butyl(diphenyl)silyl]oxy}-5′-methoxybiphenyl-3-yl)-2-(1,3-thiazol-4-yl)ethane-1,2-dione. ¹H NMR (CDCl₃) δ 8.92 (d, J=2.02 Hz, 1H), 8.32 (s, 1H), 7.69 (d, J=2.02 Hz, 1H), 7.62-7.59 (m, 1H), 7.57-7.53 (m, 1H), 7.47-7.43 (m, 1H), 6.68-6.65 (m, 1H), 6.61-6.58 (m, 1H), 6.43 (t, J=2.27 Hz, 1H), 3.84 (s, 3H), 3.35 (s, 3H); MS (ES) m/z 412 [M+1]⁺.

### Example 111

### 5-Methoxy-3′-[1-methyl-5-oxo-4-(1,3-thiazol-4-yl)-2-thioxoimidazolidin-4-yl]biphenyl-3-yl methanesulfonate

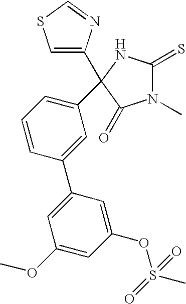

The title compound was synthesized as described for Example 91 starting from 5-(3′-hydroxy-5′-methoxybiphenyl-3-yl)-3-methyl-5-(1,3-thiazol-4-yl)-2-thioxoimidazolidin-4-one. MS (ES) m/z 490 [M+1]⁺.

### Example 112

### 3′-[2-Amino-1-methyl-5-oxo-4-(1,3-thiazol-4-yl)-4,5-dihydro-1H-imidazol-4-yl]-5-methoxybiphenyl-3-yl methanesulfonate

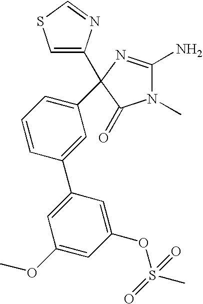

The title compound was synthesized as described for Example 92 in 49% yield starting from 5-methoxy-3′-[1-methyl-5-oxo-4-(1,3-thiazol-4-yl)-2-thioxoimidazolidin-4-yl]biphenyl-3-yl methanesulfonate. ¹H NMR (CDCl₃) δ 8.75 (d, J=2.02 Hz, 1H), 7.93-7.86 (m, 1H), 7.72-7.64 (m, 1H), 7.51-7.45 (m, 1H), 7.41-7.31 (m, 2H), 7.04-7.00 (m, 2H), 6.76 (t, J=2.15 Hz, 1H), 6.10 (br s, 2H), 3.79 (s, 3H), 3.11 (s, 3H), 3.10 (s, 3H); MS (ES) m/z 473 [M+1]⁺.

### Example 113

### 4-Bromo-1-fluoro-2-methoxybenzene

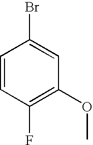

Aqueous hydrobromic acid (48%, 2.41 mL) was added to 4-fluoro-3-methoxyaniline (1.0 g, 7.1 mmol) in water (10 mL) and the resulting mixture was cooled to 0° C. in an ice bath. A solution of sodium nitrite (538 mg, 7.8 mmol) in water (5 mL) was added dropwise during 15 min while maintaining the temperature between 0-5° C. The resulting diazonium salt solution was added to a suspension of copper(I) bromide (1.12 g, 7.8 mmol) in water (5 mL) which had been pre-heated to 75° C. The mixture was shaken thoroughly, aqueous hydrobromic acid (48%, 12.07 mL) was added and the solution was stirred at ambient temperature for 16 h. Excess water was added and the product was extracted with diethyl ether and the combined organic extracts were washed with aqueous saturated sodium chloride, dried over magnesium sulfate, filtered and the solvent was evaporated in vacuo to give 1.02 g (70% yield) of the title compound: ¹H-NMR (DMSO-d₆): δ 7.36 (dd, J=7.78, 2.26 Hz, 1H), 7.23-7.17 (m, 1H), 7.14-7.09 (m, 1H), 3.86 (s, 3H); MS (EI) (m/z, %) 204, 206 (100), 189, 191 (23), 161, 163 (45)

### Example 114

### 2-(4-Fluoro-3-methoxyphenyl)-4,4,5,5-tetramethyl-1,3,2-dioxaborolane

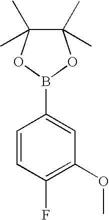

Anhydrous 1,2-dimethoxyethane (12 mL) was added to 4-bromo-1-fluoro-2-methoxybenzene (1.02 g, 5.0 mmol), tris(dibenzylideneaceton)dipalladium(0) (228 mg, 0.25 mmol), tricyclohexylphosphine (209 mg, 0.75 mmol), potassium acetate (732 mg, 7.5 mmol) and 4,4,4′,4′,5,5,5′,5′-octamethyl-2,2′-bi-1,3,2-dioxaborolane (1.14 g, 4.5 mmol), and the mixture was irradiated under an atmosphere of argon in a microwave at 150° C. for 1 h. When cooled to ambient temperature the mixture was filtered and the solvent was evaporated in vacuo to give the title compound as an oil: MS (EI) (m/z, %) 252 (81), 237 (44), 166 (78), 152 (100)

### Example 115

### 4-[(3-Bromophenyl)ethynyl]phenol

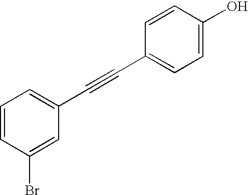

A solution of 1-bromo-3-ethynylbenzene (14.85 g, 82 mmol) in anhydrous tetrahydrofuran (34 mL) was added dropwise to a solution of 4-iodophenol (14.43 g, 65.6 mmol), copper(I) iodide (94 mg, 0.49 mmol) and bis(triphenylphosphine)palladium(II) dichloride (345 mg, 0.49 mmol) in a 2:1 mixture of tetrahydrofuran/triethylamine (230 mL). The reaction was stirred at ambient temperature under an atmosphere of argon for 16 h. More 4-iodophenol (541 mg, 2.5 mmol) was added and the mixture was stirred for another 3 h to force the reaction to completion. The reaction mixture was filtered and the solvent was evaporated in vacuo. The crude product was slurried in water (400 mL) and the product was extracted with ethyl acetate. The combined organic extracts were washed with aqueous saturated sodium chloride, dried over magnesium sulfate, filtered and the solvent was evaporated in vacuo. Purification by column chromatography, using 25-50% ethyl acetate in heptane as the eluent. The final product was dried at 40° C. in a vacuum-cabinet overnight to afford 15.25 g (68% yield) of the title compound: MS (ES) m/z 271, 273 [M−H]⁻.

### Example 116

### 1-(3-Bromophenyl)-2-(4-hydroxyphenyl)ethane-1,2-dione

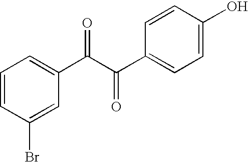

A solution of 4-[(3-bromophenyl)ethynyl]phenol (972 mg, 3.6 mmol) and palladium(II) chloride (68 mg, 0.38 mmol) in anhydrous dimethyl sulfoxide (10 mL) was heated in an oil bath at 140° C. for 3 h. The warm reaction mixture was poured into water/diethyl ether and the aqueous phase was extracted with diethyl ether. The combined organic extracts were washed with water, aqueous saturated sodium chloride, dried over magnesium sulfate, filtered and the solvent was evaporated in vacuo. The solid was purified by column chromatography, using 5-40% ethyl acetate in heptane as the eluent, to give 469 mg (43% yield) of the title compound: ¹H-NMR (DMSO-d₆): δ 10.93 (br s, 1H), 8.02 (t, J=1.63 Hz, 1H), 8.00-7.97 (m, 1H), 7.88-7.84 (m, 1H), 7.83-7.79 (m, 2H), 7.57 (t, J=7.91 Hz, 1H), 6.97-6.92 (m, 2H); MS (ES) m/z 303, 305 [M−H]⁻.

### Example 117

### 5-(3-Bromophenyl)-5-(4-hydroxyphenyl)-3-methyl-2-thioxoimidazolidin-4-one

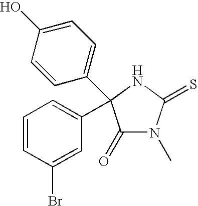

1-(3-Bromophenyl)-2-(4-hydroxyphenyl)ethane-1,2-dione (10.20 g, 33.4 mmol) and N-methylthiourea (6.03 g, 66.9 mmol) were divided equally into 11 microwave vials. Dimethyl sulphoxide (10 mL) and an aqueous solution of potassium hydroxide (5.2 mL, 1.2 M) was added to each vial. The vials were capped and irradiated in a microwave at 100° C. for 9 min. When cooled to ambient temperature the reaction mixtures were pooled, water (50 mL) and chloroform (60 mL) were added and pH was adjusted to pH 5 with a 2 M aqueous solution of hydrochloric acid. The aqueous phase was extracted with chloroform and the combined organic extracts were washed with aqueous saturated sodium chloride, dried over magnesium sulfate, filtered and the solvent was reduced in vacuo. Water was added and the extractive work up was repeated with diethyl ether instead of chloroform. The resulting crude product was purified by column chromatography, using 35% ethyl acetate in heptane as the eluent. Methanol was added and the solution was heated until the product started to crystallize. The slurry was filtered, the crystalline product was rinsed with methanol and finally dried at ambient temperature in a vacuum-cabinet overnight to afford 10.95 g (87% yield) of the title compound: ¹H-NMR (CDCl₃): δ 7.78 (br s, 1H), 7.55-7.50 (m, 2H), 7.32-7.28 (m, 2H), 7.17-7.12 (m, 2H), 6.86-6.82 (m, 2H), 5.31 (br s, 1H), 3.35 (s, 3H); MS (ES) m/z 375, 377 [M−H]⁻.

### Example 118

### 4-[4-(3-Bromophenyl)-1-methyl-5-oxo-2-thioxoimidazolidin-4-yl]phenyl methanesulfonate

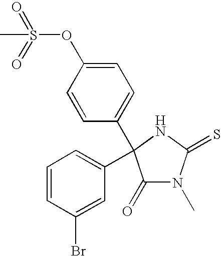

A solution of 5-(3-bromophenyl)-5-(4-hydroxyphenyl)-3-methyl-2-thioxoimidazolidin-4-one (88 mg, 0.23 mmol) and triethylamine (71 mg, 0.70 mmol) purged with argon was cooled to 0° C. Methanesulfonyl chloride (27 mg, 0.23 mmol) was added and the reaction mixture was stirred at 0° C. for 30 min and then at ambient temperature overnight. More methanesulfonyl chloride (13 mg, 0.12 mmol) and triethylamine (35 mg, 0.35 mmol) were added the following day. The mixture was stirred for 30 min resulting in full conversion to product. The solvent was evaporated in vacuo and the resulting crude product was purified by column chromatography, using 2-80% ethyl acetate in heptane as the eluent, to give 74 mg (70% yield) of the title compound: ¹H-NMR (DMSO-d₆): δ 11.71 (s, 1H), 7.65-7.61 (m, 1H), 7.54 (t, J=1.77 Hz, 1H), 7.46-7.37 (m, 6H), 3.41 (s, 3H), 3.18 (s, 3H); MS (ES) m/z 453, 455 [M−H]⁻.

### Example 119

### 4-[2-Amino-4-(3-bromophenyl)-1-methyl-5-oxo-4,5-dihydro-1H-imidazol-4-yl]phenyl methanesulfonate

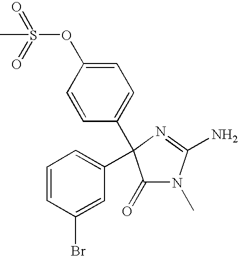

The title compound was synthesized as described for Example 4 in 63% yield starting from 4-[4-(3-bromophenyl)-1-methyl-5-oxo-2-thioxoimidazolidin-4-yl]phenyl methanesulfonate. Purification by column chromatography, using a mixture of ethyl acetate:aqueous ammonium hydroxide (33%):methanol, 94%:1%:5% respectively as the eluent: ¹H-NMR (DMSO-d₆): δ 7.64 (t, J=1.76 Hz, 1H), 7.56-7.51 (m, 2H) overlapping with 7.51-7.43 (m, 2H), 7.33-7.26 (m, 3H), 6.78 (br s, 2H), 3.35 (s, 3H), 3.00 (s, 3H); MS (ES) m/z 436, 438 [M−H]⁻.

### Example 120

### 4-[2-Amino-1-methyl-5-oxo-4-(3-pyridin-3-ylphenyl)-4,5-dihydro-1H-imidazol-4-yl]phenyl methanesulfonate 0.25 acetate

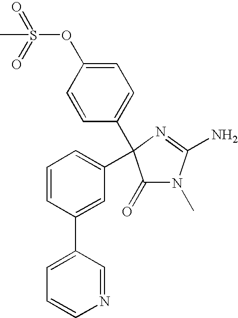

A mixture of 4-[2-amino-4-(3-bromophenyl)-1-methyl-5-oxoimidazolidin-4-yl]phenyl methanesulfonate (64 mg, 0.15 mmol), pyridin-3-ylboronic acid (23 mg, 0.19 mmol), [1,1′-bis(diphenylphosphino)ferrocene]palladium(II) chloride dichloromethane adduct (12 mg, 0.015 mmol), potassium carbonate (121 mg, 0.88 mmol) in anhydrous tetrahydrofuran (3 mL) was irradiated under an atmosphere of argon in a microwave at 150° C. for 2 h. When cooled to ambient temperature the mixture was filtered and dimethyl sulfoxide (500 μL) was added. The solution was concentrated in vacuo to remove tetrahydrofuran and purified by preparative HPLC to give 10 mg (12% yield) of the title compound: ¹H-NMR (DMSO-d₆): δ 8.77 (d, J=2.26 Hz, 1H), 8.57 (dd, J=4.77, 1.51 Hz, 1H), 7.97-7.93 (m, 1H), 7.79-7.77 (m, 1H), 7.63-7.57 (m, 3H), 7.54 (d, J=8.28 Hz, 1H), 7.51-7.41 (m, 2H), 7.29 (d, J=8.53 Hz, 2H), 3.35 (s, 3H), 3.00 (s, 3H), 1.90 (s, 0.67H); MS (ES) m/z 435 [M−H]⁻.

### Example 121

### 4-[4-(3-Bromophenyl)-1-methyl-5-oxo-2-thioxoimidazolidin-4-yl]phenyl 2-methoxyethanesulfonate

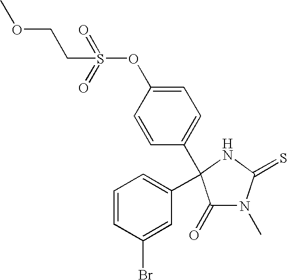

The title compound was synthesized as described for Example 118 in 71% yield starting from 5-(3-bromophenyl)-5-(4-hydroxyphenyl)-3-methyl-2-thioxoimidazolidin-4-one and 2-methoxyethanesulfonyl chloride) (described in: Matlack A. S. J. Org. Chem. 1958, 23, 729-731). Purification by column chromatography, using 40% ethyl acetate in heptane as the eluent: ¹H-NMR (DMSO-d₆): δ 11.69 (s, 1H), 7.65-7.61 (m, 1H), 7.56-7.52 (m, 1H), 7.45-7.37 (m, 6H), 3.85-3.76 (m, 4H), 3.28 (s, 3H), 3.18 (s, 3H); MS (ES) m/z 497, 499 [M−H]⁻.

### Example 122

### 4-[2-Amino-4-(3-bromophenyl)-1-methyl-5-oxo-4,5-dihydro-1H-imidazol-4-yl]phenyl 2-methoxyethanesulfonate

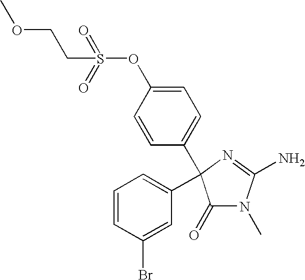

The title compound was synthesized as described for Example 4 in 52% yield starting from 4-[4-(3-bromophenyl)-1-methyl-5-oxo-2-thioxoimidazolidin-4-yl]phenyl 2-methoxyethanesulfonate. Purification by column chromatography, using a mixture of ethyl acetate:triethylamine:methanol, 94%:1%:5% respectively as the eluent: ¹H-NMR (DMSO-d₆): δ 7.63 (s, 1H), 7.55-7.43 (m, 4H), 7.32-7.24 (m, 3H), 6.78 (br s, 2H), 3.77 (s, 4H), 3.28 (s, 3H), 2.99 (s, 3H); MS (ES) m/z 480, 482 [M−H]⁻.

### Example 123

### 4-[4-(3-Bromophenyl)-1-methyl-5-oxo-2-thioxoimidazolidin-4-yl]phenyl propane-1-sulfonate

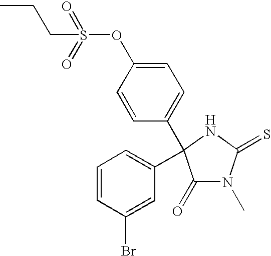

The title compound was synthesized as described for Example 118 in 86% yield starting from 5-(3-bromophenyl)-5-(4-hydroxyphenyl)-3-methyl-2-thioxoimidazolidin-4-one and propane-1-sulfonyl chloride. Purification by column chromatography, using 5% acetonitrile in dichloromethane as the eluent: ¹H-NMR (DMSO-d₆): δ 11.70 (s, 1H), 7.65-7.60 (m, 1H), 7.55-7.51 (m, 1H), 7.46-7.36 (m, 6H), 3.55-3.48 (m, 2H), 3.18 (s, 3H), 1.89-1.77 (m, 2H), 1.03 (t, J=7.40 Hz, 3H); MS (ES) m/z 481, 483 [M−H]⁻.

### Example 124

### 4-[2-Amino-4-(3-bromophenyl)-1-methyl-5-oxo-4,5-dihydro-1H-imidazol-4-yl]phenyl propane-1-sulfonate

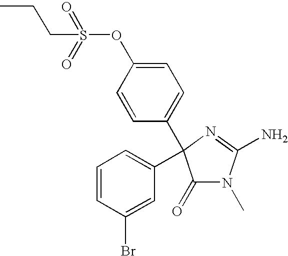

The title compound was synthesized as described for Example 4 in 65% yield starting from 4-[4-(3-bromophenyl)-1-methyl-5-oxo-2-thioxoimidazolidin-4-yl]phenyl propane-1-sulfonate. Purification by column chromatography, using 5% triethylamine in acetonitrile as the eluent: ¹H-NMR (DMSO-d₆): δ 7.64-7.62 (m, 1H), 7.55-7.43 (m, 4H), 7.32-7.24 (m, 3H), 6.79 (br s, 2H), 3.50-3.44 (s, 2H), 2.99 (s, 3H), 1.88-1.77 (m, 2H), 1.02 (t, J=7.40 Hz, 3H); MS (ES) m/z 466, 468 [M+H]⁺.

### Examples 125-169

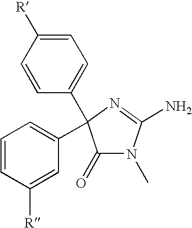

TABLE 1
Representative examples synthesized as described for 4-[2-Amino-1-methyl-5-
oxo-4-(3-pyridin-3-ylphenyl)-4,5-dihydro-1H-imidazol-4-yl]phenyl methanesulfonate 0.25
acetate

| | | | | [M − H]⁻ | |
|---|---|---|---|---|---|
| Ex | Chemical name | R′ | R″ | m/z | ¹H-NMR |
| 125 | 4-[2-Amino-4-(5′-chloro- 2′-methoxybiphenyl-3-yl)- 1-methyl-5-oxo-4,5- dihydro-1H-imidazol-4- yl]phenyl methanesulfonate 0.25 acetate | CH₃SO₃ | 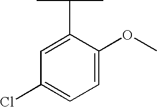 | 498, 500 | δ 7.63-7.57 (m, 3 H), 7.48-7.42 (m, 1 H), 7.38 (dd, J = 8.8, 2.8 Hz, 1 H) overlapping with 7.37-7.33 (m, 2 H), 7.33-7.27 (m, 2 H), 7.22 (d, J = 2.8 Hz, 1 H), 7.12 (d, J = 8.8 Hz, 1 H), 6.72 (br s, 2 H), 3.71 (s, 3 H), 3.35 (s, 3 H), 2.99 (s, 3 H), 1.91 (s, 0.48 H) |
| 126 | 4-[2-Amino-4-(5′-fluoro-2′- methylbiphenyl-3-yl)-1- methyl-5-oxo-4,5-dihydro- 1H-imidazol-4-yl]phenyl methanesulfonate | CH₃SO₃ | 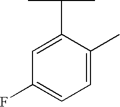 | 466 | δ 7.58 (d, J = 8.53 Hz, 2 H), 7.50-7.45 (m, 1 H), 7.45-7.37 (m, 2 H), 7.35-7.27 (m, 3 H), 7.24 (d, J = 7.28 Hz, 1 H), 7.14-7.06 (m, 1 H), 7.00-6.95 (m, 1 H), 6.71 (br s, 2 H), 3.35 (s, 3 H), 2.99 (s, 3 H), 2.11 (s, 3 H) |
| 127 | Methyl 3′-(2-amino-1- methyl-4-{4- [(methylsulfonyl)oxy]phen- yl}-5-oxo-4,5-dihydro-1H- imidazol-4-yl)biphenyl-3- carboxylate | CH₃SO₃ | 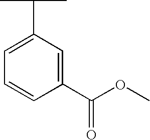 | 492 | δ 8.09 (s, 1 H), 7.96 (d, J = 7.78 Hz, 1 H), 7.85 (d, J = 8.28 Hz, 1 H), 7.80 (s, 1 H), 7.63 (t, J = 7.78 Hz, 1 H) overlapping with 7.62-7.54 (m, 4 H), 7.45 (t, J = 7.65 Hz, 1 H), 7.29 (d, J = 8.53 Hz, 2 H), 6.77 (br s, 2 H), 3.89 (s, 3 H), 3.35 (s, 3 H), 3.00 (s, 3 H) |
| 128 | 4-{2-Amino-4-[3-(1,3- benzodioxol-5-yl)phenyl]- 1-methyl-5-oxo-4,5- dihydro-1H-imidazol-4- yl}phenyl methanesulfonate | CH₃SO₃ | 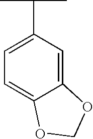 | 480 [M + H]⁺ | δ 7.68 (s, 1 H), 7.59 (d, J = 8.53 Hz, 2 H), 7.48-7.41 (m, 2 H), 7.39-7.33 (m, 1 H), 7.28 (d, J = 8.78 Hz, 2 H), 7.09 (s, 1 H), 7.00 (s, 2 H), 6.72 (br s, 2 H), 6.05 (s, 2 H), 3.35 (s, 3 H), 2.99 (s, 3 H) |
| 129 | 4-{2-Amino-4-[3-(1H- indol-5-yl)phenyl]-1- methyl-5-oxo-4,5-dihydro- 1H-imidazol-4-yl}phenyl methanesulfonate 0.25 acetate | CH₃SO₃ | 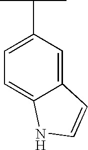 | 473 | δ 11.13 (s, 1 H), 7.78 (s, 1 H), 7.71-7.69 (m, 1 H), 7.61 (d, J = 8.78 Hz, 2 H), 7.52 (d, J = 7.28 Hz, 1 H), 7.46 (d, J = 8.53 Hz, 1 H), 7.43-7.34 (m, 3 H), 7.32-7.26 (m, 3 H), 6.73 (br s, 2 H), 6.50-6.48 (m, 1 H), 3.35 (s, 3 H), 3.00 (s, 3 H), 1.90 (s, 0.68 H) |
| 130 | 4-[2-Amino-4-(3′- cyanobiphenyl-3-yl)-1- methyl-5-oxo-4,5-dihydro- 1H-imidazol-4-yl]phenyl methanesulfonate | CH₃SO₃ | 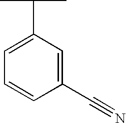 | 459 | δ 8.02 (s, 1 H), 7.89 (d, J = 7.78 Hz, 1 H), 7.86-7.79 (m, 2 H), 7.68 (t, J = 7.78 Hz, 1 H), 7.64-7.54 (m, 4 H), 7.45 (t, J = 7.78 Hz, 1 H), 7.29 (d, J = 8.78 Hz, 2 H), 6.75 (br s, 2 H), 3.35 (s, 3 H), 3.00 (s, 3 H) |
| 131 | 4-{2-Amino-1-methyl-5- oxo-4-[3′- (trifluoromethoxy)biphenyl- 3-yl]-4,5-dihydro-1H- imidazol-4-yl}phenyl methanesulfonate | CH₃SO₃ | 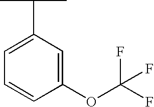 | 518 | δ 7.78 (s, 1 H), 7.64-7.52 (m, 6 H), 7.49 (s, 1 H), 7.44 (t, J = 7.78 Hz, 1 H), 7.40-7.36 (m, 1 H), 7.29 (d, J = 8.78 Hz, 2 H), 6.75 (br s, 2 H), 3.35 (s, 3 H), 3.00 (s, 3 H) |
| 132 | 4-{2-Amino-4-[3-(2- formyl-3-thienyl)phenyl]- 1-methyl-5-oxo-4,5- dihydro-1H-imidazol-4- yl}phenyl methanesulfonate | CH₃SO₃ | 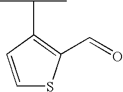 | 468 | δ 9.72 (d, J = 1.0 Hz, 1 H), 8.16 (dd, J = 5.0, 1.25 Hz, 1 H), 7.65-7.62 (m, 1 H), 7.62-7.56 (m, 3 H), 7.54-7.45 (m, 2 H), 7.36 (d, J = 5.02 Hz, 1 H), 7.32-7.27 (m, 2 H), 6.76 (br s, 2 H), 3.35 (s, 3 H), 2.99 (s, 3 H) |
| 133 | 4-{2-Amino-4-[3-(5- formyl-2-thienyl)phenyl]- 1-methyl-5-oxo-4,5- dihydro-1H-imidazol-4- yl}phenyl methanesulfonate | CH₃SO₃ | 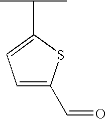 | 468 | δ 9.91 (s, 1 H), 8.03 (d, J = 4.02 Hz, 1 H), 7.88-7.86 (m, 1 H), 7.73-7.69 (m, 1 H), 7.63 (d, J = 3.76 Hz, 1 H), 7.61-7.53 (m, 3 H), 7.45 (t, J = 7.65 Hz, 1 H), 7.32-7.27 (m, 2 H), 6.79 (br s, 2 H), 3.35 (s, 3 H), 3.00 (s, 3 H) |
| 134 | 4-{2-Amino-4-[3-(2- chloropyridin-4- yl)phenyl]-1-methyl-5- oxo-4,5-dihydro-1H- imidazol-4-yl}phenyl methanesulfonate | CH₃SO₃ | 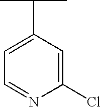 | 469, 471 | N/A |
| 135 | 4-{4-[3′- (Acetylamino)biphenyl-3- yl]-2-amino-1-methyl-5- oxo-4,5-dihydro-1H- imidazol-4-yl}phenyl methanesulfonate 0.25 acetate | CH₃SO₃ | 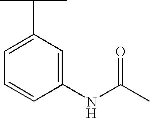 | 491 | δ 10.03 (s, 1 H), 7.78-7.71 (m, 2 H), 7.62-7.55 (m, 3 H), 7.52-7.34 (m, 4 H), 7.29 (d, J = 8.78 Hz, 2 H), 7.19 (d, J = 8.03 Hz, 1 H), 6.73 (br s, 2 H), 3.35 (s, 3 H), 3.00 (s, 3 H), 2.06 (s, 3 H), 1.88 (s, 0.67 H) |
| 136 | 4-[2-Amino-1-methyl-4- (3′-nitrobiphenyl-3-yl)-5- oxo-4,5-dihydro-1H- imidazol-4-yl]phenyl propane-1-sulfonate | CH₃CH₂CH₂SO₃ | 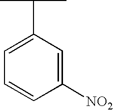 | 507 | δ 8.31 (s, 1 H), 8.23 (d, J = 8.03 Hz, 1 H), 8.03 (d, J = 7.78 Hz, 1 H), 7.85 (s, 1 H), 7.77 (t, J = 7.91 Hz, 1 H), 7.66 (d, J = 7.53 Hz, 1 H), 7.63-7.55 (m, 3 H), 7.48 (t, J = 7.78 Hz, 1 H), 7.26 (d, J = 8.78 Hz, 2 H), 6.78 (br s, 2 H), 3.50-3.43 (m, 2 H), 3.00 (s, 3 H), 1.87-1.77 (m, 2 H), 1.01 (t, J = 7.53 Hz, 3 H) |
| 137 | 4-[2-Amino-4-(3′- cyanobiphenyl-3-yl)-1- methyl-5-oxo-4,5-dihydro- 1H-imidazol-4-yl]phenyl propane-1-sulfonate | CH₃CH₂CH₂SO₃ | 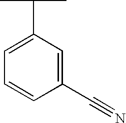 | N/A | δ 8.01 (s, 1 H), 7.89 (d, J = 8.03 Hz, 1 H), 7.86-7.78 (m, 2 H), 7.68 (t, J = 7.78 Hz, 1 H), 7.64-7.54 (m, 4 H), 7.45 (t, J = 7.78 Hz, 1 H), 7.26 (d, J = 8.78 Hz, 2 H), 6.75 (br s, 2 H), 3.49-3.43 (m, 2 H), 3.00 (s, 3 H), 1.87-1.76 (m, 2 H), 1.01 (t, J = 7.40 Hz, 3 H) |
| 138a | 4-[2-Amino-4-(2′,5′- dimethoxybiphenyl-3-yl)- 1-methyl-5-oxo-4,5- dihydro-1H-imidazol-4- yl]phenyl propane-1- sulfonate | CH₃CH₂CH₂SO₃ | 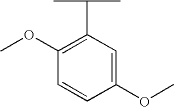 | 522 | δ 7.63-7.57 (m, 3 H), 7.43-7.37 (m, 1 H), 7.36-7.31 (m, 2 H), 7.27 (d, J = 8.78 Hz, 2 H), 7.01 (d, J = 9.03 Hz, 1 H), 6.89 (dd, J = 8.78, 3.01 Hz, 1 H), 6.76 (d, J = 3.01 Hz, 1 H), 6.69 (br s, 2 H), 3.72 (s, 3 H), 3.64 (s, 3 H), 3.49-3.43 (m, 2 H), 2.99 (s, 3 H), 1.87-1.77 (m, 2 H), 1.01 (t, J = 7.40 Hz, 3 H) |
| 138b | 4-[2-Amino-4-(3′- ethoxybiphenyl-3-yl)-1- methyl-5-oxo-4,5-dihydro- 1H-imidazol-4-yl]phenyl propane-1-sulfonate | CH₃CH₂CH₂SO₃ | 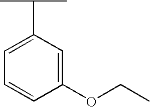 | 506 | δ 7.73 (s, 1 H), 7.59 (d, J = 8.53 Hz, 2 H), 7.52 (d, J = 7.53 Hz, 1 H), 7.50-7.45 (m, 1 H), 7.42-7.37 (m, 1 H) overlapping with 7.36 (t, J = 7.96 Hz, 1 H), 7.26 (d, J = 8.78 Hz, 2 H), 7.08 (d, J = 7.53 Hz, 1 H), 7.05-7.02 (m, 1 H), 6.93 (dd, J = 8.03, 2.26 Hz, 1 H), 6.73 (br s, 2 H), 4.07 (q, J =7.03 Hz, 2 H), 3.49-3.43 (m, 2 H), 3.00 (s, 3 H), 1.87-1.76 (m, 2 H), 1.34 (t, J = 7.03 Hz, 3 H), 1.01 (t, J = 7.53 Hz, 3 H) |
| 139 | 4-[2-Amino-4-(2′-fluoro-3′- methoxybiphenyl-3-yl)-1- methyl-5-oxo-4,5-dihydro- 1H-imidazol-4-yl]phenyl propane-1-sulfonate | CH₃CH₂CH₂SO₃ | 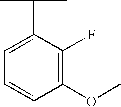 | 510 | δ 7.65 (s, 1 H), 7.58 (d, J = 8.53 Hz, 2 H), 7.52-7.47 (m, 1 H), 7.44-7.37 (m, 2 H), 7.26 (d, J = 8.53 Hz, 2 H), 7.23-7.14 (m, 2 H), 6.97-6.91 (m, 1 H), 6.73 (br s, 2 H), 3.87 (s, 3 H), 3.49-3.43 (m, 2 H), 2.99 (s, 3 H), 1.88-1.76 (m, 2 H), 1.01 (t, J = 7.40 Hz, 3 H) |
| 140 | 4-[2-Amino-4-(2′-fluoro-5′- methoxybiphenyl-3-yl)-1- methyl-5-oxo-4,5-dihydro- 1H-imidazol-4-yl]phenyl propane-1-sulfonate | CH₃CH₂CH₂SO₃ | 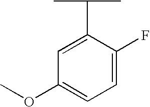 | 510 | δ 7.67 (s, 1 H), 7.58 (d, J = 8.78 Hz, 2 H), 7.52-7.47 (m, 1 H), 7.45-7.40 (m, 2 H), 7.29-7.18 (m, 3 H), 6.98-6.89 (m, 2 H), 6.73 (br s, 2 H), 3.77 (s, 3 H), 3.49-3.43 (m, 2 H), 2.99 (s, 3 H), 1.87-1.77 (m, 2 H), 1.01 (t, J = 7.40 Hz, 3 H) |
| 141 | 4-[2-Amino-4-(2′,6′- difluoro-3′- methoxybiphenyl-3-yl)-1- methyl-5-oxo-4,5-dihydro- 1H-imidazol-4-yl]phenyl propane-1-sulfonate | CH₃CH₂CH₂SO₃ | 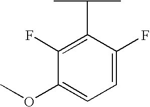 | 530 [M + H]⁺ | δ 7.60-7.51 (m, 4 H), 7.44 (t, J = 7.65 Hz, 1 H), 7.34-7.30 (m, 1 H), 7.27 (d, J = 8.78 Hz, 2 H), 7.23-7.17 (m, 1 H), 7.16-7.10 (m, 1 H), 6.74 (br s, 2 H), 3.85 (s, 3 H), 3.49-3.43 (m, 2 H), 2.98 (s, 3 H), 1.87-1.76 (m, 2 H), 1.01 (t, J = 7.40 Hz, 3 H) |
| 142 | 4-[2-Amino-4-(3′-cyano-4′- fluorobiphenyl-3-yl)-1- methyl-5-oxo-4,5-dihydro- 1H-imidazol-4-yl]phenyl propane-1-sulfonate | CH₃CH₂CH₂SO₃ | 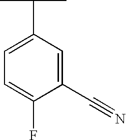 | 505 | δ 8.11 (dd, J = 6.15, 2.38 Hz, 1 H), 7.93 (ddd, J = 8.80, 5.10, 2.50 Hz, 1 H), 7.77 (dd, J = 1.90 Hz, 1 H), 7.69-7.53 (m, 5 H), 7.44 (t, J = 7.78 Hz, 1 H), 7.25 (d, J = 8.78 Hz, 2 H), 6.74 (br s, 2 H), 3.49-3.43 (m, 2 H), 3.00 (s, 3 H), 1.87-1.76 (m, 2 H), 1.01 (t, J = 7.40 Hz, 3 H) |
| 143 | 4-[2-Amino-4-(5′-cyano-2′- fluorobiphenyl-3-yl)-1- methyl-5-oxo-4,5-dihydro- 1H-imidazol-4-yl]phenyl propane-1-sulfonate | CH₃CH₂CH₂SO₃ | 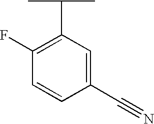 | 505 | δ 8.00 (dd, J = 7.15, 2.13 Hz, 1 H), 7.97-7.92 (m, 1 H), 7.72 (s, 1 H), 7.61-7.54 (m, 4 H), 7.51-7.45 (m, 2 H), 7.26 (d, J = 8.78 Hz, 2 H), 6.75 (br s, 2 H), 3.49-3.44 (m, 2 H), 2.99 (s, 3 H), 1.87-1.76 (m, 2 H), 1.01 (t, J = 7.53 Hz, 3 H) |
| 144 | 4-[2-Amino-1-methyl-5- oxo-4-(3-pyridin-3- ylphenyl)-4,5-dihydro-1H- imidazol-4-yl]phenyl propane-1-sulfonate | CH₃CH₂CH₂SO₃ | 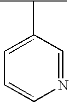 | 463 | δ 8.77(d, J = 2.01 Hz, 1 H), 8.57 (dd, J = 4.77, 1.76 Hz, 1 H), 7.97-7.92 (m, 1 H), 7.79-7.77 (m, 1 H), 7.63-7.57 (m, 3 H), 7.54 (d, J = 8.03 Hz, 1 H), 7.51-7.42 (m, 2 H), 7.26 (d, J = 8.78 Hz, 2 H), 6.75 (br s, 2 H), 3.49-3.43 (m, 2 H), 3.00 (s, 3 H), 1.87-1.76 (m, 2 H), 1.01 (t, J = 7.40 Hz, 3 H) |
| 145 | 4-{2-Amino-4-[3-(2- fluoropyridin-3-yl)phenyl]- 1-methyl-5-oxo-4,5- dihydro-1H-imidazol-4- yl}phenyl propane-1- sulfonate | CH₃CH₂CH₂SO₃ | 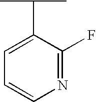 | 481 | δ 8.26-8.22 (m, 1 H), 8.06-7.99 (m, 1 H), 7.73 (s, 1 H), 7.61-7.53 (m, 3 H), 7.51-7.43 (m, 3 H), 7.26 (d, J = 8.78 Hz, 2 H), 6.75 (br s, 2 H), 3.49-3.43 (m, 2 H), 2.99 (s, 3 H), 1.87-1.76 (m, 2 H), 1.01 (t, J = 7.53 Hz, 3 H) |
| 146 | 4-{2-Amino-4-[3-(5- chloro-2-fluoropyridin-3- yl)phenyl]-1-methyl-5- oxo-4,5-dihydro-1H- imidazol-4-yl}phenyl propane-1-sulfonate | CH₃CH₂CH₂SO₃ | 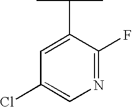 | 515, 517 | δ 8.31 (dd, J = 2.51, 1.51 Hz, 1 H), 8.19 (dd, J = 8.53, 2.51 Hz, 1 H), 7.78-7.54 (m, 1 H), 7.61-7.56 (m, 3 H), 7.55-7.51 (m, 1 H), 7.50-7.44 (m, 1 H), 7.26 (d, J = 8.78 Hz, 2 H), 6.76 (br s, 2 H), 3.49-3.43 (m, 2 H), 2.99 (s, 3 H), 1.87-1.77 (m, 2 H), 1.01 (t, J = 7.40 Hz, 3 H) |
| 147 | 4-{2-Amino-4-[3-(6- fluoropyridin-3-yl)phenyl]- 1-methyl-5-oxo-4,5- dihydro-1H-imidazol-4- yl}phenyl propane-1- sulfonate | CH₃CH₂CH₂SO₃ | 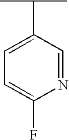 | 481 | δ 8.41 (d, J = 2.01, 1 H), 8.18-8.11 (m, 1 H), 7.75 (s, 1 H), 7.62-7.50 (m, 4H) overlapping with 7.50-7.42 (m, 1 H), 7.31-7.23 (m, 3 H), 6.75 (br s, 2 H), 3.49-3.43 (m, 2 H), 3.00 (s, 3 H), 1.87-1.77 (m, 2 H), 1.01 (t, J = 7.40 Hz, 3 H) |
| 148 | 4-[2-Amino-1-methyl-5- oxo-4-(3-pyrimidin-5- ylphenyl)-4,5-dihydro-1H- imidazol-4-yl]phenyl propane-1-sulfonate | CH₃CH₂CH₂SO₃ | 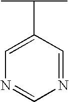 | N/A | δ 9.19 (s, 1 H), 9.01 (s, 2 H), 7.83-7.81 (m, 1 H), 7.67 (d, J = 7.78 Hz, 1 H), 7.63-7.56 (m, 3 H), 7.50 (t, J = 7.78 Hz, 1 H), 7.26 (d, J = 8.78 Hz, 2 H), 6.75 (br s, 2 H), 3.49-3.43 (m, 2 H), 3.00 (s, 3 H), 1.87-1.77 (m, 2 H), 1.01 (t, J = 7.53 Hz, 3 H) |
| 149 | 4-{2-Amino-4-[3-(2- fluoropyridin-4-yl)phenyl]- 1-methyl-5-oxo-4,5- dihydro-1H-imidazol-4- yl}phenyl propane-1- sulfonate | CH₃CH₂CH₂SO₃ | 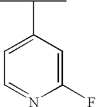 | 481 | δ 8.31 (d, J = 5.27 Hz, 1 H), 7.89 (s, 1 H), 7.72 (d, J = 7.78 Hz, 1 H), 7.63 (d, J = 7.78 Hz, 1 H) overlapping with 7.59 (d, J = 8.53 Hz, 2 H), 7.54 (d, J = 5.27 Hz, 1 H), 7.49 (t, J = 7.78 Hz, 1 H), 7.37 (s, 1 H), 7.26 (d, J = 8.78 Hz, 2 H), 6.77 (br s, 2 H), 3.49-3.43 (m, 2 H), 3.00 (s, 3 H), 1.87-1.77 (m, 2 H), 1.01 (t, J = 7.40 Hz, 3 H) |
| 150 | 4-{2-Amino-4-[3-(2- chloro-3-fluoropyridin-4- yl)phenyl]-1-methyl-5- oxo-4,5-dihydro-1H- imidazol-4-yl}phenyl propane-1-sulfonate | CH₃CH₂CH₂SO₃ | 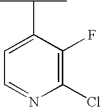 | 515, 517 | δ 8.33 (d, J = 5.02 Hz, 1 H), 7.82-7.79 (m, 1 H), 7.65 (d, J = 7.78 Hz, 1 H), 7.60-7.44 (m, 5 H), 7.27 (d, J = 8.78 Hz, 2 H), 6.77 (br s, 2 H), 3.49-3.43 (m, 2 H), 2.99 (s, 3 H), 1.87-1.76 (m, 2 H), 1.01 (t, J = 7.40 Hz, 3 H) |
| 151 | 4-{2-Amino-4-[3-(2- chloro-5-fluoropyridin-3- yl)phenyl]-1-methyl-5- oxo-4,5-dihydro-1H- imidazol-4-yl}phenyl propane-1-sulfonate | CH₃CH₂CH₂SO₃ | 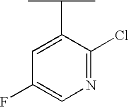 | N/A | δ 8.49 (d, J = 3.01 Hz, 1 H), 7.90 (dd, J = 8.53, 3.01 Hz, 1 H), 7.65 (s, 1 H), 7.61-7.43 (m, 4 H), 7.41-7.36 (m, 1 H), 7.27 (d, J = 8.53 Hz, 2 H), 6.80-6.70 (m, 2 H), 3.49-3.43 (m, 2 H), 2.99 (s, 3 H), 1.87-1.76 (m, 2 H), 1.01 (t, J = 7.53 Hz, 3 H) |
| 152 | 4-{2-Amino-4-[3-(2,6- difluoropyridin-3- yl)phenyl]-1-methyl-5- oxo-4,5-dihydro-1H- imidazol-4-yl}phenyl propane-1-sulfonate | CH₃CH₂CH₂SO₃ | 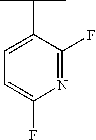 | 499 | δ 8.27-8.19 (m, 1 H), 7.71 (s, 1 H), 7.61-7.53 (m, 3 H), 7.49-7.43 (m, 2 H), 7.31-7.23 (m, 3 H), 6.75 (br s, 2 H), 3.49-3.43 (m, 2 H), 2.99 (s, 3 H), 1.87-1.76 (m, 2 H), 1.01 (t, J = 7.53 Hz, 3 H) |
| 153 | 4-[2-Amino-1-methyl-4- (3′-nitrobiphenyl-3-yl)-5- oxo-4,5-dihydro-1H- imidazol-4-yl]phenyl 2- methoxyethanesulfonate | CH₃OCH₂CH₂SO₃ | 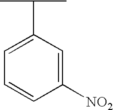 | 523 | δ 8.33-8.29 (m, 1 H), 8.25-8.20 (m, 1 H), 8.03 (d, J = 8.28 Hz, 1 H), 7.84 (s, 1 H), 7.77 (t, J = 8.03 Hz, 1 H), 7.66 (d, J = 7.53 Hz, 1 H), 7.62-7.55 (m, 3 H), 7.48 (t, J = 7.78 Hz, 1 H), 7.28 (d, J = 9.03 Hz, 2 H), 6.77 (br s, 2 H), 3.76 (s, 4 H), 3.27 (s, 3 H), 3.00 (s, 3 H) |
| 154 | 4-[2-Amino-4-(3′- cyanobiphenyl-3-yl)-1- methyl-5-oxo-4,5-dihydro- 1H-imidazol-4-yl]phenyl 2-methoxyethanesulfonate | CH₃OCH₂CH₂SO₃ | 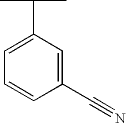 | 503 | δ 8.01 (s, 1 H), 7.92-7.86 (m, 1 H), 7.86-7.82 (m, 1 H), 7.80 (s, 1 H), 7.68 (t, J = 7.78 Hz, 1 H), 7.64-7.53 (m, 4 H), 7.45 (t, J = 7.65 Hz, 1 H), 7.27 (d, J = 8.78 Hz, 2 H), 6.75 (br s, 2 H), 3.76 (s, 4 H), 3.27 (s, 3 H), 3.00 (s, 3 H) |
| 155 | 4-[2-Amino-4-(2′,5′- dimethoxybiphenyl-3-yl)- 1-methyl-5-oxo-4,5- dihydro-1H-imidazol-4- yl]phenyl 2- methoxyethanesulfonate | CH₃OCH₂CH₂SO₃ | 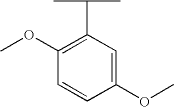 | 538 | δ 7.63-7.57 (m, 3 H), 7.40 (br s, 1 H), 7.36-7.31 (m, 2 H), 7.28 (d, J = 8.78 Hz, 2 H), 7.01 (d, J = 9.03 Hz, 1 H), 6.89 (dd, J = 8.78, 3.01 Hz, 1 H), 6.76 (d, J = 3.01 Hz, 1 H), 6.69 (br s, 2 H), 3.77 (s, 4 H), 3.72 (s, 3 H), 3.64 (s, 3 H), 3.28 (s, 3 H), 2.98 (s, 3 H) |
| 156 | 4-[2-Amino-4-(3′- ethoxybiphenyl-3-yl)-1- methyl-5-oxo-4,5-dihydro- 1H-imidazol-4-yl]phenyl 2-methoxyethanesulfonate | CH₃OCH₂CH₂SO₃ | 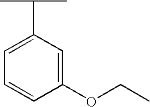 | 522 | δ 7.73 (s, 1 H), 7.58 (d, J = 8.78 Hz, 2 H), 7.52 (d, J = 7.53 Hz, 1 H), 7.50-7.45 (m, 1 H), 7.42-7.37 (m, 1 H) overlapping with 7.36 (t, J = 7.78 Hz, 1 H), 7.27 (d, J = 8.78 Hz, 2 H), 7.08 (d, J = 7.78 Hz, 1 H), 7.05-7.02 (m, 1 H), 6.93 (dd, J = 8.28, 2.01 Hz, 1 H), 6.73 (br s, 2 H), 4.07 (q, J = 7.03 Hz, 2 H), 3.76 (s, 4 H), 3.27 (s, 3 H), 3.00 (s, 3 H), 1.35 (t, J = 6.90 Hz, 3 H) |
| 157 | 4-[2-Amino-4-(2′-fluoro-3′- methoxybiphenyl-3-yl)-1- methyl-5-oxo-4,5-dihydro- 1H-imidazol-4-yl]phenyl 2-methoxyethanesulfonate | CH₃OCH₂CH₂SO₃ | 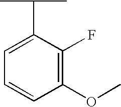 | 526 | δ 7.65 (s, 1 H), 7.58 (d, J = 8.78 Hz, 2 H), 7.52-7.47 (m, 1 H), 7.44-7.36 (m, 2 H), 7.27 (d, J = 8.78 Hz, 2 H), 7.25-7.14 (m, 2 H), 6.97-6.91 (m, 1 H), 6.73 (br s, 2 H), 3.87 (s, 3 H), 3.76 (s, 4 H), 3.27 (s, 3 H), 2.99 (s, 3 H) |
| 158 | 4-[2-Amino-4-(2′-fluoro-5′- methoxybiphenyl-3-yl)-1- methyl-5-oxo-4,5-dihydro- 1H-imidazol-4-yl]phenyl 2-methoxyethanesulfonate | CH₃OCH₂CH₂SO₃ | 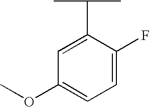 | 526 | δ 7.67 (s, 1 H), 7.58 (d, J = 8.78 Hz, 2 H), 7.53-7.47 (m, 1 H), 7.45-7.40 (m, 2 H), 7.27 (d, J = 8.78 Hz, 2 H), 7.25-7.19 (m, 1 H), 6.99-6.90 (m, 2 H), 6.73 (br s, 2 H), 3.77 (s, 3 H) overlapping with 3.77 (s, 4 H), 3.27 (s, 3 H), 2.99 (s, 3 H) |
| 159 | 4-[2-Amino-4-(2′,6′- difluoro-3′- methoxybiphenyl-3-yl)-1- methyl-5-oxo-4,5-dihydro- 1H-imidazol-4-yl]phenyl 2-methoxyethanesulfonate | CH₃OCH₂CH₂SO₃ | 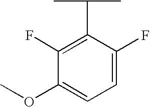 | 544 | δ 7.59-7.52 (m, 4 H), 7.44 (t, J = 7.78 Hz, 1 H), 7.34-7.30 (m, 1 H), 7.27 (d, J = 8.78 Hz, 2 H), 7.24-7.17 (m, 1 H), 7.16-7.10 (m, 1 H), 6.73 (br s, 2 H), 3.85 (s, 3 H), 3.76 (s, 4 H), 3.27 (s, 3 H), 2.98 (s, 3 H) |
| 160 | 4-[2-Amino-4-(3′-cyano-4′- fluorobiphenyl-3-yl)-1- methyl-5-oxo-4,5-dihydro- 1H-imidazol-4-yl]phenyl 2-methoxyethanesulfonate | CH₃OCH₂CH₂SO₃ | 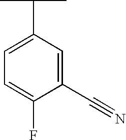 | 521 | δ 8.11 (dd, J = 6.15, 2.38 Hz, 1 H), 7.93 (ddd, J = 8.78, 5.27, 2.51 Hz, 1 H), 7.77 (s, 1 H), 7.65-7.53 (m, 5 H), 7.44 (t, J = 7.65 Hz, 1 H), 7.27 (d, J = 8.78 Hz, 2 H), 6.75 (br s, 2 H), 3.77 (s, 4 H), 3.27 (s, 3 H), 3.00 (s, 3 H) |
| 161 | 4-[2-Amino-4-(5′-cyano-2′- fluorobiphenyl-3-yl)-1- methyl-5-oxo-4,5-dihydro- 1H-imidazol-4-yl]phenyl 2-methoxyethanesulfonate | CH₃OCH₂CH₂SO₃ | 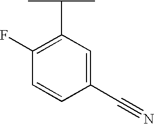 | 521 | δ 8.19-8.15 (m, 0.5 H), 8.12-8.06 (m, 0.5 H), 8.00 (dd, J = 7.28, 2.01 Hz, 1 H), 7.97-7.92 (m, 1 H), 7.72 (s, 1 H), 7.60-7.56 (m, 3 H), 7.49-7.45 (m, 2 H), 7.27 (d, J = 8.78 Hz, 2 H), 6.74 (br s, 2 H), 3.76 (s, 4 H), 3.27 (s, 3 H), 2.99 (s, 3 H) |
| 162 | 4-[2-Amino-1-methyl-5- oxo-4-(3-pyridin-3- ylphenyl)-4,5-dihydro-1H- imidazol-4-yl]phenyl 2- methoxyethanesulfonate | CH₃OCH₂CH₂SO₃ | 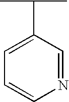 | 479 | δ 8.77 (d, J = 1.76 Hz, 1 H), 8.57 (dd, J = 4.77, 1.51 Hz, 1 H), 7.96-7.93 (m, 1 H), 7.79-7.77 (m, 1 H), 7.62-7.57 (m, 3 H), 7.56-7.41 (m, 3 H), 7.30-7.25 (m, 2 H), 6.75 (br s, 2 H), 3.76 (s, 4 H), 3.27 (s, 3 H), 3.00 (s, 3 H) |
| 163 | 4-{2-Amino-4-[3-(2- fluoropyridin-3-yl)phenyl]- 1-methyl-5-oxo-4,5- dihydro-1H-imidazol-4- yl}phenyl 2- methoxyethanesulfonate | CH₃OCH₂CH₂SO₃ | 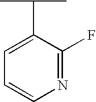 | 497 | δ 8.24 (d, J = 4.77 Hz, 1 H), 8.06-7.99 (m, 1 H), 7.73 (s, 1 H), 7.61-7.53 (m, 3 H), 7.52-7.43 (m, 3 H), 7.28 (d, J = 8.78 Hz, 2 H), 6.75 (br s, 2 H), 3.77 (s, 4 H), 3.27 (s, 3 H), 2.99 (s, 3 H) |
| 164 | 4-{2-Amino-4-[3-(5- chloro-2-fluoropyridin-3- yl)phenyl]-1-methyl-5- oxo-4,5-dihydro-1H- imidazol-4-yl}phenyl 2- methoxyethanesulfonate | CH₃OCH₂CH₂SO₃ | 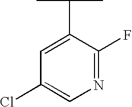 | 531, 533 | |
| 165 | 4-{2-Amino-4-[3-(6- fluoropyridin-3-yl)phenyl]- 1-methyl-5-oxo-4,5- dihydro-1H-imidazol-4- yl}phenyl 2- methoxyethanesulfonate | CH₃OCH₂CH₂SO₃ | 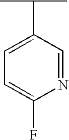 | 497 | δ 8.42 (d, J = 2.26 Hz, 1 H), 8.18-8.11 (m, 1 H), 7.75 (s, 1 H), 7.61-7.52 (m, 4 H), 7.45 (t, J = 7.65 Hz, 1 H), 7.30-7.24 (m, 3 H), 6.74 (br s, 2 H), 3.77 (s, 4 H), 3.27 (s, 3 H), 3.00 (s, 3 H) |
| 166 | 4-[2-Amino-1-methyl-5- oxo-4-(3-pyrimidin-5- ylphenyl)-4,5-dihydro-1H- imidazol-4-yl]phenyl 2- methoxyethanesulfonate | CH₃OCH₂CH₂SO₃ | 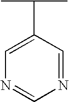 | 480 | δ 9.19 (s, 1 H), 9.01 (s, 2 H), 7.82 (s, 1 H), 7.67 (d, J = 7.53 Hz, 1 H), 7.63-7.56 (m, 3 H), 7.49 (t, J = 7.78 Hz, 1 H), 7.27 (d, J = 9.03 Hz, 2 H), 6.75 (br s, 2 H), 3.77 (s, 4 H), 3.27 (s, 3 H), 3.00 (s, 3 H) |
| 167 | 4-{2-Amino-4-[3-(2- chloro-3-fluoropyridin-4- yl)phenyl]-1-methyl-5- oxo-4,5-dihydro-1H- imidazol-4-yl}phenyl 2- methoxyethanesulfonate | CH₃OCH₂CH₂SO₃ | 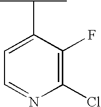 | 531, 533 | |
| 168 | 4-{2-Amino-4-[3-(2- chloro-5-fluoropyridin-3- yl)phenyl]-1-methyl-5- oxo-4,5-dihydro-1H- imidazol-4-yl}phenyl 2- methoxyethanesulfonate | CH₃OCH₂CH₂SO₃ | 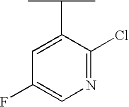 | 531, 533 | δ 8.49 (d, J = 3.01 Hz, 1 H), 7.90 (dd, J = 8.66, 2.89 Hz, 1 H), 7.65 (s, 1 H), 7.60-7.54 (m, 3 H), 7.46 (t, J = 7.78 Hz, 1 H), 7.41-7.36 (m, 1 H), 7.28 (d, J = 8.78 Hz, 2 H), 6.74 (br s, 2 H), 3.77 (s, 4 H), 3.28 (s, 3 H), 2.99 (s, 3 H) |
| 169 | 4-{2-Amino-4-[3-(2,6- difluoropyridin-3- yl)phenyl]-1-methyl-5- oxo-4,5-dihydro-1H- imidazol-4-yl}phenyl 2- methoxyethanesulfonate | CH₃OCH₂CH₂SO₃ | 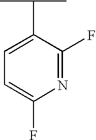 | 515 | δ 8.27-8.19 (m, 1 H), 7.71 (s, 1 H), 7.61-7.53 (m, 3 H), 7.49-7.43 (m, 2 H), 7.31-7.24 (m, 3 H), 6.75 (br s, 2 H), 3.77 (s, 4 H), 3.28 (s, 3 H), 2.99 (s, 3 H) |

### Example 170

### 3-Methoxy-5-(4,4,5,5-tetramethyl-1,3,2-dioxaborolan-2-yl)phenyl methanesulfonate

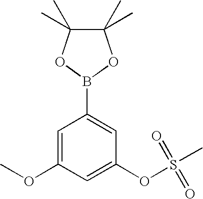

To a stirred solution of 3-methoxy-5-(4,4,5,5-tetramethyl-1,3,2-dioxaborolan-2-yl)phenol (120 mg, 0.48 mmol) in dichloromethane (3 mL) at 0° C. were added triethylamine (58 mg, 0.58 mmol) followed by methanesulfonyl chloride (71 mg, 0.62 mmol). Reaction mixture was allowed to reach ambient temperature and stirred for 18 hours and the resulting mixture was concentrated to dryness in vacuo. Purified on silica gel column using a gradient of dichloromethane/acetonitrile, (100/0 to 90/10), as the eluent. ¹H-NMR (CDCl₃): δ 7.30 (d, J=2.3 Hz, 1H), 7.28 (d, J=2.0 Hz, 1H), 6.96 (t, J=2.4 Hz, 1H), 3.86 (s, 3H), 3.16 (s, 3H), 1.35 (s, 12H).

### Example 171

### 4-(2-Amino-4-{3′-methoxy-5′-[(methylsulfonyl)oxy]biphenyl-3-yl}-1-methyl-5-oxo-4,5-dihydro-1H-imidazol-4-yl)phenyl propane-1-sulfonate

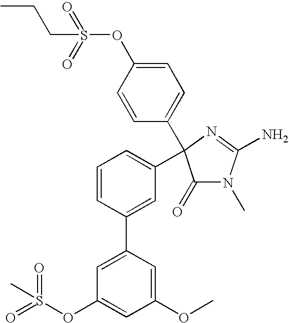

A mixture of 4-[2-Amino-4-(3-bromophenyl)-1-methyl-5-oxo-4,5-dihydro-1H-imidazol-4-yl]phenyl propane-1-sulfonate (80 mg, 0.17 mmol), 3-methoxy-5-(4,4,5,5-tetramethyl-1,3,2-dioxaborolan-2-yl)phenyl methanesulfonate (73 mg, 0.22 mmol), [1,1′-bis(diphenylphosphino)ferrocene]palladium(II) chloride dichloromethane adduct (14 mg, 0.017 mmol), potassium carbonate (142 mg, 1.03 mmol) in anhydrous tetrahydrofuran (3 mL) was irradiated under an atmosphere of argon in a microwave at 150° C. for 2 h. When cooled to ambient temperature the mixture was filtered and dimethyl sulfoxide (500 μL) was added. The solution was concentrated in vacuo to remove tetrahydrofuran and purified by preparative HPLC to give 32 mg (32% yield) of the title compound: ¹H-NMR (DMSO-d₆): δ 7.74 (s, 1H), 7.61-7.51 (m, 4H), 7.43 (t, J=7.78 Hz, 1H), 7.26 (d, J=8.78 Hz, 2H), 7.10-7.04 (m, 2H), 6.96 (t, J=2.13 Hz, 1H), 6.75 (br s, 2H), 3.85 (s, 3H), 3.49-3.43 (m, 2H), 3.42 (s, 3H), 3.00 (s, 3H), 1.87-1.77 (m, 2H), 1.01 (t, J=7.40 Hz, 3H); MS (ES) m/z 588 [M+H]⁺.

### Examples 172-175

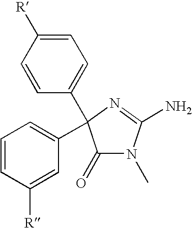

TABLE 2
Representative examples synthesized as described for 4-(2-Amino-4-{3′-methoxy-
5′-[(methylsulfonyl)oxy]biphenyl-3-yl}-1-methyl-5-oxo-4,5-dihydro-1H-imidazol-4-
yl)phenyl propane-1-sulfonate

| | | | | [M − | |
|---|---|---|---|---|---|
| | | | | H]⁻ | |
| Ex | Chemical name | R′ | R″ | m/z | ¹H-NMR |
| 172 | 4-{2-Amino-4-[3-(5- fluoropyridin-3-yl)phenyl]- 1-methyl-5-oxo-4,5- dihydro-1H-imidazol-4- yl}phenyl propane-1- sulfonate | CH₃CH₂CH₂SO₃ | 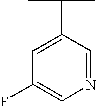 | 481 | δ 8.65 (s, 1 H), 8.59 (d, J = 2.51 Hz, 1 H), 7.94-7.89 (m, 1 H), 7.81 (s, 1 H), 7.67-7.55 (m, 4 H), 7.47 (t, J = 7.65 Hz, 1 H), 7.26 (d, J = 8.53 Hz, 2 H), 6.75 (br s, 2 H), 3.49-3.43 (m, 2 H), 3.00 (s, 3 H), 1.87-1.76 (m, 2 H), 1.01 (t, J = 7.40 Hz, 3 H) |
| 173 | 4-[2-Amino-4-(4′-fluoro-3′- methoxybiphenyl-3-yl)-1- methyl-5-oxo-4,5-dihydro- 1H-imidazol-4-yl]phenyl propane-1-sulfonate | CH₃CH₂CH₂SO₃ | 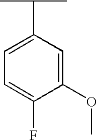 | 510 | δ 7.70 (s, 1 H), 7.61-7.52 (m, 3 H), 7.50-7.46 (m, 1 H), 7.40 (t, J = 7.65 Hz, 1 H), 7.32-7.23 (m, 4 H), 7.07-7.02 (m, 1 H), 6.73 (br s, 2 H), 3.90 (s, 3 H), 3.49-3.43 (m, 2 H), 3.00 (s, 3 H), 1.87-1.76 (m, 2 H), 1.01 (t, J = 7.53 Hz, 3 H) |
| 174 | 4-{2-Amino-4-[3-(5- fluoropyridin-3-yl)phenyl]- 1-methyl-5-oxo-4,5- dihydro-1H-imidazol-4- yl}phenyl 2- methoxyethanesulfonate | CH₃OCH₂CH₂SO₃ | 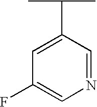 | 497 | δ 8.67-8.64 (m, 1 H), 8.59 (d, J = 2.76 Hz, 1 H), 7.95-7.89 (m, 1 H), 7.82 (s, 1 H), 7.67-7.55 (m, 4 H), 7.47 (t, J = 7.65 Hz, 1 H), 7.27 (d, J = 8.78 Hz, 2 H), 6.75 (br s, 2 H), 3.77 (s, 4 H), 3.27 (s, 3 H), 3.00 (s, 3 H) |
| 175 | 4-[2-Amino-4-(4′-fluoro-3′- methoxybiphenyl-3-yl)-1- methyl-5-oxo-4,5-dihydro- 1H-imidazol-4-yl]phenyl 2-methoxyethanesulfonate | CH₃OCH₂CH₂SO₃ | 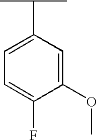 | 526 | δ 7.70 (s, 1 H), 7.61-7.51 (m, 3 H), 7.51-7.45 (m, 1 H), 7.40 (t, J = 7.65 Hz, 1 H), 7.32-7.24 (m, 4 H), 7.08-7.02 (m, 1 H), 6.73 (br s, 2 H), 3.90 (s, 3 H), 3.76 (s, 4 H), 3.27 (s, 3 H), 3.00 (s, 3 H) |

### Example 176

### 4-[2-Amino-1-methyl-5-oxo-4-(3-pyrazin-2-ylphenyl)-4,5-dihydro-1H-imidazol-4-yl]phenyl 2-methoxyethanesulfonate 0.25 acetate

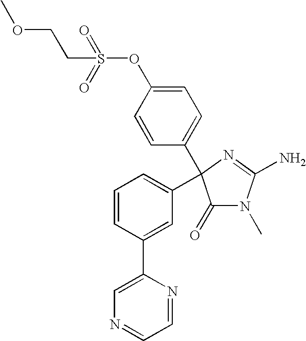

Anhydrous tetrahydrofuran (3 mL) was added to 4-[2-amino-4-(3-bromophenyl)-1-methyl-5-oxo-4,5-dihydro-1H-imidazol-4-yl]phenyl 2-methoxyethanesulfonate (75 mg, 0.16 mmol), 2-(tributylstannyl)pyrazine (86 mg, 0.23 mmol) and bis(triphenylphosphine)palladium(II) dichloride (5.5 mg, 0.008 mmol), and the mixture was irradiated under an atmosphere of argon in a microwave at 130° C. for 1 h. When cooled to ambient temperature the mixture was filtered and the solvent was evaporated in vacuo. The product was purified by preparative HPLC to give 29 mg (31% yield) of the title compound: ¹H-NMR (DMSO-d₆): δ 9.16 (d, J=1.51 Hz, 1H), 8.73-8.70 (m, 1H), 8.61 (d, J=2.51 Hz, 1H), 8.32-8.29 (m, 1H), 8.01-7.97 (m, 1H), 7.64-7.60 (m, 1H), 7.57 (d, J=8.78 Hz, 2H), 7.49 (t, J=7.78 Hz, 1H), 7.28 (d, J=8.78 Hz, 2H), 6.76 (br s, 2H), 3.77 (s, 4H), 3.27 (s, 3H), 3.00 (s, 3H), 1.89 (s, 0.85H); MS (ES) m/z 480 [M−H]⁻.

### Example 177

### 4-[2-Amino-1-methyl-5-oxo-4-(3-pyrazin-2-ylphenyl)-4,5-dihydro-1H-imidazol-4-yl]phenyl propane-1-sulfonate 0.25 acetate

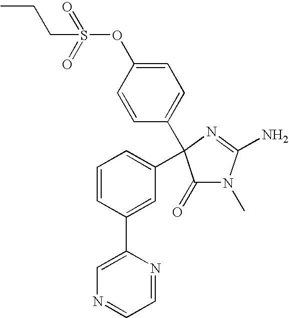

The title compound was synthesized as described for Example 176 in 36% yield starting from 4-[2-amino-4-(3-bromophenyl)-1-methyl-5-oxo-4,5-dihydro-1H-imidazol-4-yl]phenyl propane-1-sulfonate. Purification by preparative HPLC: ¹H-NMR (DMSO-d₆): δ 9.16 (d, J=1.51 Hz, 1H), 8.73-8.70 (m, 1H), 8.61 (d, J=2.51 Hz, 1H), 8.33-8.30 (m, 1H), 8.02-7.97 (m, 1H), 7.64-7.60 (m, 1H), 7.60-7.55 (m, 2H), 7.49 (t, J=7.78 Hz, 1H), 7.29-7.24 (m, 2H), 6.76 (br s, 2H), 3.49-3.44 (m, 2H), 3.00 (s, 3H), 1.90 (s, 0.86H), 1.87-1.76 (m, 2H), 1.01 (t. J=7.40 Hz, 3H); MS (ES) m/z 464 [M−H]⁻.

### Example 178 and 179

### (R)- and (S)-4-[2-Amino-1-methyl-5-oxo-4-(3-pyridin-3-ylphenyl)-4,5-dihydro-1H-imidazol-4-yl]phenyl methanesulfonate

Chromatographic separation of the enantiomers of 4-[2-amino-1-methyl-5-oxo-4-(3-pyridin-3-ylphenyl)-4,5-dihydro-1H-imidazol-4-yl]phenyl methanesulfonate

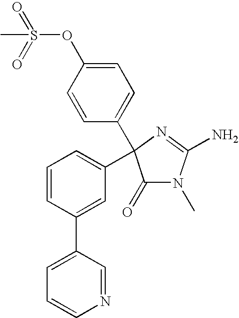

4-[2-Amino-1-methyl-5-oxo-4-(3-pyridin-3-ylphenyl)-4,5-dihydro-1H-imidazol-4-yl]phenyl methanesulfonate (7.5 mg) was dissolved in isopropanol (2 mL) and the resulting solution was injected on a Chiralpak AD column (21.2×250 mm), using isopropanol:heptane (20:80) as eluent at a flow rate of 16 mL/min. Detection was monitored at 254 nm and the two isomers were collected and concentrated in vacuo.

Isomer 1, example 178: 2.1 mg with unknown absolute configuration was collected: MS (ESI) m/z 435 [M−H]⁻.

Isomer 2, example 179: 2.1 mg with unknown absolute configuration was collected: MS (ESI) m/z 435 [M−H]⁻.

### Example 180

### 3′-(2-Amino-1-methyl-5-oxo-4-phenyl-4,5-dihydro-1H-imidazol-4-yl)-5-methoxybiphenyl-3-yl methanesulfonate hydrochloride

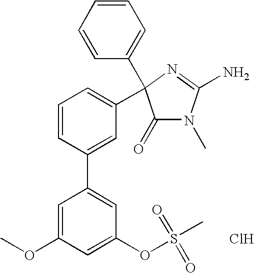

The title compound was synthesized as described for Example 5 and the salt of the base was prepared as described in Example 6 in 14% yield starting from 2-amino-5-(3-bromo-phenyl)-3-methyl-5-phenyl-3,5-dihydro-imidazol-4-one and 3-methoxy-5-(4,4,5,5-tetramethyl-1,3,2-dioxaborolan-2-yl)phenyl methanesulfonate. The crude product was purified on flash chromatography using acetonitrile/triethylamine, (90/10), as eluent, The formed hydrochloride salt precipitated after addition of diethyl ether. ¹H-NMR (DMSO-d₆): δ 11.67 (s, 1H), 9.61 (br s, 1H), 7.76 (d, J=7.8 Hz, 1H), 7.69 (s, 1H), 7.56 (t, J=7.8 Hz, 1H), 7.49-7.41 (m, 4H), 7.41-7.35 (m, 2H), 7.22-7.19 (m, 1H), 7.17-7.15 (m, 1H), 6.99 (t, J=2.1 Hz, 1H), 3.86 (s, 3H), 3.43 (s, 3H), 3.20 (s, 3H); MS (ESI) m/z 466 [M+1]⁺.

### Example 181

### 3-[(3-Bromophenyl)ethynyl]phenol

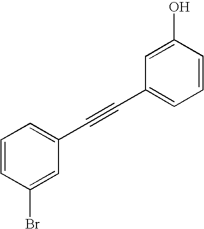

A solution of 1-bromo-3-ethynylbenzene (16.5 g, 90.9 mmol, described in Wettergren, J., Minidis, A. B. E. Tetrahedron Letters. 2003, 44, 7611-7612) in anhydrous tetrahydrofuran (25 mL) was added to a solution of 3-iodophenol (19 g, 86.4 mmol), bis(triphenylphosphine)palladium(II) chloride (383 mg, 0.55 mmol), copper(I) iodide (104 mg, 0.55 mmol) and triethylamine (75 mL, 538 mmol) in anhydrous tetrahydrofuran (125 mL) at 0° C. under an atmosphere of argon. The mixture was stirred for 10 minutes at 0° C., allowed to reach ambient temperature and stirred overnight. The solvents were evaporated in vacuo and the residue partitioned between dichloromethane (100 mL) and water (100 mL). The organic phase was washed with water (2×150 mL), brine (100 mL), dried over magnesium sulfate and concentrated in vacuo. Purification by column chromatography, using 50% dichloromethane in heptane as the eluent, followed by recrystallization in dichloromethane gave 17.16 g (69% yield) of the title compound: ¹H NMR (DMSO-d₆) δ 9.72 (s, 1H), 7.77-7.74 (m, 1H), 7.64-7.60 (m, 1H), 7.57-7.54 (m, 1H), 7.41-7.36 (m, 1H), 7.26-7.20 (m, 1H), 7.01-6.96 (m, 1H), 6.94-6.91 (m, 1H), 6.84 (ddd, J=8.28, 2.51, 1.00 Hz, 1H); MS (ES) m/z 271, 273 [M−H]⁻.

### Example 182

### 1-(3-Bromophenyl)-2-(3-hydroxyphenyl)ethane-1,2-dione

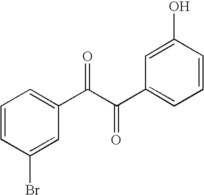

A solution of 3-[(3-bromophenyl)ethynyl]phenol (17.16 g, 62.83 mmol) and palladium(II) dichloride (1.11 g, 6.28 mmol) in dimethyl sulfoxide (300 mL) was stirred at 140° C. for 5 h. When cooled to room temperature the mixture was diluted with water (900 mL) and extracted with diethyl ether (3×200 mL). The combined organics were washed with water (400 mL) and brine (400 mL), dried over magnesium sulfate and concentrated to give 19.4 g (quantitative yield) of the title compound: MS (ES) m/z 303,305 [M−H]⁻.

### Example 183

### 5-(3-Bromophenyl)-5-(3-hydroxyphenyl)-3-methyl-2-thioxoimidazolidin-4-one

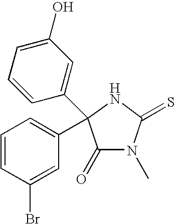

N-Methylthiourea (11.2 g, 124 mmol) was added to a solution of 1-(3-bromophenyl)-2-(3-hydroxyphenyl)ethane-1,2-dione (19.4 g, 62 mmol) in dimethyl sulfoxide (62 mL) and heated to 100° C. An aqueous solution of potassium hydroxide (1.5 M, 58 mL, 86.6 mmol) was added slowly and the resulting solution was stirred at 100° C. for 10 min. When cooled to room temperature the mixture was diluted with water (300 mL), 6 M hydrochloric acid (50 mL) was added and the aqueous phase was extracted with chloroform (3×150 mL). The combined organics were washed with water (250 mL), brine (250 mL), dried over sodium sulfate and concentrated in vacuo. Purification by column chromatography, using 25% ethyl acetate in heptane as the eluent, gave 16.33 g (70% yield) of the title compound: MS (ES) m/z 375, 377 [M−H]⁻.

### Example 184

### 3-[4-(3-Bromophenyl)-1-methyl-5-oxo-2-thioxoimidazolidin-4-yl]phenyl methanesulfonate

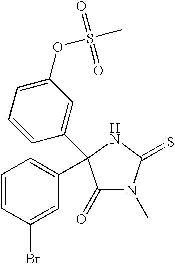

Methanesulfonyl chloride (0.39 mL, 4.97 mmol) was added to a cooled (0° C.) solution of 5-(3-bromophenyl)-5-(3-hydroxyphenyl)-3-methyl-2-thioxoimidazolidin-4-one (1.25 g, 3.31 mmol) and triethylamine (1.4 mL, 9.94 mmol) in anhydrous dichloromethane (30 mL) under an atmosphere of argon. The resulting mixture was allowed to reach ambient temperature and stirred for 1.5 h. The solvents were evaporated in vacuo and the residue was dissolved in ethyl acetate which resulted in the precipitation of triethylamine salt. The salt was filtered off and the filtrate was concentrated and purified by column chromatography, using 5-40% ethyl acetate in heptane as the eluent, to give 940 mg (62% yield) of the title compound: ¹H NMR (DMSO-d₆) δ 11.72 (s, 1H), 7.65-7.61 (m, 1H), 7.58 (t, J=8.03 Hz, 1H), 7.50 (m, 1H), 7.45-7.39 (m, 2H), 7.39-7.33 (m, 2H), 7.30-7.26 (m, 1H), 3.41 (s, 3H), 3.18 (s, 3H); MS (ES) m/z 453, 455 [M−H]⁻.

### Example 185

### 3-[2-Amino-4-(3-bromophenyl)-1-methyl-5-oxo-4,5-dihydro-1H-imidazol-4-yl]phenyl methanesulfonate

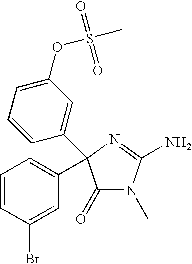

An aqueous solution (70%) of t-butylhydroperoxide (3 mL, 31 mmol) was added to a solution of 3-[4-(3-bromophenyl)-1-methyl-5-oxo-2-thioxoimidazolidin-4-yl]phenyl methanesulfonate (940 mg, 2.06 mmol) in methanol (30 mL) and 33% ammonia (6 mL) and the resulting mixture was stirred at room temperature for 6 h. The methanol was removed in vacuo, the residue diluted with water (75 mL) and extracted with chloroform (3×50 mL). The combined organic phases were washed with brine (75 mL), dried over sodium sulfate and concentrated in vacuo to give 890 mg (99%) of the title compound: MS (ES) m/z 438, 440 [M+H]⁺.

### Example 186

### 3-[4-(3-Bromophenyl)-1-methyl-5-oxo-2-thioxoimidazolidin-4-yl]phenyl 2-methoxyethanesulfonate

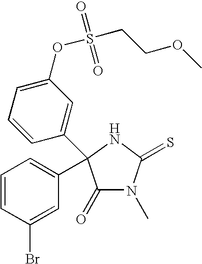

The title compound was synthesized as described for Example 184 in 71% yield starting from 5-(3-bromophenyl)-5-(3-hydroxyphenyl)-3-methyl-2-thioxoimidazolidin-4-one (and 2-methoxyethanesulfonyl chloride (described in: Matlack A. S. J. Org. Chem. 1958, 23, 729-731) except that the reaction mixture was stirred for 1 h: MS (ES) m/z 497, 499 [M−H]⁻.

### Example 187

### 3-[2-Amino-4-(3-bromophenyl)-1-methyl-5-oxo-4,5-dihydro-1H-imidazol-4-yl]phenyl 2-methoxyethanesulfonate

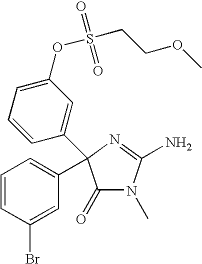

The title compound was synthesized as described for Example 185 in 77% yield from 3-[4-(3-bromophenyl)-1-methyl-5-oxo-2-thioxoimidazolidin-4-yl]phenyl 2-methoxyethanesulfonate, 33% ammonia and t-butylhydroperoxide: MS (ES) m/z 482, 484 [M+H]⁺.

### Example 188

### 3-[4-(3-Bromophenyl)-1-methyl-5-oxo-2-thioxoimidazolidin-4-yl]phenyl propane-1-sulfonate

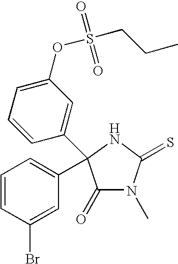

The title compound was synthesized as described for Example 184 in 94% yield from 5-(3-bromophenyl)-5-(3-hydroxyphenyl)-3-methyl-2-thioxoimidazolidin-4-one and propane-1-sulfonyl chloride: ¹H NMR (DMSO-d₆) δ 11.73 (s, 1H), 7.71 (m, 1H), 7.66-7.55 (m, 2H), 7.51-7.31 (m, 4H), 7.27-7.22 (m, 1H), 3.56-3.44 (m, 2H), 3.18 (s, 3H), 1.89-1.75 (m, 2H), 1.05-0.95 (m, 3H); MS (ES) m/z 481, 483 [M−H]⁻.

### Example 189

### 3-[2-Amino-4-(3-bromophenyl)-1-methyl-5-oxo-4,5-dihydro-1H-imidazol-4-yl]phenyl propane-1-sulfonate

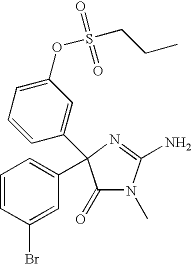

The title compound was synthesized as described for Example 185 in 94% yield starting from 3-[4-(3-bromophenyl)-1-methyl-5-oxo-2-thioxoimidazolidin-4-yl]phenyl propane-1-sulfonate, 33% ammonia and t-butylhydroperoxide: MS (ES) m/z 466, 468 [M+H]⁺.

### Example 190

### 3-[4-(3-Bromophenyl)-1-methyl-5-oxo-2-thioxoimidazolidin-4-yl]phenyl propane-2-sulfonate

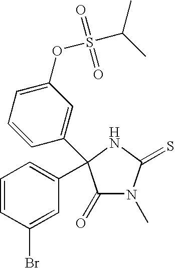

The title compound was synthesized as described for Example 184 in 55% yield from 5-(3-bromophenyl)-5-(3-hydroxyphenyl)-3-methyl-2-thioxoimidazolidin-4-one and propane-2-sulfonyl chloride, except that the reaction mixture was stirred for 3 h: MS (ES) m/z 481, 483 [M−H]⁻.

### Example 191

### 3-[2-Amino-4-(3-bromophenyl)-1-methyl-5-oxo-4,5-dihydro-1H-imidazol-4-yl]phenyl propane-2-sulfonate

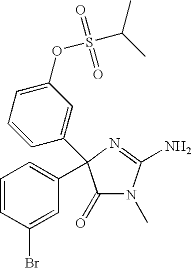

The title compound was synthesized as described for Example 185 in 84% yield starting from 3-[4-(3-bromophenyl)-1-methyl-5-oxo-2-thioxoimidazolidin-4-yl]phenyl propane-2-sulfonate, 33% ammonia and t-butylhydroperoxide: MS (ES) m/z 466, 468 [M+H]⁺.

### Example 192

### 3-[4-(3-Bromophenyl)-1-methyl-5-oxo-2-thioxoimidazolidin-4-yl]phenyl cyclopropanesulfonate

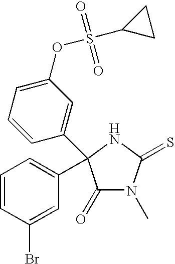

The title compound was synthesized as described for Example 184 in 60% yield starting from 5-(3-bromophenyl)-5-(3-hydroxyphenyl)-3-methyl-2-thioxoimidazolidin-4-one (and cyclopropanesulfonyl chloride, except that the reaction mixture was stirred for 23 h: MS (ES) m/z 479, 481 [M−H]⁻.

### Example 193

### 3-[2-Amino-4-(3-bromophenyl)-1-methyl-5-oxo-4,5-dihydro-1H-imidazol-4-yl]phenyl cyclopropanesulfonate

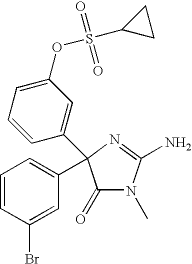

The title compound was synthesized as described for Example 185 in 93% yield starting from 3-[4-(3-bromophenyl)-1-methyl-5-oxo-2-thioxoimidazolidin-4-yl]phenyl cyclopropanesulfonate, 33% ammonia and t-butylhydroperoxide: MS (ES) m/z 462, 464 [M+H]⁺.

### Example 194

### 3-[4-(3-Bromophenyl)-1-methyl-5-oxo-2-thioxoimidazolidin-4-yl]phenyl dimethylsulfamate

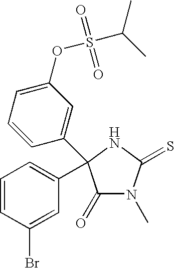

The title compound was synthesized as described for Example 184 in 72% yield starting from 5-(3-bromophenyl)-5-(3-hydroxyphenyl)-3-methyl-2-thioxoimidazolidin-4-one and dimethylsulfamoyl chloride, except that the reaction mixture was stirred over weekend: MS (ES) m/z 482, 484 [M−H]⁻.

### Example 195

### 3-[2-Amino-4-(3-bromophenyl)-1-methyl-5-oxo-4,5-dihydro-1H-imidazol-4-yl]phenyl dimethylsulfamate

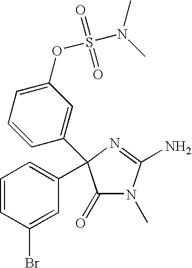

The title compound was synthesized as described for Example 185 in 93% yield starting from 3-[4-(3-bromophenyl)-1-methyl-5-oxo-2-thioxoimidazolidin-4-yl]phenyl dimethylsulfamate, 33% ammonia and t-butylhydroperoxide: MS (ES) m/z 467, 469 [M+H]⁺.

### Example 196

### 3-[4-(3-Bromophenyl)-1-methyl-5-oxo-2-thioxoimidazolidin-4-yl]phenyl morpholine-4-sulfonate

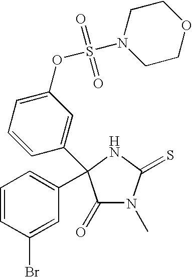

The title compound was synthesized as described for Example 184 in 34% yield starting from 5-(3-bromophenyl)-5-(3-hydroxyphenyl)-3-methyl-2-thioxoimidazolidin-4-one and morpholine-4-sulfonyl chloride, except that the reaction mixture was stirred overnight: ¹H NMR (DMSO-d₆) δ 11.75 (s, 1H), 7.65-7.54 (m, 2H), 7.54-7.47 (m, 1H), 7.45-7.33 (m, 4H), 7.32-7.26 (m, 1H), 3.64-3.57 (m, 4H), 3.27-3.22 (m, 4H), 3.18 (s, 3H); MS (ES) m/z 524, 526 [M−H]⁻.

### Example 197

### 3-[2-Amino-4-(3-bromophenyl)-1-methyl-5-oxo-4,5-dihydro-1H-imidazol-4-yl]phenyl morpholine-4-sulfonate

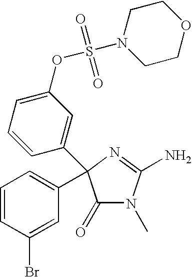

The title compound was synthesized as described for Example 185 in 85% yield starting from 3-[4-(3-bromophenyl)-1-methyl-5-oxo-2-thioxoimidazolidin-4-yl]phenyl morpholine-4-sulfonate, 33% ammonia and t-butylhydroperoxide: MS (ES) m/z 509, 511 [M+H]⁺.

### Example 198

### 3-[4-(3-Bromophenyl)-1-methyl-5-oxo-2-thioxoimidazolidin-4-yl]phenyl ethanesulfonate

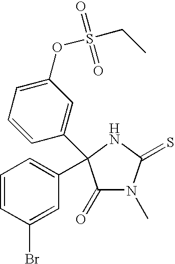

The title compound was synthesized as described for Example 184 in 88% yield atrating from 5-(3-bromophenyl)-5-(3-hydroxyphenyl)-3-methyl-2-thioxoimidazolidin-4-one and ethanesulfonyl chloride: ¹H NMR (DMSO-d₆) δ11.73 (s, 1H), 7.64-7.61 (m, 1H), 7.57 (t, J=8.03 Hz, 1H), 7.50-7.48 (m, 1H), 7.48-7.44 (m, 1H), 7.42-7.32 (m, 3H), 7.26 (t, J=2.01 Hz, 1H), 3.58-3.50 (m, 2H), 3.18 (s, 3H), 1.37-1.29 (m, 3H); MS (ES) m/z 467, 469 [M−H]⁻.

### Example 199

### 3-[2-Amino-4-(3-bromophenyl)-1-methyl-5-oxo-4,5-dihydro-1H-imidazol-4-yl]phenyl ethanesulfonate

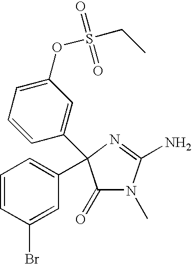

The title compound was synthesized as described for Example 185 in 87% yield starting from 3-[4-(3-bromophenyl)-1-methyl-5-oxo-2-thioxoimidazolidin-4-yl]phenyl ethanesulfonate, 33% ammonia and t-butylhydroperoxide. Purified by column chromatography, using a mixture of 10% 0.1 M ammonia in methanol and 90% dichloromethane as the eluent: ¹H NMR (DMSO-d₆) δ7.62-7.58 (m, 1H), 7.50-7.42 (m, 4H), 7.42-7.38 (m, 1H), 7.32-7.27 (m, 1H), 7.25-7.20 (m, 1H), 6.83 (br. s., 2H), 3.50 (q, J=7.28 Hz, 2H), 2.99 (s, 3H), 1.37-1.31 (m, 3H); MS (ES) m/z 450, 452 [M−H]⁻.

### Example 200

### 2-Amino-5-(3-bromophenyl)-5-(3-hydroxyphenyl)-3-methyl-3,5-dihydro-4H-imidazol-4-one

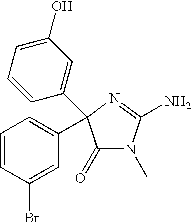

The title compound was synthesized as described for Example 185 in 57% yield starting from 5-(3-bromophenyl)-5-(3-hydroxyphenyl)-3-methyl-2-thioxoimidazolidin-4-one, 33% ammonia and t-butylhydroperoxide. Purified by column chromatography, using a mixture of 10% 0.1 M ammonia in methanol and 90% dichloromethane as the eluent: ¹H NMR (DMSO-d₆) δ 9.33 (br. s., 1H), 7.62-7.59 (m, 1H), 7.49-7.39 (m, 2H), 7.28 (m, 1H), 7.07 (m, 1H), 6.86-6.81 (m, 2H), 6.69 (br. s., 2H), 6.63-6.58 (m, 1H), 2.98 (s, 3H); MS (ES) m/z 360, 362 [M+H]⁺.

### Example 201

### 3-{2-Amino-4-[3-(5-methoxypyridin-3-yl)phenyl]-1-methyl-5-oxo-4,5-dihydro-1H-imidazol-4-yl}phenyl ethanesulfonate 0.75 acetate

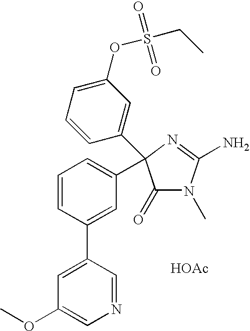

The title compound was synthesized as described for Example 171 in 14% yield starting from 2-amino-5-(3-bromophenyl)-5-(3-hydroxyphenyl)-3-methyl-3,5-dihydro-4H-imidazol-4-one. Purified by preparative HPLC and freeze-dried: ¹H NMR (DMSO-d₆) δ 8.34 (d, J=1.76 Hz, 1H), 8.30 (d, J=2.76 Hz, 1H), 7.71-7.74 (m, 1H), 7.61 (d, J=7.53 Hz, 1H), 7.50-7.56 (m, 2H), 7.42-7.48 (m, 4H), 7.19-7.25 (m, 1H), 6.82 (br. s., 2H), 3.89 (s, 3H), 3.46-3.53 (m, 2H), 3.01 (s, 3H), 1.91 (s, 1H), 1.34 (t, J=7.28 Hz, 3H); MS (ES) m/z 479 [M−H]⁻.

### Example 202

### 4-{2-Amino-4-[3-(5-methoxypyridin-3-yl)phenyl]-1-methyl-5-oxo-4,5-dihydro-1H-imidazol-4-yl}phenyl methanesulfonate acetic acid

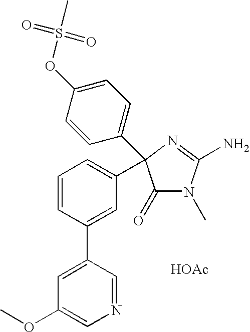

The title compound was synthesized as described for Example 171 in 16% yield starting from 4-[2-amino-4-(3-bromophenyl)-1-methyl-5-oxo-4,5-dihydro-1H-imidazol-4-yl]phenyl methanesulfonate. Purified by preparative HPLC and freeze-dried: ¹H NMR (DMSO-d₆) δ 8.34 (d, J=2.01 Hz, 1H), 8.30 (d, J=2.76 Hz, 1H), 7.79-7.76 (m, 1H), 7.63-7.58 (m, 3H), 7.56-7.52 (m, 1H), 7.49-7.42 (m, 2H), 7.31-7.27 (m, 2H), 6.77 (br. s., 2H), 3.89 (s, 3H), 3.35 (s, 3H), 3.00 (s, 3H), 1.91 (s, 3H); MS (ES) m/z 465 [M−H]⁻.

### Example 203

### 2-Amino-5-[3-(2-fluoropyridin-3-yl)phenyl]-5-(3-hydroxyphenyl)-3-methyl-3,5-dihydro-4H-imidazol-4-one

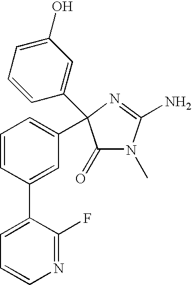

The title compound was synthesized as described for Example 171 in 67% yield starting from 2-amino-5-(3-bromophenyl)-5-(3-hydroxyphenyl)-3-methyl-3,5-dihydro-4H-imidazol-4-one. Purified by column chromatography, using a mixture of 10% 0.1 M ammonia in methanol and 90% dichloromethane as the eluent: ¹H NMR (DMSO-d₆) δ 9.33 (s, 1H), 8.28-8.23 (m, 1H), 8.04-7.98 (m, 1H), 7.70 (s, 1H), 7.58-7.39 (m, 4H), 7.13-7.05 (m, 1H), 6.93-6.82 (m, 2H), 6.65-6.59 (m, 1H), 2.99 (s, 3H); MS (ES) m/z 377 [M+H]⁺.

### Example 204

### 2-Amino-5-(3-hydroxyphenyl)-3-methyl-5-(3-pyrimidin-5-ylphenyl)-3,5-dihydro-4H-imidazol-4-one

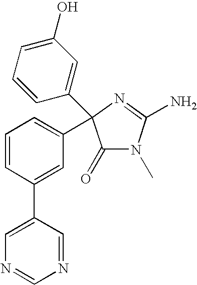

The title compound was synthesized as described for Example 171 in 39% yield starting from 2-amino-5-(3-bromophenyl)-5-(3-hydroxyphenyl)-3-methyl-3,5-dihydro-4H-imidazol-4-one. Purified by column chromatography, using a mixture of 10% 0.1 M ammonia in methanol and 90% dichloromethane as the eluent: MS (ES) m/z 360 [M+H]⁺.

### Example 205

### 2-Amino-5-(3-hydroxyphenyl)-3-methyl-5-(3-pyridin-3-ylphenyl)-3,5-dihydro-4H-imidazol-4-one

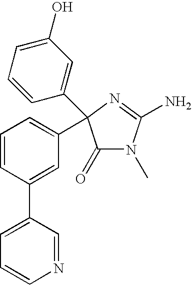

The title compound was synthesized as described for Example 171 in 38% yield starting from 2-amino-5-(3-bromophenyl)-5-(3-hydroxyphenyl)-3-methyl-3,5-dihydro-4H-imidazol-4-one. Purified by column chromatography, using a mixture of 10% 0.1 M ammonia in methanol and 90% dichloromethane as the eluent: MS (ES) m/z 359 [M+H]⁺.

### Example 206

### 2-Amino-5-(3-hydroxyphenyl)-5-[3-(5-methoxypyridin-3-yl)phenyl]-3-methyl-3,5-dihydro-4H-imidazol-4-one

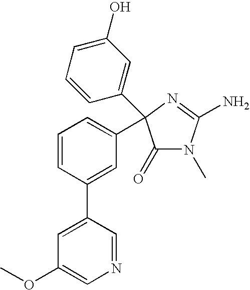

The title compound was synthesized as described for Example 171 in 19% yield starting from 2-amino-5-(3-bromophenyl)-5-(3-hydroxyphenyl)-3-methyl-3,5-dihydro-4H-imidazol-4-one. Purified by column chromatography, using a mixture of 10% 0.1 M ammonia in methanol and 90% dichloromethane as the eluent: MS (ES) m/z 389 [M+H]⁺.

### Example 207

### 3-{2-Amino-4-[3-(2-fluoropyridin-3-yl)phenyl]-1-methyl-5-oxo-4,5-dihydro-1H-imidazol-4-yl}phenyl trifluoromethanesulfonate hydrochloride

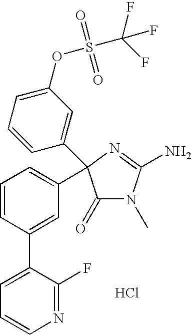

A mixture of 2-amino-5-[3-(2-fluoropyridin-3-yl)phenyl]-5-(3-hydroxyphenyl)-3-methyl-3,5-dihydro-4H-imidazol-4-one (140 mg, 0.37 mmol), N-phenyl-bis(trifluoromethanesulfonimide) (133 mg, 0.37 mmol), anhydrous potassium carbonate (310 mg, 2.22 mmol) in anhydrous tetrahydrofuran (4 mL) were irradiated in a microwave at 120° C. for 7 min. The mixture was filtrated, the solvent was evaporated and the residue purified by preparative HPLC, to give 32 mg of the base: ¹H NMR (DMSO-d₆) δ 8.29-8.19 (m, 2H), 8.03-7.98 (m, 1H), 7.70-7.67 (m, 2H), 7.56-7.45 (m, 5H), 7.40 (dd, J=8.03, 2.01 Hz, 1H), 6.85 (br. s., 2H), 3.01-2.98 (m, 3H).

The base was dissolved in anhydrous dichloromethane (3 mL) and hydrochloric acid (1 M in diethyl ether, 0.1 mL) was added. The mixture was stirred at room temperature for 5 min and the solvent was evaporated to give 91 mg (45% yield) of the title compound: MS (ES) m/z 509 [M+H]⁺.

### Example 208

### 3-[2-Amino-1-methyl-5-oxo-4-(3-pyrimidin-5-ylphenyl)-4,5-dihydro-1H-imidazol-4-yl]phenyl trifluoromethanesulfonate hydrochloride

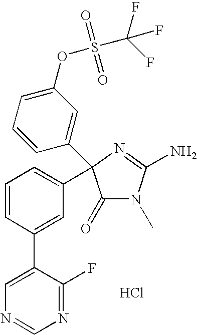

The title compound was synthesized as described for Example 207 in 30% yield starting from 2-amino-5-(3-hydroxyphenyl)-3-methyl-5-(3-pyrimidin-5-ylphenyl)-3,5-dihydro-4H-imidazol-4-one: ¹H NMR of free base (DMSO-d₆) δ 9.19 (s, 1H), 9.00 (s, 2H), 7.77-7.75 (m, 1H), 7.73-7.66 (m, 2H), 7.60-7.57 (m, 1H), 7.57-7.48 (m, 3H), 7.43-7.39 (m, 1H), 6.86 (br. s., 2H), 3.01 (s, 3H); MS (ES) m/z 492 [M+H]⁺.

### Example 209

### 3-[2-Amino-1-methyl-5-oxo-4-(3-pyridin-3-ylphenyl)-4,5-dihydro-1H-imidazol-4-yl]phenyl trifluoromethanesulfonate hydrochloride

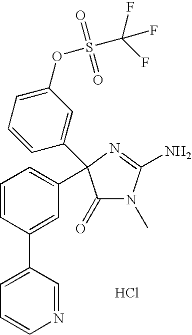

The title compound was synthesized as described for Example 207 in 31% yield starting from 2-amino-5-(3-hydroxyphenyl)-3-methyl-5-(3-pyridin-3-ylphenyl)-3,5-dihydro-4H-imidazol-4-one: ¹H NMR of free base (DMSO-d₆) δ 8.69 (d, J=2.01 Hz, 1H), 8.51 (dd, J=4.77, 1.51 Hz, 1H), 7.92-7.83 (m, 1H), 7.69-7.60 (m, 2H), 7.58-7.32 (m, 7H), 6.83 (br. s., 2H), 2.95 (s, 3H); MS (ES) m/z 491 [M+H]⁺.

### Example 210

### 3-{2-Amino-4-[3-(5-methoxypyridin-3-yl)phenyl]-1-methyl-5-oxo-4,5-dihydro-1H-imidazol-4-yl}phenyl trifluoromethanesulfonate hydrochloride

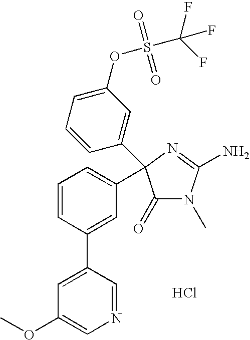

The title compound was synthesized as described for Example 207 in 34% yield starting from 2-amino-5-(3-hydroxyphenyl)-5-[3-(5-methoxypyridin-3-yl)phenyl]-3-methyl-3,5-dihydro-4H-imidazol-4-one: ¹H NMR of free base (DMSO-d₆) δ 8.32 (d, J=15.66 Hz, 2H), 7.73-7.61 (m, 3H), 7.61-7.51 (m, 3H), 7.51-7.41 (m, 3H), 7.04 (br. s., 2H), 3.89 (s, 3H), 3.03 (s, 3H); MS (ES) m/z 521 [M+H]⁺.

### Examples 211 and 212

### (R)- and (S)-3′-(2-amino-1-methyl-5-oxo-4-phenyl-4,5-dihydro-1H-imidazol-4-yl)-5-methoxybiphenyl-3-yl methanesulfonate hydrochloride

Chromatographic separation of the enantiomers of 3′-(2-amino-1-methyl-5-oxo-4-phenyl-4,5-dihydro-1H-imidazol-4-yl)-5-methoxybiphenyl-3-yl methanesulfonate.

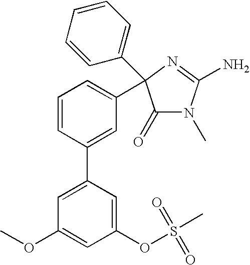

The free base of 3′-(2-amino-1-methyl-5-oxo-4-phenyl-4,5-dihydro-1H-imidazol-4-yl)-5-methoxybiphenyl-3-yl methanesulfonate (1.02 g) was dissolved in methanol (50 mL) and the resulting solution was injected (5 separate injections) on a Chiralcel OJ column (4.6×250 mm), using methanol as eluent at a flow rate of 0.8 mL/min. Detection was monitored at 254, 220 and 280 nm and the two isomers were collected and concentrated in vacuo. The residue was dissolved in anhydrous dichloromethane (4 mL) and hydrochloric acid (1 M in diethyl ether, 1 mL) was added. The resulting mixture was stirred for 5 min and concentrated by nitrogen blow followed by vacuum oven overnight.

Isomer 1, Example 211: 307 mg with unknown absolute configuration was collected: ¹H NMR (DMSO-d₆) δ 11.73 (br. s., 1 μl), 9.61 (br. s., 2H), 7.76 (d, J=7.78 Hz, 1H), 7.72-7.69 (m, 1H), 7.56 (t, J=7.78 Hz, 1H), 7.48-7.38 (m, 6H), 7.22-7.20 (m, 1H), 7.18-7.16 (m, 1H), 7.00-6.97 (m, 1H), 3.86 (s, 3H), 3.43 (s, 3H), 3.21 (s, 3H).

Isomer 2, Example 212: 404 mg with unknown absolute configuration was collected: ¹H NMR (DMSO-d₆) δ 11.75 (br. s., 1H), 9.63 (br. s., 2H), 7.76 (d, J=7.78 Hz, 1H), 7.72-7.69 (m, 1H), 7.56 (t, J=7.78 Hz, 1H), 7.48-7.38 (m, 6H), 7.22-7.20 (m, 1H), 7.18-7.16 (m, 1H), 6.99 (t, J=2.26 Hz, 1H), 3.86 (s, 3H), 3.43 (s, 3H), 3.21 (s, 3H).

### Example 213

### 4-[2-Amino-4-(3′,5′-dichlorobiphenyl-3-yl)-1-methyl-5-oxo-4,5-dihydro-1H-imidazol-4-yl]phenyl methanesulfonate hydrochloride

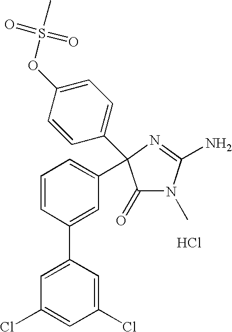

A mixture of 4-[2-amino-4-(3-bromophenyl)-1-methyl-5-oxo-4,5-dihydro-1H-imidazol-4-yl]phenyl methanesulfonate (100 mg, 0.23 mmol), 2-(3,5-dichlorophenyl)-4,4,5,5-tetramethyl-1,3,2-dioxaborolane (81 mg, 0.3 mmol; described in: Jian-Yang; Tse. et al. Science 2002, 295(5553), 305-308), [1,1′-bis(diphenylphosphino)ferrocene]palladium(II) chloride dichloromethane adduct (19 mg, 0.02 mmol), potassium carbonate (189 mg, 1.37 mmol) in anhydrous tetrahydrofuran (3 mL) was irradiated under an atmosphere of argon in a microwave at 130° C. for 2 h. When cooled to ambient temperature another 2-(3,5-dichlorophenyl)-4,4,5,5-tetramethyl-1,3,2-dioxaborolane (50 mg, 0.18 mmol) was added and the reaction mixture irradiated under an atmosphere of argon in a microwave at 130° C. for 3 h. When cooled to ambient temperature the mixture was filtered and purified by preparative HPLC to give the acetic acid salt: ¹H NMR (DMSO-d₆) δ 7.82-7.76 (m, 1H), 7.65-7.55 (m, 7H), 7.48-7.41 (m, 1H), 7.33-7.26 (m, 2H), 6.79 (br. s., 2H), 3.35 (s, 3H, obscured by H₂O), 3.00 (s, 3H), 1.91 (s, 3H).

The acetic acid salt was dissolved in anhydrous dichloromethane (1 mL) and hydrochloric acid (1 M in diethyl ether, 35 μL) was added. The resulting mixture was concentrated by nitrogen blow followed by vacuum oven overnight to give 18 mg (14% yield) of the title compound: MS (ES) m/z 504, 506 [M+H]⁺.

### Example 214

### 2-(4,4,5,5-Tetramethyl-1,3,2-dioxaborolan-2-yl)pyrimidine

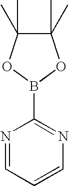

A mixture of 2-bromopyrimidine (760 mg, 4.78 mmol), 4,4,4′,4′,5,5,5′,5′-octamethyl-2,2′-bi-1,3,2-dioxaborolane (1.46 g, 5.74 mmol), [1,1′-bis(diphenylphosphino)ferrocene]palladium(II) chloride dichloromethane adduct (114 mg, 0.14 mmol), potassium acetate (1.41 g, 14.3 mmol) in 1,2-dimethoxyethane (16 mL) and water (1 mL) was irradiated in a microwave at 150° C. for 15 min. When cooled to ambient temperature the mixture was evaporated in vacuo, slurried in ethyl acetate, filtered through celite, passed through a silica pad and concentrated in vacuo to give 660 mg (67% yield) of the title compound: MS (EI) m/z 207 [M+1]⁺.

### Examples 215-246

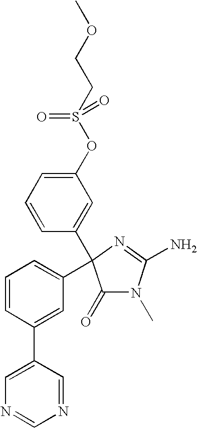

TABLE 3
Representative examples synthesized as described for examples were synthesized
as described for 4-[2-Amino-1-methyl-5-oxo-4-(3-pyridin-3-ylphenyl)-4,5-dihydro-1H-
imidazol-4-yl]phenyl methanesulfonate 0.25 acetate.

| | | | | [M − | |
|---|---|---|---|---|---|
| | Chemical | | | H]⁻ | |
| Ex | name | R″ | R′′′ | m/z | ¹H-NMR |
| 215 | 3-[2-Amino-1- methyl-5-oxo-4- (3-pyridin-3- ylphenyl)-4,5- dihydro-1H- imidazol-4- yl]phenyl methanesulfonate | 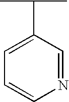 | CH₃SO₃ | N/A | 8.79-8.74 (m, 1 H), 8.59-8.55 (m, 1 H), 7.97-7.91 (m, 1 H), 7.76-7.72 (m, 1 H), 7.62-7.39 (m, 7 H), 7.27-7.22 (m, 1 H), 6.79 (br. s., 2 H), 3.35 (s, 3 H, obscured by H₂O), 3.03-2.96 (s, 3 H) |
| 216 | 3-{2-Amino-4- [3-(6- fluoropyridin-3- yl)phenyl]-1- methyl-5-oxo- 4,5-dihydro-1H- imidazol-4- yl}phenyl methanesulfonate | 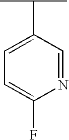 | CH₃SO₃ | 455 [M + H]⁺ | 8.43-8.38 (m, 1 H), 8.17-8.10 (m, 1 H), 7.73-7.69 (m, 1 H), 7.60-7.40 (m, 5 H), 7.30-7.22 (m, 3 H), 6.78 (br. s., 2 H), 3.35 (s, 3 H, obscured by H₂O), 3.03-2.96 (s, 3 H) |
| 217 | 3-{2-Amino-4- [3-(2- fluoropyridin-3- yl)phenyl]-1- methyl-5-oxo- 4,5-dihydro-1H- imidazol-4- yl}phenyl methanesulfonate | 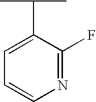 | CH₃SO₃ | 453 | 8.24 (d, J = 4.77 Hz, 1 H), 8.05-7.97 (m, 1 H), 7.69 (s, 1 H), 7.56-7.51 (m, 2 H), 7.50-7.41 (m, 5 H), 7.27-7.22 (m, 1 H), 6.79 (br. s., 2 H), 3.35 (s, 3 H, obscured by H₂O), 2.99 (s, 3 H) |
| 218 | 3-{2-Amino-4- [3-(5-chloro-2- fluoropyridin-3- yl)phenyl]-1- methyl-5-oxo- 4,5-dihydro-1H- imidazol-4- yl}phenyl methanesulfonate | 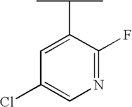 | CH₃SO₃ | 487 | 8.33-8.29 (m, 1 H), 8.19-8.15 (m, 1 H), 7.74-7.70 (m, 1 H), 7.59-7.50 (m, 3 H), 7.49-7.40 (m, 3 H), 7.27-7.22 (m, 1 H), 6.79 (br. s., 2 H), 3.35 (s, 3 H, obscured by H₂O), 2.99 (s, 3 H) |
| 219 | 3-[2-Amino-1- methyl-5-oxo-4- (3-pyrimidin-5- ylphenyl)-4,5- dihydro-1H- imidazol-4- yl]phenyl methanesulfonate | 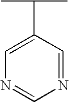 | CH₃SO₃ | 436 | 9.21-9.17 (m, 1 H), 9.02-8.99 (m, 1 H), 7.81-7.75 (m, 1 H), 7.70-7.65 (m, 1 H), 7.59-7.53 (m, 2 H), 7.53-7.41 (m, 3 H), 7.28-7.21 (m, 1 H), 6.79 (br. s., 2 H), 3.35 (s, 3 H, obscured by H₂O), 3.00 (s, 3 H) |
| 220 | 3-[2-Amino-1- methyl-5-oxo-4- (3-pyridin-3- ylphenyl)-4,5- dihydro-1H- imidazol-4- yl]phenyl propane-1- sulfonate | 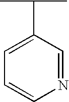 | CH₃CH₂CH₂SO₃ | 463 | 8.77-8.73 (m, 1 H), 8.57 (dd, J = 4.77, 1.76 Hz, 1 H), 7.96-7.91 (m, 1 H), 7.76-7.71 (m, 1 H), 7.59 (d, J = 7.53 Hz, 1 H), 7.56-7.49 (m, 2 H), 7.49-7.41 (m, 4 H), 7.24-7.17 (m, 1 H), 6.78 (br. s., 2 H), 3.48-3.41 (m, 2 H), 3.00 (s, 3 H), 1.83-1.77 (m, 2 H), 0.98 (t, J = 7.53 Hz, 3 H) |
| 221 | 3-{2-Amino-4- [3-(6- fluoropyridin-3- yl)phenyl]-1- methyl-5-oxo- 4,5-dihydro-1H- imidazol-4- yl}phenyl propane-1- sulfonate | 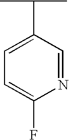 | CH₃CH₂CH₂SO₃ | 481 | 8.43-8.38 (m, 1 H), 8.17-8.10 (m, 1 H), 7.74-7.68 (m, 1 H), 7.60-7.50 (m, 3 H), 7.47-7.41 (m, 3 H), 7.30-7.25 (m, 1 H), 7.23-7.18 (m, 1 H), 6.79 (br. s., 2 H), 3.48-3.42 (m, 2 H), 3.00 (s, 3 H), 1.85-1.75 (m, 2 H), 1.01-0.96 (m, 3 H) |
| 222 | 3-{2-Amino-4- [3-(2- fluoropyridin-3- yl)phenyl]-1- methyl-5-oxo- 4,5-dihydro-1H- imidazol-4- yl}phenyl propane-1- sulfonate | 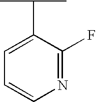 | CH₃CH₂CH₂SO₃ | 483 [M + H]⁺ | 8.32-8.15 (m, 1 H), 8.11-7.93 (m, 1 H), 7.76-7.65 (m, 1 H), 7.59-7.51 (m, 2 H), 7.51-7.40 (m, 5 H), 7.27-7.15 (m, 1 H), 6.79 (br. s., 2 H), 3.48-3.42 (m, 2 H), 2.99 (s, 3 H), 1.83-1.75 (m, 2 H), 1.00-0.96 (m, 3 H) |
| 223 | 3-{2-Amino-4- [3-(5-chloro-2- fluoropyridin-3- yl)phenyl]-1- methyl-5-oxo- 4,5-dihydro-1H- imidazol-4- yl}phenyl propane-1- sulfonate | 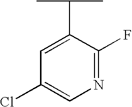 | CH₃CH₂CH₂SO₃ | 515 | 8.33-8.29 (m, 1 H), 8.20-8.14 (m, 1 H), 7.74-7.70 (m, 1 H), 7.58-7.51 (m, 3 H), 7.49-7.41 (m, 3 H), 7.24-7.18 (m, 1 H), 6.80 (br. s., 2 H), 3.47-3.41 (m, 2 H), 2.99 (s, 3 H), 1.85-1.76 (m, 2 H), 1.01-0.96 (m, 3 H) |
| 224 | 3-[2-Amino-1- methyl-5-oxo-4- (3-pyrimidin-5- ylphenyl)-4,5- dihydro-1H- imidazol-4- yl]phenyl propane-1- sulfonate | 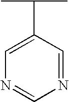 | CH₃CH₂CH₂SO₃ | 466 [M + H]⁺ | δ ppm 9.20-9.17 (s, 1 H), 9.00 (s, 2 H), 7.79-7.76 (m, 1 H), 7.70-7.65 (m, 1 H), 7.58-7.53 (m, 2 H), 7.52-7.41 (m, 3 H), 7.24-7.18 (m, 1 H), 6.80 (br. s., 2 H), 3.48-3.43 (m, 2 H), 3.00 (s, 3 H), 1.85-1.76 (m, 2 H), 1.01-0.96 (m, 3 H) |
| 225 | 3-[2-Amino-1- methyl-5-oxo-4- (3-pyridin-3- ylphenyl)-4,5- dihydro-1H- imidazol-4- yl]phenyl propane-2- sulfonate | 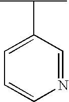 | (CH₃)₂CHSO₃ | 463 | δ ppm 8.79-8.74 (m, 1 H), 8.60-8.55 (m, 1 H), 7.97-7.91 (m, 1 H), 7.76-7.71 (m, 1 H), 7.62-7.57 (m, 1 H), 7.51-7.44 (m, 6 H), 7.22-7.16 (m, 1 H), 6.80 (br. s., 2 H), 3.73-3.64 (m, 1 H), 3.00 (s, 3 H), 1.41-1.38 (m, 6 H) |
| 226 | 3-{2-Amino-4- [3-(6- fluoropyridin-3- yl)phenyl]-1- methyl-5-oxo- 4,5-dihydro-1H- imidazol-4- yl}phenyl propane-2- sulfonate | 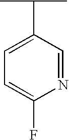 | (CH₃)₂CHSO₃ | 483 [M + H]⁺ | δ ppm 8.42-8.38 (m, 1 H), 8.17-8.10 (m, 1 H), 7.72-7.68 (m, 1 H), 7.59-7.49 (m, 4 H), 7.47-7.42 (m, 2 H), 7.30-7.25 (m, 1 H), 7.22-7.16 (m, 1 H), 6.79 (br. s., 2 H), 3.73-3.64 (m, 1 H), 3.00 (s, 3 H), 1.41-1.38 (m, 6 H) |
| 227 | 3-{2-Amino-4- [3-(2- fluoropyridin-3- yl)phenyl]-1- methyl-5-oxo- 4,5-dihydro-1H- imidazol-4- yl}phenyl propane-2- sulfonate | 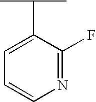 | (CH₃)₂CHSO₃ | 481 | 8.25-8.22 (m, 1 H), 8.04-7.98 (m, 1 H), 7.71-7.67 (m, 1 H), 7.55-7.51 (m, 2 H), 7.49-7.40 (m, 5 H), 7.21-7.17 (m, 1 H), 6.79 (br. s., 2 H), 3.72-3.64 (m, 1 H), 2.99 (s, 3 H), 1.41-1.37 (m, 6 H) |
| 228 | 3-[2-Amino-1- methyl-5-oxo-4- (3-pyrimidin-5- ylphenyl)-4,5- dihydro-1H- imidazol-4- yl]phenyl propane-2- sulfonate | 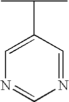 | (CH₃)₂CHSO₃ | 466 [M + H]⁺ | 9.20-9.17 (s, 1 H), 9.00 (s, 2 H), 7.79-7.74 (m, 1 H), 7.70-7.65 (m, 1 H), 7.57-7.53 (m, 2 H), 7.52-7.47 (m, 2 H), 7.46-7.39 (m, 1 H), 7.22-7.17 (m, 1 H), 6.80 (br. s., 2 H), 3.74-3.65 (m, 1 H), 3.00 (s, 3 H), 1.42-1.38 (m, 6 H) |
| 229 | 3-[2-Amino-1- methyl-5-oxo-4- (3-pyridin-3- ylphenyl)-4,5- dihydro-1H- imidazol-4- yl]phenyl 2- methoxyethane- sulfonate | 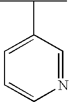 | CH₃OCH₂CH₂SO₃ | 479 | 8.77-8.75 (m, 1 H), 8.58-8.56 (m, 1 H), 7.96-7.92 (m, 1 H), 7.76-7.74 (m, 1 H), 7.61-7.57 (m, 1 H), 7.55-7.50 (m, 2 H), 7.50-7.46 (m, 2 H), 7.40-7.46 (m, 2 H), 7.24-7.19 (m, 1 H), 6.78 (br. s., 2 H), 3.76 (s, 4 H), 3.25 (s, 3 H), 3.00 (s, 3 H) |
| 230 | 3-{2-Amino-4- [3-(6- fluoropyridin-3- yl)phenyl]-1- methyl-5-oxo- 4,5-dihydro-1H- imidazol-4- yl}phenyl 2- methoxyethane- sulfonate | 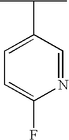 | CH₃OCH₂CH₂SO₃ | 499 [M + H]⁺ | 8.43-8.39 (m, 1 H), 8.17-8.10 (m, 1 H), 7.74-7.70 (m, 1 H), 7.60-7.56 (m, 1 H), 7.54-7.50 (m, 2 H), 7.49-7.40 (m, 3 H), 7.30-7.26 (m, 1 H), 7.24-7.19 (m, 1 H), 6.78 (br. s., 2 H), 3.76 (s, 4 H), 3.25 (s, 3 H), 3.00 (s, 3 H) |
| 231 | 3-{2-Amino-4- [3-(2- fluoropyridin-3- yl)phenyl]-1- methyl-5-oxo- 4,5-dihydro-1H- imidazol-4- yl}phenyl 2- methoxyethane- sulfonate | 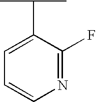 | CH₃OCH₂CH₂SO₃ | 497 | 8.26-8.21 (m, 1 H), 8.05-7.97 (m, 1 H), 7.72-7.67 (m, 1 H), 7.55-7.44 (m, 7 H), 7.25-7.18 (m, 1 H), 6.78 (br. s., 2 H), 3.75 (s, 4 H), 3.25 (s, 3 H), 2.99 (s, 3 H) |
| 232 | 3-{2-Amino-4- [3-(5-chloro-2- fluoropyridin-3- yl)phenyl]-1- methyl-5-oxo- 4,5-dihydro-1H- imidazol-4- yl}phenyl 2- methoxyethane- sulfonate acetate | 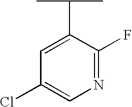 | CH₃OCH₂CH₂SO₃ | 533 [M + H]⁺ | 8.29-8.33 (m, 1 H), 8.14-8.19 (m, 1 H), 7.70-7.74 (m, 1 H), 7.48-7.58 (m, 3 H), 7.39-7.48 (m, 2 H), 7.19-7.24 (m, 1 H), 6.75-6.87 (m, 1 H), 3.75 (s, 4 H), 3.25 (s, 3 H), 2.99 (s, 3 H), 1.90 (s, 3 H) |
| 233 | 3-[2-Amino-1- methyl-5-oxo-4- (3-pyrimidin-5- ylphenyl)-4,5- dihydro-1H- imidazol-4- yl]phenyl 2- methoxyethane- sulfonate | 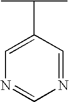 | CH₃OCH₂CH₂SO₃ | 480 | 9.19 (s, 1 H), 9.00 (s, 2 H), 7.80-7.76 (m, 1 H), 7.70-7.65 (m, 1 H), 7.58-7.47 (m, 4 H), 7.46-7.40 (m, 1 H), 7.24-7.19 (m, 1 H), 6.79 (br. s., 2 H), 3.76 (s, 4 H), 3.25 (s, 3 H), 3.00 (s, 3 H) |
| 234 | 3-[2-Amino-1- methyl-5-oxo-4- (3-pyridin-3- ylphenyl)-4,5- dihydro-1H- imidazol-4- yl]phenyl dimethylsulfamate | 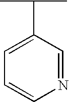 | (CH₃)₂NSO₃ | 464 | 8.77-8.73 (m, 1 H), 8.58-8.55 (m, 1 H), 7.95-7.91 (m, 1 H), 7.74-7.71 (m, 1 H), 7.62-7.57 (m, 1 H), 7.53-7.42 (m, 6 H), 7.22-7.17 (m, 1 H), 6.79 (br. s., 2 H), 3.00 (s, 3 H), 2.82 (s, 6 H) |
| 235 | 3-{2-Amino-4- [3-(6- fluoropyridin-3- yl)phenyl]-1- methyl-5-oxo- 4,5-dihydro-1H- imidazol-4- yl}phenyl dimethylsulfamate | 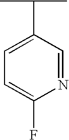 | (CH₃)₂NSO₃ | 482 | 8.42-8.38 (m, 1 H), 8.16-8.09 (m, 1 H), 7.72-7.68 (m, 1 H), 7.60-7.56 (m, 1 H), 7.54-7.50 (m, 2 H), 7.47-7.39 (m, 3 H), 7.31-7.25 (m, 1 H), 7.22-7.17 (m, 1 H), 6.79 (br. s., 2 H), 3.00 (s, 3 H), 2.82 (s, 6 H) |
| 336 | 3-{2-Amino-4- [3-(5-chloro-2- fluoropyridin-3- yl)phenyl]-1- methyl-5-oxo- 4,5-dihydro-1H- imidazol-4- yl}phenyl dimethylsulfamate | 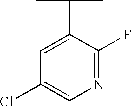 | (CH₃)₂NSO₃ | 518 [M + H]⁺ | 8.33-8.30 (m, 1 H), 8.18-8.14 (m, 1 H), 7.73-7.70 (m, 1 H), 7.57-7.47 (m, 5 H), 7.45-7.41 (m, 1 H), 7.22-7.17 (m, 1 H), 6.80 (br. s., 2 H), 2.99 (s, 3 H), 2.82 (s, 6 H) |
| 337 | 3-[2-Amino-1- methyl-5-oxo-4- (3-pyrimidin-5- ylphenyl)-4,5- dihydro-1H- imidazol-4- yl]phenyl dimethylsulfamate | 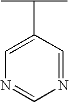 | (CH₃)₂NSO₃ | 465 | 9.19 (s, 1 H), 8.99 (s, 2 H), 7.78-7.75 (m, 1 H), 7.69-7.65 (m, 1 H), 7.57-7.47 (m, 4 H), 7.42 (t, J = 8.03 Hz, 1 H), 7.23-7.17 (m, 1 H), 6.80 (br. s., 2 H), 3.01 (s, 3 H), 2.83 (s, 6 H) |
| 238 | 3-[2-Amino-1- methyl-5-oxo-4- (3-pyridin-3- ylphenyl)-4,5- dihydro-1H- imidazol-4- yl]phenyl cyclopropane- sulfonate | 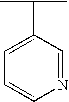 | 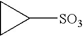 | 461 | 8.77-8.74 (m, 1 H), 8.57 (dd, J = 4.77, 1.76 Hz, 1 H), 7.96-7.91 (m, 1 H), 7.75-7.70 (m, 1 H), 7.62-7.58 (m, 1 H), 7.53-7.43 (m, 6 H), 7.25-7.21 (m, 1 H), 6.78 (br. s., 2 H) 3.00 (s, 3 H), 2.99 (s, 1 H), 1.11-1.03 (m, 2 H), 0.98-0.90 (m, 2 H) |
| 239 | 3-{2-Amino-4- [3-(6- fluoropyridin-3- yl)phenyl]-1- methyl-5-oxo- 4,5-dihydro-1H- imidazol-4- yl}phenyl cyclopropane- sulfonate | 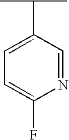 | 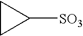 | 481 [M + H]⁺ | 8.42-8.38 (m, 1 H), 8.16-8.09 (m, 1 H), 7.72-7.68 (m, 1 H), 7.61-7.55 (m, 1 H), 7.55-7.49 (m, 3 H), 7.47-7.40 (m, 2 H), 7.30-7.25 (m, 1 H), 7.25-7.20 (m, 1 H), 6.78 (br. s., 2 H), 3.00 (s, 3 H), 2.99 (s, 1 H), 1.09-1.05 (m, 2 H), 0.95-0.91 (m, 2 H) |
| 240 | 3-{2-Amino-4- [3-(2- fluoropyridin-3- yl)phenyl]-1- methyl-5-oxo- 4,5-dihydro-1H- imidazol-4- yl}phenyl cyclopropane- sulfonate | 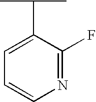 | 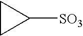 | 479 | 8.26-8.21 (m, 1 H), 8.04-7.97 (m, 1 H), 7.70-7.66 (m, 1 H), 7.55-7.50 (m, 2 H), 7.50-7.41 (m, 5 H), 7.25-7.21 (m, 1 H), 6.78 (br. s., 2 H), 3.00 (s, 3 H), 2.99 (s, 1 H), 1.10-1.04 (m, 2 H), 0.95-0.90 (m, 2 H), |
| 241 | 3-{2-Amino-4- [3-(5-chloro-2- fluoropyridin-3- yl)phenyl]-1- methyl-5-oxo- 4,5-dihydro-1H- imidazol-4- yl}phenyl cyclopropane- sulfonate | 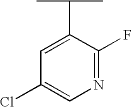 | 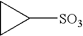 | N/A | 8.33-8.30 (m, 1 H), 8.16 (dd, J = 8.53, 2.51 Hz, 1 H), 7.73-7.69 (m, 1 H), 7.58-7.46 (m, 5 H), 7.46-7.40 (m, 1 H), 7.25-7.21 (m, 1 H), 6.79 (br. s., 2 H), 3.00 (s, 3 H), 2.99 (s, 1 H), 1.11-1.05 (m, 2 H), 0.95-0.91 (m, 2 H) |
| 242 | 3-[2-Amino-1- methyl-5-oxo-4- (3-pyrimidin-5- ylphenyl)-4,5- dihydro-1H- imidazol-4- yl]phenyl cyclopropane- sulfonate | 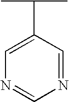 | 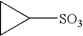 | 462 | 9.21-9.17 (s, 1 H), 9.00 (s, 2 H), 7.78-7.75 (m, 1 H), 7.70-7.65 (m, 1 H), 7.56-7.49 (m, 3 H), 7.47-7.42 (m, 2 H), 7.32-7.21 (m, 1 H), 6.84-6.75 (m, 2 H), 3.00 (s, 3 H), 2.99 (s, 1 H), 1.12-1.05 (m, 2 H), 0.97-0.90 (m, 2 H) |
| 243 | 3-[2-Amino-1- methyl-5-oxo-4- (3-pyridin-3- ylphenyl)-4,5- dihydro-1H- imidazol-4- yl]phenyl morpholine-4- sulfonate | 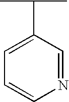 | 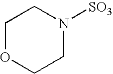 | 506 | 8.77-8.74 (m, 1 H), 8.57 (dd, J = 4.77, 1.51 Hz, 1 H), 7.96-7.91 (m, 1 H), 7.76-7.71 (m, 1 H), 7.62-7.56 (m, 1 H), 7.55-7.43 (m, 6 H), 7.25-7.18 (m, 1 H), 6.80 (br. s., 2 H), 3.58-3.54 (m, 4 H), 3.22-3.19 (m, 4 H), 3.00 (s, 3 H) |
| 244 | 3-{2-Amino-4- [3-(6- fluoropyridin-3- yl)phenyl]-1- methyl-5-oxo- 4,5-dihydro-1H- imidazol-4- yl}phenyl morpholine-4- sulfonate | 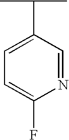 | 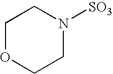 | 524 | 8.42-8.38 (m, 1 H), 8.16-8.10 (m, 1 H), 7.73-7.69 (m, 1 H), 7.60-7.56 (m, 1 H), 7.54-7.50 (m, 2 H), 7.49-7.43 (m, 3 H), 7.30-7.25 (m, 1 H), 7.24-7.19 (m, 1 H), 6.79 (br. s., 2 H), 3.58-3.55 (m, 4 H), 3.22-3.19 (m, 4 H), 3.00 (s, 3 H) |
| 245 | 3-{2-Amino-4- [3-(2- fluoropyridin-3- yl)phenyl]-1- methyl-5-oxo- 4,5-dihydro-1H- imidazol-4- yl}phenyl morpholine-4- sulfonate | 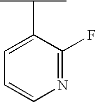 | 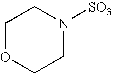 | 524 | 8.25-8.21 (m, 1 H), 8.01 (ddd, J = 10.29, 7.53, 2.01 Hz, 1 H), 7.71-7.68 (m, 1 H), 7.55-7.50 (m, 2 H), 7.49-7.41 (m, 5 H), 7.24-7.19 (m, 1 H), 6.79 (br. s., 2 H), 3.58-3.55 (m, 4 H), 3.23-3.19 (m, 4 H), 3.00 (s, 3 H) |
| 246 | 3-[2-Amino-1- methyl-5-oxo-4- (3-pyrimidin-5- ylphenyl)-4,5- dihydro-1H- imidazol-4- yl]phenyl morpholine-4- sulfonate | 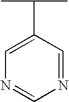 | 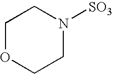 | 507 | 9.19 (s, 1 H), 9.00 (s, 2 H), 7.80-7.76 (m, 1 H), 7.70-7.65 (m, 1 H), 7.57-7.53 (m, 2 H), 7.52-7.47 (m, 2 H), 7.46-7.40 (m, 1 H), 7.25-7.19 (m, 1 H), 6.80 (br. s., 2 H), 3.58-3.55 (m, 4 H), 3.23-3.19 (m, 4 H), 3.01 (s, 3 H) |

### Examples 247-257

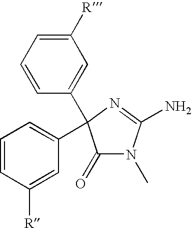

TABLE 4
Representative examples synthesized as described for examples were synthesized
as described for 4-(2-Amino-4-{3′-methoxy-5′-[(methylsulfonyl)oxy]biphenyl-3-yl}-1-
methyl-5-oxo-4,5-dihydro-1H-imidazol-4-yl)phenyl propane-1-sulfonate.

| | Chemical | | | [M − H]⁻ | |
|---|---|---|---|---|---|
| Ex | name | R″ | R′′′ | m/z | ¹H-NMR |
| 247 | 3-{2-Amino-4- [3-(5- methoxypyridin- 3-yl)phenyl]-1- methyl-5-oxo- 4,5-dihydro-1H- imidazol-4- yl}phenyl methanesulfonate | 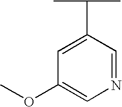 | CH₃SO₃ | 465 | 8.36-8.32 (m, 1 H), 8.30-8.27 (m, 1 H), 7.75-7.72 (m, 1 H), 7.63-7.58 (m, 1 H), 7.57-7.50 (m, 2 H), 7.49-7.39 (m, 4 H), 7.26-7.22 (m, 1 H), 6.78 (br. s., 2 H), 3.89 (s, 3 H), 3.35 (s, 3H, obscured by H₂O), 3.00 (s, 3 H) |
| 248 | 3-[2-Amino-1- methyl-5-oxo-4- (3-pyrimidin-2- ylphenyl)-4,5- dihydro-1H- imidazol-4- yl]phenyl methanesulfonate | 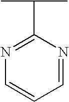 | CH₃SO₃ | 437 [M + H]⁺ | 7.62-7.59 (m, 1 H), 7.50-7.43 (m, 5 H), 7.42-7.39 (m, 1 H), 7.33-7.30 (m, 1 H), 7.29-7.24 (m, 1 H), 6.82 (br. s., 2 H), 3.36 (s, 3 H, obscured by H₂O), 2.99 (s, 3 H) 2 H missing |
| 249 | 3-{2-Amino-4- [3-(5- methoxypyridin- 3-yl)phenyl]-1- methyl-5-oxo- 4,5-dihydro-1H- imidazol-4- yl}phenyl propane-1- sulfonate | 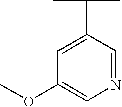 | CH₃CH₂CH₂SO₃ | 493 | 8.35-8.31 (m, 1 H), 8.30-8.27 (m, 1 H), 7.75-7.70 (m, 1 H), 7.64-7.58 (m, 1 H), 7.56-7.49 (m, 1 H), 7.48-7.39 (m, 4 H), 7.33-7.26 (m, 1 H), 7.16-7.26 (m, 1 H), 6.78 (br. s., 2 H), 3.90 (s, 3 H), 3.48-3.41 (m, 2 H), 3.00 (s, 3 H), 1.84-1.75 (m, 2 H), 1.02-0.97 (m, 3 H) |
| 250 | 3-[2-Amino-1- methyl-5-oxo-4- (3-pyrimidin-2- ylphenyl)-4,5- dihydro-1H- imidazol-4- yl]phenyl propane-1- sulfonate | 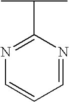 | CH₃CH₂CH₂SO₃ | 465 | δ ppm 7.61-7.58 (m, 1 H), 7.48-7.41 (m, 5 H), 7.40-7.36 (m, 1 H), 7.32-7.26 (m, 1 H), 7.24-7.19 (m, 1 H), 6.83 (br. s., 2 H), 3.48-3.43 (m, 2 H, obscured by H₂O), 2.99 (s, 3 H), 1.84-1.73 (m, 2 H), 1.03-0.98 (m, 3 H) 2 H missing |
| 251 | 3-{2-Amino-4- [3-(5- methoxypyridin- 3-yl)phenyl]-1- methyl-5-oxo- 4,5-dihydro-1H- imidazol-4- yl}phenyl propane-2- sulfonate | 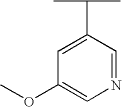 | (CH₃)₂CHSO₃ | 494 | 8.34-8.32 (m, 1 H), 8.30-8.28 (m, 1 H), 7.74-7.70 (m, 1 H), 7.63-7.58 (m, 1 H), 7.55-7.44 (m, 3 H), 7.43-7.39 (m, 2 H), 7.33-7.27 (m, 1 H), 7.25-7.16 (m, 1 H), 6.80 (br. s., 2 H), 3.89 (s, 3 H), 3.72-3.64 (m, 1 H), 3.00 (s, 3 H), 1.41-1.38 (m, 6 H) |
| 252 | 3-[2-Amino-1- methyl-5-oxo-4- (3-pyrimidin-2- ylphenyl)-4,5- dihydro-1H- imidazol-4- yl]phenyl propane-2- sulfonate | 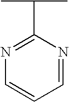 | (CH₃)₂CHSO₃ | 464 | 7.61-7.58 (m, 1 H), 7.49-7.38 (m, 6 H), 7.32-7.27 (m, 1 H), 7.22-7.17 (m, 1 H), 6.83 (br. s., 2 H), 3.73-3.65 (m, 1 H), 2.99 (s, 3 H), 1.42-1.39 (m, 6 H) 2 H missing |
| 253 | 3-{2-Amino-4- [3-(5- methoxypyridin- 3-yl)phenyl]-1- methyl-5-oxo- 4,5-dihydro-1H- imidazol-4- yl}phenyl 2- methoxyethane- sulfonate | 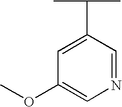 | CH₃OCH₂CH₂SO₃ | 509 | 8.35-8.32 (m, 1 H), 8.31-8.27 (m, 1 H), 7.76-7.71 (m, 1 H), 7.63-7.57 (m, 1 H), 7.54-7.51 (m, 1 H), 7.49-7.40 (m, 4 H), 7.33-7.26 (m, 1 H), 7.25-7.18 (m, 1 H), 6.78 (br. s., 2 H), 3.90-3.88 (m, 3 H), 3.77-3.75 (m, 4 H), 3.26-3.24 (m, 3 H), 3.00 (s, 3 H) |
| 254 | 3-{2-Amino-4- [3-(5- methoxypyridin- 3-yl)phenyl]-1- methyl-5-oxo- 4,5-dihydro-1H- imidazol-4- yl}phenyl dimethylsulfamate | 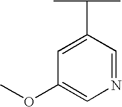 | (CH₃)₂NSO₃ | 496 [M + H]⁺ | 8.34-8.28 (m, 1 H), 7.74-7.69 (m, 1 H), 7.63-7.58 (m, 1 H), 7.54-7.49 (m, 1 H), 7.48-7.38 (m, 5 H), 7.34-7.27 (m, 1 H), 7.25-7.17 (m, 1 H), 6.83-6.69 (m, 2 H), 3.89 (s, 3 H), 3.00 (s, 3 H), 2.82 (s, 6 H) |
| 255 | 3-[2-Amino-1- methyl-5-oxo-4- (3-pyrimidin-2- ylphenyl)-4,5- dihydro-1H- imidazol-4- yl]phenyl dimethylsulfamate | 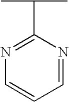 | (CH₃)₂NSO₃ | 465 | 7.60-7.58 (m, 1 H), 7.48-7.43 (m, 4 H), 7.42-7.38 (m, 2 H), 7.32-7.27 (m, 1 H), 7.22-7.18 (m, 1 H), 6.83 (br. s., 2 H), 2.99 (s, 3 H), 2.83 (s, 6 H) missing 2 H |
| 256 | 3-{2-Amino-4- [3-(5- methoxypyridin- 3-yl)phenyl]-1- methyl-5-oxo- 4,5-dihydro-1H- imidazol-4- yl}phenyl cyclopropane- sulfonate | 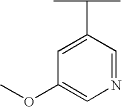 | 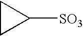 | 491 | 8.35-8.27 (m, 1 H), 7.74-7.69 (m, 1 H), 7.64-7.58 (m, 1 H), 7.55-7.49 (m, 2 H), 7.48-7.39 (m, 4 H), 7.33-7.26 (m, 1 H), 7.25-7.20 (m, 1 H), 6.78 (br. s., 2 H), 3.89 (s, 3 H), 3.00 (s, 3 H), 2.99 (s, 1 H), 1.11-1.05 (m, 2 H), 0.96-0.91 (m, 2 H) |
| 257 | 3-[2-Amino-1- methyl-5-oxo-4- (3-pyrimidin-2- ylphenyl)-4,5- dihydro-1H- imidazol-4- yl]phenyl cyclopropane- sulfonate | 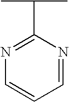 | 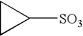 | 462 | 7.61-7.57 (m, 1 H), 7.48-7.41 (m, 6 H), 7.32-7.27 (m, 1 H), 7.25-7.22 (m, 1 H), 6.82 (br. s., 2 H), 2.99 (s, 3 H), 2.99 (s, 1 H), 1.13-1.08 (m, 2 H), 0.97-0.92 (m, 2 H), missing 2 H |

Due to the fast Xtime, 5.2 s, aromatic signals will show lower intensities than expected.

### Example 258

### 1-Ethynyl-3-(3-methoxyphenoxy)benzene

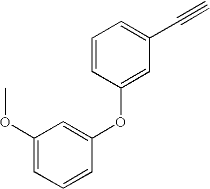

3-Hydroxyphenylacetylene (0.2 g, 1.7 mmol), 3-methoxy-2-(trimethylsilyl)phenyl trifluoromethanesulfonate (0.54 g, 1.65 mmol; described in: Pena, D.; Perez, D.; Guitian, E.; Castedo, L. J. Am. Chem. Soc. 1999, 121, 5827-5828) and cesium fluoride (0.78 g, 5.1 mmol) in acetonitrile (25 mL) was stirred for 48 h. The solvent was removed in vacuo and the resulting residue was dissolved in ethyl acetate and washed with saturated aqueous sodium carbonate, water and brine. The organic phase was dried over sodium sulfate and concentrated to give 0.32 g (87% yield) of the title compound: ¹H NMR (DMSO-d₆) δ 7.39 (t, J=7.91 Hz, 1H), 7.31 (t, J=8.16 Hz, 1H), 7.23-7.25 (m, 1H), 7.03-7.08 (m, 2H), 6.75-6.78 (m, 1H), 6.62 (t, J=2.38 Hz, 1H) 6.56-6.59 (m, 1H), 4.23 (s, 1H), 3.74 (s, 3H).

### Example 259

### 3-[(4-Methoxyphenyl)ethynyl]phenol

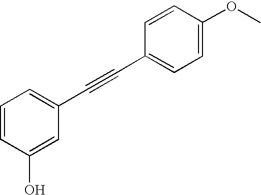

Triethylamine (10 mL) was added to a solution of 3-iodophenol (1.83 g, 8.3 mmol), 4-ethynanisol (1.08 g, 8.18 mmol), dichlorobis(triphenylphosphine)palladium(II) (29 mg, 0.04 mmol) and copper(I) iodide (8 mg, 0.04 mmol) in dry tetrahydrofuran (30 mL). The mixture was stirred at room temperature overnight then concentrated. The residue was dissolved in dichloromethane, washed with hydrochloric acid (0.5 M) and brine. The organic phase was dried over sodium sulfate and concentrated. Purification by column chromatography, using 0-15% ethyl acetate in n-heptane as the eluent, afforded 0.78 g (43% yield) of the title compound: ¹H NMR (DMSO-d₆) δ 9.64 (s, 1H), 7.46-7.49 (m, 2H), 7.19 (t, J=7.91 Hz, 1H), 6.96-6.99 (m, 2H), 6.92-6.95 (m, 1H), 6.86-6.88 (m, 1H), 6.77-6.81 (m, J=8.28, 2.51, 1.00 Hz, 1H), 3.79 (s, 3H).

### Example 260

### 4-{[3-(3-Methoxyphenoxy)phenyl]ethynyl}phenol

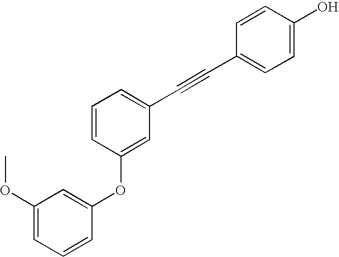

1-Ethynyl-3-(3-methoxyphenoxy)benzene (0.32 g, 1.44 mmol), 4-iodophenol (1.5 mmol, 0.33 g), copper(I) iodide (1.5 mg, 0.007 mmol) and dichlorobis(triphenylphosphine)palladium(II) (5 mg, 0.007 mmol) was stirred in dry tetrahydrofuran (10 mL) and triethylamine (3 mL) for 16 h and then concentrated. The residue was dissolved in dichloromethane, washed with hydrochloric acid (0.5 M) and brine. The organic phase was dried over sodium sulfate and concentrated. Purification by column chromatography, using 0-15% ethyl acetate in n-heptane as the eluent, afforded 0.09 g (19% yield) of the title compound: ¹H NMR (DMSO-d₆) δ 9.92 (s, 1H), 7.40-7.43 (m, 1H), 7.29-7.39 (m, 3H), 7.23-7.27 (m, 1H), 7.02-7.06 (m, 2H), 6.75-6.80 (m, 3H), 6.63 (t, J=2.38 Hz, 1H) 6.57-6.61 (m, 1H), 3.75 (s, 3H).

### Example 261

### 1-(3-Hydroxyphenyl)-2-(4-methoxyphenyl)ethane-1,2-dione

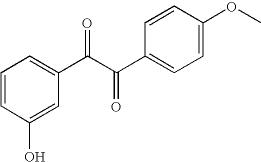

3-[(4-Methoxyphenyl)ethynyl]phenol (0.78 g, 3.47 mmol) and palladium dichloride (61 mg, 0.35 mmol) in dimethyl sulfoxide (35 mL) was heated at 130° C. for 5 h. On cooling to room temperature, water was added and the mixture extracted with ethyl acetate. The combined organic phases were washed with water, dried over sodium sulfate and concentrated to give 0.9 g (100% yield) of the title compound: ¹H NMR (DMSO-d₆) δ 10.54 (s, 1H), 8.35-8.39 (m, 2H), 7.92 (t, J=7.65 Hz, 1H), 7.77-7.81 (m, 2H), 7.64-7.68 (m, 3H), 4.39 (s, 3H).

### Example 262

### 5-(3-Hydroxyphenyl)-5-(4-methoxyphenyl)-3-methyl-2-thioxoimidazolidin-4-one

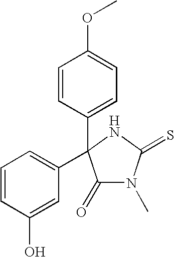

1-(3-Hydroxyphenyl)-2-(4-methoxyphenyl)ethane-1,2-dione (0.9 g, 3.5 mmol) and N-methylthiourea (0.63 g, 7 mmol) in dimethyl sulfoxide (25 mL)was heated to 100° C. Potassium hydroxide (1.2 M in water, 5.8 mL, 7 mmol) was added and the mixture was kept at 100° C. for 30 min. Water was added and pH adjusted to 4 with hydrochloric acid (2 M). The mixture was extracted with ethyl acetate and the combined organic phases were dried over sodium sulfate and concentrated. Purification by column chromatography, using 20-30% ethyl acetate in n-heptane as eluent, afforded 1.04 g (90% yield) of the title compound: ¹H NMR (DMSO-d₆) δ 11.50 (s, 1H), 9.57 (s, 1H), 7.22-7.26 (m, 2H), 7.19 (t, J=7.78 Hz, 1H), 6.97 (d, J=8.78 Hz, 2H), 6.68-6.75 (m, 3H), 3.75 (s, 3H), 3.16 (s, 3H).

### Example 263

### 1-(4-Hydroxyphenyl)-2-[3-(3-methoxyphenoxy)phenyl]ethane-1,2-dione

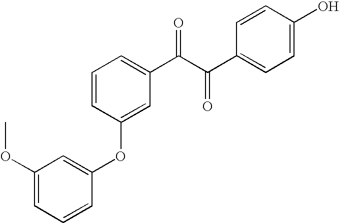

4-{[3-(3-methoxyphenoxy)phenyl]ethynyl}phenol (88 mg, 0.27 mmol) and palladium(II) dichloride (5 mg, 0.03 mmol) in dimethyl sulfoxide (3 mL) was heated at 130° C. for 5 h. After cooling to room temperature water was added and the mixture was extracted with ethyl acetate. The combined organic phases were washed with water, dried over sodium sulfate and concentrated to give 0.07 g (76% yield) of the title compound: ¹H NMR (DMSO-d₆) δ 10.90 (s, 1H), 7.75-7.79 (m, 2H), 7.55-7.63 (m, 2H), 7.41-7.44 (m, 2H), 7.33 (t, J=8.16 Hz, 1H), 6.91-6.95 (m, 2H), 6.80 (dd, J=8.28, 2.51 Hz, 1H), 6.68 (t, J=2.26 Hz, 1H), 6.62-6.65 (m, J=8.16, 2.13 Hz, 1H), 3.74 (s, 3H).

### Example 264

### 5-(4-Hydroxyphenyl)-5-[3-(3-methoxyphenoxy)phenyl]-3-methyl-2-thioxoimidazolidin-4-one

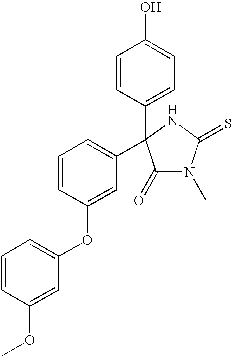

1-(4-Hydroxyphenyl)-2-[3-(3-methoxyphenoxy)phenyl]ethane-1,2-dione (71 mg, 0.2 mmol) and N-methylthiourea (0.04 g, 0.4 mmol) in dimethyl sulfoxide (5 mL) was heated to 100° C. Potassium hydroxide (1.2 M solution in water, 0.3 mL, 0.4 mmol) was added and the mixture was kept at 100° C. for 1 h. Water was added and the mixture was extracted with ethyl acetate. The combined organic phases were washed with water and brine, dried over sodium sulfate, filtered and concentrated to afford 0.085 g (100% yield) of the title compound: ¹H NMR (DMSO-d₆) δ 7.43 (t, J=8.03 Hz, 1H), 7.27 (t, J=8.16 Hz, 1H), 7.12-7.15 (m, 1H), 7.01-7.08 (m, 3H), 6.96-7.00 (m, 1H), 6.74-6.78 (m, 2H), 6.70-6.73 (m, 1H), 6.52-6.57 (m, 2H), 3.71 (s, 3H), 3.15 (s, 3H).

### Example 265

### 1-(4-Methoxyphenyl)-2-(3-phenoxyphenyl)ethane-1,2-dione

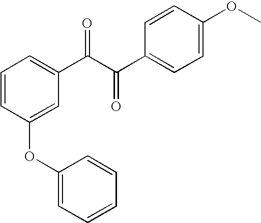

1-(3-hydroxyphenyl)-2-(4-methoxyphenyl)ethane-1,2-dione (0.27 g, 1.05 mmol) and cesium flouride (0.48 g, 3.15 mmol) was dissolved in acetonitrile (15 mL). 2-(trimethylsilyl)phenyl trifluoromethanesulfonate (0.38 mL, 1.58 mmol) was added and the mixture was stirred overnight. Ethyl acetate was added and the organic phase was washed with water, brine, dried over sodium sulfate and concentrated. Silica chromatography using 0-15% ethyl acetate in n-heptane as eluents afforded the title compound 0.225 g (64% yield.): ¹H NMR (DMSO-d₆) δ 7.86-7.89 (m, 2H), 7.56-7.64 (m, 2H), 7.42-7.47 (m, 4H), 7.21-7.25 (m, 1H), 7.09-7.16 (m, 4H), 3.88 (s, 3H).

### Example 266

### 5-(4-Methoxyphenyl)-3-methyl-5-(3-phenoxyphenyl)-2-thioxoimidazolidin-4-one

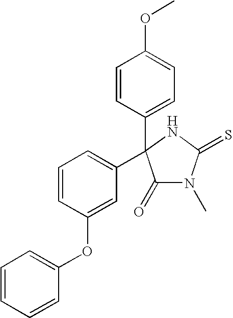

1-(4-methoxyphenyl)-2-(3-phenoxyphenyl)ethane-1,2-dione (0.22 g, 0.66 mmol) and N-methylthiourea (0.12 g, 1.32 mmol) was heated at 100° C. in dimethyl sulfoxide (8 mL). Potassium hydroxide (1.2 M solution in water, 1.1 mL, 1.32 mmol) was added and the mixture was kept at 100° C. for 30 min. Water was added and the mixture was extracted with ethyl acetate. The combined organic phases were washed with water and brine, dried over sodium sulfate, filtered and concentrated to afford 0.245 g (91% yield) of the title compound: ¹H NMR (DMSO-d₆) δ11.57 (s, 1H), 7.36-7.44 (m, 3H), 7.19-7.23 (m, 2H), 7.11-7.17 (m, 2H), 6.94-7.02 (m, 6H), 3.74 (s, 3H), 3.16 (s, 3H).

### Example 267

### 5-(4-Hydroxyphenyl)-3-methyl-5-(3-phenoxyphenyl)-2-thioxoimidazolidin-4-one

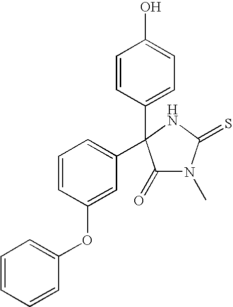

5-(4-Methoxyphenyl)-3-methyl-5-(3-phenoxyphenyl)-2-thioxoimidazolidin-4-one (0.245 g, 0.6 mmol) was dissolved in dichloromethane (10 mL) and cooled to 0° C. Boron tribromide (0.08 mL, 0.8 mmol) was added and the mixture was allowed to reach room temperature Additional boron tribromide (0.08 mL, 0.8 mmol) was added and the mixture was stirred for 6 h. Brine and ethyl acetate was added, the organic phase was collected, dried over sodium sulfate and concentrated to afford 0.24 g (102% yield) of the title product: ¹H NMR (DMSO-d₆) δ 11.52 (s, 1H), 9.67 (s, 1H), 7.38-7.43 (m, 2H), 7.11-7.15 (m, 2H), 7.06-7.08 (m, 2H), 6.94-7.03 (m, 5H), 6.75-6.78 (m, 2H), 3.15 (s, 3H).

General Method for Preparation of Sulfonyl Chlorides

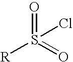

### Example 268

### 3-Methoxypropane-1-sulfonyl chloride

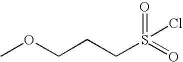

A mixture of 1-bromo-3-methoxypropane (2.5 g, 16.34 mmol) and sodium sulfite (2.06 g, 16.34 mmol) in water (25 mL) was heated at reflux for 24 h. The solvent was evaporated and the formed solid was washed with a mixture of diethyl ether/toluene (8:2) and dried in vacuo. The solid was added in portions to phosphorus oxychloride (25 mL) and heated at 80° C. for 5 h and then at 100° C. for 2 h. The mixture was cooled to room temperature, diluted with dichloromethane and filtrated. The filtrate was concentrated, ethyl acetate was added followed by ice. The ice was allowed to melt and the organic phase was collected, washed with water and brine, dried over sodium sulfate and concentrated to give 2.8 g (100% yield) of the title compound: 3.35 (t, J=6.53 Hz, 2H), 3.20 (s, 3H), 2.44-2.49 (m, 2H), 1.74-1.81 (m, 2H).

### Example 269

### 3-[2-Amino-4-(4-methoxyphenyl)-1-methyl-5-oxo-4,5-dihydro-1H-imidazol-4-yl]phenyl 3-methoxypropane-1-sulfonate hydrochloride

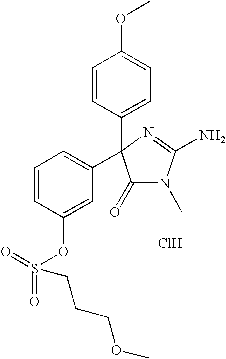

A solution of 5-(3-hydroxyphenyl)-5-(4-methoxyphenyl)-3-methyl-2-thioxoimidazolidin-4-one (45 mg, 0.14 mmol), triethylamine (28 μL, 0.2 mmol) and 3-methoxypropane-1-sulfonyl chloride (35 mg 0.2 mmol) in dichloromethane (5 mL) was stirred overnight. The solvent was evaporated and the residue dissolved in ethyl acetate and washed with hydrochloric acid (0.1 M), aqueous sodium hydroxide (0.1 M) and brine. The organic phase was dried over sodium sulfate, concentrated and the resulting residue was dissolved in methanol (2 mL). Aqueous ammonium hydroxide (conc. 1 mL) was added together with t-butyl hydroperoxide (0.15 mL, 70% in water, 1.5 mmol). The mixture was heated at 35° C. for 4 h and the product was isolated by preparative HPLC. The fractions were pooled and acetonitrile evaporated, aqueous sodium carbonate (sat.) was added and the mixture was extracted with ethyl acetate. The organic phase was dried over sodium sulfate, concentrated and resulting residue was dissolved in dichloromethane. Hydrochloric acid (0.5 mL, 1 M in diethyl ether) was added and the solvents were evaporated to afford 28 mg (41% yield) of the title compound. ¹H NMR (DMSO-d₆) δ 7.55-7.61 (m, 1H), 7.39-7.43 (m, 2H), 7.30-7.32 (m, 1H), 7.21-7.25 (m, 2H), 6.98-7.02 (m, 2H), 3.76 (s, 3H), 3.52-3.57 (m, 2H), 3.41-3.45 (m, 2H), 3.23 (s, 3H), 3.18 (s, 3H), 1.99-2.06 (m, 2H).

### Examples 270-273

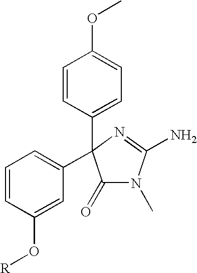

TABLE 5
Representative examples synthesized as described for 3-[2-Amino-4-(4-methoxyphenyl)-1-methyl-
5-oxo-4,5-dihydro-1H-imidazol-4-yl]phenyl 3-methoxypropane-1-sulfonate hydrochloride

| | | | [M − 1]⁻ | ¹H-NMR |
|---|---|---|---|---|
| Ex | Chemical name | R | m/z | (DMSO-d₆) δ |
| 270 | 3-[2-Amino-4-(4- methoxyphenyl)-1-methyl- 5-oxo-4,5-dihydro-1H- imidazol-4-yl]phenyl methanesulfonate hydrochloride | 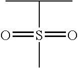 | 388 | 11.60 (br. s, 1H), 9.63 (s, 1H), 7.58 (t, J = 7.91 Hz, 1H), 7.39-7.45 (m, 2H), 7.35 (s, 1H), 7.22-7.27 (m, 2H), 6.97-7.02 (m, 2H), 3.76 (s, 3H), 3.41 (s, 3H), 3.19 (s, 3H). |
| 271 | 3-[2-Amino-4-(4- methoxyphenyl)-1-methyl- 5-oxo-4,5-dihydro-1H- imidazol-4-yl]phenyl 2- ethoxyethanesulfonate hydrochloride | 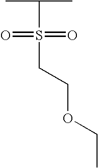 | 446 | 7.57 (t, J = 8.03 Hz, 1H), 7.39-7.43 (m, 2H), 7.32-7.33 (m, J = 2.01, 2.01 Hz, 1H), 7.21-7.25 (m, 2H), 6.98-7.02 (m, 2H), 3.79-3.82 (m, 4H), 3.76 (s, 3H), 3.45 (q, J = 7.03 Hz, 2H), 3.18 (s, 3H), 1.07 (t, J = 7.03 Hz, 3H). |
| 272 | 3-[2-Amino-4-(4- methoxyphenyl)-1-methyl- 5-oxo-4,5-dihydro-1H- imidazol-4-yl]phenyl 2- methoxyethanesulfonate hydrochloride | 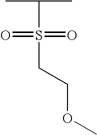 | 432 | 11.68 (br. s, 1H), 9.68 (br. s, 2H), 7.57 (t, J = 8.08 Hz, 1H), 7.38-7.43 (m, 2H), 7.33-7.36 (m, 1H), 7.23-7.28 (m, 2H), 6.98-7.02 (m, 2H), 3.77-3.84 (m, 4H), 3.76 (s, 3H), 3.26 (s, 3H), 3.20 (s, 3H). |

### Example 273

### 4-[2-Amino-1-methyl-5-oxo-4-(3-phenoxyphenyl)-4,5-dihydro-1H-imidazol-4-yl]phenyl methanesulfonate hydrochloride

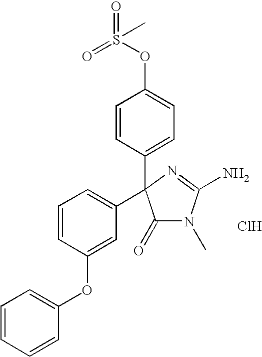

The title compound was synthesized as described for example 269 in 39% yield starting from 5-(4-hydroxyphenyl)-3-methyl-5-(3-phenoxyphenyl)-2-thioxoimidazolidin-4-one: ¹H NMR (DMSO-d₆) δ 11.79 (br. s, 1H), 9.75 (br. s, 2H), 7.48-7.51 (m, 2H), 7.37-7.46 (m, 5H), 7.14-7.19 (m, 2H), 7.10 (t, J=1.89 Hz, 1H), 6.98-7.03 (m, 3H), 3.41 (s, 3H), 3.19 (s, 3H); MS (ES) m/z 450 [M−1]⁻.

### Example 274

### 4-[2-Amino-1-methyl-5-oxo-4-(3-phenoxyphenyl)-4,5-dihydro-1H-imidazol-4-yl]phenyl propane-1-sulfonate hydrochloride

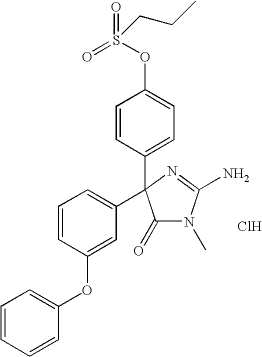

The title compound was synthesized as described for example 269 in 39% yield starting from 5-(4-hydroxyphenyl)-3-methyl-5-(3-phenoxyphenyl)-2-thioxoimidazolidin-4-one: ¹H NMR (DMSO-d₆) δ 7.37-7.49 (m, 7H), 7.14-7.18 (m, 2H), 7.09 (t, J=2.02 Hz, 1H), 6.98-7.03 (m, 3H), 3.50-3.55 (m, 2H), 3.17 (s, 3H), 1.80-1.89 (m, 2H), 1.03 (t, J=7.45 Hz, 3H); MS (ES) m/z 478 [M−1]⁻.

### Example 275

### 4-{2-Amino-4-[3-(3-methoxyphenoxy)phenyl]-1-methyl-5-oxo-4,5-dihydro-1H-imidazol-4-yl}phenyl methanesulfonate hydrochloride

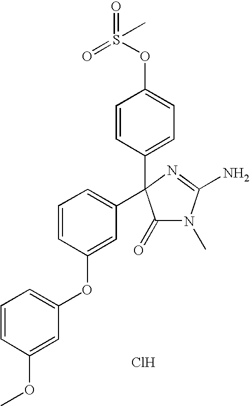

The title compound was synthesized as described for example 269 in 14% yield starting from 5-(4-hydroxyphenyl)-5-[3-(3-methoxyphenoxy)phenyl]-3-methyl-2-thioxoimidazolidin-4-one. ¹H NMR (DMSO-d₆) δ 7.42-7.50 (m, 5H), 7.28 (t, J=8.16 Hz, 1H), 7.15-7.18 (m, 1H), 7.08 (t, J=2.13 Hz, 1H), 7.02-7.06 (m, 1H), 6.72-6.75 (m, 1H), 6.53-6.58 (m, 2H), 3.72 (s, 3H), 3.41 (s, 3H), 3.18 (s, 3H); MS (ESI) m/z 480 [M−1]⁻.

### Example 276

### 3-[4-(3′-Methoxybiphenyl-3-yl)-1-methyl-5-oxo-2-thioxoimidazolidin-4-yl]phenyl propane-2-sulfonate

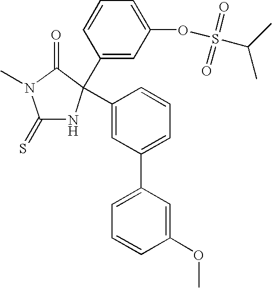

The title compound was synthesized as described for example 170 in 19% yield starting from 5-(3-hydroxyphenyl)-5-(3′-methoxybiphenyl-3-yl)-3-methyl-2-thioxoimidazolidin-4-one and propane-2-sulfonyl chloride. Purified by column chromatography, using dichloromethane/acetonitrile (95/5), as the eluent: ¹H NMR (CDCl₃, 400 MHz): δ 8.24 (s, 1H), 7.59 (d, J=8.1 Hz, 1H), 7.49 (t, J=1.8 Hz, 1H), 7.48-7.41 (m, 2H), 7.38-7.27 (m, 5H), 7.09 (d, J=7.8 Hz, 1H), 7.06-7.02 (m, 1H), 6.90 (dd, J=8.0, 2.2 Hz, 1H), 3.84 (s, 3H), 3.48-3.39 (m, 1H), 3.35 (s, 3H), 1.51 (dd, J=6.8, 1.3 Hz, 6H); MS (ES) m/z 511 [M+1]⁺.

### Example 277

### 3-[2-Amino-4-(3′-methoxybiphenyl-3-yl)-1-methyl-5-oxo-4,5-dihydro-1H-imidazol-4-yl]phenyl propane-2-sulfonate hydrochloride

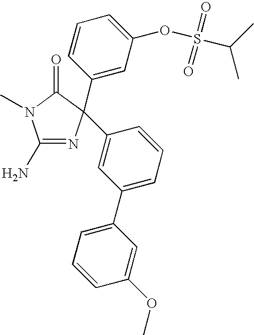

t-Butylhydroperoxide 70% in water (0.06 mL, 0.06 mmol) was added to a solution of 3-[4-(3′-methoxybiphenyl-3-yl)-1-methyl-5-oxo-2-thioxoimidazolidin-4-yl]phenyl propane-2-sulfonate (0.030 g, 0.059 mmol) in ethanol/aqueous ammonium hydroxide (33% (2:1, 3.8 mL). The resulting mixture was stirred at 35° C. for overnight. The solvents were evaporated and the residue was poured into water and extracted with dichloromethane. The combined organic extracts were dried over magnesium sulfate, and the solvent was removed in vacuo to afford 30 mg. Purification by column chromatography, using a gradient of acetonitril/triethylamine, (100/0 to 90/10) as the eluent, gave 35 mg of the base. The base was dissolved in dichloromethane and treated with hydrochloric acid (4 M, in diethyl ether), additional diethyl ether (30 mL) was added. The solvents were evaporated and the residue dried at 45° C. in a vacuum-cabinet overnight to afford 45 mg (27% yield) of the title compound: ¹H NMR (DMSO-d₆, 400 MHz): δ 11.79 (br s, 1H), 9.73 (br s, 2H), 7.74 (d, J=8.0 Hz, 1H), 7.68 (d, J=8.3 Hz, 1H), 7.63-7.58 (m, 3H), 7.56-7.52 (m, 2H), 7.42-7.34 (m, 2H), 7.19-7.13 (m, 2H), 6.97 (dd, J=8.2, 1.9 Hz, 1H), 3.81 (s, 3H), 3.20 (s, 3H); MS (ES) m/z 518 [M−1]⁻.

### Example 278

### 3-[4-(3′-Methoxybiphenyl-3-yl)-1-methyl-5-oxo-2-thioxoimidazolidin-4-yl]phenyl morpholine-4-sulfonate

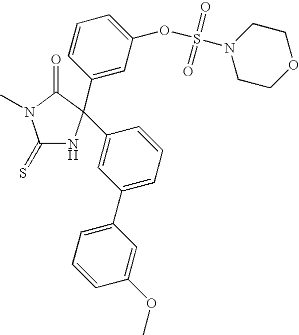

The title compound was synthesized as described for example 170 in 47% yield starting from 5-(3-hydroxyphenyl)-5-(3′-methoxybiphenyl-3-yl)-3-methyl-2-thioxoimidazolidin-4-one and morpholine-4-sulfonyl chloride. The crude product was poured into water and extracted with dichloromethane. The combined extracts were evaporated in vacuo. Purification by column chromatography, using dichloromethane/acetonitrile (95/5) as the eluent: ¹H NMR (CDCl₃, 400 MHz): δ 8.45 (s, 1H), 7.59 (d, J=7.8 Hz, 1H), 7.50 (t, J=1.8 Hz, 1H), 7.48-7.40 (m, 2H), 7.38-7.28 (m, 5H), 7.09 (d, J=7.6 Hz, 1H), 7.05-7.02 (m, 1H), 6.89 (dd, J=8.0, 2.2 Hz, 1H), 3.83 (s, 3H), 3.73-3.68 (m, 4H), 3.37-3.31 (m, 7H); MS (ES) m/z 552 [M−1]⁻.

### Example 279

### 3-[2-Amino-4-(3′-methoxybiphenyl-3-yl)-1-methyl-5-oxo-4,5-dihydro-1H-imidazol-4-yl]phenyl morpholine-4-sulfonate hydrochloride

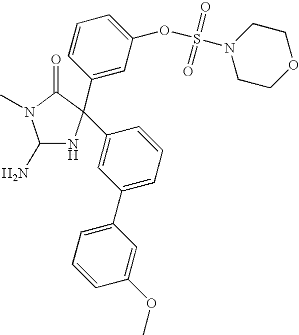

The title compound was synthesized as described for example 277 in 41% yield starting from 3-[4-(3′-methoxybiphenyl-3-yl)-1-methyl-5-oxo-2-thioxoimidazolidin-4-yl]phenyl morpholine-4-sulfonate, Purification by column chromatography, using acetonitrile/triethylamine (95/5) as the eluent: ¹H NMR (DMSO-d₆, 400 MHz): δ 11.71 (s, 1H), 9.67 (s, 2H), 7.72 (d, J=7.5 Hz, 1H), 7.63-7.51 (m, 3H), 7.49-7.34 (m, 5H), 7.20-7.12 (m, 2H), 6.97 (d, J=7.8 Hz, 1H), 3.81 (s, 3H), 3.60-3.55 (m, 4H), 3.26-3.21 (m, 4H), 3.20 (s, 3H); MS (ES) m/z 537 [M+1]⁺.

### Example 280

### 2-Amino-5-(3′-hydroxy-5′-methoxybiphenyl-3-yl)-3-methyl-5-phenyl-3,5-dihydro-4H-imidazol-4-one

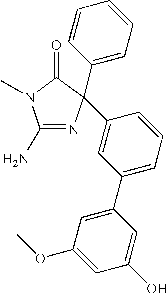

The title compound was synthesized as described for example 5 in 33% yield starting from 2-amino-5-(3-bromo-phenyl)-3-methyl-5-phenyl-3,5-dihydro-imidazol-4-one and 3-methoxy-5-(4,4,5,5-tetramethyl-1,3,2-dioxaborolan-2-yl)phenol. Purified by column chromatography, using ethyl acetate/methanol/aqueous ammonium hydroxide (33%), (94/5/1), as the eluent: ¹H-NMR (DMSO-d₆, 400 MHz): δ 9.56 (s, 1H), 7.67 (s, 1H), 7.49-7.42 (m, 4H), 7.38-7.27 (m, 3H), 7.25-7.19 (m, 1H), 6.66 (br s, 2H), 6.53-6.48 (m, 2H), 6.33 (t, J=2.1 Hz, 1H), 3.73 (s, 3H), 2.99 (s, 3H); MS (ES) m/z 388 [M+1]⁺.

### Example 281

### 3′-(2-Amino-1-methyl-5-oxo-4-phenyl-4,5-dihydro-1H-imidazol-4-yl)-5-methoxybiphenyl-3-yl trifluoromethanesulfonate hydrochloride

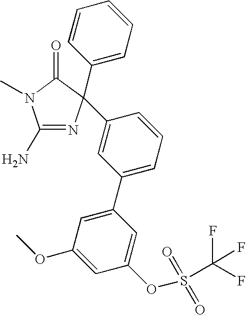

A mixture of 2-amino-5-(3′-hydroxy-5′-methoxybiphenyl-3-yl)-3-methyl-5-phenyl-3,5-dihydro-4H-imidazol-4-one (0.050 g, 0.13 mmol), 1,1,1-trifluoro-N-phenyl-N-[trifluoromethyl)sulfonyl]methanesulfonamide (0.046 g, 0.13 mmol), potassium carbonate (0.110 g, 0.81 mmol) in anhydrous tetrahydrofuran (3 mL) was irradiated in a microwave at 120° C. for 6 min. Additional 1,1,1-trifluoro-N-phenyl-N-[(trifluoromethyl)sulfonyl]methanesulfonamide (0.020 g, 0.05 mmol) was added and the reaction was irradiated for additional 6 min at 120° C. in a microwave. The mixture was filtered and the filtrate was concentrated in vacuo, purified by column chromatography, using acetonitrile/triethylamine, (95/5), as the eluent. The crude product was then poured into water and extracted with dichloromethane, the combined organic extracts were dried over sodium sulfate, and the solvent was evaporated in vacuo. The base was dissolved in dichloromethane and treated with 4 M hydrochloric acid in diethyl ether, additional diethyl ether (20 mL) was added. The mixture was evaporated and the residue was dried at room temperature in a vacuum-cabinet over night to afford 18 mg (25% yield) of the title compound: ¹H NMR (DMSO-d₆, 400 MHz): δ 11.63 (br s, 1H), 9.59 (br s, 2H), 7.78 (d, J=7.5 Hz, 1H), 7.68 (s, 1H), 7.58 (t, J=7.8 Hz, 1H), 7.50-7.36 (m, 6H), 7.32 (s, 1H), 7.25 (s, 1H), 7.16-7.12 (m, 1H), 3.88 (s, 3H), 3.20 (s, 3H); MS (ES) m/z 520 [M+1]⁺.

### Example 282

### 4-{2-Amino-4-[3-(5-methoxypyridin-3-yl)phenyl]-1-methyl-5-oxo-4,5-dihydro-1H-imidazol-4-yl}phenyl propane-1-sulfonate hydrochloride

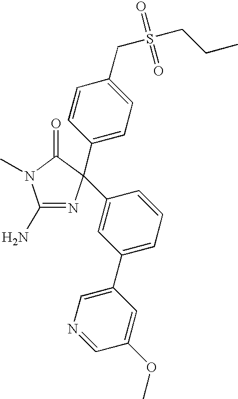

The title compound was synthesized as described for example 5 in 12% yield starting from 4-[2-amino-4-(3-bromophenyl)-1-methyl-5-oxo-4,5-dihydro-1H-imidazol-4-yl]phenyl propane-1-sulfonate and 3-methoxy-5-(4,4,5,5-tetramethyl-1,3,2-dioxaborolan-2-yl)pyridine. Purification by column chromatography, using ethyl acetate/methanol/aqueous ammonium hydroxide (33%) (94/5/1) as the eluent: ¹H-NMR (DMSO-d₆, 400 MHz): δ 11.92 (s, 1H), 9.67 (s, 2H), 8.59 (s, 1H), 8.43 (d, J=2.5 Hz, 1H), 7.89-7.82 (m, 3H), 7.61 (t, J=7.8 Hz, 1H), 7.56-7.51 (m, 2H), 7.47 (d, J=8.0, 1H), 7.44-7.39 (m, 2H), 3.96 (s, 3H), 3.55-3.49 (m, 2H) obscured by H₂O, 3.20 (s, 3H), 1.90-1.78 (m, 2H), 1.02 (t, J=7.4 Hz, 3H); MS (ES) m/z 494 [M+1]⁺.

### Example 283

### 6-Iodo-1,2,3,4-tetrahydronaphthalene

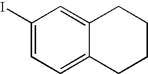

1,2,3,4-Tetrahydronaphthalene (6.10 g, 46.14 mmol), silver nitrate (7.84 g, 46.14 mmol) and iodide (11.71 g, 46.14 mmol) was added in portions to dichloromethane (500 mL) cooled to 0° C. and the reaction was stirred for 70 h at room temperature. The resulting precipitate was filtered off and the filtrate was washed with aqueous sodium thiosulfate (1 M) and saturated aqueous sodium hydrogen carbonate. The solvent was evaporated and the product was purified by column chromatography, using 0 to 5% ethyl acetate in n-heptane as the eluent, to give 5.0 g (42% yield) of the title compound: ¹H NMR (CDCl₃) δ 7.42-7.36 (m, 2H), 6.79 (d, J=7.83 Hz, 1H), 2.68-2.64 (m, 4H), 1.82-1.72 (m, 4H); MS (EI) m/z 258 [M]+•

### Example 284

### 6-[(3-Bromophenyl)ethynyl]-1,2,3,4-tetrahydronaphthalene

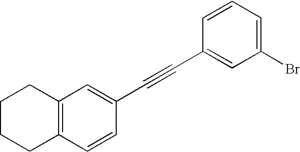

1-Bromo-3-ethynylbenzene (2.11 g, 11.63 mmol) was added to a solution of 6-iodo-1,2,3,4-tetrahydronaphthalene (3.0 g, 11.63 mmol), copper iodide (41 mg, 0.058 mmol) and dichlorobis(triphenylphosphine)palladium(II) (11 mg, 0.058 mmol) in anhydrous tetrahydrofuran (30 mL) and triethylamine (15 mL) and the reaction was stirred at room temperature overnight. Hydrochloric acid (30 mL, 2 M) was added and the aqueous phase was extracted with dichloromethane (×3). The combined organic phases were evaporated and the product was purified by column chromatography, using 0 to 5% ethyl acetate in n-heptane as the eluent to give 3.0 g (83% yield) of the title compound: ¹H NMR (CDCl₃) δ 7.68 (m, 1H), 7.50-7.410 (m, 2H), 7.28-7.19 (m, 3H), 7.05 (d, J=8.34 Hz, 1H), 2.78 (m, 4H), 1.81 (m, 4H); MS (EI) m/z 310, 312 [M]^{+•}

### Example 285

### 1-(3-Bromophenyl)-2-(5,6,7,8-tetrahydronaphthalen-2-yl)ethane-1,2-dione

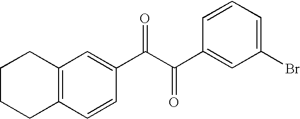

A solution of 6-[(3-bromophenyl)ethynyl]-1,2,3,4-tetrahydronaphthalene (3.0 g, 9.60 mmol) and palladium chloride (170 mg, 0.96 mmol in anhydrous dimethyl sulfoxide (30 mL) was heated at 150° C. for 4 h. Water was added and the aqueous phase was extracted with dichloromethane (×3). The combined organic phases were washed with water and concentrated in vacuo. Purification by column chromatography, using 0-20% ethyl acetate in n-heptane as the eluent, to give 2.24 g (68% yield) of the title compound: ¹H NMR (CDCl₃) δ 7.68 (dt, J=3.28, 1.64 Hz, 1H), 7.50-7.41 (m, 2H), 7.28-7.19 (m, 3H), 7.05 (d, J=8.34 Hz, 1H), 2.78 (d, J=5.81 Hz, 4H), 1.81 (ddd, J=6.44, 3.41, 3.28 Hz, 4H); MS (ES) m/z 341, 343 [M−1]⁻.

### Example 286

### 5-(3-Bromophenyl)-3-methyl-5-(5,6,7,8-tetrahydronaphthalen-2-yl)-2-thioxoimidazolidin-4-one

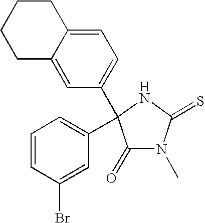

A solution of 1-(3-bromophenyl)-2-(5,6,7,8-tetrahydronaphthalen-2-yl)ethane-1,2-dione (2.24 g, 5.68 mmol) and N-methylthiourea (1.02 g, 11.36 mmol) in dimethyl sulfoxide (40 mL) was heated to 100° C. and potassium hydroxide (9.70 ml, 1.2 M) was added dropwise. After the addition the reaction was stirred at 100° C. for 5 min and was then allowed to cool to room temperature. The solution was diluted with water and made acidic using concentrated hydrochloric acid followed by extraction with dichloromethane (3×). The combined organic phases were washed with water and concentrated in vacuo to give 2.77 g (117% yield) of the title compound; MS (ES) m/z 415, 417 [M+1]⁺

### Example 287

### 2-Amino-5-(3-bromophenyl)-3-methyl-5-(5,6,7,8-tetrahydronaphthalen-2-yl)imidazolidin-4-one

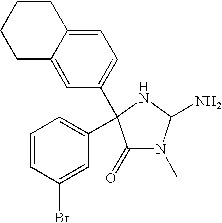

tert-Butyl hydroperoxide (9.0 g, 100 mmol) was added to a solution of 5-(3-bromophenyl)-3-methyl-5-(5,6,7,8-tetrahydronaphthalen-2-yl)-2-thioxoimidazolidin-4-one (2.77 g, 6.67 mmol) in methanol/ammonium hydroxide (3:1, 60 mL). The mixture was stirred at room temperature overnight and was then concentrated until approximately 50% of the volume remained. Water was added and the aqueous phase was extracted with dichloromethane (×3). The combined organic phases were washed with water and concentrated in vacuo. Purification by column chromatography, using 0-10% ammonium hydroxide/methanol (1:9) in dichloromethane as the eluent, to give 1.16 g (44% yield) of the title compound: ¹H NMR (CDCl₃) δ 7.69 (t, 1H), 7.44 (d, 1H), 7.40 (d, 1H), 7.18 (t, J=7.96 Hz, 1H), 7.13-7.10 (m, 2H), 7.01 (d, 1H), 3.11 (s, 3H), 2.76-2.69 (m, 4H), 1.79-1.74 (m, 4H); MS (ES) m/z 398, 400 [M+1]⁺

### Example 288

### 6-Bromo-1,2,3,4-tetrahydronaphthalen-2-yl methyl ether

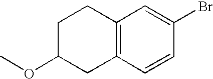

6-Bromo-1,2,3,4-tetrahydronaphthalen-2-ol (4.99 g, 21.98 mmol; described in Tschaen, D. et al. J. Org. Chem. 1995, 60(14), 4324-4330) was added to a stirred slurry of potassium hydroxide (4.93 g, 87.93 mmol) in anhydrous dimethyl sulfoxide (30 mL). The mixture was stirred for 5 min and iodomethane (6.24 g, 43.96 mmol) was added. The reaction was stirred overnight at room temperature, diluted with brine and extracted with dichloromethane (×3). The combined organic phases were washed with water (×3) and the concentrated in vacuo. Purification by column chromatography, using 0-100% ethyl acetate in n-heptane as the eluent, to give 3.79 g (72% yield) of the title compound: ¹H NMR (CDCl₃) δ 7.251-7.21 (m, 2H), 6.95 (d, J=8.08 Hz, 1H), 3.73-3.63 (m, 1H), 3.43 (s, 3H), 2.99-2.87 (m, 2H), 2.79-2.70 (m, 2H), 2.08-1.99 (m, 1H), 1.90-1.80 (m, 1H); MS (EI) m/z 240, 242 [M]^{+•}

### Example 289

### [(6-Methoxy-5,6,7,8-tetrahydronaphthalen-2-yl)ethynyl](trimethyl)silane

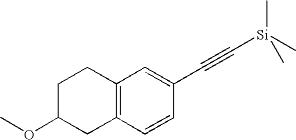

Tri-tert-butylphosphine (190 mg, 0.94 mmol), isopropylamine (1.91 g, 18.7 mmol) and (trimethylsilyl)acetylene (1.73 g, 19.65 mmol) was added to a solution of bis(benzonitrile)palladium (II) chloride (180 mg, 0.47 mmol) and copper iodide (59 mg, 0.31 mmol) in methanol (40 mL) under an atmosphere of argon. A solution of 6-bromo-1,2,3,4-tetrahydronaphthalen-2-yl methyl ether (3.79 g, 15.72 mmol), in anhydrous dioxane (10 mL) was added and the reaction was stirred at room temperature overnight. The reaction mixture was filtered through a short silica plug and concentrated in vacuo. Purification by column chromatography, using 0-20% ethyl acetate in n-heptane as the eluent, to give 3.93 g (93% yield) of the title compound. ¹H NMR (CDCl₃) δ 7.22 (t, J=7.58 Hz, 2H), 7.03-6.94 (m, 1H), 3.66 (ddd, J=10.99, 4.67, 2.78 Hz, 1H), 3.42 (s, 3H), 3.10-2.96 (m, 1H), 2.96-2.84 (m, 1H), 2.81-2.70 (m, 1H), 2.11-1.99 (m, 1H), 1.90-1.78 (m, 1H), 0.27-0.18 (s, 9H); MS (EI) m/z 258 [M]^{+•}

### Example 290

### 6-Ethynyl-1,2,3,4-tetrahydronaphthalen-2-yl methyl ether

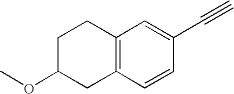

A solution of [(6-methoxy-5,6,7,8-tetrahydronaphthalen-2-yl)ethynyl](trimethyl)silane (3.93 g, 15.2 mmol) and potassium carbonate (8.09 g, 58.55 mmol) in methanol (40 mL) was heated at refluxed for 2 h and then cooled to room temperature. The mixture was filtered and the methanol was evaporated in vacuo. The resulting residue was diluted with chloroform, washed with brine (×3) and filtration through a celite plug. Purification by column chromatography, using 0-100% ethyl acetate in n-heptane as the eluent, to give 1.75 g (80% yield) of the title compound; MS (EI) m/z 186 [M]^{+•}

### Example 291

### 6-[(3-Bromophenyl)ethynyl]-1,2,3,4-tetrahydronaphthalen-2-yl methyl ether

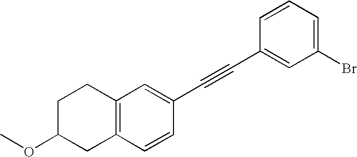

A solution of 6-Ethynyl-1,2,3,4-tetrahydronaphthalen-2-yl methyl ether (1.75 g, 6.95 mmol, 74% HPLC purity) in anhydrous tetrahydrofuran (5 mL) was added to a solution of 1-bromo-5-iodo-benzene (1.97 g, 6.95 mmol), dichlorobis(triphenylphosphine)palladium (25 mg, 0.035 mmol) and copper iodide (7 mg, 0.035 mmol) in anhydrous tetrahydrofuran (30 mL) and triethylamine (15 mL). The reaction mixture was stirred at room temperature over the weekend. The mixture was acidified using 2 M hydrochloric acid and the aqueous phase was extracted with dichloromethane (×3). Purification by column chromatography, using 0 to 20% ethyl acetate in n-heptane as the eluent, to give 1.68 g (71% yield) of the title compound; MS (EI) m/z 340, 342 [M]^{+•}

### Example 292

### 1-(3-Bromophenyl)-2-(6-methoxy-5,6,7,8-tetrahydronaphthalen-2-yl)ethane-1,2-dione

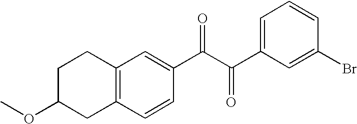

The title compound was prepared as described for Example 285 in 11% yield starting from 6-[(3-bromophenyl)ethynyl]-1,2,3,4-tetrahydronaphthalen-2-yl methyl ether; MS (ES) m/z 371, 373 [M−1]⁻

### Example 293

### 5-(3-Bromophenyl)-5-(6-methoxy-5,6,7,8-tetrahydronaphthalen-2-yl)-3-methyl-2-thioxoimidazolidin-4-one

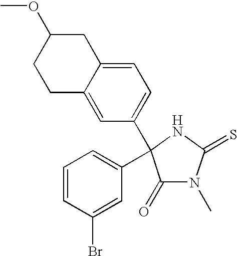

The title compound was prepared as described for Example 286 in 104% yield starting from 1-(3-bromophenyl)-2-(6-methoxy-5,6,7,8-tetrahydronaphthalen-2-yl)ethane-1,2-dione; MS (ES) m/z 443, 445 [M−1]⁻

### Example 294

### 2-Amino-5-(3-bromophenyl)-5-(6-methoxy-5,6,7,8-tetrahydronaphthalen-2-yl)-3-methyl-3,5-dihydro-4H-imidazol-4-one

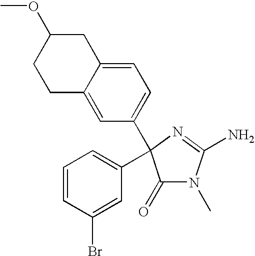

The title compound was prepared as described for Example 287 in 61% yield starting from 5-(3-bromophenyl)-5-(6-methoxy-5,6,7,8-tetrahydronaphthalen-2-yl)-3-methyl-2-thioxoimidazolidin-4-one; MS (ES) m/z 428, 430 [M+1]⁺

### Example 295

### 1-Acetyl-6-iodo-1,2,3,4-tetrahydroquinoline

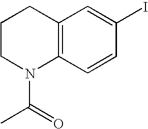

The title compound was prepared as described for Example 283 in 77% yield starting from 1-acetyl-1,2,3,4-tetrahydroquinoline (described in: Heyde C. et al. Eur. J. Org. Chem. 2000, 19, 3273-3278.): ¹H NMR (CDCl₃) δ 7.56-7.47 (m, 2H), 7.25-6.85 (m, 1H), 3.82-3.73 (m, 2H), 2.72 (dq, J=6.69, 6.44 Hz, 2H), 2.23 (s, 3H), 2.01-1.91 (m, 2H); MS (EI) m/z 301 [M]^{+•}

### Example 296

### 1-Acetyl-6-[(3-bromophenyl)ethynyl]-1,2,3,4-tetrahydroquinoline

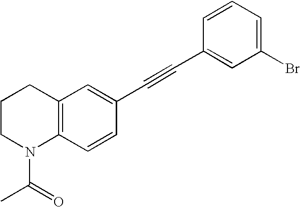

The title compound was prepared as described for Example 284 in 107% yield starting from 1-acetyl-6-iodo-1,2,3,4-tetrahydroquinoline; ¹H NMR (CDCl₃) δ 7.68 (t, J=1.77 Hz, 1H), 7.51-7.40 (m, 2H), 7.39-7.30 (m, 2H), 7.23 (t, J=7.83 Hz, 1H), 7.02-6.91 (m, 1H), 3.81 (t, J=6.44 Hz, 2H), 2.75 (t, J=6.57 Hz, 2H), 2.27 (s, 3H), 2.05-1.95 (m, 2H); MS (ES) m/z 354, 356 [M+1]⁺

### Example 297

### 1-(1-Acetyl-1,2,3,4-tetrahydroquinolin-6-yl)-2-(3-bromophenyl)ethane-1,2-dione

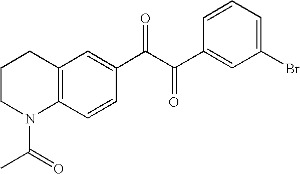

The title compound was prepared as described for Example 285 in 95% yield starting from 1-acetyl-6-[(3-bromophenyl)ethynyl]-1,2,3,4-tetrahydroquinoline: ¹H NMR (CDCl₃) δ 8.13 (t, J=1.77 Hz, 1H), 7.90 (dt, J=7.83, 1.26 Hz, 1H), 7.82-7.75 (m, 2H), 7.55 (d, J=8.34 Hz, 1H), 7.41 (t, J=7.83 Hz, 1H), 7.22-7.08 (m, 1H), 3.86-3.78 (m, 2H), 2.81 (t, J=6.57 Hz, 2H), 2.31 (s, 3H), 2.05-1.96 (m, 2H); MS (ES) m/z 386, 388 [M+1]⁺

### Example 298

### 5-(1-Acetyl-1,2,3,4-tetrahydroquinolin-6-yl)-5-(3-bromophenyl)-3-methyl-2-thioxoimidazolidin-4-one

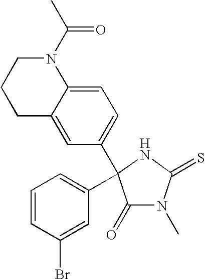

The title compound was prepared as described for Example 286 in 83% yield starting from 1-(1-acetyl-1,2,3,4-tetrahydroquinolin-6-yl)-2-(3-bromophenyl)ethane-1,2-dione; MS (ES) m/z 458, 460 [M+1]⁺

### Example 299

### 5-(1-Acetyl-1,2,3,4-tetrahydroquinolin-6-yl)-2-amino-5-(3-bromophenyl)-3-methyl-3,5-dihydro-4H-imidazol-4-one

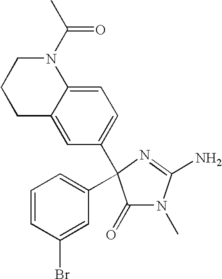

The title compound was prepared as described for Example 287 in 50% yield starting from 5-(1-acetyl-1,2,3,4-tetrahydroquinolin-6-yl)-5-(3-bromophenyl)-3-methyl-2-thioxoimidazolidin-4-on; MS (ES) m/z 441, 443 [M+1]⁺

### Example 300

### 5-[(3-Bromophenyl)ethynyl]indane

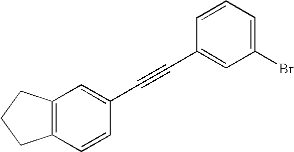

The title compound was prepared as described for Example 284 in 93% yield starting from 5-iodoindane (described in: Walser, A. et al. J. Med. Chem. 1991, 34(4), 1440-1446.): ¹H NMR (CDCl₃) δ 7.68 (t, J=1.64 Hz, 1H), 7.45 (dt, J=7.89, 1.86 Hz, 2H), 7.40 (s, 1H), 7.32 (d, J=7.83 Hz, 1H), 7.25-7.17 (m, 2H), 2.92 (ddd, J=11.49, 7.71, 4.29 Hz, 4H), 2.15-2.04 (m, 2H); MS (EI) m/z 296, 298 [M]^{+•}

### Example 301

### 1-(3-Bromophenyl)-2-(2,3-dihydro-1H-inden-5-yl)ethane-1,2-dione

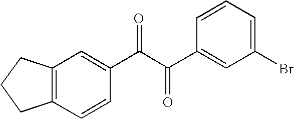

The title compound was prepared as described for Example 285 in 101% yield starting from 5-[(3-bromophenyl)ethynyl]indane: ¹H NMR (CDCl₃) δ 8.13 (t, J=1.77 Hz, 1H), 7.89 (d, 1H), 7.83-7.73 (m, 3H), 7.42-7.34 (m, 2H), 3.02-2.92 (m, 4H), 2.19-2.09 (m, 2H); MS (ES) m/z 327, 329 [M−1]⁻

### Example 302

### 5-(3-Bromophenyl)-5-(2,3-dihydro-1H-inden-5-yl)-3-methyl-2-thioxoimidazolidin-4-one

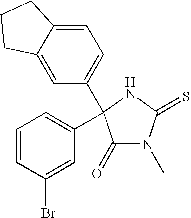

The title compound was prepared as described for Example 286 in 115% yield starting from 1-(3-bromophenyl)-2-(2,3-dihydro-1H-inden-5-yl)ethane-1,2-dione; MS (ES) m/z 401, 403 [M+1]⁺

### Example 303

### 2-Amino-5-(3-bromophenyl)-5-(2,3-dihydro-1H-inden-5-yl)-3-methylimidazolidin-4-one

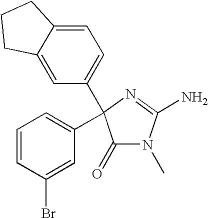

The title compound was prepared as described for Example 287 in 43% yield starting from 5-(3-bromophenyl)-5-(2,3-dihydro-1H-inden-5-yl)-3-methyl-2-thioxoimidazolidin-4-one: MS (ES) m/z 384, 386 [M+1]⁺

### Example 304

### 2-Amino-5-(3′-methoxybiphenyl-3-yl)-5-(6-methoxy-5,6,7,8-tetrahydronaphthalen-2-yl)-3-methyl-3,5-dihydro-4H-imidazol-4-one 0.25 acetate

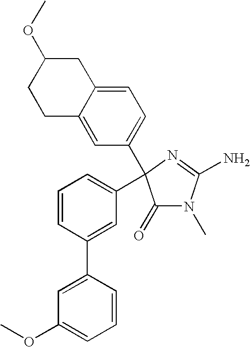

2-Amino-5-(3-bromophenyl)-5-(6-methoxy-5,6,7,8-tetrahydronaphthalen-2-yl)-3-methyl-3,5-dihydro-4H-imidazol-4-one (50 mg, 0.117 mmol), (3-methoxyphenyl)boronic acid (23 mg, 0.152 mmol), [1,1′-bis(diphenylphosphino)ferrocene]palladium(II) chloride dichloromethane adduct (10 mg, 0.012 mmol), potassium carbonate (102 mg, 0.738 mmol), 100 μL water in anhydrous tetrahydrofuran (3 mL) was irradiated in a microwave at 150° C. for 15 minutes. When cooled to ambient temperature the mixture was filtered and dimethyl sulfoxide (500 μL) was added. The solution was concentrated in vacuo to remove tetrahydrofuran and purified by preparative HPLC to give 12 mg (22% yield) of the title compound: ¹H NMR (DMSO-d₆) δ 7.72 (s, 1H), 7.52 (d, J=7.53 Hz, 1H), 7.44-7.48 (m, 1H), 7.36-7.42 (m, 2H), 7.16-7.21 (m, 2H), 7.11 (d, J=7.78 Hz, 1H), 7.05-7.08 (m, J=2.26 Hz, 1H), 6.93-7.02 (m, 2H), 3.82 (s, 3H), 3.57-3.65 (m, 1H), 3.28 (s, 3H,), 2.90-3.01 (m, 4H), 2.71-2.80 (m, 1H), 2.60-2.69 (m, 2H), 1.89-1.97 (m, 1H), 1.67-1.76 (m, 1H); MS (ES) m/z 456 [M+1]⁺

### Examples 305-325

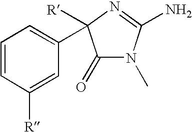

TABLE 6
Representative examples synthesized as described for 2-Amino-5-(3′-methoxybiphenyl-3-yl)-5-(6-
methoxy-5,6,7,8-tetrahydronaphthalen-2-yl)-3-methyl-3,5-dihydro-4H-imidazol-4-one 0.25 acetate

| | | | | [M − H]⁻ | ¹H-NMR |
|---|---|---|---|---|---|
| Ex | Chemical name | R′ | R″ | m/z | (DMSO-d₆) δ |
| 305 | 2-Amino-5-(2′-fluoro-5′- methoxybiphenyl-3-yl)-5- (6-methoxy-5,6,7,8- tetrahydronaphthalen-2-yl)- 3-methyl-3,5-dihydro-4H- imidazol-4-one | 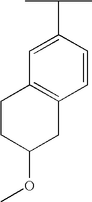 | 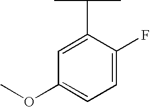 | 474 | 7.66 (s, 1H) 7.46-7.52 (m, 1H) 7.38-7.44 (m, 2H) 7.13-7.34 (m, 3H) 6.89-7.08 (m, 3H) 3.79 (s, 3H) 3.56-3.66 (m, 1H) 3.26-3.29 (m, 3H) 2.90-3.02 (m, 4H) 2.70-2.80 (m, 1H) 2.58-2.70 (m, 2H) 1.89-2.00 (m, 1H) 1.64-1.78 (m, 1H) |
| 306 | 5-(1-Acetyl-1,2,3,4- tetrahydroquinolin-6-yl)-2- amino-5-(3′- methoxybiphenyl-3-yl)-3- methyl-3,5-dihydro-4H- imidazol-4-one | 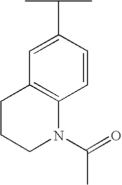 | 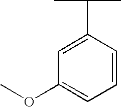 | 469 | 7.75 (s, 1H) 7.51 (dd, J = 12.80 Hz, 2H) 7.36-7.43 (m, 3H) 7.25-7.31 (m, 2H) 7.11 (d, J = 8.03 Hz, 1H) 7.05-7.08 (m, J = 2.26 Hz, 1H) 6.96 (dd, J = 2.26 Hz, 1H) 3.82 (s, 3H) 3.64 (t, J = 6.27 Hz, 2H) 3.00 (s, 3H) 2.66 (t, J = 6.65 Hz, 2H) 2.13 (s, 3H) 1.79-1.87 (m, 2H) |
| 307 | 5-(1-Acetyl-1,2,3,4- tetrahydroquinolin-6-yl)-2- amino-5-(2′,5′- dimethoxybiphenyl-3- methyl-3,5-dihydro-4H- imidazol-4-one 0.25 acetate | 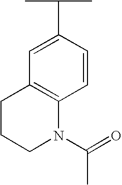 | 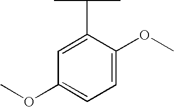 | 499 | 7.62 (s, 1H) 7.27-7.44 (m, 6H) 7.03 (d, J = 8.78 Hz, 1H) 6.91 (dd, J = 3.14 Hz, 1H) 6.77 (d, J = 3.01 Hz, 1H) 3.74 (s, 3H) 3.62-3.68 (m, 5H) 2.99 (s, 3H) 2.67 (t, J = 6.40 Hz, 2H) 2.14 (s, 3H) 1.91 (s, 0.6H) 1.80-1.88 (m, 2H) |
| 308 | 3′-[4-(1-Acetyl-1,2,3,4- tetrahydroquinolin-6-yl)-2- amino-1-methyl-5-oxo-4,5- dihydro-1H-imidazol-4- yl]biphenyl-3-carbonitrile | 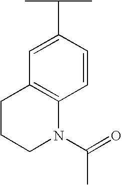 | 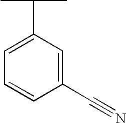 | 464 | 8.02 (s, 1H) 7.90 (d, J = 8.03 Hz, 1H) 7.80-7.87 (m, 2H) 7.70 (t, J = 7.78 Hz, 1H) 7.55-7.64 (m, J = 7.65 Hz, 3H) 7.46 (t, 1H) 7.24-7.30 (m, 2H) 3.64 (t, J = 6.27 Hz, 2H) 3.00 (s, 3H) 2.67 (t, J = 6.65 Hz, 2H) 2.13 (s, 3H) 1.81-1.87 (m, 2H) |
| 309 | 3′-[4-(1-Acetyl-1,2,3,4- tetrahydroquinolin-6-yl)-2- amino-1-methyl-5-oxo-4,5- dihydro-1H-imidazol-4-yl]- 4-fluorobiphenyl-3- carbonitrile | 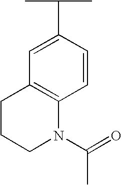 | 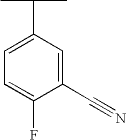 | 482 | 8.11 (dd, J = 2.13 Hz, 1H) 7.91-7.96 (m, 1H) 7.77 (s, 1H) 7.53-7.65 (m, 4H) 7.43 (t, J = 7.65 Hz, 1H) 7.23-7.29 (m, 2H) 3.63 (t, J = 6.27 Hz, 2H) 2.99 (s, 3H) 2.65 (t, J = 6.53 Hz, 2H) 2.12 (s, 3H) 1.79-1.86 (m, 2H) |
| 310 | 5-(1-Acetyl-1,2,3,4- tetrahydroquinolin-6-yl)-2- amino-5-(2′-fluoro-5′- methoxybiphenyl-3-yl)-3- methyl-3,5-dihydro-4H- imidazol-4-one 0.25 acetate | 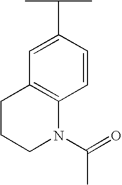 | 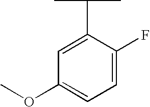 | 487 | 7.69 (s, 1H) 7.50-7.55 (m, 1H) 7.20-7.45 (m, 6H) 6.91-6.99 (m, 2H) 3.79 (s, 3H) 3.65 (t, J = 6.40 Hz, 2H) 2.99 (s, 3H) 2.66 (t, J = 6.65 Hz, 2H) 2.14 (s, 3H) 1.91 (s, 0.6H) 1.80-1.88 (m, 2H) |
| 311 | 5-(1-Acetyl-1,2,3,4- tetrahydroquinolin-6-yl)-2- amino-3-methyl-5-(3′- nitrobiphenyl-3-yl)-3,5- dihydro-4H-imidazol-4-one 0.25 acetate | 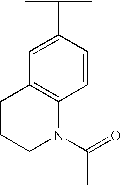 | 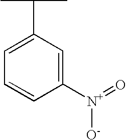 | 484 | 8.30 (s, 1H) 8.23 (dd, J = 1.51 Hz, 1H) 8.04 (d, J = 8.03 Hz, 1H) 7.84 (s, 1H) 7.77 (t, J = 7.91 Hz, 1H) 7.65 (d, J = 7.53 Hz, 1H) 7.60 (d, J = 8.03 Hz, 1H) 7.31-7.50 (m, J = 7.65 Hz, 2H) 7.22-7.29 (m, 2H) 3.63 (t, J = 6.27 Hz, 2H) 2.99 (s, 3H) 2.65 (t, J = 6.65 Hz, 2H) 2.12 (s, 3H) 1.91 (s, 0.6H) 1.78-1.86 (m, 2H) |
| 312 | 5-(1-Acetyl-1,2,3,4- tetrahydroquinolin-6-yl)-2- amino-5-(2′-fluoro-3′- methoxybiphenyl-3-yl)-3- methyl-3,5-dihydro-4H- imidazol-4-one | 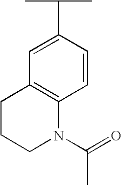 | 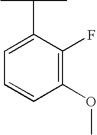 | 487 | 7.67 (s, 1H) 7.52 (d, J = 7.03 Hz, 1H) 7.36-7.45 (m, 3H) 7.15-7.31 (m, 4H) 6.93-6.98 (m, 1H) 3.88 (s, 3H) 3.64 (t, J = 6.27 Hz, 2H) 2.99 (s, 3H) 2.66 (t, J = 6.65 Hz, 2H) 2.13 (s, 3H) 1.79-1.88 (m, 2H) |
| 313 | 5-(1-Acetyl-1,2,3,4- tetrahydroquinolin-6-yl)-2- amino-5-[3-(3- furyl)phenyl]-3-methyl-3,5- dihydro-4H-imidazol-4-one | 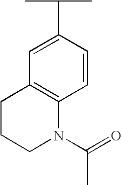 | 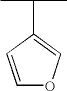 | 429 | 8.04 (s, 1H) 7.73 (t, J = 1.63 Hz, 1H) 7.63 (s, 1H) 7.19-7.49 (m, 6H) 6.79 (s, 1H) 3.63 (t, J = 6.27 Hz, 2H) 2.98 (s, 3H) 2.65 (t, J = 6.65 Hz, 2H) 2.12 (s, 3H) 1.79-1.85 (m, 2H) |
| 314 | 2-Amino-5-(3′- methoxybiphenyl-3-yl)-3- methyl-5-(5,6,7,8- tetrahydronaphthalen-2-yl)- 3,5-dihydro-4H-imidazol-4- one | 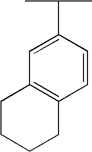 | 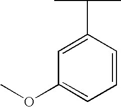 | 426 | 7.70 (s, 1H) 7.50 (d, J = 7.53 Hz, 1H) 7.45 (d, J = 8.03 Hz, 1H) 7.33-7.41 (m, 2H) 7.02-7.23 (m, 4H) 6.92-6.99 (m, 2H) 3.80 (s, 3H) 2.98 (s, 3H) 2.60-2.68 (m, 4H) 1.68 (s, 4H) |
| 315 | 3′-[2-Amino-1-methyl-5- oxo-4-(5,6,7,8- tetrahydronaphthalen-2-yl)- 4,5-dihydro-1H-imidazol-4- yl]-6-fluorobiphenyl-3- carbonitrile | 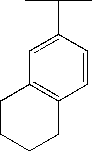 | 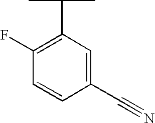 | 439 | 7.98 (dd, J = 2.13 Hz, 1H) 7.91-7.96 (m, 1H) 7.69 (s, 1H) 7.38-7.63 (m, 4H) 7.07-7.18 (m, 2H) 6.93-6.99 (m, J = 8.53 Hz, 1H) 2.92-3.01 (m, 3H) 2.59-2.69 (m, 4H) 1.69 (s, 4H) |
| 316 | 2-Amino-5-(2′,5′- dimethoxybiphenyl-3-yl)-3- methyl-5-(5,6,7,8- tetrahydronaphthalen-2-yl)- 3,5-dihydro-4H-imidazol-4- one | 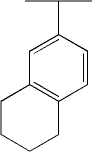 | 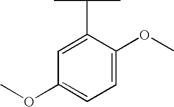 | 456 | 7.59 (s, 1H) 7.28-7.42 (m, 3H) 7.17-7.22 (m, 2H) 6.95-7.04 (m, 2H) 6.91 (dd, J = 3.14 Hz, 1H) 6.76 (d, J = 3.01 Hz, 1H) 3.73-3.75 (m, 3H) 3.66 (s, 3H) 2.98 (s, 3H) 2.62-2.70 (m, 4H) 1.67-1.75 (m, 4H) |
| 317 | 2-Amino-5-[3-(1,3- benzodioxol-5-yl)phenyl]- 3-methyl-5-(5,6,7,8- tetrahydronaphthalen-2-yl)- 3,5-dihydro-4H-imidazol-4- one 0.25 acetate | 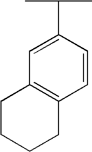 | 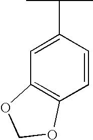 | 440 | 7.65 (s, 1H) 7.40 (dd, J = 1.64 Hz, 2H) 7.28-7.36 (m, J = 7.58 Hz, 1H) 7.12-7.18 (m, 2H) 7.08 (s, 1H) 6.92-7.01 (m, 3H) 6.05 (s, 2H) 2.97 (s, 3H) 2.59-2.67 (m, 4H) 1.91 (s, 1H) 1.65-1.73 (m, 4H) |
| 318 | 2-Amino-5-(3′- ethoxybiphenyl-3-yl)-3- methyl-5-(5,6,7,8- tetrahydronaphthalen-2-yl)- 3,5-dihydro-4H-imidazol-4- one | 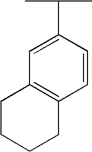 | 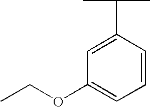 | 440 | 7.71 (s, 1H) 7.43-7.54 (m, 2H) 7.34-7.41 (m, 2H) 7.12-7.21 (m, 2H) 7.09 (d, J = 8.03 Hz, 1H) 7.02-7.06 (m, 1H) 6.90-7.00 (m, 2H) 4.09 (q, J = 6.94 Hz, 2H) 2.99 (s, 3H) 2.60-2.70 (m, 4H) 1.64-1.75 (m, 4H) 1.36 (t, J = 7.03 Hz, 3H) |
| 319 | 3′-[2-Amino-1-methyl-5- oxo-4-(5,6,7,8- tetrahydronaphthalen-2-yl)- 4,5-dihydro-1H-imidazol-4- yl]biphenyl-3-carbonitrile | 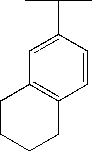 | 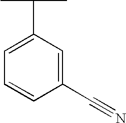 | 421 | 7.94-8.01 (m, J = 19.71 Hz, 1H) 7.81-7.90 (m, J = 7.83 Hz, 2H) 7.75-7.78 (m, 1H) 7.69 (t, 1H) 7.39-7.61 (m, 3H) 7.06-7.19 (m, 2H) 6.94-7.00 (m, J = 8.34 Hz, 1H) 2.98 (s, 3H) 2.60-2.67 (m, 4H) 1.64-1.72 (m, 4H) |
| 320 | 3′-[2-Amino-1-methyl-5- oxo-4-(5,6,7,8- tetrahydronaphthalen-2-yl)- 4,5-dihydro-1H-imidazol-4- yl]-4-fluorobiphenyl-3- carbonitrile | 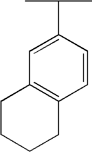 | 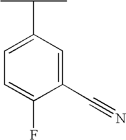 | 439 | 8.10 (dd, J = 2.38 Hz, 1H) 7.90-7.95 (m, 1H) 7.74 (s, 1H) 7.37-7.67 (m, 4H) 7.10-7.18 (m, 2H) 6.92-6.99 (m, J = 8.03 Hz, 1H) 2.98 (s, 3H) 2.59-2.69 (m, 4H) 1.64-1.72 (m, 4H) |
| 321 | 2-Amino-5-(2′-fluoro-5′- methoxybiphenyl-3-yl)-3- methyl-5-(5,6,7,8- tetrahydronaphthalen-2-yl)- 3,5-dihydro-4H-imidazol-4- one | 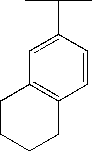 | 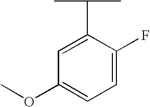 | 444 | 7.66 (s, 1H) 7.46-7.51 (m, 1H) 7.36-7.45 (m, 2H) 7.11-7.28 (m, 3H) 6.89-7.04 (m, 3H) 3.79 (s, 3H) 2.98 (s, 3H) 2.59-2.71 (m, 4H) 1.70 (s, 4H) |
| 322 | 2-Amino-3-methyl-5-(3′- nitrobiphenyl-3-yl)-5- (5,6,7,8- tetrahydronaphthalen-2-yl)- 3,5-dihydro-4H-imidazol-4- one | 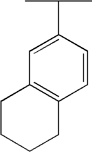 | 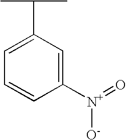 | 441 | 8.31 (s, 1H) 8.24 (dd, J = 1.38 Hz, 1H) 8.04 (d, J = 7.78 Hz, 1H) 7.73-7.86 (m, 2H) 7.65 (d, J = 7.53 Hz, 1H) 7.59 (d, J = 7.78 Hz, 1H) 7.47 (t, J = 7.65 Hz, 1H) 7.13-7.20 (m, 2H) 6.94-7.00 (m, J = 8.53 Hz, 1H) 3.00 (s, 3H) 2.59-2.72 (m, 4H) 1.64-1.73 (m, 4H) |
| 323 | 2-Amino-5-(2′-fluoro-3′- methoxybiphenyl-3-yl)-3- methyl-5-(5,6,7,8- tetrahydronaphthalen-2-yl)- 3,5-dihydro-4H-imidazol-4- one | 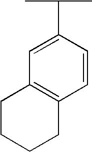 | 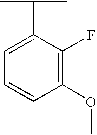 | 444 | 7.62 (s, 1H) 7.43-7.54 (m, J = 5.52 Hz, 1H) 7.34-7.41 (m, 2H) 7.12-7.25 (m, 4H) 6.90-6.98 (m, 2H) 3.86 (s, 3H) 2.96 (s, 3H) 2.59-2.69 (m, 4H) 1.64-1.73 (m, 4H) |
| 324 | 2-Amino-5-[3-(3- furyl)phenyl]-3-methyl-5- (5,6,7,8- tetrahydronaphthalen-2-yl)- 3,5-dihydro-4H-imidazol-4- one | 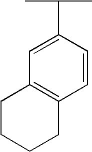 | 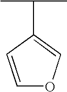 | 386 | 8.05 (s, 1H) 7.74 (t, J = 1.76 Hz, 1H) 7.62 (s, 1H) 7.46 (d, J = 7.28 Hz, 1H) 7.27-7.41 (m, 2H) 7.11-7.16 (m, 2H) 6.96 (d, J = 8.28 Hz, 1H) 6.79 (s, 1H) 2.99 (s, 3H) 2.62-2.69 (m, 4H) 1.66-1.74 (m, 4H) |
| 325 | 2-Amino-5-(2,3-dihydro- 1H-inden-5-yl)-5-(3′- methoxybiphenyl-3-yl)-3- methyl-3,5-dihydro-4H- imidazol-4-one 0.25 acetate | 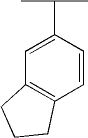 | 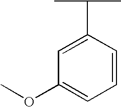 | 412 | 7.73 (s, 1H) 7.52 (d, J = 7.78 Hz, 1H) 7.47 (d, J = 7.78 Hz, 1H) 7.35-7.43 (m, 2H) 7.29-7.32 (m, 1H) 7.19-7.24 (m, 1H) 7.04-7.17 (m, 3H) 6.95 (dd, J = 2.13 Hz, 1H) 3.82 (s, 3H) 3.00 (s, 3H) 2.75-2.86 (m, J = 7.40 Hz, 4H) 1.91 (s, 0.4H) 1.90-2.04 (m, 2H) |

### Example 326

### 3′-[2-Amino-4-(6-methoxy-5,6,7,8-tetrahydronaphthalen-2-yl)-1-methyl-5-oxo-4,5-dihydro-1H-imidazol-4-yl]-5-methoxybiphenyl-3-yl methanesulfonate

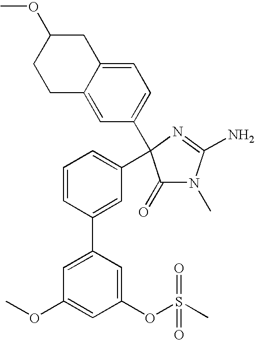

2-Amino-5-(3-bromophenyl)-5-(6-methoxy-5,6,7,8-tetrahydronaphthalen-2-yl)-3-methyl-3,5-dihydro-4H-imidazol-4-one (50 mg, 0.117 mmol), 3-methoxy-5-(4,4,5,5-tetramethyl-1,3,2-dioxaborolan-2-yl)phenyl methanesulfonate (50 mg, 0.152 mmol), [1,1′-bis(diphenylphosphino)ferrocene]palladium(II) chloride dichloromethane adduct (10 mg, 0.012 mmol), potassium carbonate (102 mg, 0.738 mmol) and in anhydrous tetrahydrofuran (3 mL) was irradiated in a microwave at 130° C. for 2 h. When cooled to ambient temperature the mixture was filtered and dimethyl sulfoxide (500 μL) was added. The solution was concentrated in vacuo to remove tetrahydrofuran and purified by preparative HPLC to give 12 mg (22% yield) of the title compound: ¹H NMR (DMSO-d₆) δ 7.72 (s, 1H) 7.47-7.62 (m, 2H) 7.42 (t, J=7.65 Hz, 1H) 7.16-7.21 (m, 2H) 7.08-7.11 (m, 1H) 7.05-7.07 (m, 1H) 6.95-7.02 (m, 2H) 3.86 (s, 3H) 3.56-3.64 (m, 1H) 3.43 (s, 3H) 3.28 (s, 3H) 2.88-3.04 (m, 4H) 2.58-2.81 (m, 3H) 1.87-1.98 (m, 1H) 1.66-1.76 (m, 1H); MS (ES) m/z 550 [M+1]⁺

### Examples 327-329

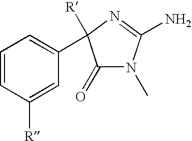

TABLE 7
Representative examples synthesized as described for 3′-[2-Amino-4-(6-methoxy-5,6,7,8-tetrahydronaphthalen-
2-yl)-1-methyl-5-oxo-4,5-dihydro-1H-imidazol-4-yl]-5-methoxybiphenyl-3-yl methanesulfonate

| | | | | [M − H]⁻ | ¹H-NMR |
|---|---|---|---|---|---|
| Ex | Chemical name | R′ | R″ | m/z | (DMSO-d₆) δ |
| 327 | 3′-[4-(1-Acetyl-1,2,3,4- tetrahydroquinolin-6-yl)-2- amino-1-methyl-5-oxo-4,5- dihydro-1H-imidazol-4-yl]-5- methoxybiphenyl-3-yl methanesulfonate | 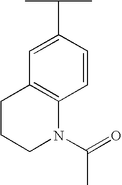 | 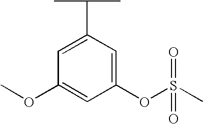 | 563 | 7.73 (s, 1H) 7.51-7.58 (m, 2H) 7.36-7.47 (m, 2H) 7.27 (s, 2H) 7.07-7.10 (m, 1H) 7.03-7.06 (m, 1H) 6.96 (t, J = 2.26 Hz, 1H) 3.85 (s, 3H) 3.63 (t, J = 6.27 Hz, 2H) 3.42 (s, 3H) 2.99 (s, 3H) 2.65 (t, J = 6.53 Hz, 2H) 2.12 (s, 3H) 1.79-1.86 (m, 2H) |
| 328 | 3′-[2-Amino-1-methyl-5-oxo-4- (5,6,7,8-tetrahydronaphthalen-2- yl)-4,5-dihydro-1H-imidazol-4- yl]-5-methoxybiphenyl-3-yl methanesulfonate | 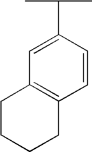 | 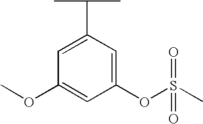 | 520 | 7.72 (s, 1H) 7.50-7.58 (m, 2H) 7.42 (t, J = 7.78 Hz, 1H) 7.13-7.18 (m, 2H) 7.07-7.10 (m, J = 2.01 Hz, 1H) 7.05-7.07 (m, J = 1.76 Hz, 1H) 6.95-6.98 (m, J = 2.13 Hz, 2H) 3.86 (s, 3H) 3.43 (s, 3H) 2.99 (s, 3H) 2.61-2.70 (m, 4H) 1.70 (s, 4H) |
| 329 | 3′-[2-Amino-4-(2,3-dihydro-1H- inden-5-yl)-1-methyl-5-oxo-4,5- dihydro-1H-imidazol-4-yl]-5- methoxybiphenyl-3-yl methanesulfonate | 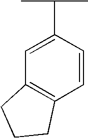 | 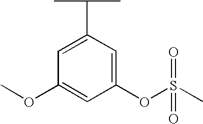 | 506 | 7.73 (s, 1H) 7.50-7.58 (m, 2H) 7.39-7.45 (m, J = 7.78 Hz, 1H) 7.19-7.31 (m, 3H) 7.02-7.17 (m, 2H) 6.96-7.00 (m, 1H) 3.86 (s, 3H) 3.43 (s, 3H) 3.00 (s, 3H) 2.74-2.86 (m, 4H) 1.90-2.04 (m, 2H) |

### Example 330

### 2,3-Dihydro-1-benzofuran-5-yl[2-(3′-methoxybiphenyl-3-yl)-1,3-dithian-2-yl]methanol

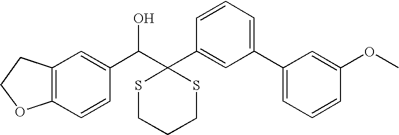

n-Butyl lithium (0.94 mL, 2.5 M) was added dropwise to a cooled (−78° C.) solution of 2-(3′-methoxybiphenyl-3-yl)-1,3-dithiane (327.4 mg, 2.21 mmol) in dry tetrahydrofuran (5 mL) under an atmosphere of argon. The solution was stirred at −78° C. for 20 min and was then treated with 4-methyl-3,4-dihydro-2H-1,4-benzoxazine-7-carbaldehyde (500 mg, 2.82 mmol). The reaction was stirred for another 20 min and then allowed to reach room temperature. Silica gel was added and the solvent was evaporated and the residue was purified by column chromatography, using a gradient of 0 to 30% ethyl acetate in n-heptane as the eluent, to give 426 mg (43% yield) the title compound: ¹H NMR (CDCl₃) δ7.88-7.79 (m, 2H) 7.55-7.49 (m, 1H) 7.43 (t, J=7.71 Hz, 1H) 7.34 (t, J=7.96 Hz, 1H) 7.03 (d, J=7.83 Hz, 1H) 6.99-6.93 (m, 1H) 6.89 (dd, J=7.83, 2.27 Hz, 1H) 6.70 (dd, J=8.34, 1.52 Hz, 1H) 6.64 (s, 1H) 6.59 (d, J=8.34 Hz, 1H) 4.98 (d, J=3.54 Hz, 1H) 4.49 (t, J=8.72 Hz, 2H) 3.86 (s, 3H) 3.02 (t, J=8.59 Hz, 2H) 2.88 (d, J=3.28 Hz, 1H) 2.77-2.67 (m, 4H); MS (ES) m/z 433 [M-water]⁺

### Example 331

### 1-(2,3-Dihydro-1-benzofuran-5-yl)-2-(3′-methoxybiphenyl-3-yl)ethane-1,2-dione

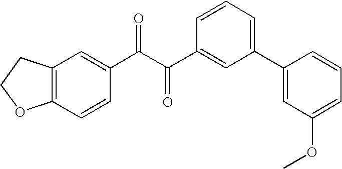

Dess-Martin periodinane (903 mg, 2.13 mmol) was added to a solution of 2,3-dihydro-1-benzofuran-5-yl[2-(3′-methoxybiphenyl-3-yl)-1,3-dithian-2-yl]methanol (460 mg, 0.95 mmol) and tert-butanol (246 mg, 3.33 mmol) in dichloromethane (20 mL) under an atmosphere of nitrogen and the mixture was stirred overnight. Sodium thiosulfate (1 M, 10 mL) and saturated solution of sodium hydrogen carbonate (10 mL) was added followed by addition of dichloromethane. The phases were separated and the organic phase concentrated in vacuo. Purification by column chromatography, using a gradient with 0 to 100% ethyl acetate in n-heptane as the eluent, to give 377 mg (111% yield) the title compound: ¹H NMR (CDCl₃) δ 8.20 (t, J=1.64 Hz, 1H) 8.00-7.81 (m, 4H) 7.58 (t, J=7.71 Hz, 1H) 7.38 (t, J=7.96 Hz, 1H) 7.23-7.16 (m, 1H) 7.15-7.09 (m, 1H) 6.94 (dd, J=6.95, 1.39 Hz, 1H) 6.86 (d, J=8.59 Hz, 1H) 4.71 (t, J=8.84 Hz, 2H) 3.88 (s, 3H) 3.27 (t, J=8.84 Hz, 2H); MS (ES) m/z 359 [M+1]⁺

### Example 332

### 5-(2,3-Dihydro-1-benzofuran-5-yl)-5-(3′-methoxybiphenyl-3-yl)-3-methyl-2-thioxoimidazolidin-4-one

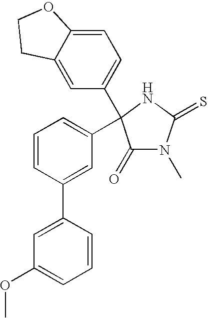

A solution of 1-(2,3-dihydro-1-benzofuran-5-yl)-2-(3′-methoxybiphenyl-3-yl)ethane-1,2-dione (377 mg, 1.05 mmol) and N-methylthiourea (189 mg, 2.10 mmol) in dimethyl sulfoxide (20 mL) was heated to 100° C. Potassium hydroxide (1.80 mL, 1.2 M) was added dropwise and the reaction was stirred at 100° C. for five minutes and was then allowed to cool to room temperature. The solution was diluted with 30 mL water and carefully acidified to pH 5 with concentrated hydrochloric acid and the aqueous phase was extracted three times with dichloromethane (×3). The combined organic phases were washed twice with water and then concentrated in vacuo to give 540 mg (100% yield) of the title compound; MS (ES) m/z 431 [M+1]⁺

### Example 333

### 2-Amino-5-(2,3-dihydro-1-benzofuran-5-yl)-5-(3′-methoxybiphenyl-3-yl)-3-methyl-3,5-dihydro-4H-imidazol-4-one hydrochloride

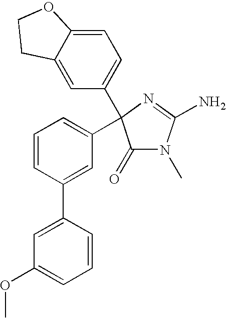

tert-Butyl hydroperoxide (1.43 g, 70%, 15.9 mmol) was added to a mixture of 5-(2,3-dihydro-1-benzofuran-5-yl)-5-(3′-methoxybiphenyl-3-yl)-3-methyl-2-thioxoimidazolidin-4-one (455 mg, 1.06 mmol) in methanol/ammonium hydroxide (3:1, 40 mL). The mixture was stirred at room temperature overnight and reduced in vacuo until approximately 50% of the volume remained. The mixture was diluted with water (30 mL) and dichloromethane (40 mL). The phases were separated and the organic phase was concentrated in vacuo. Purification by preparative HPLC. The base treated with hydrochloric acid (1 M, in diethyl ether) to give 110 mg (23% yield) of the title compound: ¹H NMR (DMSO-d₆) δ 7.72 (d, J=7.83 Hz, 1H), 7.66 (d, J=1.77 Hz, 1H), 7.54 (t, J=7.83 Hz, 1H), 7.48-7.36 (m, 2H), 7.25-7.14 (m, 3H), 7.07-6.96 (m, 2H), 6.82 (d, J=8.59 Hz, 1H), 4.55 (t, J=8.72 Hz, 2H), 3.82 (s, 3H), 3.33 (s, 3H), 3.25-3.14 (t, 2H); MS (ES) m/z 414 [M+1]⁺

### Example 334

### 2-Acetyl-4-chloroisoindoline

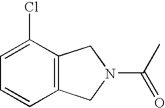

Acetic anhydride (115 mg, 1.13 mmol) was added to a cooled (0° C.) solution of 4-chloroisoindoline (136 mg, 0.89 mmol, described in: Clark, R. et al. J. Med. Chem. 1990, 33(2), 596-600.), dimethyl-amino-pyridine (14 mg, 0.113 mmol) and triethylamine (228 mg, 2.26 mmol) in anhydrous dichloromethane (20 mL) and the reaction mixture was stirred at room temperature overnight. Sodium hydroxide (40 mL, 2 M) was added and the phases were separated. The organic phase was concentrated in vacuo and the residue was purified by column chromatography, using a 0 to 30% ammonium hydroxide/methanol (1:9) in dichloromethane to give 126 mg (73% yield) of the title compound; MS (ES) m/z 196, 198 [M+1]⁺

### Example 335

### 5-[3-(2-Acetyl-2,3-dihydro-1H-isoindol-4-yl)phenyl]-2-amino-3-methyl-5-phenyl-3,5-dihydro-4H-imidazol-4-one acetate

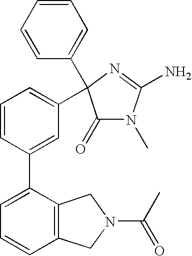

A solution of tris(dibenzylideneacetone)dipalladium (17 mg, 0.019 mmol) and tricyclohexylphosphine (25 mg, 0.090 mmol) in 1,2-dimethoxyethane (3 mL) under an atmosphere of argon was stirred at room temperature for 30 min. 2-Acetyl-4-chloroisoindoline (126 mg, 0.644 mmol), 4,4,4′,4′,5,5,5′,5′-octamethyl-2,2′-bi-1,3,2-dioxaborolane (180 mg, 0.71 mmol) and potassium acetate (95 mg, 0.966 mmol) were added and the reaction was irradiated in a microwave at 150° C. for 1.5 h. The reaction mixture was diluted with sodium hydroxide (20 mL, 0.5 M) and extracted with dichloromethane (2×30 mL). The combined organic phases were evaporated and the residue was dissolved in 1,2-dimethoxyethane/water/ethanol (6:3:1, 4 mL) and 2-amino-5-(3-bromophenyl)-3-methyl-5-phenyl-3,5-dihydro-4H-imidazol-4-one (58 mg, 0.17 mmol), [1,1′-bis(diphenylphosphino)ferrocene]palladium(II) chloride dichloromethane (83 mg, 0.254 mmol) and cesium carbonate (5 mg, 0.006 mmol) was added. The mixture was irradiated in a microwave at 150° C. for 15 min. The reaction was diluted with water (20 mL) and extracted with dichloromethane (3×30 mL). The combined organic phases were concentrated in vacuo and the product was purified by preparative HPLC to give 4 mg (1% yield) of the title compound: ¹H NMR (CD₃OD) δ 7.49-7.33 (m, 12H), 3.20-3.17 (m, 4H), 2.18 (s, 3H), 2.11 (s, 3H); MS (ES) m/z 425 [M+1]⁺

### Example 336

### 5-[(3-Bromophenyl)ethynyl]-2,3-dihydro-1-benzofuran

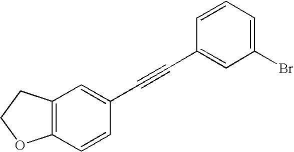

To a mixture of bis(triphenylphosphine)palladium(II) dichloride (154 mg, 0.219 mmol) and copper iodide (42 mg, 0.219 mmol) in anhydrous tetrahydrofuran (50 mL) and under an atmosphere of argon was sequentially added triethylamine (15 mL), a solution of 5-ethynyl-2,3-dihydro-1-benzofuran (3.151 g, 21.86 mmol; described in: Walser A. et. al. J. Med. Chem. 1991, 34, 1440-46) in anhydrous tetrahydrofuran (10 mL) and finally 1-bromo-3-iodobenzene (6.18 g, 21.86 mmol). The resulting solution was stirred at room temperature for 20 h. The crude mixture was diluted with ethyl acetate, washed sequentially with 1 M hydrochloric acid, water and saturated sodium hydrogen carbonate solution and dried over magnesium sulfate. The solvent was evaporated to give a solid material which was suspended in hexane, the solid was filtered, washed with hexane and dried in vacuo to give the title product (5.30 g, 81% yield). ¹H NMR (CDCl₃) δ 7.66 (m, 1H), 7.43 (m, 2H), 7.37 (m, 1H), 7.31 (m, 1H), 7.20 (t, J=7.83 Hz, 1H), 6.77 (d, J=8.34, 1H), 4.62 (t, J=8.72 Hz, 2H), 3.23 (t, J=8.72 Hz, 2H); MS (EI) m/z 298, 300 [M+H]⁺.

### Example 337

### 1-(3-Bromophenyl)-2-(2,3-dihydro-1-benzofuran-5-yl)ethane-1,2-dione

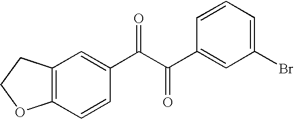

5-[(3-Bromophenyl)ethynyl]-2,3-dihydro-1-benzofuran (5.30 g, 17.71 mmol) and palladium(II) dichloride (314 mg, 1.77 mmol) in dimethyl sulfoxide (75 mL) were heated at 150° C. under an atmosphere of argon for 6 h. Water was added after cooling to room temperature. The mixture was extracted with dichloromethane (×3). The combined extracts were washed sequentially with 1 M hydrochloric acid, water and saturated sodium hydrogen carbonate solution, dried over sodium sulfate and evaporated. Purification by column chromatography, using 0-30% ethyl acetate in hexane as the eluent, gave 4.37 g (74% yield) of the title compound. ¹H NMR (CDCl₃) 8.13 (m, 1H), 7.89 (m, 1H), 7.85 (m, 1H), 7.76-7.81 (m, 2H), 7.39 (t, J=7.96 Hz, 1H), 6.87 (d, J=8.59 Hz, 1H), 4.71 (t, J=8.84 Hz, 2H), 3.28 (t, J=8.72 Hz, 2H); MS (ES) m/z 331.2, 333.2 [M+H]⁺.

### Example 338

### 5-(3-Bromophenyl)-5-(2,3-dihydro-1-benzofuran-5-yl)-3-methyl-2-thioxoimidazolidin-4-one

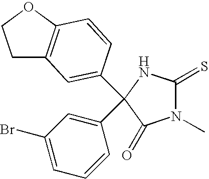

N-Methylthiourea (2.36 g, 26.21 mmol) was added to a solution of 1-(3-bromophenyl)-2-(2,3-dihydro-1-benzofuran-5-yl)ethane-1,2-dione (4.34 g, 13.11 mmol) in dimethyl sulfoxide (50 mL). The solution was heated at 100° C. for 5 min and 1.2 M aqueous potassium hydroxide (22.4 mL, 26.82 mmol) was added dropwise over 6-7 min. The mixture was heated for another 10 min and then cooled to room temperature. Water was added and the pH was adjusted to 5 by addition of 1 M hydrochloric acid. The mixture was extracted with dichloromethane (×3). The combined extracts were washed with water (×2), dried over sodium sulfate and evaporated to give 5.48 g (100% yield) of the title compound; ¹H NMR (CDCl₃) δ 7.72 (br. s., 1H), 7.49-7.53 (m, 2H), 7.29-7.33 (m, 1H), 7.24-7.28 (m, 1H), 7.07-7.09 (m, 1H), 6.97-7.01 (m, 1H), 6.76 (d, J=8.34 Hz, 1H), 4.60 (t, J=8.72 Hz, 2H), 3.34 (s, 3H), 3.20 (t, J=8.72 Hz, 2H); MS (ES) m/z 401, 403 [M+H]⁻.

### Example 339

### 2-Amino-5-(3-bromophenyl)-5-(2,3-dihydro-1-benzofuran-5-yl)-3-methyl-3,5-dihydro-4H-imidazol-4-one

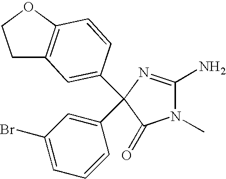

To a solution of 5-(3-bromophenyl)-5-(2,3-dihydro-1-benzofuran-5-yl)-3-methyl-2-thioxoimidazolidin-4-one (5.48 g, 13.59 mmol) in methanol (100 mL) were added 70% aqueous tert-butylhydroperoxide (28 mL, 204 mmol) and 33% aqueous ammonia (50 mL). The resulting mixture was stirred at room temperature for 16 h. The methanol was evaporated and the aqueous residue was extracted with 3 aliquots of dichloromethane. The combined extracts were washed with water, dried over sodium sulfate and evaporated. The crude product was purified by column chromatography, using 0-10% methanol in dichloromethane and in the end substituting methanol for 0.1 M ammonia in methanol as the eluent, gave 2.29 g (44% yield) of the title compound. ¹H NMR (CDCl₃) δ 7.66 (t, J=1.77 Hz, 1H), 7.38-7.43 (m, 2H), 7.21-7.23 (m, 1H), 7.14-7.20 (m, 2H), 6.71 (d, J=8.34 Hz, 1H), 4.55 (t, J=8.72 Hz, 2H), 3.15 (t, J=8.08 Hz, 2H), 3.11 (s, 3H); MS (ES) m/z 386.2, 388.2 [M+H]⁺, 384.4, 386.4 [M−H]⁻.

### Example 340

### 6-[(3-Bromophenyl)ethynyl]chromane

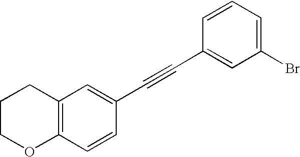

The title compound was prepared as described for Example 336 in 92% yield starting from 6-iodochromane (described in: Togo H. et. al. J. Chem. Soc. Perkin Trans. 1, 1997, 787-793). Purification by column chromatography, using 0-30% ethyl acetate in n-heptane as the eluent. ¹H NMR (CDCl₃) δ7.66 (t, J=1.77 Hz, 1H) 7.40-7.45 (m, 2H) 7.23-7.27 (m, 2H) 7.20 (t, J=7.96 Hz, 1H) 6.77 (d, J=8.34 Hz, 1H) 4.20-4.24 (m, 2H) 2.79 (t, J=6.57 Hz, 2H) 1.99-2.06 (m, 2H); MS (EI) m/z 312, 314 [M+H]⁺.

### Example 341

### 1-(3-Bromophenyl)-2-(3,4-dihydro-2H-chromen-6-yl)ethane-1,2-dione

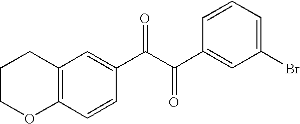

The title compound was prepared as described for Example 337 in 73% yield staring from 6-[(3-bromophenyl)ethynyl]chromane: ¹H NMR (CDCl₃) δ 8.12 (t, J=1.64 Hz, 1H), 7.89 (m, 1H), 7.77 (m, 1H), 7.73 (dd, J=8.59, 2.27 Hz, 1H), 7.69 (m, 1H), 7.39 (t, J=7.83 Hz, 1H), 6.88 (d, J=8.59 Hz, 1H), 4.29 (m, 2H), 2.83 (t, J=6.44 Hz, 2H), 2.05 (m, 2H); MS (ES) m/z 345.2, 347.3 [M+H]⁺.

### Example 342

### 5-(3-Bromophenyl)-5-(3,4-dihydro-2H-chromen-6-yl)-3-methyl-2-thioxoimidazolidin-4-one

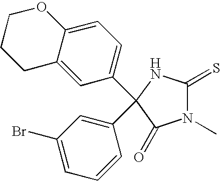

The title compound was prepared as described for Example 338 in 100% yield starting from 1-(3-bromophenyl)-2-(3,4-dihydro-2H-chromen-6-yl)ethane-1,2-dione; ¹H NMR (CDCl₃) δ 7.62 (br. s., 1H), 7.49-7.53 (m, 2H), 7.28-7.32 (m, 1H), 7.26 (t, J=7.96 Hz, 1H), 6.91-6.95 (m, 2H), 6.76-6.80 (m, 1H), 4.17-4.21 (m, 2H), 3.33 (s, 3H), 2.75 (t, J=6.44 Hz, 2H), 1.97-2.04 (m, 2H); MS (ES) m/z 415.3, 417.3 [M−H]⁻.

### Example 343

### 2-Amino-5-(3-bromophenyl)-5-(3,4-dihydro-2H-chromen-6-yl)-3-methyl-3,5-dihydro-4H-imidazol-4-one

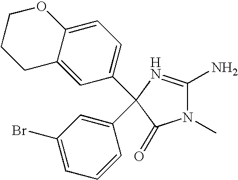

The title compound was prepared as described for Example 339 in 75% yield starting from 5-(3-bromophenyl)-5-(3,4-dihydro-2H-chromen-6-yl)-3-methyl-2-thioxoimidazolidin-4-one; ¹H NMR (CDCl₃) δ 7.66 (t, J=1.89 Hz, 1H), 7.38-7.44 (m, 2H), 7.18 (t, J=7.96 Hz, 1H), 7.06-7.12 (m, 2H), 6.73 (d, J=8.34 Hz, 1H), 4.14-4.18 (m, 2H), 3.11 (s, 3H), 2.73 (t, J=6.57 Hz, 2H), 1.94-2.01 (m, 2H); MS (ES) m/z 400.2, 402.2 [M+H]⁺, 398.4, 400.4 [M−H]⁻.

### Example 344

### (3,4-Dihydro-1H-isochromen-7-ylethynyl)(trimethyl)silane

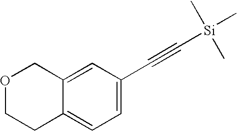

Bis(benzonitrile)palladium(II) dichloride (100 mg, 0.260 mmol) and copper(I) iodide (33 mg, 0.174 mmol) was added to a dried round bottomed flask flushed with argon and equipped with a magnetic stirring bar and a rubber septum. Anhydrous 1,4-dioxane (20 mL), tri-tert-butylphosphine (105 mg, 0.521 mmol) as a 10 weight % solution in hexane, diisopropylamine (1.48 mL, 10.42 mmol), trimethylsilylacetylene (1.066 g, 10.85 mmol) and 7-bromo-3,4-dihydro-1H-isochromen (1.850 g, 8.682 mmol, described in: Unterhalt B. and Jöstingmeier R., Pharmazie 1996, 51, 641-644) were sequentially added via syringe. The resulting dark solution was stirred for 18 h at room temperature. The mixture was diluted with ethyl acetate, washed with 1 M hydrochloric acid, water and saturated aqueous sodium hydrogen carbonate and dried over sodium sulfate. After removal of the solvents the crude material was purified by column chromatography, using 0-15% ethyl acetate in n-heptane as the eluent. 1.67 g (84% yield) of the title compound was isolated; ¹H NMR (CDCl₃) δ 7.26 (m, 1H) 7.12 (m, 1H) 7.05 (d, J=7.83 Hz, 1H) 4.73 (s, 2H) 3.97 (t, J=5.68 Hz, 2H) 2.85 (t, J=5.68 Hz, 2H); MS (EI) m/z 230 [M+H]⁺.

### Example 345

### 7-Ethynyl-3,4-dihydro-1H-isochromene

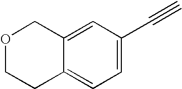

A mixture of (3,4-Dihydro-1H-isochromen-7-ylethynyl)(trimethyl)silane and powdered potassium carbonate (5.01 g, 36.25 mmol) in a mixture of tetrahydrofuran and methanol (1:2, 60 mL) was stirred at room temperature overnight. The mixture was filtered and concentrated. The residue was dissolved in dichloromethane and washed with water. After drying over sodium sulfate and removal of solvents the crude product was purified by column chromatography, using 0-10% ethyl acetate in n-heptane as the eluent, to afford 0.93 g, (81% yield) of the title compound. ¹H NMR (CDCl₃) δ 7.28-7.32 (m, 1H), 7.14 (br. s., 1H), 7.08 (d, J=8.08 Hz, 1H), 4.74 (s, 2H), 3.94-4.00 (m, 2H), 3.03 (s, 1H), 2.78-2.89 (m, 2H); MS (EI) m/z 158 [+H]⁺.

### Example 346

### 7-[(3-Bromophenyl)ethynyl]-3,4-dihydro-1H-isochromene

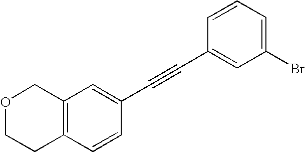

The title compound was prepared as described for Example 336 in 65% yield starting from 7-ethynyl-3,4-dihydro-1H-isochromene. Purification by column chromatography, using 0-30% ethyl acetate in n-heptane. ¹H NMR (CDCl₃) δ 7.68 (dd, J=2.02, 1.52 Hz, 1H), 7.42-7.48 (m, 2H), 7.31-7.35 (m, 1H), 7.22 (t, J=7.96 Hz, 1H), 7.17 (br. s., 1H), 7.12 (d, J=7.83 Hz, 1H), 4.77 (s, 2H), 3.99 (t, J=5.81 Hz, 2H), 2.88 (t, J=5.68 Hz, 2H); MS (EI) m/z 312, 314 [M+H]⁺.

### Example 347

### 1-(3-Bromophenyl)-2-(3,4-dihydro-1H-isochromen-7-yl)ethane-1,2-dione

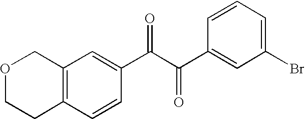

The title compound was prepared as described for Example 337 in 100% yield starting from 7-[(3-bromophenyl)ethynyl]-3,4-dihydro-1H-isochromene. The product was used in the following reaction without chromatographic purification; MS (ESI) m/z 343.4, 345.5 [M−H]⁻.

### Example 348

### 5-(3-Bromophenyl)-5-(3,4-dihydro-1H-isochromen-7-yl)-3-methyl-2-thioxoimidazolidin-4-one

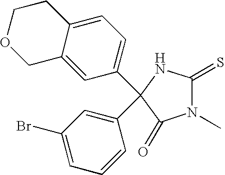

The title compound was prepared as described for Example 338 in 95% yield starting from 1-(3-bromophenyl)-2-(3,4-dihydro-1H-isochromen-7-yl)ethane-1,2-dione; ¹H NMR (CDCl₃) δ 7.78 (br. s., 1H), 7.48-7.54 (m, 2H), 7.24-7.30 (m, 2H), 7.13-7.17 (m, 1H), 7.04-7.08 (m, 1H), 6.89-6.91 (m, J=2.02 Hz, 1H), 4.72 (s, 2H), 3.98 (t, J=5.81 Hz, 2H), 3.33 (s, 3H), 2.86 (t, J=5.56 Hz, 2H); MS (ES) m/z 415.4, 417.3 [M−H]⁻.

### Example 349

### 2-Amino-5-(3-bromophenyl)-5-(3,4-dihydro-1H-isochromen-7-yl)-3-methyl-3,5-dihydro-4H-imidazol-4-one

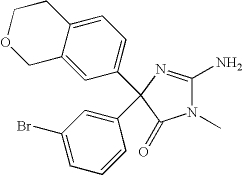

The title compound was prepared as described for Example 339 in 17% yield starting from 5-(3-bromophenyl)-5-(3,4-dihydro-1H-isochromen-7-yl)-3-methyl-2-thioxoimidazolidin-4-one; ¹H NMR (CDCl₃) δ 7.66 (t, J=1.89 Hz, 1H), 7.39-7.44 (m, 2H), 7.22-7.26 (m, 1H), 7.07-7.10 (m, 2H), 4.74 (br. s., 2H), 3.95 (t, J=5.68 Hz, 2H), 3.13 (s, 3H), 2.83 (t, J=5.43 Hz, 2H); MS (ES) m/z 400.2, 402.2 [+H]⁺, 398.4, 400 [M−H]⁻.

### Example 350

### 2-Amino-5-(2,3-dihydro-1-benzofuran-5-yl)-5-(3′-ethoxybiphenyl-3-yl)-3-methyl-3,5-dihydro-4H-imidazol-4-one 0.25 acetate

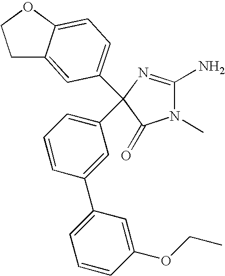

2-Amino-5-(3-bromophenyl)-5-(2,3-dihydro-1-benzofuran-5-yl)-3-methyl-3,5-dihydro-4H-imidazol-4-one (60 mg, 0.155 mmol), (3-ethoxyphenyl)boronic acid (33.5 mg, 0.202 mmol), [1,1′-bis(diphenylphosphino)ferrocene]palladium(II) chloride dichloromethane adduct (13 mg, 0.015 mmol), cesium carbonate (151 mg, 0.465 mmol) in 1,2-dimethoxyethane, water and ethanol (6:3:1, 3 mL) was irradiated under an atmosphere of argon in a microwave at 130° C. for 15 min. When cooled to ambient temperature the mixture was filtered and dimethyl sulfoxide (500 μL) was added. The solution was concentrated in vacuo to remove the reaction solvents and purified by preparative HPLC to give 20.8 mg (31% yield) of the title compound. ¹H NMR (DMSO-d₆) δ 7.69-7.71 (m, 1H), 7.48-7.52 (m, 1H), 7.43-7.46 (m, 1H), 7.33-7.39 (m, 2H), 7.27-7.30 (m, 1H), 7.15-7.19 (m, 1H), 7.06-7.10 (m, 1H), 7.02-7.04 (m, 1H), 6.90-6.94 (m, 1H), 6.66 (d, J=8.28 Hz, 1H), 4.47 (t, J=8.66 Hz, 2H), 4.08 (q, J=7.03 Hz, 2H), 3.11 (t, J=8.78 Hz, 2H), 2.98 (s, 3H), 1.91 (s, 0.6H), 1.35 (t, J=6.90 Hz, 3H); MS (ES) m/z 428.1 [M+H]⁺.

### Examples 351-376

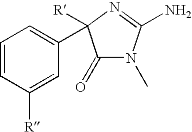

TABLE 8
Representative examples synthesized as described for 2-Amino-5-(2,3-dihydro-1-benzofuran-5-
yl)-5-(3′-ethoxybiphenyl-3-yl)-3-methyl-3,5-dihydro-4H-imidazol-4-one 0.25 acetate.

| | | | | [M + H]⁺ | ¹H-NMR |
|---|---|---|---|---|---|
| Ex | Chemical name | R′ | R″ | m/z | (DMSO-d₆) δ |
| 351 | 2-Amino-5-(2,3-dihydro-1- benzofuran-5-yl)-5-(2′,5′- dimethoxybiphenyl-3-yl)-3- methyl-3,5-dihydro-4H- imidazol-4-one 0.25 acetate | 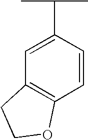 | 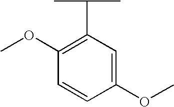 | 444.1 | 7.57 (br. s., 1H) 7.35-7.39 (m, 1H) 7.27-7.35 (m, 3H) 7.17-7.22 (m, 1H) 7.01 (d, J = 9.03 Hz, 1H) 6.87-6.91 (m, 1H) 6.75 (d, J = 3.01 Hz, 1H) 6.67 (d, J = 8.28 Hz, 1H) 4.47 (t, J = 8.66 Hz, 2H) 3.73 (s, 3H) 3.65 (s, 3H) 3.12 (t, J = 8.53 Hz, 2H) 2.97 (s, 3H) 1.91 (s, 0.4H) |
| 352 | 2-Amino-5-[3-(1,3- benzodioxol-5-yl)phenyl]- 5-(2,3-dihydro-1- benzofuran-5-yl)-3-methyl- 3,5-dihydro-4H-imidazol-4- one 0.25 acetate | 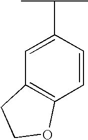 | 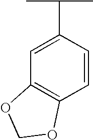 | 428.1 | 7.63-7.65 (m, 1H) 7.42-7.45 (m, 1H) 7.38-7.41 (m, 1H) 7.31-7.36 (m, 1H) 7.27-7.29 (m, 1H) 7.14-7.19 (m, 1H) 7.08-7.10 (m, 1H) 6.99-7.00 (m, 2H) 6.66 (d, J = 8.28 Hz, 1H) 6.05 (s, 2H) 4.47 (t, J = 8.66 Hz, 2H) 3.11 (t, J = 8.78 Hz, 2H) 2.98 (s, 3H) 1.91 (s, 0.6H) |
| 353 | 2-Amino-5-(2,3-dihydro-1- benzofuran-5-yl)-5-(2′- fluoro-5′-methoxybiphenyl- 3-yl)-3-methyl-3,5-dihydro- 4H-imidazol-4-one 0.25 acetate | 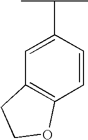 | 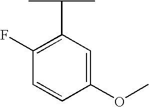 | 432.1 | 7.62-7.65 (m, 1H) 7.45-7.50 (m, 1H) 7.38-7.42 (m, 2H) 7.28-7.30 (m, 1H) 7.15-7.25 (m, 2H) 6.89-6.98 (m, 2H) 6.66 (d, J = 8.28 Hz, 1H) 4.47 (t, J = 8.78 Hz, 2H) 3.78 (s, 3H) 3.11 (t, J = 8.66 Hz, 2H) 2.97 (s, 3H) 1.91 (s, 0.4H) |
| 354 | 2-Amino-5-(2,3-dihydro-1- benzofuran-5-yl)-5-(2′- fluoro-3′methoxybiphenyl- 3-yl)-3-methyl-3,5-dihydro- 4H-imidazol-4-one 0.25 acetate | 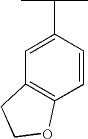 | 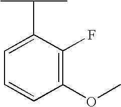 | 432.1 | 7.60-7.63 (m, 1H) 7.45-7.49 (m, 1H) 7.35-7.41 (m, 2H) 7.28-7.30 (m, 1H) 7.13-7.23 (m, 3H) 6.91 6.96 (m, 1H) 6.66 (d, J = 8.53 Hz, 1H) 4.47 (t, J = 8.78 Hz, 2H) 3.86 (s, 3H) 3.11 (t, J = 8.78 Hz, 2H) 2.97 (s, 3H) 1.91 (s, 0.4H) |
| 355 | 3′-[2-Amino-4-(2,3- dihydro-1-benzofuran-5-yl)- 1-methyl-5-oxo-4,5- dihydro-1H-imidazol-4-yl]- 6-fluorobiphenyl-3- carbonitrile 0.25 acetate | 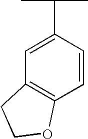 | 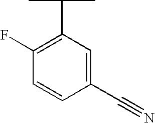 | 427.1 | 7.99 (dd, J = 7.28, 2.01 Hz, 1H) 7.91-7.96 (m, 1H) 7.67-7.69 (m, 1H) 7.52-7.58 (m, 2H) 7.41-7.47 (m, 2H) 7.27-7.30 (m, 1H) 7.15-7.19 (m, 1H) 4.47 (t, J = 8.66 Hz, 2H) 3.11 (t, J = 8.66 Hz, 2H) 2.97 (s, 3H) 1.91 (s, 0.4H) |
| 356 | 2-Amino-5-(2,3-dihydro-1- benzofuran-5-yl)-3-methyl- 5-(3′-nitrobiphenyl-3-yl)- 3,5-dihydro-4H-imidazol-4- one 0.25 acetate | 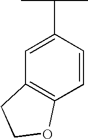 | 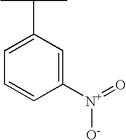 | 429.1 | 8.29-8.32 (m, 1H) 8.20-8.24 (m, 1H) 8.01-8.04 (m, 1H) 7.80-7.82 (m, 1H) 7.77 (t, J = 8.03 Hz, 1H) 7.62-7.66 (m, 1H) 7.55-7.59 (m, 1H) 7.46 (t, J = 7.78 Hz, 1H) 7.28 (br. s., 1H) 7.14-7.19 (m, 1H) 6.67 (d, J = 8.28 Hz, 1H) 4.47 (t, J = 8.66 Hz, 2H) 3.11 (t, J = 8.66 Hz, 2H) 2.99 (s, 3H) 1.91 (s, 0.4H) |
| 357 | 2-Amino-5-(2,3-dihydro-1- benzofuran-5-yl)-5-[3-(3- furyl)phenyl]-3-methyl-3,5- dihydro-4H-imidazol-4-one 0.25 acetate | 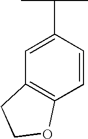 | 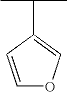 | 374.1 | 8.04 (br. s., 1H) 7.72-7.74 (m, 1H) 7.60 (br. s., 1H) 7.43-7.47 (m, 1H) 7.34-7.38 (m, 1H) 7.24-7.33 (m, 2H) 7.13-7.17 (m, 1H) 6.78 (br. s., 1H) 6.66 (d, J = 8.28 Hz, 1H) 4.47 (t, J = 8.53 Hz, 2H) 3.11 (t, J = 8.66 Hz, 2H) 2.98 (s, 3H) 1.91 (0.4H) |
| 358 | 2-Amino-5-[3-(1- benzofuran-2-yl)phenyl]-5- (2,3-dihydro-1-benzofuran- 5-yl)-3-methyl-3,5-dihydro- 4H-imidazol-4-one 0.25 acetate | 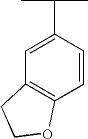 | 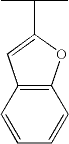 | 424.1 | 8.01-8.03 (m, 1.63 Hz, 1H) 7.77-7.81 (m, 1H) 7.62 7.67 (m, 2H) 7.51-7.55 (m, 1H) 7.43 (t, J = 7.78 Hz, 1H) 7.34-7.36 (m, 1H) 7.29-7.34 (m, 1H) 7.23-7.28 (m, 2H) 7.14-7.18 (m, 1H) 6.67 (d, J = 8.53 Hz, 1H) 4.47 (t, J = 8.78 Hz, 2H) 3.12 (t, J = 8.66 Hz, 2H) 3.00 (s, 3H) 1.91 (s, 0.5H) |
| 359 | 2-Amino-5-(2,3-dihydro-1- benzofuran-5-yl)-3-methyl- 5-[3′-(trifluoromethoxy) biphenyl-3-yl]-3,5-dihydro- 4H-imidazol-4-one 0.25 acetate | 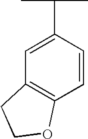 | 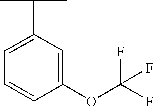 | 468.1 | 7.73 (br. s., 1H) 7.46-7.64 (m, 5H) 7.35-7.44 (m, 2H) 7.28 (br. s., 1H) 7.14-7.19 (m, 1H) 6.66 (d, J = 8.53 Hz, 1H) 4.47 (t, J = 8.66 Hz, 2H) 3.11 (t, J = 8.66 Hz, 2H) 2.98 (s, 3H) 1.91 (s, 0.4H) |
| 360 | 2-Amino-5-(3′- chlorobiphenyl-3-yl)-5- (2,3-dihydro-1-benzofuran- 5-yl)-3-methyl-3,5-dihydro- 4H-imidazol-4-one x 0.25 acetate | 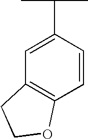 | 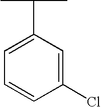 | 418.1, 420.0 | 7.72-7.74 (m, 1H) 7.56-7.58 (m, 1H) 7.52-7.56 (m, 1H) 7.47-7.52 (m, 3H) 7.38-7.46 (m, 2H) 7.28 (br. s., 1H) 7.14-7.18 (m, 1H) 6.66 (d, J = 8.28 Hz, 1H) 4.47 (t, J = 8.66 Hz, 2H) 3.11 (t, J = 8.78 Hz, 2H) 2.98 (s, 3H) 1.91 (s, 0.5H) |
| 361 | 2-Amino-5-(3,4-dihydro- 2H-chromen-6-yl)-5-(3′- methoxybiphenyl-3-yl)-3- methyl-3,5-dihydro-4H- imidazol-4-one 0.25 acetate | 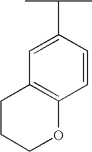 | 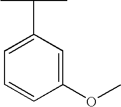 | 428.1 | 7.69 (br. s., 1H) 7.48-7.52 (m, 1H) 7.42-7.46 (m, 1H) 7.34-7.41 (m, 2H) 7.11-7.15 (m, 2H) 7.07-7.11 (m, 1H) 7.03-7.06 (m, 1H) 6.92-6.96 (m, 1H) 6.64 (d, J = 9.03 Hz, 1H) 4.05-4.09 (m, 2H) 3.80 (s, 3H) 2.97 (s, 3H) 2.67 (t, J = 6.40 Hz, 2H) 1.91 (s, 0.5H) 1.83-1.90 (m, 2H) |
| 362 | 2-Amino-5-(3,4-dihydro- 2H-chromen-6-yl)-5-(3′- ethoxybiphenyl-3-yl)-3- methyl-3,5-dihydro-4H- imidazol-4-one | 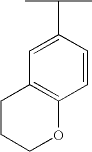 | 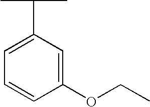 | 442.1 | 7.69 (br. s., 1H) 7.49 (d, J = 7.78 Hz, 1H) 7.42-7.46 (m, 1H) 7.33-7.39 (m, 2H) 7.11-7.15 (m, 2H) 7.08 (d, J = 7.78 Hz, 1H) 7.01-7.04 (m, 1H) 6.90-6.94 (m, 1H) 6.64 (d, J = 9.29 Hz, 1H) 4.04-4.11 (m, 4H) 2.97 (s, 3H) 2.67 (t, J = 6.40 Hz, 2H) 1.83-1.90 (m, 2H) 1.35 (t, J = 6.90 Hz, 3H) |
| 363 | 2-Amino-5-(3,4-dihydro- 2H-chromen-6-yl)-5-(2′,5′- dimethoxybiphenyl-3-yl)-3- methyl-3,5-dihydro-4H- imidazol-4-one 0.25 acetate | 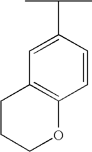 | 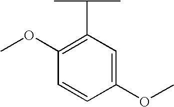 | 458.1 | 7.56 (br. s., 1H) 7.35-7.38 (m, 1H) 7.27-7.35 (m, 2H) 7.13-7.18 (m, 2H) 7.01 (d, J = 9.03 Hz, 1H) 6.89 (dd, J = 8.91, 3.14 Hz, 1H) 6.65 (d, J = 9.03 Hz, 1H) 4.05-4.10 (m, 2H) 3.73 (s, 3H) 2.96 (s, 3H) 2.64-2.72 (m, 2H) 1.91 (s, 0.5H) 1.84-1.90 (m, 2H) |
| 364 | 2-Amino-5-[3-(1,3- benzodioxol-5-yl)phenyl]- 5-(3,4-dihydro-2H- chromen-6-yl)-3-methyl- 3,5-dihydro-4H-imidazol-4- one 0.25 acetate | 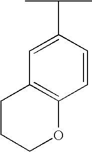 | 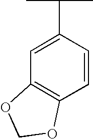 | 442.1 | 7.64 (br. s., 1H) 7.41-7.45 (m, 1H) 7.37-7.41 (m, 1H) 7.33 (t, J = 7.53 Hz, 1H) 7.10-7.14 (m, 2H) 7.08 (br. s., 1H) 6.99-7.00 (m, 2H) 6.64 (d, J = 9.03 Hz, 1H) 6.05 (s, 2H) 4.05-4.09 (m, 2H) 2.97 (s, 3H) 2.67 (t, J = 6.40 Hz, 2H) 1.91 (s, 0.4H) 1.83-1.90 (m, 2H) |
| 365 | 2-Amino-5-(3,4-dihydro- 2H-chromen-6-yl)-5-(2′- fluoro-5′-methoxybiphenyl- 3-yl)-3-methyl-3,5-dihydro- 4H-imidazol-4-one | 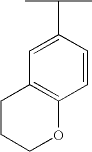 | 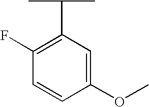 | 446.1 | 7.63 (br. s., 1H) 7.44-7.49 (m, 1H) 7.38-7.41 (m, 2H) 7.22 (dd, J = 10.16, 8.91 Hz, 1H) 7.10-7.16 (m, 2H) 6.89-6.98 (m, 2H) 6.64 (d, J = 8.78 Hz, 1H) 4.05-4.09 (m, 2H) 3.78 (s, 3H) 2.97 (s, 3H) 2.67 (t, J = 6.53 Hz, 2H) 1.83-1.90 (m, 2H) |
| 366 | 2-Amino-5-(3,4-dihydro- 2H-chromen-6-yl)-5-(2′- fluoro-3′-methoxybiphenyl- 3-yl)-3-methyl-3,5-dihydro- 4H-imidazol-4-one | 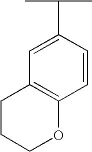 | 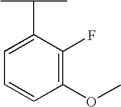 | 446.1 | 7.60-7.63 (m, 1H) 7.44-7.49 (m, 1H) 7.35-7.41 (m, 2H) 7.10-7.23 (m, 4H) 6.90-6.96 (m, 1H) 6.64 (d, J = 9.29 Hz, 1H) 4.05-4.10 (m, 2H) 3.87 (s, 3H) 2.97 (s, 3H) 2.64-2.70 (m, 2H) 1.83-1.90 (m, 2H) |
| 367 | 3′-[2-Amino-4-(3,4- dihydro-2H-chromen-6-yl)- 1-methyl-5-oxo-4,5- dihydro-1H-imidazol-4-yl]- 4-fluorobiphenyl-3- carbonitrile | 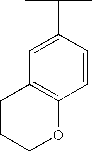 | 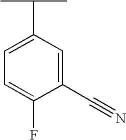 | 441.1 | 8.10 (dd, J = 6.02, 2.51 Hz, 1H) 7.90-7.95 (m, 1H) 7.71-7.74 (m, 1H) 7.62 (t, J = 9.03 Hz, 1H) 7.54-7.58 (m, 1H) 7.49-7.53 (m, 1H) 7.42 (t, J = 7.78 Hz, 1H) 7.10-7.15 (m, 2H) 6.64 (d, J = 9.29 Hz, 1H) 4.05-4.09 (m, 2H) 2.98 (s, 3H) 2.67 (t, J = 6.40 Hz, 2H) 1.83-1.90 (m, 2H) |
| 368 | 3′-[2-Amino-4-(3,4- dihydro-2H-chromen-6-yl)- 1-methyl-5-oxo-4,5- dihydro-1H-imidazol-4-yl]- 6-fluorobiphenyl-3- carbonitrile 0.25 acetate | 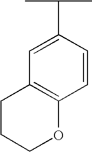 | 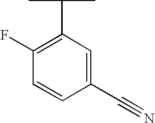 | 441.1 | ppm 7.99 (dd, J = 7.15, 1.88 Hz, 1H) 7.91-7.96 (m, 1H) 7.68 (br. s., 1H) 7.52-7.59 (m, 1H) 7.41-7.47 (m, 2H) 7.29-7.38 (m, 1H) 7.10-7.15 (m, 2H) 6.64 (d, J = 8.53 Hz, 1H) 4.05-4.10 (m, 2H) 2.97 (s, 3H) 2.64-2.69 (m, 2H) 1.91 (s, 0.4H) 1.83-1.90 (m, 2H) |
| 369 | 2-Amino-5-(3,4-dihydro- 2H-chromen-6-yl)-3- methyl-5-(3′-nitrobiphenyl- 3-yl)-3,5-dihydro-4H- imidazol-4-one 0.25 acetate | 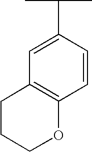 | 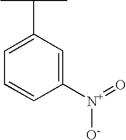 | 443.1 | 8.30 (t, J = 2.01 Hz, 1H) 8.20-8.24 (m, 1H) 8.01-8.05 (m, 1H) 7.80-7.81 (m, 1.63 Hz, 1H) 7.77 (t, J = 8.03 Hz, 1H) 7.62-7.65 (m, 1H) 7.54-7.58 (m, 1H) 7.46 (t, J = 7.78 Hz, 1H) 7.10-7.15 (m, 2H) 6.64 (d, J = 9.03 Hz, 1H) 4.05-4.09 (m, 2H) 2.98 (s, 3H) 2.67 (t, J = 6.27 Hz, 2H) 1.91 (s, 0.5H) 1.83-1.90 (m, 2H) |
| 370 | 2-Amino-5-(3,4-dihydro- 2H-chromen-6-yl)-5-[3-(3- furyl)phenyl]-3-methyl-3,5- dihydro-4H-imidazol-4-one 0.25 acetate | 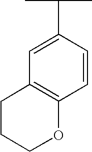 | 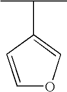 | 388.1 | 8.03-8.04 (m, 1H) 7.73 (t, J = 1.76 Hz, 1H) 7.60 (t, J = 1.63 Hz, 1H) 7.43-7.47 (m, 1H) 7.33-7.37 (m, 1H) 7.30 (t, J = 7.65 Hz, 1H) 7.08-7.12 (m, 2H) 6.78 (dd, J = 1.76, 0.75 Hz, 1H) 6.61-6.65 (m, 1H) 4.05-4.09 (m, 2H) 2.97 (s, 3H) 2.67 (t, J = 6.53 Hz, 2H) 1.91 (s, 0.4H) 1.83-1.90 (m, 2H) |
| 371 | 2-Amino-5-[3-(1- benzofuran-2-yl)phenyl]-5- (3,4-dihydro-2H-chromen- 6-yl)-3-methyl-3,5-dihydro- 4H-imidazol-4-one | 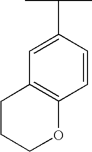 | 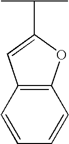 | 438.1 | 8.02 (br. s., 1H) 7.79 (d, J = 7.78 Hz, 1H) 7.62-7.67 (m, 2H) 7.52 (d, J = 8.03 Hz, 1H) 7.43 (t, J = 7.78 Hz, 1H) 7.23-7.35 (m, 3H) 7.10-7.14 (m, 2H) 6.65 (d, J = 9.03 Hz, 1H) 4.05 4.10 (m, 2H) 2.99 (s, 3H) 2.67 (t, J = 6.27 Hz, 2H) 1.83-1.90 (m, 2H) |
| 372 | 2-Amino-5-(3,4-dihydro- 2H-chromen-6-yl)-3- methyl-5-[3′- (trifluoromethoxy)biphenyl- 3-yl]-3,5-dihydro-4H- imidazol-4-one 0.25 acetate | 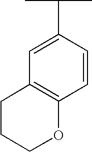 | 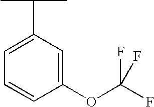 | 482.1 | 7.73 (t, J = 1.63 Hz, 1H) 7.53-7.64 (m, 3H) 7.49-7.52 (m, 1H) 7.46-7.48 (m, 1H) 7.41 (t, J = 7.65 Hz, 1H) 7.35-7.39 (m, 1H) 7.11-7.15 (m, 2H) 6.62-6.66 (m, 1H) 4.05-4.09 (m, 2H) 2.98 (s, 3H) 2.67 (t, J = 6.27 Hz, 2H) 1.91 (0.5H) 1.83-1.90 (m, 2H) |
| 373 | 2-Amino-5-(3′- chlorobiphenyl-3-yl)-5- (3,4-dihydro-2H-chromen- 6-yl)-3-methyl-3,5-dihydro- 4H-imidazol-4-one | 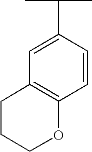 | 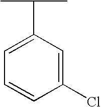 | 432.1, 434.0 | 7.71-7.73 (m, 1H) 7.56-7.58 (m, 1H) 7.52-7.55 (m, 1H) 7.47-7.52 (m, 3H) 7.42-7.46 (m, 1H) 7.40 (t, J = 7.65 Hz, 1H) 7.10-7.14 (m, 2H) 6.62-6.65 (m, 1H) 4.05-4.09 (m, 2H) 2.97 (s, 3H) 2.67 (t, J = 6.40 Hz, 2H) 1.83-1.90 (m, 2H) |
| 374 | 2-Amino-5-(3,4-dihydro- 1H-isochromen-7-yl)-5-(2′- fluoro-5′-methoxybiphenyl- 3-yl)-3-methyl-3,5-dihydro- 4H-imidazol-4-one | 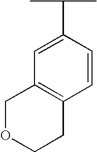 | 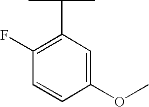 | 446.1 | 7.62-7.65 (m, 1H) 7.45-7.49 (m, 1H) 7.38-7.42 (m, 2H) 7.23-7.27 (m, 1H) 7.22 (dd, J = 10.16, 8.91 Hz, 1H) 7.12-7.14 (m, 1H) 7.06 (d, J = 8.03 Hz, 1H) 6.89-6.99 (m, 2H) 4.62 (s, 2H) 3.83 (t, J = 5.77 Hz, 2H) 3.77 (s, 3H) 2.97 (s, 3H) 2.71 (t, J = 5.77 Hz, 2H) |
| 375 | 3′-[2-Amino-4-(3,4- dihydro-1H-isochromen-7- yl)-1-methyl-5-oxo-4,5- dihydro-1H-imidazol-4-yl]- 6-fluorobiphenyl-3- carbonitrile 0.25 acetate | 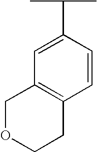 | 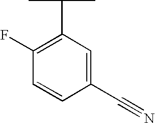 | 441.1 | 7.99 (dd, J = 7.28, 2.26 Hz, 1H) 7.92-7.96 (m, 1H) 7.67-7.70 (m, 1H) 7.52-7.58 (m, 2H) 7.41-7.48 (m, 2H) 7.23-7.27 (m, 1H) 7.11-7.13 (m, 1H) 7.06 (d, J = 8.03 Hz, 1H) 4.62 (s, 2H) 3.83 (t, J = 5.77 Hz, 2H) 2.97 (s, 3H) 2.71 (t, J = 5.77 Hz, 2H) 1.91 (s, 0.5H) |
| 376 | 2-Amino-5-(3,4-dihydro- 1H-isochromen-7-yl)-5-[3- (3-furyl)phenyl]-3-methyl- 3,5-dihydro-4H-imidazol-4- one 0.25 acetate | 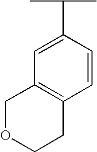 | 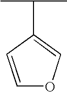 | 388.1 | 8.03-8.04 (m, 1H) 7.73 (t, J = 1.76 Hz, 1H) 7.59-7.61 (m, 1H) 7.43-7.47 (m, 1H) 7.33-7.37 (m, 1H) 7.30 (t, J = 7.65 Hz, 1H) 7.21-7.24 (m, 1H) 7.09-7.11 (m, 1H) 7.05 (d, J = 8.28 Hz, 1H) 6.78 (dd, J = 1.88, 0.88 Hz, 1H) 4.62 (s, 2H) 3.83 (t, J = 5.65 Hz, 2H) 2.98 (s, 3H) 2.71 (t, J = 5.65 Hz, 2H) 1.91 (s, 0.5H) |

### Example 377

### 3′-[2-Amino-4-(2,3-dihydro-1-benzofuran-5-yl)-1-methyl-5-oxo-4,5-dihydro-1H-imidazol-4-yl]-5-methoxybiphenyl-3-yl methanesulfonate 0.25 acetate

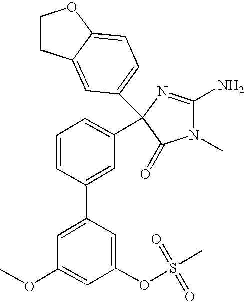

2-Amino-5-(3-bromophenyl)-5-(2,3-dihydro-1-benzofuran-5-yl)-3-methyl-3,5-dihydro-4H-imidazol-4-one (60 mg, 0.155 mmol), {3-methoxy-5-[(methylsulfonyl)oxy]phenyl}boronic acid (50 mg, 0.202 mmol), [1,1′-bis(diphenylphosphino)ferrocene]palladium(II) chloride dichloromethane adduct (13 mg, 0.015 mmol), potassium carbonate (107 mg, 0.465 mmol) in anhydrous tetrahydrofuran (3 mL) was irradiated under an atmosphere of argon in a microwave at 130° C. for 2 h. When cooled to ambient temperature the mixture was filtered and dimethyl sulfoxide (500 μL) was added. The solution was concentrated in vacuo to remove the reaction solvents and purified by preparative HPLC to give 32 mg (41% yield) of the title compound. ¹H NMR (DMSO-d₆) δ 7.69-7.71 (m, 1H), 7.52-7.56 (m, 1H), 7.48-7.52 (m, 1H), 7.40 (t, J=7.78 Hz, 1H), 7.27-7.29 (m, 1H), 7.15-7.19 (m, 1H), 7.06-7.08 (m, 1H), 7.03-7.05 (m, 1H), 6.95 (t, J=2.26 Hz, 1H), 6.66 (d, J=8.53 Hz, 1H), 4.47 (t, J=8.66 Hz, 2H), 3.85 (s, 3H), 3.42 (s, 3H), 3.11 (t, J=8.66 Hz, 2H), 2.98 (s, 3H), 1.91 (s, 0.4H); MS (ES) m/z 508 [M+H]⁺.

### Examples 378-379

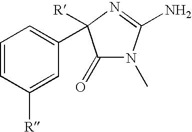

TABLE 9
Representative examples synthesized as described for 3′-[2-Amino-4-(2,3-dihydro-1-benzofuran-5-yl)-1-
methyl-5-oxo-4,5-dihydro-1H-imidazol-4-yl]-5-methoxybiphenyl-3-yl methanesulfonate 0.25 acetate.

| | | | | [M + H]⁺ | ¹H-NMR |
|---|---|---|---|---|---|
| Ex | Chemical name | R′ | R″ | m/z | (DMSO-d₆) δ |
| 378 | 3′-[2-Amino-4-(3,4-dihydro- 2H-chromen-6-yl)-1-methyl- 5-oxo-4,5-dihydro-1H- imidazol-4-yl]-5- methoxybiphenyl-3-yl methanesulfonate 0.25 acetate | 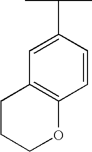 | 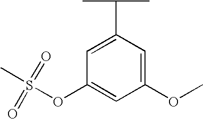 | 522.1 | 7.68-7.70 (m, 1H) 7.52-7.56 (m, 1H) 7.48-7.51 (m, 1H) 7.40 (t, J = 7.78 Hz, 1H) 7.10-7.15 (m, 2H) 7.06-7.08 (m, 1H) 7.03-7.05 (m, 1H) 6.95 (t, J = 2.13 Hz, 1H) 6.64 (d, J = 8.78 Hz, 1H) 4.05-4.09 (m, 2H) 3.85 (s, 3H) 3.42 (s, 3H) 2.97 (s, 3H) 2.67 (t, J = 6.40 Hz, 2H) 1.91 (s, 0.4H) 1.83-1.90 (m, 2H) |
| 379 | 3′-[2-Amino-4-(3,4-dihydro- 1H-isochromen-7-yl)-1- methyl-5-oxo-4,5-dihydro-1H- imidazol-4-yl]-5- methoxybiphenyl-3-yl methanesulfonate | 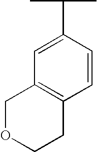 | 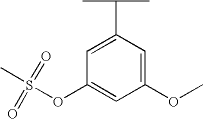 | 522.1 | 7.69-7.71 (m, 1H) 7.52-7.56 (m, 1H) 7.48-7.52 (m, 1H) 7.40 (t, J = 7.65 Hz, 1H) 7.23-7.26 (m, 1H) 7.12 (br. s., 1H) 7.03-7.08 (m, 3H) 6.95 (t, J = 2.13 Hz, 1H) 4.62 (s, 2H) 3.85 (s, 3H) 3.83 (t, J = 5.90 Hz, 2H) 3.42 (s, 3H) 2.98 (s, 3H) 2.71 (t, J = 5.77 Hz, 2H) |

### Example 380

### 4-{[tert-Butyl(diphenyl)silyl]oxy}benzaldehyde

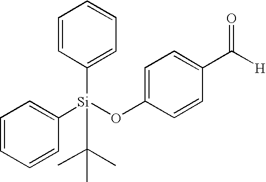

Imidazole (6.1 g, 90.0 mmol) was added to a solution of 4-hydroxybenzaldehyde (10 g, 81.9 mmol; described in: George R. Pettit et al, J. Med. Chem., 2002, 45, 2534-2542) in dimethylformamide (150 ml). The solution was stirred for 1 h, tert-butyldiphenylsilylchloride (23 mL, 79.0 mmol) was added dropwise and the resulting mixture was stirred for 48 h. The reaction mixture was poured into water and extracted with ethyl acetate (×3). The combined organic phases were washed with water, dried over magnesium sulfate and the solvent was evaporated. The residue was purified by column chromatography, using heptane/ethyl acetate (8:1) to give 26.8 g (94% yield) of the title compound. ¹H NMR (CDCl₃) δ 9.82 (s, 1H), 7.72 (d, J=1.52 Hz, 2H), 7.70 (d, J=1.52 Hz, 2H), 7.64-7.68 (m, 2H), 7.44-7.49 (m, 2H), 7.37-7.42 (m, 4H), 6.85-6.90 (m, 2H), 1.13 (s, 9H).

### Example 381

### tert-Butyl[4-(1,3-dithian-2-yl)phenoxy]diphenylsilane

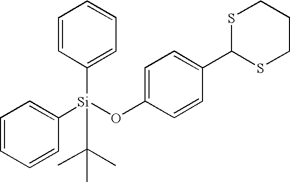

Boron trifluoride-diethyl etherate (25 mL, 199 mmol) was added dropwise to a cooled (0° C.) solution of 4-{[tert-butyl(diphenyl)silyl]oxy}benzaldehyde (18.4 g, 51.0 mmol; described in: Mor, M. et al. J Med. Chem. 2004, 47 4998-5008 and Kojima, S et al. ITE Letters on Batteries, New technologies & Medicine 2002, 2(2), 241-244) and 1,3-propanedithiol (5.1 mL, 51.0 mmol) in dichloromethane (150 mL). The resulting mixture was stirred at 0° C. for 2 h. Saturated aqueous sodium bicarbonate was added and the aqueous phase was extracted with dichloromethane. The combined organic phases were washed with water, aqueous potassium hydroxide (10%) and water, dried over magnesium sulfate and the solvent was evaporated. The residue was purified by column chromatography, using heptane/ethyl acetate (8:1 to 2:1) as the eluent, to give 17.0 g (74% yield) of the title compound. ¹H NMR (CDCl₃) δ 7.71 (dd, J=7.96, 1.39 Hz, 4H), 7.40-7.46 (m, 2H), 7.35-7.40 (m, 4H), 7.17-7.23 (m, 3H), 6.68-6.75 (m, 2H), 5.07 (s, 1H), 2.98-3.08 (m, 2H), 2.84-2.92 (m, 2H), 2.10-2.20 (m, 1H), 1.83-1.96 (m, 1H), 1.09-1.12 (m, 9H).

### Example 382

### (3-Bromo-4-fluorophenyl)[2-(4-{[tert-butyl(diphenyl)silyl]oxy}phenyl)-1,3-dithian-2-yl]methanol

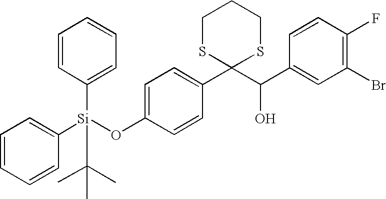

To a solution of tert-butyl[4-(1,3-dithian-2-yl)phenoxy]diphenylsilane (2.0 g, 4.44 mmol; described in: Philip C. Bulman Page et al, Tetrahedron, 1992 48, 7265-7274) in tetrahydrofuran (50 mL) was added a solution of n-butyllithium in hexane (3.05 mL, 4.88 mmol, 1.6 M) at −78° C. and the mixture was stirred at −78° C. for 2 h. To the resulting anion at −78° C. was added 3-bromo-4-fluorobenzaldehyde (0.95 g, 4.66 mmol) and the reaction was allowed to reach room temperature overnight. The reaction mixture was poured onto saturated ammonium chloride solution and the aqueous layer extracted with dichloromethane (×3). The combined organic layers were dried over magnesium sulfate and the solvent was evaporated. The residue was purified by column chromatography, using heptane/ethyl acetate (7:1 to 5:1) as the eluent, to give 1.95 g (67% yield) of the title compound. ¹H NMR (CDCl₃) δ 7.74 (dd, J=6.82, 1.26 Hz, 4H), 7.43-7.48 (m, 2H), 7.37-7.43 (m, 4H), 7.31-7.36 (m, 2H), 7.10 (dd, J=6.69, 2.15 Hz, 1H), 6.68-6.74 (m, 3H), 6.54-6.61 (m, 1H), 4.84 (s, 1H), 2.61-2.76 (m, 4H), 1.86-1.95 (m, 2H), 1.09-1.17 (m, 9H). HR MS (ES) m/z 635.0881, 637.0852 [M−18]⁺.

### Example 383

### 1-(3-Bromo-4-fluorophenyl)-2-(4-{[tert-butyl(diphenyl)silyl]oxy}phenyl)ethane-1,2-dione

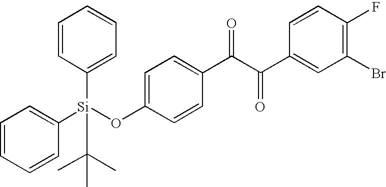

Dess-Martin periodinane (3.1 g, 7.23 mmol) was added to a solution of (3-bromo-4-fluorophenyl)[2-(4-{[tert-butyl(diphenyl)silyl]oxy}phenyl)-1,3-dithian-2-yl]methanol (1.90 g, 2.91 mmol) and tert-butanol (0.93 mL, 10.2 mmol) in dichloromethane (40 mL) under an argon atmosphere and the reaction mixture was stirred overnight. Sodium thiosulfate (2.5 g) dissolved in saturated aqueous sodium hydrogencarbonate (40 mL) was added and the resulting mixture was stirred for 30 minutes. More dichloromethane was added and the organic phase separated. The aqueous phase was extracted with dichloromethane (×3), the combined organic phases were dried over magnesium sulfate and the solvent was evaporated. The residue was purified by column chromatography, using heptane/ethyl acetate (9:1 to 8:1) as the eluent, to give 1.54 g (94% yield) of the title compound. HR MS (ES) m/z 561.0924, 563.0913 [M+H]⁺.

### Example 384

### 5-(3-Bromo-4-fluorophenyl)-5-(4-hydroxyphenyl)-3-methyl-2-thioxoimidazolidin-4-one

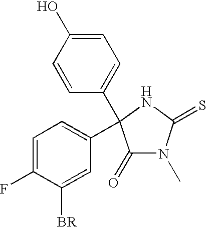

Aqueous potassium hydroxide (1.2 M, 4.45 mL, 5.34 mmol) was added to a solution of 1-(3-bromo-4-fluorophenyl)-2-(4-{[tert-butyl(diphenyl)silyl]oxy}phenyl)ethane-1,2-dione (1.50 g, 2.67 mmol) and methyl-2-thiourea (0.48 g, 5.34 mmol) in dimethyl sulfoxide (10 mL) at 100° C. and the reaction mixture was allowed cool down to room temperature after 1 h of stirring. The reaction mixture was diluted with water and dichloromethane, The pH was adjusted to 3-4 by addition of aqueous hydrochloric acid (2 M). The organic phase was separated and the aqueous phase was washed with dichloromethane (×3). The combined organic phases were dried over magnesium sulfate and the solvent was evaporated. The residue was purified by column chromatography, using heptane/ethyl acetate (3:1 to 2:1) as the eluent, to give 0.73 g (69% yield) of the title compound. ¹H NMR (CDCl₃) δ 8.75 (s, 1H), 7.57 (dd, J=6.19, 2.15 Hz, 1H), 7.22-7.39 (m, 1H), 7.01-7.17 (m, 3H), 6.78 (d, J=8.59 Hz, 2H), 3.33 (s, 3H): MS (ES) m/z 392.97, 394.94 [M−H]⁻.

### Example 385

### 4-[4-(3-Bromo-4-fluorophenyl)-1-methyl-5-oxo-2-thioxoimidazolidin-4-yl]phenyl methanesulfonate

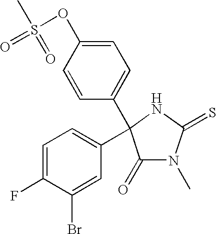

Methanesulfonyl chloride (0.96 mL, 12.5 mmol) was added to a cooled (0° C.) solution of 5-(3-bromo-4-fluorophenyl)-5-(4-hydroxyphenyl)-3-methyl-2-thioxoimidazolidin-4-one (3.98 g, 10.1 mmol) and triethylamine (1.74 mL, 12.6 mmol) in dichloromethane (100 mL) and the reaction was kept at 8° C. overnight. The reaction mixture was washed with water and brine. The organic phase was dried over magnesium sulfate and the solvent was evaporated to give 4.77 g (9.7% yield) of the title compound. MS (ES) m/z 470.95, 472.95 [M−H]⁻.

### Example 386

### 4-[2-Amino-4-(3-bromo-4-fluorophenyl)-1-methyl-5-oxo-4,5-dihydro-1H-imidazol-4-yl]phenyl methanesulfonate

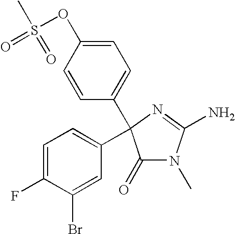

tert-Butylperoxide (23.4 mL, 151.5 mmol, 70 wt % aq) was added to a solution of 4-[4-(3-bromo-4-fluorophenyl)-1-methyl-5-oxo-2-thioxoimidazolidin-4-yl]phenyl methanesulfonate (4.77 g, 10.1 mmol) in a mixture of methanol/ammonium hydroxide (25 mL:75 mL). The reaction was stirred at room temperature for 3 h and at 8° C. overnight. The reaction mixture was concentrated and the residue was dissolved in chloroform. The organic phase was washed with water, dried over magnesium sulfate and the solvent was evaporated. The residue was purified by column chromatography, using ethyl acetate/methanol (20:1+1% triethylamine) as the eluent, to give 2.6 g (60% yield) of the title compound. ¹H NMR (CDCl₃) δ 7.72 (dd, J=6.57, 2.27 Hz, 1H), 7.49-7.56 (m, 2H), 7.38-7.45 (m, 1H), 7.19-7.25 (m, 2H), 7.05 (t, J=8.46 Hz, 1H), 3.11-3.14 (m, 3H), 3.10 (s, 3H): MS (ES) m/z 453.96, 455.98 [M−H]⁻.

### Example 387

### 4-[2-Amino-4-(3′-cyano-6-fluorobiphenyl-3-yl)-1-methyl-5-oxo-4,5-dihydro-1H-imidazol-4-yl]phenyl methanesulfonate

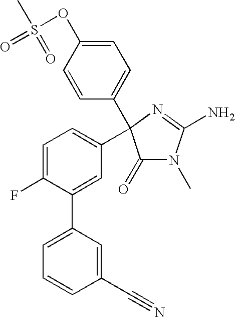

4-[2-Amino-4-(3-bromo-4-fluorophenyl)-1-methyl-5-oxo-4,5-dihydro-1H-imidazol-4-yl]phenyl methanesulfonate (80 mg, 0.18 mmol), (3-cyanophenyl)boronic acid (0.034 g, 0.23 mol), [1,1′-bis(diphenylphosphino)ferrocene]palladium(II) chloride dichloromethane adduct (0.014 g, 0.018 mol), potassium carbonate (0.145 mg, 1.05 mmol) in anhydrous tetrahydrofuran (3 mL) was irradiated under argon atmosphere in a microwave at 130° C. for 2 h. When cooled to ambient temperature the mixture was filtered and dimethyl sulfoxide (0.5 mL) was added. The solution was concentrated in vacuo to remove the tetrahydrofuran and purified by preparative HPLC to give 0.025 g (30% yield) of the title compound: ¹H NMR (DMSO-d₆) δ 7.94 (s, 1H), 7.87-7.92 (m, 1H), 7.78-7.84 (m, 1H), 7.68-7.73 (m, 1H), 7.65-7.68 (m, 1H), 7.55-7.61 (m, 3H), 7.30-7.36 (m, 1H), 7.30 (s, 1H), 7.28 (s, 1H), 6.77 (s, 2H), 3.35 (s, 3H), 2.99 (s, 3H): MS (ES) m/z 479.1 [M+H]⁺.

### Examples 388-405

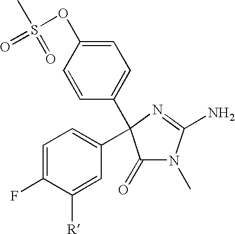

TABLE 10
Representative examples synthesized as described for 4-[2-Amino-4-(3′-cyano-6-fluorobiphenyl-
3-yl)-1-methyl-5-oxo-4,5-dihydro-1H-imidazol-4-yl]phenyl methanesulfonate

| | | | [M + H]⁺ | ¹H-NMR |
|---|---|---|---|---|
| Ex | Chemical name | R′ | m/z | (DMSO-d₆) δ |
| 388 | 4-[2-Amino-4-(3′-cyano-6- fluorobiphenyl-3-yl)-5-oxo- 4,5-dihydro-1H-imidazol-4- yl]phenyl methanesulfonate | 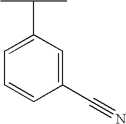 | 479.1 | 7.94 (s, 1H), 7.87-7.92 (m, 1H), 7.78-7.84 (m, 1H), 7.68-7.73 (m, 1H), 7.65-7.68 (m, 1H), 7.55-7.61 (m, 3H), 7.30-7.36 (m, 1H), 7.30 (s, 1H), 7.28 (s, 1H), 6.77 (br. s., 2H), 3.35 (s, 3H), 2.99 (s, 3H) |
| 389 | 4-[2-Amino-4-(2′,6-difluoro- 3′-methoxybiphenyl-3-yl)-5- oxo-4,5-dihydro-1H-imidazol- 4-yl]phenyl methanesulfonate | 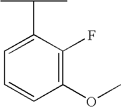 | 502.0 | N/A |
| 390 | 4-[2-Amino-4-(2′,6-difluoro- 5′-methoxybiphenyl-3-yl)-5- oxo-4,5-dihydro-1H-imidazol- 4-yl]phenyl methanesulfonate | 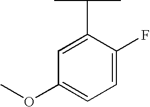 | 502.0 | 7.53-7.61 (m, 4H), 7.21-7.32 (m, 4H), 6.99-7.06 (m, 1H), 6.89 (dd, J = 5.90, 3.14 Hz, 1H), 6.75 (br. s., 2H), 3.77 (s, 3H), 3.35 (s, 3H), 2.99 (s, 3H) |
| 391 | 4-[2-Amino-4-(3′-cyano-4′,6- difluorobiphenyl-3-yl)-5-oxo- 4,5-dihydro-1H-imidazol-4- yl]phenyl methanesulfonate | 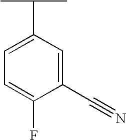 | 497.0 | 8.05 (dd, J = 6.15, 1.38 Hz, 1H), 7.84-7.91 (m, 1H), 7.61-7.68 (m, 2H), 7.55-7.59 (m, J = 8.78 Hz, 3H), 7.30-7.35 (m, 1H), 7.30 (s, 1H), 7.26-7.29 (m, 1H), 6.77 (br. s., 2H), 3.35 (s, 3H), 2.99 (s, 3H) |
| 392 | 4-[2-Amino-4-(5′-cyano-2′,6- difluorobiphenyl-3-yl)-5-oxo- 4,5-dihydro-1H-imidazol-4- yl]phenyl methanesulfonate | 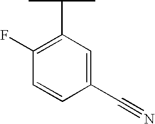 | 497.0 | 7.99-8.06 (m, 2H), 7.60-7.66 (m, 2H), 7.54-7.60 (m, 3H), 7.31-7.37 (m, 1H), 7.29-7.31 (m, 1H), 7.28 (s, 1H), 6.77 (br. s., 2H), 3.35 (s, 3H), 2.97 (s, 3H) |
| 393 | 4-[2-Amino-4-(4-fluoro-3- pyridin-3-ylphenyl)-5-oxo- 4,5-dihydro-1H-imidazol-4- yl]phenyl methanesulfonate | 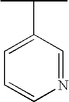 | 455.0 | 8.67 (s, 1H), 8.61 (dd, J = 4.89, 1.63 Hz, 1H), 7.86-7.93 (m, 1H), 7.66 (dd, J = 7.65, 2.13 Hz, 1H), 7.55-7.61 (m, 3H), 7.52 (dd, J = 8.03, 4.77 Hz, 1H), 7.31-7.36 (m, 1H), 7.30 (s, 1H), 7.28 (s, 1H), 6.77 (br. s., 2H), 3.35 (s, 3H), 2.99 (s, 3H) |
| 394 | 4-{ 2-Amino-4-[4-fluoro-3-(2- fluoropyridin-3-yl)phenyl]-5- oxo-4,5-dihydro-1H-imidazol- 4-yl}phenyl methanesulfonate | 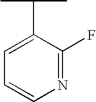 | 473.0 | 8.31 (d, J = 5.02 Hz, 1H), 7.98-8.05 (m, 1H), 7.59-7.64 (m, 2H), 7.58 (s, 1H), 7.56 (s, 1H), 7.47-7.52 (m, 1H), 7.31-7.36 (m, 1H), 7.30 (s, 1H), 7.28 (s, 1H), 6.77 (br. s., 2H), 3.35 (s, 3H), 2.99 (s, 3H) |
| 395 | 4-{2-Amino-4-[3-(5-chloro-2- fluoropyridin-3-yl)-4- fluorophenyl]-5-oxo-4,5- dihydro-1H-imidazol-4- yl}phenyl methanesulfonate | 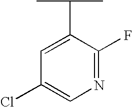 | 507.0 | 8.40 (dd, J = 2.51, 1.26 Hz, 1H), 8.24 (dd, J = 8.03, 2.51 Hz, 1H), 7.61-7.68 (m, 2H), 7.54-7.60 (m, 2H), 7.32-7.38 (m, 1H), 7.27-7.31 (m, 2H), 6.77 (br. s., 2H), 3.35 (s, 3H), 2.99 (s, 3H) |
| 396 | 4-{2-Amino-4-[4-fluoro-3-(6- fluoropyridin-3-yl)phenyl]-5- oxo-4,5-dihydro-1H-imidazol- 4-yl}phenyl methanesulfonate | 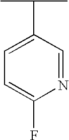 | 473.0 | 8.34 (s, 1H), 8.05-8.14 (m, 1H), 7.66 (dd, J = 7.78, 2.26 Hz, 1H), 7.54-7.61 (m, 3H), 7.29-7.36 (m, 3H), 7.28 (s, 1H), 6.77 (br. s., 2H), 3.5 (s, 3H), 2.99 (s, 3H) |
| 397 | 4-{2-Amino-4-[4-fluoro-3-(2- fluoropyridin-4-yl)phenyl]-5- oxo-4,5-dihydro-1H-imidazol- 4-yl}phenyl methanesulfonate | 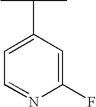 | 473.0 | 8.35 (d, J = 5.02 Hz, 1H), 7.74 (dd, J = 7.53, 2.26 Hz, 1H), 7.62-7.69 (m, 1H), 7.58 (d, J = 8.78 Hz, 2H), 7.47 (dd, J = 3.51, 1.76 Hz, 1H) 7.36 (dd, J = 10.54, 8.78 Hz, 1H), 7.27-7.32 (m, 3H), 6.78 (br. s., 2H), 3.35 (s, 3H), 2.99 (s, 3H) |
| 398 | 4-{2-Amino-4-[3-(2-chloro-3- fluoropyridin-4-yl)-4- fluorophenyl]-5-oxo-4,5- dihydro-1H-imidazol-4- yl}phenyl methanesulfonate | 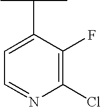 | 507.0 | 8.38 (d, J = 5.02 Hz, 1H), 7.65-7.72 (m, 2H), 7.54-7.60 (m, 3H), 7.39 (t, J = 9.41 Hz, 1H), 7.29 (d, J = 8.78 Hz, 2H), 6.79 (br. s., 2H), 3.35 (s, 3H), 2.99 (s, 3H) |
| 399 | 4-{2-Amino-4-[3-(2-chloro-5- fluoropyridin-3-yl)-4- fluorophenyl]-5-oxo-4,5- dihydro-1H-imidazol-4- yl}phenyl methanesulfonate | 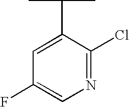 | 507.0 | 8.56 (d, J = 3.01 Hz, 1H), 8.04 (dd, J = 8.28, 3.01 Hz, 1H), 7.60-7.66 (m, 1H), 7.53-7.59 (m, 3H), 7.30-7.37 (m, 2H), 7.28 (s, 1H), 6.76 (br. s., 2H), 3.35 (s, 3H), 2.98 (s, 3H) |
| 400 | 4-{2-Amino-4-[3-(2,6- difluoropyridin-3-yl)-4- fluorophenyl]-5-oxo-4,5- dihydro-1H-imidazol-4- yl}phenyl methanesulfonate | 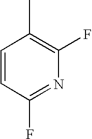 | 491.0 | 8.18-8.28 (m, 1H), 7.62 (dd, J = 7.53, 1.76 Hz, 2H), 7.57 (d, J = 8.78 Hz, 2H), 7.26-7.39 (m, 4H), 6.77 (br. s., 2H), 3.35 (s, 3H), 2.99 (s, 3H) |
| 401 | 4-{2-Amino-4-[6-fluoro-3′- (trifluoromethoxy) biphenyl- 3-yl]-5-oxo-4,5-dihydro-1H- imidazol-4-yl}phenyl methanesulfonate | 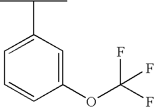 | 538.0 | 7.61-7.68 (m, 2H), 7.54-7.60 (m, 3H), 7.50 (d, J = 7.78 Hz, 1H), 7.44 (br. s., 2H), 7.25-7.35 (m, 3H), 6.76 (br. s., 2H), 3.35 (s, 3H), 2.99 (s, 3H) |
| 402 | 4-[2-Amino-4-(3′-chloro-6- fluorobiphenyl-3-yl)-5-oxo- 4,5-dihydro-1H-imidazol-4- yl]phenyl methanesulfonate | 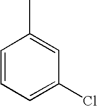 | 488.0 | 7.64 (dd, J = 7.58, 2.27 Hz, 1H), 7.53-7.59 (m, 3H), 7.49-7.53 (m, 3H), 7.41-7.45 (m, 1H), 7.27-7.31 (m, 3H), 6.81 (br. s., 2H), 3.35 (s, 3H), 2.99 (s, 3H) |
| 403 | 4-{2-Amino-4-[3-(1,3- benzodioxol-5-yl)-4- fluorophenyl]-5-oxo-4,5- dihydro-1H-imidazol-4- yl}phenyl methanesulfonate | 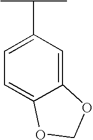 | 498.0 | 7.54-7.61 (m, 3H), 7.43-7.50 (m, 1H), 7.30 (s, 1H), 7.28 (s, 1H), 7.23 (dd, J = 10.54, 8.78 Hz, 1H), 6.99-7.04 (m, 2H), 6.91 (d, J = 8.03 Hz, 1H), 6.75 (br. s., 2H), 6.07 (s, 2H), 3.35 (s, 3H), 2.99 (s, 3H) |
| 404 | 4-[2-Amino-4-(4-fluoro-3- pyridin-4-ylphenyl)-5-oxo- 4,5-dihydro-1H-imidazol-4- yl]phenyl methanesulfonate | 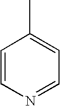 | 455.0 | 8.67 (d, J = 4.52 Hz, 2H), 7.70 (d, J = 7.53 Hz, 1H), 7.52-7.66 (m, 3H), 7.49 (d, J = 5.77 Hz, 2H), 7.25-7.39 (m, 3H), 6.78 (br. s., 2H), 3.35 (s, 3H), 2.99 (s, 3H) |
| 405 | 4-[2-Amino-4-(4-fluoro-3- pyrimidin-5-ylphenyl)-5-oxo- 4,5-dihydro-1H-imidazol-4- yl]phenyl methanesulfonate | 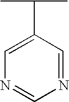 | 454.1 [M − H] | 9.23 (s, 1H), 8.95 (d, J = 1.26 Hz, 2H), 7.72 (dd, J = 7.58, 2.27 Hz, 1H), 7.55-7.67 (m, 3H), 7.37 (dd, J = 10.11, 8.84 Hz, 1H), 7.29 (d, J = 8.84 Hz, 2H), 6.78 (br. s., 2H), 3.36 (s, 3H), 2.99 (s, 3H) |

### Example 406

### 4-{2-Amino-4-[3-(2-chloro-5-methoxypyridin-3-yl)-4-fluorophenyl]-5-oxo-4,5-dihydro-1H-imidazol-4-yl}phenyl methanesulfonate

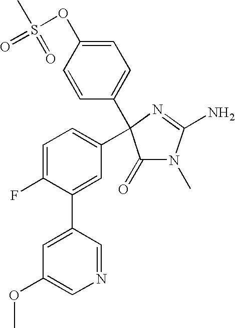

4-[2-Amino-4-(3-bromo-4-fluorophenyl)-1-methyl-5-oxo-4,5-dihydro-1H-imidazol-4-yl]phenyl methanesulfonate (0.080 g, 0.18 mmol), 3-methoxy-5-(4,4,5,5-tetramethyl-1,3,2-dioxaborolan-2-yl)pyridine (0.054 g, 0.23 mmol), [1,1′-bis(diphenylphosphino) ferrocene]palladium(II) chloride dichloromethane adduct (0.014 g, 0.018 mmol), potassium carbonate (0.145 g, 1.05 mmol) and anhydrous tetrahydrofuran (3 mL) was irradiated under argon atmosphere in a microwave at 130° C. for 2 h. When cooled to ambient temperature the mixture was filtered and dimethyl sulfoxide (0.5 mL) was added. The solution was concentrated in vacuo to remove the tetrahydrofuran and purified by preparative HPLC to give 0.009 g (10% yield) of the title compound: ¹H-NMR (DMSO-d₆): δ 8.34 (d, J=2.76 Hz, 1H), 8.24 (s, 1H), 7.65 (dd, J=7.53, 2.26 Hz, 1H), 7.55-7.62 (m, 3H), 7.42-7.45 (m, 1H), 7.25-7.36 (m, 3H), 6.76 (br. s., 2H), 3.87 (s, 3H), 3.35 (s, 3H), 2.99 (s, 3H); MS (ES) m/z 485.0 [M+H]⁺.

### Examples 407-409

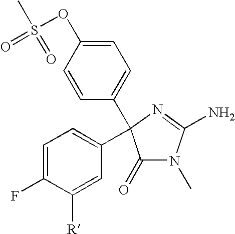

TABLE 11
Representative examples synthesized as described for 4-{2-Amino-4-{3-(2-chloro-5-methoxypyridin-
3-yl)-4-fluorophenyl]-5-oxo-4,5-dihydro-1H-imidazol-4-yl}phenyl methanesulfonate

| | | | [M + H]⁺ | ¹H-NMR |
|---|---|---|---|---|
| Ex | Chemical name | R′ | m/z | (DMSO-d₆) δ |
| 407 | 4-{2-Amino-4-[3-(2-chloro-5- methoxypyridin-3-yl)-4- fluorophenyl]-5-oxo-4,5- dihydro-1H-imidazol-4- yl}phenyl methanesulfonate | 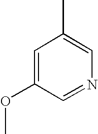 | 485.0 | 8.34 (d, J = 2.76 Hz, 1H), 8.24 (s, 1H), 7.65 (dd, J = 7.53, 2.26 Hz, 1H), 7.55-7.62 (m, 3H), 7.42-7.45 (m, 1H), 7.25-7.36 (m, 3H), 6.76 (br. s., 2H), 3.87 (s, 3H), 3.35 (s, 3H), 2.99 (s, 3H) |
| 408 | 4-(2-Amino-4-{6-fluoro-3′- methoxy-5′- [(methylsulfonyl)oxy]biphenyl- 3-yl}-5-oxo-4,5-dihydro-1H- imidazol-4-yl)phenyl methanesulfonate | 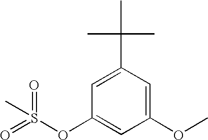 | 578.0 | 7.63 (dd, J = 7.53, 2.26 Hz, 1H), 7.52-7.60 (m, 3H), 7.24-7.35 (m, 3H), 6.97-7.05 (m, 3H), 6.77 (br. s., 2H), 3.83 (s, 3H), 3.42 (s, 3H), 3.35 (s, 3H), 2.99 (s, 3H) |
| 409 | 4-{2-Amino-4-[4-fluoro-3-(5- fluoropyridin-3-yl)phenyl]-5- oxo-4,5-dihydro-1H-imidazol-4- yl}phenyl methanesulfonate | 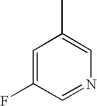 | 471.0 [M − H] | 8.64 (d, J = 2.78 Hz, 1H), 8.56 (d, J = 1.52 Hz, 1H), 7.89 (d, J = 9.85 Hz, 1H), 7.70 (dd, J = 7.58, 2.27 Hz, 1H), 7.55-7.64 (m, 3H), 7.35 (dd, J = 10.36, 8.84 Hz, 1H), 7.29 (d, J = 8.84 Hz, 2H), 6.78 (br. s., 2H), 3.35 (s, 3H), 2.99 (s, 3H) |

### Example 410

### 4-[2-Amino-4-(4-fluoro-3-pyrazin-2-ylphenyl)-5-oxo-4,5-dihydro-1H-imidazol-4-yl]phenyl methanesulfonate

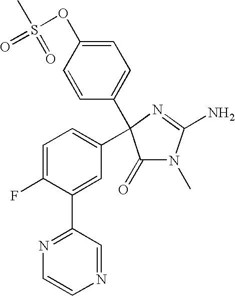

4-[2-Amino-4-(3-bromo-4-fluorophenyl)-1-methyl-5-oxo-4,5-dihydro-1H-imidazol-4-yl]phenyl methanesulfonate (0.080 g, 0.18 mmol), 2-(tributylstannanyl)pyrazine (0.097 g, 0.26 mmol), bis(triphenylposphino) palladium(II) (0.006 g, 0.009 mmol) in anhydrous tetrahydrofuran (3 mL) was irradiated under argon atmosphere in a microwave at 130° C. for 3 h. When cooled to ambient temperature the mixture was filtered and dimethyl sulfoxide (0.5 mL) was added. The solution was concentrated in vacuo to remove the tetrahydrofuran and purified by preparative HPLC to give 0.020 g (25% yield) of the title compound: ¹H NMR (DMSO-d₆) δ 9.01 (dd, J=2.91, 1.64 Hz, 1H), 8.79-8.82 (m, 1H), 8.66 (d, J=2.53 Hz, 1H), 8.17 (dd, J=7.45, 2.40 Hz, 1H), 7.63-7.70 (m, 1H), 7.57 (s, 1H), 7.54 (s, 1H), 7.37 (dd, J=10.86, 8.84 Hz, 1H), 7.30 (s, 1H), 7.28 (s, 1H), 6.80 (br. s., 2H), 3.35 (s, 3H), 2.99 (s, 3H); (ES) m/z 454.08 [M−H]⁻.

### Example 411

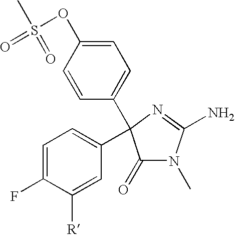

TABLE 11
Representative examples synthesized as described for examples were
prepared as described for 4-[2-Amino-4-(4-fluoro-3-pyrazin-2-ylphenyl)-
5-oxo-4,5-dihydro-1H-imidazol-4-yl]phenyl methanesulfonate.

| | | | [M + H]⁺ | ¹H-NMR |
|---|---|---|---|---|
| Ex | Chemical name | R′ | m/z | (DMSO-d₆) δ |
| 411 | 4-[2-Amino-4-(4- fluoro-3-pyrazin-2- ylphenyl)-5-oxo-4,5- dihydro-1H-imidazol- 4-yl]phenyl methanesulfonate | 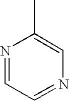 | 454.1 [M − H] | 9.01 (dd, J = 2.91, 1.64 Hz, 1H), 8.77-8.83 (m, 1H), 8.66 (d, J = 2.53 Hz, 1H), 8.17 (dd, J = 7.45, 2.40 Hz, 1H), 7.62-7.73 (m, 1H), 7.56 (d, J = 8.84 Hz, 2H), 7.37 (dd, J = 10.86, 8.84 Hz, 1H), 7.29 (d, J = 8.59 Hz, 2H), 6.80 (br. s., 2H), 3.35 (s, 3H), 2.99 (s, 3H) |

### Example 412

### 2-Amino-5-(4-hydroxyphenyl)-5-(3′-methoxybiphenyl-3-yl)-3-methyl-3,5-dihydro-4H-imidazol-4-one

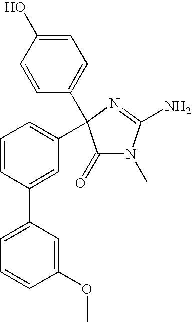

tert-Butylperoxide (6.0 mL, 47.4 mmol, 70 wt % aq) was added to a solution of 5-(4-hydroxyphenyl)-5-(3′-methoxybiphenyl-3-yl)-3-methyl-2-thioxoimidazolidin-4-one (1.27 g, 3.16 mmol) in a mixture of methanol/ammonium hydroxide (5 mL:15 mL). The resulting mixture was heated at 35° C. for 2.5 h and allowed to reach room temperature The reaction mixture was concentrated and the residue was dissolved in chloroform. The organic phase was washed with water, dried over magnesium sulfate and the solvent was evaporated. The residue was purified by column chromatography, using ethyl acetate/methanol (10:1+1% triethylamine) to give 0.95 g (78% yield) of the title compound. ¹H NMR (CDCl₃) δ 7.64 (s, 1H), 7.45 (d, J=7.58 Hz, 1H), 7.34-7.39 (m, 1H), 7.30 (d, J=7.83 Hz, 1H), 7.24 (t, J=7.96 Hz, 1H), 7.02-7.12 (m, 4H), 6.82 (dd, J=8.21, 2.15 Hz, 1H), 6.62 (d, J=8.59 Hz, 2H), 6.02 (br. s., 3H), 3.77 (s, 3H), 3.00 (s, 3H), MS (ES) m/z 386.14 [M−H]⁻.

### Example 413

### 4-[2-Amino-4-(3-methoxybiphenyl-3-yl)-1-methyl-5-oxo-4,5-dihydro-1H-imidazol-4-yl]phenyl propane-1-sulfonate hydrochloride

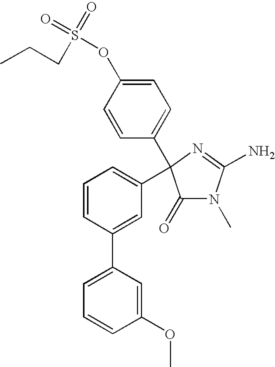

1-Propane sulfonyl chloride (0.012 mL, 0.103 mmol) was added to a cooled (0° C.) solution of 2-amino-5-(4-hydroxyphenyl)-5-(3′-methoxybiphenyl-3-yl)-3-methyl-3,5-dihydro-4H-imidazol-4-one (0.040 g, 0.103 mmol) and triethylamine (0.015 mL, 0.103 mmol) in dichloromethane (5 mL) and stirred for 1.5 h. The reaction mixture was concentrated and the residue was purified by column chromatography, using ethyl acetate/methanol (20:1) as the eluent, to give 0.031 g (61% yield) of the base. The base was dissolved in dichloromethane (0.5 mL) and treated with hydrochloric acid (4.0 M in diethyl ether). The formed hydrochloride salt precipitated after addition of diethyl ether (2 mL) to yield 0.013 g (39% yield) of the title compound. ¹H NMR (CDCl₃) δ 11.15 (s, 1H), 9.94 (s, 1H), 9.10 (s, 1H), 7.64 (s, 1H), 7.58 (d, J=7.33 Hz, 1H), 7.50-7.56 (m, 2H), 7.38-7.47 (m, 2H), 7.25-7.34 (m, 3H), 7.13 (d, J=7.58 Hz, 1H), 7.09 (d, J=2.27 Hz, 1H), 6.87 (dd, J=8.34, 2.27 Hz, 1H), 3.83 (s, 3H), 3.41 (s, 3H), 3.18-3.23 (m, 2H), 1.93-2.05 (m, 2H), 1.11 (t, J=7.45 Hz, 3H), MS (ES) m/z 493.17 [M−H]⁻.

### Example 414

### 4-[2-Amino-4-(3′-methoxybiphenyl-3-yl)-1-methyl-5-oxo-4,5-dihydro-1H-imidazol-4-yl]phenyl propane-2-sulfonate hydrochloride

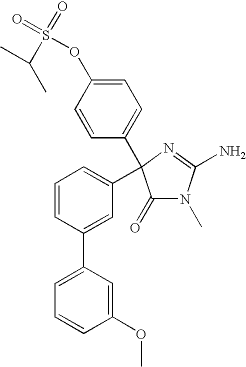

2-Propanesulfonyl chloride (0.012 mL, 0.103 mmol) was added to a cooled (0° C.) solution of 2-amino-5-(4-hydroxyphenyl)-5-(3′-methoxybiphenyl-3-yl)-3-methyl-3,5-dihydro-4H-imidazol-4-one (0.040 g, 0.103 mmol) and triethylamine (0.015 mL, 0.103 mmol) in dichloromethane (5 mL). After stirring the reaction mixture for 48 h at room temperature more triethylamine (0.006 mL, 0.043 mmol) was added. The reaction mixture was stirred for another 5 h and then concentrated The residue was purified by column chromatography, using ethyl acetate/methanol (30:1) as the eluent, to give 0.013 g (26% yield) of the base. The base was dissolved in dichloromethane (0.25 mL) and treated with hydrochloric acid (0.029 mL, 1.0 M in diethyl ether). The formed hydrochloride salt precipitated after addition of diethyl ether (2 mL) to yield 0.012 g (87% yield) of the title compound. ¹H NMR (CDCl₃) δ 11.51 (s, 1H), 9.68 (br. s., 1H), 9.56 (br. s., 1H), 7.70-7.78 (m, 1H), 7.63 (s, 1H), 7.55 (t, J=7.83 Hz, 1H), 7.47-7.52 (m, 2H), 7.34-7.45 (m, 4H), 7.12-7.21 (m, 2H), 6.98 (dd, J=7.83, 2.27 Hz, 1H), 3.81 (s, 3H), 3.71-3.78 (m, 1H), 3.20 (s, 3H), 1.43 (s, 3H), 1.42 (s, 3H); MS (ES) m/z 493.17 [M−H]⁻.

### Example 415

### 4-[2-Amino-4-(3′-methoxybiphenyl-3-yl)-1-methyl-5-oxo-4,5-dihydro-1H-imidazol-4-yl]phenyl dimethylsulfamate hydrochloride

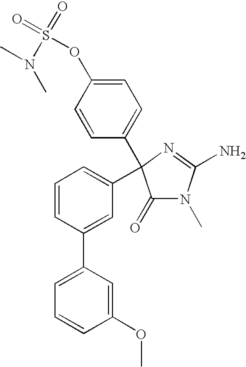

N,N-dimethylsulfamoyl chloride (0.017 mL, 0.154 mmol) was added to a cooled (0° C.) solution of 2-amino-5-(4-hydroxyphenyl)-5-(3′-methoxybiphenyl-3-yl)-3-methyl-3,5-dihydro-4H-imidazol-4-one (0.040 g, 0.103 mmol) and triethylamine (0.022 mL, 0.0.154 mmol) in dichloromethane (5 mL). After stirring the reaction mixture for 20 h at room temperature more triethylamine (0.017 mL, 0.154 mmol) and N,N-dimethylsulfamoyl chloride (0.017 mL, 0.154 mmol) were added. The reaction mixture was stirred for another 3 h and then concentrated until 2 mL of solvent remained and stirred for another 48 h. The residue was purified by column chromatography, using ethyl acetate/methanol (40:1 to 30:1) as the eluent. The pooled product was washed with saturated aqueous sodium hydrogencarbonate, dried over magnesium sulfate and the solvent was evaporated to give 0.015 g (29% yield) of the base. The base was dissolved in dichloromethane (0.25 mL) and treated with hydrochloric acid (0.033 mL, 1.0 M in diethyl ether). The formed hydrochloride salt precipitated after addition of diethyl ether (2 mL) to yield 0.010 g (62% yield) of the title compound. ¹H NMR (DMSO-d₆) δ 11.54 (s, 1H), 9.68 (br. s., 1H), 9.57 (br. s., 1H), 7.74 (d, J=7.83 Hz, 1H), 7.59-7.62 (m, 1H), 7.55 (t, J=7.83 Hz, 1H), 7.34-7.52 (m, 6H), 7.12-7.20 (m, 2H), 6.98 (dd, J=8.08, 2.02 Hz, 1H), 3.81 (s, 3H), 3.17-3.24 (m, 3H), 2.91 (s, 6H), MS (ES) m/z 494.99 [M+H]⁺

### Example 416

### 4-[2-Amino-4-(3′-methoxybiphenyl-3-yl)-1-methyl-5-oxo-4,5-dihydro-1H-imidazol-4-yl]phenyl morpholine-4-sulfonate

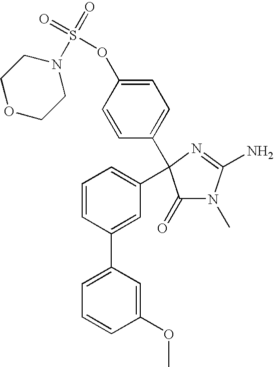

Morpholine-4-sulfonyl chloride (0.029 g, 0.154 mmol) was added to a cooled (0° C.) solution of 2-amino-5-(4-hydroxyphenyl)-5-(3′-methoxybiphenyl-3-yl)-3-methyl-3,5-dihydro-4H-imidazol-4-one (0.040 g, 0.103 mmol) and triethylamine (0.022 mL, 0.0.154 mmol) in dichloromethane (5 mL). After stirring the reaction mixture for 20 h at room temperature more triethylamine (0.017 mL, 0.154 mmol) and N,N-dimethylsulfamoyl chloride (0.017 mL, 0.154 mmol) was added. The reaction mixture was stirred for 48 h and then concentrated. The residue was purified by column chromatography, using ethyl acetate/methanol (50:1 to 40:1) as the eluent, to give 0.011 g (20% yield) of the base. The base was dissolved in dichloromethane (0.25 mL) and treated with hydrochloric acid (0.033 mL, 1.0 M in diethyl ether). The formed hydrochloride salt precipitated after addition of diethyl ether (2 mL) to yield 0.009 g (82% yield) of the title compound ¹H NMR (DMSO-d₆) δ 11.46 (s, 1H), 9.68 (br. s., 1H), 9.54 (br. s., 1H), 7.74 (d, J=7.58 Hz, 1H), 7.32-7.66 (m, 8H), 7.11-7.21 (m, 2H), 6.98 (dd, J=8.34, 2.02 Hz, 1H), 3.81 (s, 3H), 3.60-3.70 (m, 4H), 3.32-3.35 (m, 4H), 3.20 (s, 3H); MS (ES) m/z 537.01 [M+H]⁺

### Example 417

### 4-[2-Amino-4-(3′-methoxybiphenyl-3-yl)-1-methyl-5-oxo-4,5-dihydro-1H-imidazol-4-yl]phenyl 2-methoxyethanesulfonate hydrochloride

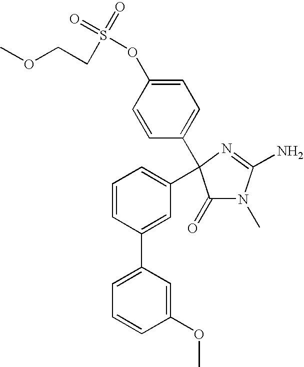

2-methoxyethanesulfonyl chloride (0.036 g, 0.227 mmol) was added to a cooled (0° C.) solution of 2-amino-5-(4-hydroxyphenyl)-5-(3′-methoxybiphenyl-3-yl)-3-methyl-3,5-dihydro-4H-imidazol-4-one (0.080 g, 0.206 mmol) and triethylamine (0.032 mL, 0.227 mmol) in dichloromethane (5 mL). The reaction mixture was concentrated after 1 h of stirring. The residue was purified by column chromatography, using ethyl acetate/methanol (40:1 to 30:1+1% triethylamine). The pooled product was washed with saturated aqueous sodium hydrogencarbonate, dried over magnesium sulfate and the solvent was evaporated to give 0.026 g (25% yield) of the base. The base was dissolved in dichloromethane (0.25 mL) and treated with hydrochloric acid (0.033 mL, 1.0 M in diethyl ether). The formed hydrochloride salt precipitated after addition of diethyl ether (2 mL) to yield 0.016 g (58% yield) of the title compound. ¹H NMR (DMSO-d₆) δ 11.69 (s, 1H), 9.68 (br. s., 2H), 7.74 (d, J=7.83 Hz, 1H), 7.66 (s, 1H), 7.33-7.60 (m, 7H), 7.14-7.22 (m, 2H), 6.98 (dd, J=8.21, 1.89 Hz, 1H), 3.73-3.90 (m, 7H), 3.28 (s, 3H), 3.20 (s, 3H); MS (ES) m/z 509.97 [M+H]⁺

### Assays

Compounds were tested in at least one of the following assays:

### β-Secretase Enzyme

The enzyme used in the IGEN Cleavage-, Fluorescent-, TR-FRET- and the BiaCore assay is described as follows:

The soluble part of the human β-Secretase (AA 1-AA 460) was cloned into the ASP2-Fc10-1-IRES-GFP-neoK mammalian expression vector. The gene was fused to the Fc domain of IgG1 (affinity tag) and stably cloned into HEK 293 cells. Purified sBACE-Fc is stored in Tris buffer, pH 9.2 and has a purity of 95%.

### IGEN Cleavage Assay

Enzyme is diluted 1:30 in 40 mM MES pH 5.0. Stock substrate is diluted to 12 μM in 40 mM MES pH 5.0. Compounds are diluted to the desired concentration in dimethylsulphoxide (final dimethylsulphoxide concentration in assay is 5%). The assay is done in a 96 well PCR plate from Greiner (#650201). Compound in dimethylsulphoxide (3 μL) is added to the plate, and then enzyme is added (27 μL) and pre-incubated with compound for 10 minutes. The reaction is started with substrate (30 μL). The final dilution of enzyme is 1:60 and the final concentration of substrate is 6 μM. After a 20 minute reaction at room temperature, the reaction is stopped by removing 10 μl of the reaction mix and diluting it 1:25 in 0.2 M Trizma-HCl, pH 8.0. Compounds are diluted and added to the plate by the Biomek FX or by hand, then all the rest of the liquid handling is done with on the Biomek 2000 instrument.

All antibodies and the streptavidin coated beads are diluted in PBS containing 0.5% BSA and 0.5% Tween20. The product is quantified by adding 50 μL of a 1:5000 dilution of the neoepitope antibody to 50 μL of the 1:25 dilution of the reaction mix. Then, 100 μL of PBS (0.5% BSA, 0.5% Tween20) containing 0.2 mg/mL IGEN beads (Dynabeads M-280) and a 1:5000 dilution of ruthinylated goat anti-rabbit (Ru-GαR) antibody is added. The final dilution of neoepitope antibody is 1:20,000, the final dilution of Ru-GAR is 1:10,000 and the final concentration of beads is 0.1 mg/mL. The mixture is read on the IGEN instrument (BioVeris) with the Abbiochemial assay program after a 2-hour incubation with shaking at room temperature.

### Fluorescent Assay

Enzyme is diluted 1:25 in 40 mM MES pH 5.0. Stock substrate (Dabcyl) is diluted to 30 μM in 40 mM MES pH 5.0. Enzyme and substrate stock solutions are kept on ice until placed in the stock plates. The Biomek FX instrument is used to do all liquid handling. Enzyme (9 μL) together with 1 μL of compound in dimethylsulphoxide is added to the plate and pre-incubated for 10 minutes. When a dose response curve is being tested for a compound, the dilutions are done in neat dimethylsulphoxide. Substrate (10 μL) is added and the reaction proceeds in the dark for 25 minutes at room temperature. The assay is done in a Corning 384 well round bottom, low volume, non-binding surface (Corning #3676). The final dilution of enzyme is 1:50, and the final concentration of substrate is 15 μM (Km of 25 μM). The fluorescence of the product is measured on a Victor II plate reader with an excitation wavelength of 360 nm and an emission wavelength of 485 nm using the protocol for labelled Edans peptide. The dimethylsulphoxide control defines 100% activity level and 0% activity is defined by exclusion of the enzyme (using 40 mM MES pH 5.0 buffer instead).

### TR-FRET Assay

Dilute the enzyme (truncated form) to 6 μg/mL (stock 1.3 mg/mL) and the substrate (Europium)CEVNLDAEFK(Qsy7) to 200 nM (stock 60 μM) in reaction buffer (NaAcetate, chaps, triton x-100, EDTA pH4.5). The Biomek FX is used for all liquid handling and the enzyme and substrate solutions are kept on ice until they are placed in Biomek FX. Enzyme (9 μl) is added to the plate then 1 μl of compound in dimethylsulphoxide is added, mixed and pre-incubated for 10 minutes. Substrate (10 μl) is then added, mixed and the reaction proceeds in the dark for 15 minutes at room temperature. The reaction is stopped with the addition of Stop solution (7 μl, NaAcetate pH 9). The fluorescence of the product is measured on a Victor II plate reader with an excitation wavelength of 340 nm and an emission wavelength of 615 nm. The assay is done in a Costar 384 well round bottom, low volume, non-binding surface (Corning #3676). The final concentration of the enzyme is 0.3 nM; the final concentration of substrate is 100 nM (Km of ˜250 nM). The dimethylsulphoxide control defines the 100% activity level and 0% activity is defined by only addition of the peptide substrate. A control inhibitor is also used in dose response assays and has an IC50 of 575 nM.

### Beta-Secretase Whole Cell Assay

### Generation of HEK293-APP695

The pcDNA3.1 plasmid encoding the cDNA of human full-length APP695 was stably transfected into HEK-293 cells using the Lipofectamine transfection reagent according to manufacture's protocol (Invitrogen). Colonies were selected with 0.1-0.5 mg/mL of zeocin. Limited dilution cloning was performed to generate homogeneous cell lines. Clones were characterized by levels of APP expression and Aβ secreted in the conditioned media using an ELISA assay developed in-house.

### Cell Culture

HEK293 cells stably expressing human wild-type APP (HEK293-APP695) were grown at 37° C. in DMEM containing 4500 μL glucose, GlutaMAX and sodium pyruvate supplemented with 10% FBS, 1% non-essential amino acids and 0.1 mg/mL of the selection antibiotic zeocin.

### Aβ40 Release Assay

Cells were harvested at 80-90% confluence and seeded at a concentration of 0.2×10⁶ cells/mL, 100 μL cell suspension/well, onto a black clear bottom 96-well poly-D-lysine coated plate. After over night incubation at 37° C., 5% CO₂, the cell medium was replaced with cell culture medium with penicillin and streptomycin (100 U/mL, 100 μg/mL, respectively) and containing test compounds in a final dimethylsulphoxide concentration of 1%. Cells were exposure to test compounds for 24 h at 37° C., 5% CO2. To quantify the amount of released Aβ, 100 μL cell medium was transferred to a round bottom polypropylene 96-well plate (assay plate). The cell plate was saved for ATP assay as described in ATP assay below. To the assay plate, 50 μL of primary detection solution containing 0.5 μg/mL of the rabbit anti-Aβ40 antibody and 0.5 μg/mL of the biotinylated monoclonal mouse 6E10 antibody in DPBS with 0.5% BSA and 0.5% Tween-20 was added per well and incubated over night at 4° C. Then, 50 μL of secondary detection solution containing 0.5 μg/mL of a ruthenylated goat anti-rabbit antibody and 0.2 mg/mL of streptavidin coated Dynabeads was added per well. The plate was vigorously shaken at room temperature for 1-2 h. The plate was then measured for electro-chemiluminescence counts in an IGEN M8 Analyzer. An Aβ standard curve was obtained using standards at concentrations 20, 10, 2 and 0.2 ng Aβ/mL in the cell culture medium with penicillin and streptomycin (100 U/mL, 100 μg/mL, respectively).

### ATP Assay

As indicated above, after transferring 100 μL medium from the cell plate for Aβ40 detection, the plate was used to analyse cytotoxicity using the ViaLight™ Plus cell proliferation/cytotoxicity kit from Cambrex BioScience that measures total cellular ATP. The assay was performed according to the manufacture's protocol. Briefly, 50 μL cell lysis reagent was added per well. The plates were incubated at room temperature for 10 min. Two min after addition of 100 μL reconstituted ViaLight™ Plus ATP reagent, the luminescence was measured in a Wallac Victor² 1420 multilabel counter.

### BACE Biacore Protocol

### Sensor Chip Preparation:

BACE was assayed on a Biacore3000 instrument by attaching either a peptidic transition state isostere (TSI) or a scrambled version of the peptidic TSI to the surface of a Biacore CM5 sensor chip. The surface of a CM5 sensor chip has 4 distinct channels that can be used to couple the peptides. The scrambled peptide KFES-statine-ETIAEVENV was coupled to channel 1 and the TSI inhibitor KTEEISEVN-statine-VAEF was couple to channel 2 of the same chip. The two peptides were dissolved at 0.2 mg/mL in 20 mM Na Acetate pH 4.5, and then the solutions were centrifuged at 14K rpm to remove any particulates. Carboxyl groups on the dextran layer were activated by injecting a one to one mixture of 0.5M N-ethyl-N′(3-dimethylaminopropyl)-carbodiimide (EDC) and 0.5M N-hydroxysuccinimide (NHS) at 5 uL/minute for 7 minutes. Then the stock solution of the control peptide was injected in channel 1 for 7 minutes at 5 uL/min., and then the remaining activated carboxyl groups were blocked by injecting 1M ethanolamine for 7 minutes at 5 uL/minute.

### Assay Protocol

The BACE Biacore assay was done by diluting BACE to 0.5 μm in Na Acetate buffer at pH 4.5 (running buffer minus dimethylsulphoxide). The diluted BACE was mixed with dimethylsulphoxide or compound diluted in dimethylsulphoxide at a final concentration of 5% dimethylsulphoxide. The BACE/inhibitor mixture was incubated for 1 hour at 4° C. then injected over channel 1 and 2 of the CM5 Biacore chip at a rate of 20 μL/minute. As BACE bound to the chip the signal was measured in response units (RU). BACE binding to the TSI inhibitor on channel 2 gave a certain signal. The presence of a BACE inhibitor reduced the signal by binding to BACE and inhibiting the interaction with the peptidic TSI on the chip. Any binding to channel 1 was non-specific and was subtracted from the channel 2 responses. The dimethylsulphoxide control was defined as 100% and the effect of the compound was reported as percent inhibition of the dimethylsulphoxide control.

hERG Assay

### Cell Culture

The hERG-expressing Chinese hamster ovary K1 (CHO) cells described by (Persson, Carlsson, Duker, & Jacobson, 2005) were grown to semi-confluence at 37° C. in a humidified environment (5% CO₂) in F-12 Ham medium containing L-glutamine, 10% foetal calf serum (FCS) and 0.6 mg/ml hygromycin (all Sigma-Aldrich). Prior to use, the monolayer was washed using a pre-warmed (37° C.) 3 ml aliquot of Versene 1:5,000 (Invitrogen). After aspiration of this solution the flask was incubated at 37° C. in an incubator with a further 2 ml of Versene 1:5,000 for a period of 6 minutes. Cells were then detached from the bottom of the flask by gentle tapping and 10 ml of Dulbecco's Phosphate-Buffered Saline containing calcium (0.9 mM) and magnesium (0.5 mM) (PBS; Invitrogen) was then added to the flask and aspirated into a 15 ml centrifuge tube prior to centrifugation (50 g, for 4 mins). The resulting supernatant was discarded and the pellet gently re-suspended in 3 ml of PBS. A 0.5 ml aliquot of cell suspension was removed and the number of viable cells (based on trypan blue exclusion) was determined in an automated reader (Cedex; Innovatis) so that the cell re-suspension volume could be adjusted with PBS to give the desired final cell concentration. It is the cell concentration at this point in the assay that is quoted when referring to this parameter. CHO-Kv1.5 cells, which were used to adjust the voltage offset on IonWorks™ HT, were maintained and prepared for use in the same way.

### Electrophysiology

The principles and operation of this device have been described by (Schroeder, Neagle, Trezise, & Worley, 2003). Briefly, the technology is based on a 384-well plate (PatchPlate™) in which a recording is attempted in each well by using suction to position and hold a cell on a small hole separating two isolated fluid chambers. Once sealing has taken place, the solution on the underside of the PatchPlate™ is changed to one containing amphotericin B. This permeablises the patch of cell membrane covering the hole in each well and, in effect, allows a perforated, whole-cell patch clamp recording to be made.

A β-test IonWorks™ HT from Essen Instrument was used. There is no capability to warm solutions in this device hence it was operated at room temperature (˜21° C.), as follows. The reservoir in the “Buffer” position was loaded with 4 ml of PBS and that in the “Cells” position with the CHO-hERG cell suspension described above. A 96-well plate (V-bottom, Greiner Bio-one) containing the compounds to be tested (at 3-fold above their final test concentration) was placed in the “Plate 1” position and a PatchPlate™ was clamped into the PatchPlate™ station. Each compound plate was laid-out in 12 columns to enable ten, 8-point concentration-effect curves to be constructed; the remaining two columns on the plate were taken up with vehicle (final concentration 0.33% DMSO), to define the assay baseline, and a supra-maximal blocking concentration of cisapride (final concentration 10 μM) to define the 100% inhibition level. The fluidics-head (F-Head) of IonWorks™ HT then added 3.5 μl of PBS to each well of the PatchPlate™ and its underside was perfused with “internal” solution that had the following composition (in mM): K-Gluconate 100, KCl 40, MgCl₂ 3.2, EGTA3 and HEPES 5 (all Sigma-Aldrich; pH 7.25-7.30 using 10 M KOH). After priming and de-bubbling, the electronics-head (E-head) then moved round the PatchPlate™ performing a hole test (i.e. applying a voltage pulse to determine whether the hole in each well was open). The F-head then dispensed 3.5 μl of the cell suspension described above into each well of the PatchPlate™ and the cells were given 200 seconds to reach and seal to the hole in each well. Following this, the E-head moved round the PatchPlate™ to determine the seal resistance obtained in each well. Next, the solution on the underside of the PatchPlate™ was changed to “access” solution that had the following composition (in mM): KCl 140, EGTA 1, MgCl₂ 1 and HEPES 20 (pH 7.25-7.30 using 10 M KOH) plus 100 μg/ml of amphotericin B (Sigma-Aldrich). After allowing 9 minutes for patch perforation to take place, the E-head moved round the PatchPlate™ 48 wells at a time to obtain pre-compound hERG current measurements. The F-head then added 3.5 μl of solution from each well of the compound plate to 4 wells on the PatchPlate™ (the final DMSO concentration was 0.33% in every well). This was achieved by moving from the most dilute to the most concentrated well of the compound plate to minimise the impact of any compound carry-over. After approximately 3.5 mins incubation, the E-head then moved around all 384-wells of the PatchPlate™ to obtain post-compound hERG current measurements. In this way, non-cumulative concentration-effect curves could be produced where, providing the acceptance criteria were achieved in a sufficient percentage of wells (see below), the effect of each concentration of test compound was based on recording from between 1 and 4 cells.

The pre- and post-compound hERG current was evoked by a single voltage pulse consisting of a 20 s period holding at −70 mV, a 160 ms step to −60 mV (to obtain an estimate of leak), a 100 ms step back to −70 mV, a 1 s step to +40 mV, a 2 s step to −30 mV and finally a 500 ms step to −70 mV. In between the pre- and post-compound voltage pulses there was no clamping of the membrane potential. Currents were leak-subtracted based on the estimate of current evoked during the +10 mV step at the start of the voltage pulse protocol. Any voltage offsets in IonWorks™ HT were adjusted in one of two ways. When determining compound potency, a depolarising voltage ramp was applied to CHO-Kv1.5 cells and the voltage noted at which there was an inflection point in the current trace (i.e. the point at which channel activation was seen with a ramp protocol). The voltage at which this occurred had previously been determined using the same voltage command in conventional electrophysiology and found to be −15 mV (data not shown); thus an offset potential could be entered into the IonWorks™ HT software using this value as a reference point. When determining the basic electrophysiological properties of hERG, any offset was adjusted by determining the hERG tail current reversal potential in IonWorks™ HT, comparing it with that found in conventional electrophysiology (−82 mV; see FIG. 1c) and then making the necessary offset adjustment in the IonWorks™ HT software. The current signal was sampled at 2.5 kHz.

Pre- and post-scan hERG current magnitude was measured automatically from the leak subtracted traces by the IonWorks™ HT software by taking a 40 ms average of the current during the initial holding period at −70 mV (baseline current) and subtracting this from the peak of the tail current response. The acceptance criteria for the currents evoked in each well were: pre-scan seal resistance >60 MΩ, pre-scan hERG tail current amplitude >150 pA; post-scan seal resistance >60 MΩ. The degree of inhibition of the hERG current was assessed by dividing the post-scan hERG current by the respective pre-scan hERG current for each well.

### Results

Typical Kᵢ values for the compounds of the present invention are in the range of about 1 to about 100,000 nM. Biological data on two of the examples is given below in Table 1.

TABLE 1

| Example No. | IC50 in TR-FRET assay |
|---|---|
| 26 | 743 nM |
| 45 | 258 nM |

1. A compound of Formula I 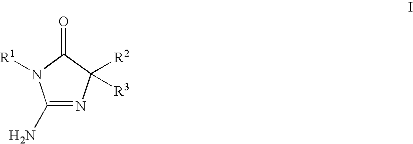
wherein
R¹ is selected from hydrogen, C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl, wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl or C₁₋₆alkylheterocyclyl is optionally substituted with one, two or three A;
R² is selected from hydrogen, nitro, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, and -Q-C₁₋₆alkylheterocyclyl, wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl is optionally substituted by one, two or three R⁷;
-Q- is a direct bond, —CONH—, —CO—, —CON(C₁₋₆alkyl)-, —CON(C₃₋₆cycloalkyl)-, —SO—, —SO₂—, —SO₂NH—, —SO₂N(C₁₋₆alkyl)-, —SO₂N(C₃₋₆cycloalkyl)-, —NHSO₂—, —N(C₁₋₆alkyl)SO₂—, —NHCO—, —N(C₁₋₆alkyl)CO—, —N(C₃₋₆cycloalkyl)CO— or —N(C₃₋₆cycloalkyl)SO₂—;
R³ is (C(R⁴)(R⁵))ₙR⁶, C₂₋₄alkenylR⁶, C₂₋₄alkynylR⁶, C₅₋₇cycloalkenylR⁶, nitro or cyano and if n>1 then each C(R⁴)(R⁵) is independent of the others;
R⁴ and R⁵ are independently selected from hydrogen, C₁₋₆alkyl, cyano, halo and nitro;
or R⁴ and R⁵ together form oxo, C₃₋₆cycloalkyl or heterocyclyl;
R⁶ is selected from methyl, C₃₋₆cycloalkyl, heterocyclyl, aryl and heteroaryl wherein each of said methyl, C₃₋₆cycloalkyl, heterocyclyl, aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four A with the proviso that the bicyclic ring is not an indane, benzo[1,3]dioxole or 2,3-dihydrobenzo[1,4]-dioxine ring system;
R⁷ is selected from halogen, nitro, CHO, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸ (CO)R⁹, O(CO)NR⁸R⁹, NR⁸(CO)OR⁹, NR⁸(CO)NR⁸R⁹, O(CO)OR⁸, O(CO)R⁸, C₀₋₆alkylCOR⁸, OC₁₋₆alkylCOR³, NR⁸(CO)(CO)R⁸, NR⁸(CO)(CO)NR⁸R⁹, C₀₋₆alkylSR⁸, C₀₋₆alkyl(SO₂)NR⁸R⁹, OC₁₋₆alkylNR³(SO₂)R⁹, OC₀₋₆alkyl(SO₂)NR⁸R⁹, C₀₋₆alkyl(SO)NR⁸R⁹, OC₁₋₆alkyl(SO)NR³R⁹, OSO₂R⁸, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl, wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and four A with the proviso that said bicyclic ring system is not an indane, benzo[1,3]dioxole or 2,3-dihydrobenzo[1,4]-dioxine ring system;
R¹⁴ is selected from halogen, nitro, CHO, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸ (CO)R⁹, O(CO)NR⁸R⁹, NR⁸(CO)OR⁹, NR⁸(CO)NR⁸R⁹, OR⁸, O(CO)OR⁸, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₂₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, OSO₂R⁸R⁹, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl wherein any C₁₋₁₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl may be optionally substituted by between one and four A;
R⁸ and R⁹ are independently selected from hydrogen, C₁₋₆alkyl, C₂₋₆alkenyl, C₂₋₆alkynyl, fluoromethyl, difluoromethyl, trifluoromethyl, fluoromethoxy, difluoromethoxy, trifluoromethoxy, C₀₋₆alkylC₃₋₆cycloalkyl, C₀₋₆alkylaryl, C₀₋₆alkylheteroaryl, C₀₋₆alkylheterocyclyl and C₀₋₆alkylNR¹⁰R¹¹, wherein the C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl or C₀₋₆alkylheterocyclyl are optionally substituted by A; or
R⁸ and R⁹ may together form a 4 to 6 membered heterocyclic ring containing one or more heteroatoms selected from N, O and S that is optionally substituted by A; whenever two R⁸ groups occur in the structure then they may optionally together form a 5 or 6 membered heterocyclic ring containing one or more heteroatoms selected from N, O and S, that is optionally substituted by A;
R¹⁰ and R¹¹ are independently selected from hydrogen, C₁₋₆alkyl, C₃₋₆alkenyl, C₃₋₆alkynyl, C₀₋₆alkylC₃₋₆cycloalkyl, C₀₋₆alkylaryl, C₀₋₆alkylheterocyclyl and C₀₋₆alkylheteroaryl, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R¹⁰ and R¹¹ may together form a 4 to 6 membered heterocyclic ring containing one or more heteroatoms selected from N, O and S optionally substituted by A;
n is 0, 1, 2 or 3
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, OC₂₋₆alkylNR¹²R¹³, NR¹²R¹³, CONR¹²R¹³, NR¹²(CO)R¹³, O(CO)C₁₋₆alkyl, (CO)OC₁₋₆alkyl, COR¹², (SO₂)NR¹²R¹³, NSO₂R¹², SO₂R¹², SOR¹², (CO)C₁₋₆alkylNR¹²R¹³, (SO₂)C₁₋₆alkylNR¹²R¹³, OSO₂R¹², and SO₃R¹² wherein the C₁₋₆alkyl, C₂₋₆alkenyl, C₂₋₆alkynyl, C₀₋₆alkylaryl, C₀₋₆alkylheteroaryl, C₀₋₆alkylheterocyclyl and C₀₋₆alkylC₃₋₆cycloalkyl groups may be optionally substituted with halo, OSO₂R¹², SO₃R¹², nitro, cyano, OR¹², C₁₋₆alkyl, fluoromethyl, difluoromethyl, trifluoromethyl, fluoromethoxy, difluoromethoxy or trifluoromethoxy;
R¹² and R¹³ are independently selected from hydrogen, C₁₋₆alkyl, C₃₋₆cycloalkyl, aryl, heteroaryl and heterocyclyl wherein said C₁₋₆alkyl, C₃₋₆cycloalkyl, aryl, heteroaryl or heterocyclyl is optionally substituted by one, two or three hydroxy, cyano, halo or C₁₋₃alkyloxy; or
R¹² and R¹³ may together form a 4 to 6 membered heterocyclic ring containing one or more heteroatoms selected from N, O and S optionally substituted by hydroxy, C₁₋₃alkyloxy, cyano or halo;
provided that either any of the aryl or heteroaryl groups in R¹, R² or R³ is substituted with a OSO₂R⁸, SO₃R⁸, OSO₂R¹² or SO₃R¹² group; or
provided that any of the individual aryl or heteroaryl groups in R¹, R² or R³ are fused with a 4, 5, 6 or 7 membered cycloalkyl, cycloalkenyl or heterocyclyl group to form a bicyclic ring system where the bicyclic ring system is optionally substituted with between one and four A with the proviso that the bicyclic ring is not an indane, benzo[1,3]dioxole or 2,3-dihydrobenzo[1,4]-dioxine ring system;
or
provided that R¹ is C₃₋₆alkynyl or C₅₋₇cycloalkenyl optionally substituted with one, two or three A;
or
provided that Q is selected from —NHSO₂—, —N(C₁₋₆alkyl)SO₂—, —SO₂NH—, —SO₂N(C₁₋₆alkyl)-SO₂N(C₃₋₆cycloalkyl)-, —SO—, —SO₂— and —N(C₃₋₆cycloalkyl)SO₂—;
or
provided that R³ is selected from C₂₋₄alkenylR⁶, C₂₋₄alkynylR⁶, C₅₋₇cycloalkenylR⁶, nitro and cyano;
or
provided that R² is selected from nitro, cyano, C₂₋₆alkynyl, C₅₋₇cycloalkenyl and C₂₋₆alkenyl group where the C₂₋₆alkynyl, C₅₋₇cycloalkenyl or C₂₋₆alkenyl group is optionally substituted by one, two or three R⁷;
as a free base or a pharmaceutically acceptable salt, solvate or solvate of a salt thereof.

2. A compound according to claim 1, wherein
R¹ is selected from C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl and C₁₋₁₆alkylheterocyclyl wherein the C₁₋₁₆alkyl, C₃₋₆alkenyl, C₃₋₆alkynyl, C₃₋₆cycloalkyl, C₅₋₇cycloalkenyl, aryl, heteroaryl, heterocyclyl, C₁₋₁₆alkylC₃₋₆cycloalkyl, C₁₋₁₆alkylaryl, C₁₋₆alkylheteroaryl or C₁₋₆alkylheterocyclyl is optionally substituted with one or two A;
R² is selected from hydrogen, cyano, -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, and -Q-C₁₋₆alkylheterocyclyl wherein said -Q-C₁₋₆alkyl, -Q-C₂₋₆alkenyl, -Q-C₂₋₆alkynyl, -Q-C₃₋₆cycloalkyl, -Q-C₅₋₇cycloalkenyl, -Q-C₁₋₆alkylC₃₋₆cycloalkyl, -Q-aryl, -Q-heteroaryl, -Q-C₁₋₆alkylaryl, -Q-C₁₋₆alkylheteroaryl, -Q-heterocyclyl, or -Q-C₁₋₆alkylheterocyclyl is optionally substituted by one, two or three R⁷;
-Q- is a direct bond, —CONH—, —CO—, —CON(C₁₋₆alkyl)-, —CON(C₃₋₆cycloalkyl)-, —NHCO—, —N(C₁₋₆alkyl)CO— or —N(C₃₋₆cycloalkyl)CO—;
R³ is (C(R⁴)(R⁵))ₙR⁶, C₂₋₄alkenylR⁶, C₂₋₄alkynylR⁶ or C₅₋₇cycloalkenylR⁶ and if n>1 then each C(R⁴)(R⁵) is independent of the others;
R⁴ and R⁵ are independently selected from hydrogen, C₁₋₆alkyl, cyano, and halo; or
R⁴ and R⁵ together form oxo, C₃₋₆cycloalkyl or heterocyclyl;
R⁶ is selected from methyl, C₃₋₆cycloalkyl, heterocyclyl, aryl and heteroaryl wherein each of said methyl, C₃₋₆cycloalkyl, heterocyclyl, aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that the bicyclic ring is not an indane, benzo[1,3]dioxole or 2,3-dihydrobenzo[1,4]-dioxine ring system;
R⁷ is selected from halogen, nitro, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸ (CO)R⁹, O(CO)NR⁸R⁹, NR⁸(CO)OR⁹, NR⁸(CO)NR⁸R⁹, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₁₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl, and OC₂₋₆alkylheterocyclyl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that said bicyclic ring system is not an indane, benzo[1,3]dioxole or 2,3-dihydrobenzo[1,4]-dioxine ring system;
R¹⁴ is selected from halogen, nitro, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylNR⁸R⁹, OC₂₋₆alkylNR⁸R⁹, OC₂₋₆alkylOC₂₋₆alkylNR⁸R⁹, NR⁸OR⁹, C₀₋₆alkylCO₂R⁸, OC₁₋₆alkylCO₂R⁸, C₀₋₆alkylCONR⁸R⁹, OC₁₋₆alkylCONR⁸R⁹, OC₂₋₆alkylNR⁸(CO)R⁹, C₀₋₆alkylNR⁸(CO)R⁹, O(CO)NR⁸R⁹, NR⁸(CO)OR⁹, NR⁸(CO)NR⁸R⁹, OR⁸, O(CO)R⁸, C₀₋₆alkylCOR⁸, OC₁₋₆alkylCOR⁸, NR⁸(CO)(CO)R⁸, NR⁸(CO)(CO)NR⁸R⁹, C₀₋₆alkylSR⁸, C₀₋₆alkyl(SO₂)NR⁸R⁹, OC₂₋₆alkylNR⁸(SO₂)R⁹, OC₀₋₆alkyl(SO₂)NR⁸R⁹, C₀₋₆alkyl(SO)NR⁸R⁹, OC₁₋₆alkyl(SO)NR⁸R⁹, OSO₂R⁸, OSO₂R⁸R⁹, SO₃R⁸, C₀₋₆alkylNR⁸(SO₂)NR⁸R⁹, C₀₋₆alkylNR⁸(SO)R⁹, OC₂₋₆alkylNR⁸(SO)R⁸, OC₁₋₆alkylSO₂R⁸, C₁₋₆alkylSO₂R⁸, C₀₋₆alkylSOR⁸, C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl wherein any C₁₋₆alkyl, C₂₋₆alkenyl, C₂₋₆alkynyl, C₀₋₆alkylC₃₋₆cycloalkyl, C₀₋₆alkylaryl, C₀₋₆alkylheteroaryl, C₀₋₆alkylheterocyclyl and OC₂₋₆alkylheterocyclyl may be optionally substituted by between one and three A;
R⁸ and R⁹ are independently selected from hydrogen, C₁₋₆alkyl, C₃₋₆alkenyl, C₃₋₆alkynyl, fluoromethyl, difluoromethyl, trifluoromethyl, C₀₋₆alkylC₃₋₆cycloalkyl, C₀₋₆alkylaryl, C₀₋₆alkylheteroaryl, C₀₋₆alkylheterocyclyl, C₀₋₆alkylNR¹⁰R¹¹ and C₁₋₆alkylNR¹⁰R¹¹, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R⁸ and R⁹ may together form a 4 to 6 membered heterocyclic ring containing one or more heteroatoms selected from N, O or S that is optionally substituted by A; whenever two R⁸ groups occur in the structure then they may together form a 5 or 6 membered heterocyclic ring containing one or more heteroatoms selected from N, O and S, that is optionally substituted by A;
R¹⁰ and R¹¹ are independently selected from hydrogen, C₁₋₆alkyl, C₃₋₆alkenyl, C₃₋₆alkynyl, C₀₋₆alkylC₃₋₆cycloalkyl, C₀₋₆alkylaryl, C₀₋₆alkylheterocyclyl and C₀₋₆alkylheteroaryl, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl or C₀₋₆alkylheterocyclyl are optionally substituted by A; or
R¹⁰ and R¹¹ may together form a 4 to 6 membered heterocyclic ring containing one or more heteroatoms selected from N, O or S optionally substituted by A;
n is 0, 1, or 2;
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy, OC₂₋₆alkylNR¹²R¹³, NR¹²R¹³, CONR¹²R¹³, NR¹²(CO)R¹³, O(CO)C₁₋₆alkyl, (CO)OC₁₋₆alkyl, COR¹², (SO₂)NR¹²R¹³, NSO₂R¹², SO₂R¹², SOR¹², (CO)C₁₋₆alkylNR¹²R¹³, (SO₂)C₁₋₆alkylNR¹²R¹³, OSO₂R¹², SO₃R¹² wherein the C₁₋₆alkyl, C₂₋₆alkenyl, C₂₋₆alkynyl, C₀₋₆alkylaryl, C₀₋₆alkylheteroaryl, C₀₋₆alkylheterocyclyl and C₀₋₆alkylC₃₋₆cycloalkyl groups may be optionally substituted with halo, OSO₂R¹², SO₃R¹², nitro, cyano, OR¹², C₁₋₆alkyl, fluoromethyl, difluoromethyl, trifluoromethyl, fluoromethoxy, difluoromethoxy and trifluoromethoxy;
R¹² and R¹³ are independently selected from hydrogen, C₁₋₆alkyl, C₃₋₆cycloalkyl, aryl, heteroaryl and heterocyclyl wherein said C₁₋₆alkyl, C₃₋₆cycloalkyl, aryl, heteroaryl or heterocyclyl is optionally substituted by one or two hydroxy, cyano, halo or C₁₋₃alkyloxy; or
R¹² and R¹³ may together form a 4 to 6 membered heterocyclic ring containing one or more heteroatoms selected from N, O and S optionally substituted by hydroxy, cyano, C₁₋₃alkyloxy or halo;
as a free base or a pharmaceutically acceptable salt, solvate or solvate of a salt thereof.

3. A compound according to claim 1, wherein
R¹ is selected from C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₆alkylC₃₋₆cycloalkyl, C₁₋₆alkylaryl, C₁₋₆alkylheteroaryl and C₁₋₆alkylheterocyclyl wherein the C₁₋₆alkyl, C₃₋₆alkenyl, C₃₋₆alkynyl, C₃₋₆cycloalkyl, C₅₋₇cycloalkenyl, aryl, heteroaryl, heterocyclyl, C₁₋₁₆alkylC₃₋₆cycloalkyl, C₁₋₁₆alkylaryl, C₁₋₆alkylheteroaryl or C₁₋₆alkylheterocyclyl is optionally substituted with one or two A;
R² is selected from -Q-aryl and -Q-heteroaryl, wherein said -Q-aryl or -Q-heteroaryl is optionally substituted by one, two or three R⁷;
-Q- is a direct bond, —CONH—, —CO—, —CON(C₁₋₆alkyl)-, —CON(C₃₋₆cycloalkyl)-, —NHCO—, —N(C₁₋₆alkyl)CO— or —N(C₃₋₆cycloalkyl)CO—;
R³ is (C(R⁴)(R⁵))ₙR⁶;
R⁶ is selected aryl or heteroaryl wherein each of the said aryl or heteroaryl is optionally substituted with between one and four R⁷, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that the bicyclic ring is not an indane, benzo[1,3]dioxole or 2,3-dihydrobenzo[1,4]-dioxine ring system;
R⁷ is selected from halogen, nitro, C₀₋₆alkylCN, OC₁₋₆alkylCN, C₀₋₆alkylOR⁸, OC₂₋₆alkylOR⁸, fluoromethyl, difluoromethyl, trifluoromethyl, fluoromethoxy, difluoromethoxy, trifluoromethoxy, OSO₂R⁸, C₀₋₆alkylaryl and C₀₋₆alkylheteroaryl, wherein any C₀₋₆alkylaryl or C₀₋₆alkylheteroaryl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A with the proviso that said bicyclic ring system is not an indane, benzo[1,3]dioxole or 2,3-dihydrobenzo[1,4]-dioxine ring system;
R¹⁴ is selected from halogen, nitro, C₀₋₆alkylCN, OC₁₋₆alkylCN, C₀₋₆alkylOR⁸, OC₁₋₆alkylOR⁸, fluoromethyl, difluoromethyl, trifluoromethyl, fluoromethoxy, difluoromethoxy, trifluoromethoxy, C₀₋₆alkylCO₂R⁸, C₀₋₆alkylNR⁸(CO)R⁹, OR⁸, O(CO)R⁸, C₀₋₆alkylCOR⁸, OSO₂R⁸, and OSO₂R⁸R⁹C₁₋₆alkyl;
R⁸ and R⁹ are independently selected from hydrogen, C₁₋₆alkyl, C₃₋₆alkenyl, C₃₋₆alkynyl, fluoromethyl, difluoromethyl, trifluoromethyl, C₀₋₆alkylC₃₋₆cycloalkyl, C₀₋₆alkylaryl, C₀₋₆alkylheteroaryl, C₀₋₆alkylheterocyclyl, C₀₋₆alkylNR¹⁰R¹¹ and C₁₋₆alkylNR¹⁰R¹¹, wherein the C₁₋₆alkyl, C₃₋₆alkenyl, C₃₋₆alkynyl, C₀₋₆alkylC₃₋₆cycloalkyl, C₀₋₆alkylaryl, C₀₋₆alkylheteroaryl, C₀₋₆alkylheterocyclyl are optionally substituted by A; or
R⁸ and R⁹ may together form a 4 to 6 membered heterocyclic ring containing one or more heteroatoms selected from N, O and S that is optionally substituted by A; whenever two R⁸ groups occur in the structure then they may together form a 5 or 6 membered heterocyclic ring containing one or more heteroatoms selected from N, O and S, that is optionally substituted by A;
R¹⁰ and R¹¹ are independently selected from hydrogen and C₁₋₆alkyl; or
R¹⁰ and R¹¹ may together form a 4 to 6 membered heterocyclic ring containing one or more heteroatoms selected from N, O and S optionally substituted by A;
n is 0;
A is selected from oxo, halogen, nitro, CN, OR¹², C₁₋₆alkyl, C₂₋₆alkenyl, C₂₋₆alkynyl, C₀₋₆alkylaryl, C₀₋₆alkylheteroaryl, C₀₋₆alkylC₃₋₆cycloalkyl, C₀₋₆alkylheterocyclyl, fluoromethyl, difluoromethyl, trifluoromethyl, fluoromethoxy, difluoromethoxy, trifluoromethoxy and COR¹²;
R¹² and R¹³ are independently selected from hydrogen and C₁₋₆alkyl;
as a free base or a pharmaceutically acceptable salt, solvate or solvate of a salt thereof.

4. A compound according to claim 1, wherein R¹ is C₁₋₆alkyl.

5. A compound according to claim 1, wherein -Q- in R² represents a direct bond.

6. A compound according to claim 1, wherein R² is -Q-aryl, said aryl, optionally is substituted with R⁷.

7. A compound according to claim 1, wherein R² is -Q-heteroaryl, said heteroaryl, being optionally substituted by one, two or three R⁷.

8. A compound according to claim 1, wherein R⁷ is selected from OSO₂R⁸ and C₀₋₆alkylaryl and wherein said C₀₋₆alkylaryl may be optionally substituted by one or more R¹⁴, and wherein any of the individual aryl groups may be optionally fused with a 4, 5, 6 or 7 membered cycloalkyl, a 5, 6 or 7 membered cycloalkenyl or heterocyclyl group to form a bicyclic ring system where the bicyclic ring system is optionally substituted with between one and three A.

9. A compound according to claim 1, wherein R⁷ is selected from halogen, nitro, C₀₋₆alkylCN, OC₂₋₆alkylOR⁸, trifluoromethyl, fluoromethoxy, trifluoromethoxy, OSO₂R⁸, C₀₋₆alkylaryl and C₀₋₆alkylheteroaryl, wherein any C₀₋₆alkylaryl or C₀₋₆alkylheteroaryl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A.

10. A compound according to claim 8, wherein said C₀₋₆alkylaryl represents phenyl.

11. A compound according to claim 8, wherein said R¹⁴ represents C₀₋₆alkylOR⁸ or OSO₂R⁸.

12. A compound according to claim 9, wherein said R¹⁴ is selected from halogen, nitro, C₀₋₆alkylCN, C₀₋₆alkylOR⁸, fluoromethyl, difluoromethyl, trifluoromethyl, fluoromethoxy, difluoromethoxy, trifluoromethoxy, C₀₋₆alkylCO₂R⁸, C₀₋₆alkylNR⁸(CO)R⁹, OR⁸, O(CO)R⁸, C₀₋₆alkylCOR⁸, OSO₂R⁸ and OSO₂R⁸R⁹C₁₋₆alkyl.

13. A compound according to claim 8, wherein said R⁸ represents C₁₋₆alkyl or trifluoromethyl.

14. A compound according to claim 9, wherein said R⁸ and R⁹ are independently selected from hydrogen, C₁₋₆alkyl, trifluoromethyl, C₀₋₆alkylaryl and C₀₋₆alkylNR¹⁰R¹¹, wherein the C₁₋₆alkyl, or C₀₋₆alkylaryl, C₀₋₆alkylheteroaryl, C₀₋₆alkylheterocyclyl are optionally substituted by A; or
R⁸ and R⁹ may together form a 4 to 6 membered heterocyclic ring containing one or more heteroatoms selected from N, O and S.

15. A compound according to claim 1, wherein n is 0.

16. A compound according to claim 1, wherein A is selected from OR¹², C₁₋₆alkyl and COR¹²; and R¹² is C₁₋₆alkyl.

17. A compound according to claim 1, wherein R⁶ is aryl, wherein said aryl is optionally substituted with between one and four R⁷, and wherein said aryl may be optionally fused with a 4, 5, 6 or 7 membered cycloalkyl, a 5, 6 or 7 membered cycloalkenyl or heterocyclyl group to form a bicyclic ring system where the bicyclic ring system is optionally substituted with between one and three A.

18. A compound according to claim 17, wherein R⁷ is selected from OSO₂R⁸ and C₀₋₆alkylaryl and wherein said C₀₋₆alkylaryl may be optionally substituted by one or more R¹⁴.

19. A compound according to claim 12, wherein said R¹⁴ represents C₀₋₆alkylOR⁸ or OSO₂R⁸.

20. A compound according to claim 17, wherein said aryl may be optionally fused with a 5 or 6 membered heterocyclyl group to form a bicyclic ring system where the bicyclic ring system is optionally substituted with between one and three A.

21. A compound according to claim 20, wherein said A is COR¹² and R¹² is C₁₋₆alkyl.

22. A compound according to claim 1, wherein R⁶ is aryl or heteroaryl wherein each of said aryl or heteroaryl is optionally substituted with between one and four R⁷, and wherein any of the individual aryl or heteroaryl groups may be optionally fused with a 4, 5, 6 or 7 membered cycloalkyl or heterocyclyl group to form a bicyclic ring system where the bicyclic ring system is optionally substituted with between one and three A.

23. A compound according to claim 22, wherein R⁷ is selected from halogen, nitro, C₀₋₆alkylCN, OC₂₋₆alkylOR⁸, trifluoromethyl, fluoromethoxy, trifluoromethoxy, OSO₂R⁸, C₀₋₆alkylaryl and C₀₋₆alkylheteroaryl, wherein any C₀₋₆alkylaryl or C₀₋₆alkylheteroaryl may be optionally substituted by one or more R¹⁴, and wherein any of the individual aryl or heteroaryl groups may be optionally fused with a 4, 5, 6 or 7 membered cycloalkyl, cycloalkenyl or heterocyclyl group to form a bicyclic ring system where the bicyclic ring system is optionally substituted with between one and three A.

24. A compound according to claim 23, wherein R¹⁴ is selected from halogen, nitro, C₀₋₆alkylCN, C₀₋₆alkylOR⁸, fluoromethyl, difluoromethyl, trifluoromethyl, fluoromethoxy, difluoromethoxy, trifluoromethoxy, C₀₋₆alkylCO₂R⁸, C₀₋₆alkylNR⁸(CO)R⁹, OR⁸, C₀₋₆alkylCOR⁸, OSO₂R⁸ and OSO₂R⁸R⁹C₁₋₆alkyl.

25. A compound according to claim 23, wherein R⁸ and R⁹ are independently selected from hydrogen, C₁₋₆alkyl, trifluoromethyl, C₀₋₆alkylaryl, and C₀₋₆alkylNR¹⁰R¹¹, wherein the C₁₋₆alkyl or C₀₋₆alkylaryl, are optionally substituted by A; or R⁸ and R⁹ may together form a 4 to 6 membered heterocyclic ring containing one or more heteroatoms selected from N, O and S.

26. A compound according to claim 25, wherein R¹⁰ and R¹¹ are independently hydrogen or C₁₋₆alkyl; or R¹⁰ and R¹¹ may together form a 4 to 6 membered heterocyclic ring containing one or more heteroatoms selected from N, O and S.

27. A compound according to claim 23, wherein A is selected from OR¹², C₁₋₆alkyl, and COR¹².

28. A compound according to claim 27, wherein R¹² is C₁₋₆alkyl.

29. A compound, said compound being:
2-Amino-5-(3-bromophenyl)-3-methyl-5-phenyl-3,5-dihydro-imidazol-4-one;
5-[3-(2-Acetyl-1,2,3,4-tetrahydroisoquinolin-7-yl)phenyl]-2-amino-3-methyl-5-phenyl-3,5-dihydro-4H-imidazol-4-one;
5-[3-(2-Acetyl-1,2,3,4-tetrahydroisoquinolin-7-yl)phenyl]-2-amino-3-methyl-5-phenyl-3,5-dihydro-4H-imidazol-4-one hydrochloride;
5-[3-(1-Acetyl-2,3-dihydro-1H-indol-5-yl)phenyl]-2-amino-3-methyl-5-phenyl-3,5-dihydro-4H-imidazol-4-one;
5-[3-(1-Acetyl-2,3-dihydro-1H-indol-5-yl)phenyl]-2-amino-3-methyl-5-phenyl-3,5-dihydro-4H-imidazol-4-one hydrochloride;
2-Amino-5-[3-(2,3-dihydro-1-benzofuran-6-yl)phenyl]-3-methyl-5-phenyl-3,5-dihydro-4H-imidazol-4-one;
2-Amino-5-[3-(2,3-dihydro-1-benzofuran-6-yl)phenyl]-3-methyl-5-phenyl-3,5-dihydro-4H-imidazol-4-one hydrochloride;
4-[2-Amino-4-(3′-methoxybiphenyl-3-yl)-1-methyl-5-oxo-4,5-dihydro-1H-imidazol-4-yl]phenyl methanesulfonate;
4-[2-Amino-4-(3′-methoxybiphenyl-3-yl)-1-methyl-5-oxo-4,5-dihydro-1H-imidazol-4-yl]phenyl methanesulfonate hydrochloride;
4-[2-Amino-4-(3′-methoxybiphenyl-3-yl)-1-methyl-5-oxo-4,5-dihydro-1H-imidazol-4-yl]phenyl trifluoromethanesulfonate;
4-[2-Amino-4-(3′-methoxybiphenyl-3-yl)-1-methyl-5-oxo-4,5-dihydro-1H-imidazol-4-yl]phenyl trifluoromethanesulfonate hydrochloride;
2-Amino-5-(3′-hydroxybiphenyl-3-yl)-3-methyl-5-phenyl-3,5-dihydro-4H-imidazol-4-one;
3′-(2-Amino-1-methyl-5-oxo-4-phenyl-4,5-dihydro-1H-imidazol-4-yl)biphenyl-3-yl trifluoromethanesulfonate hydrochloride;
2-Amino-5-(2′-hydroxy-5′-methoxybiphenyl-3-yl)-3-methyl-5-phenyl-3,5-dihydro-4H-imidazol-4-one;
3′-(2-Amino-1-methyl-5-oxo-4-phenyl-4,5-dihydro-1H-imidazol-4-yl)-5-methoxybiphenyl-2-yl trifluoromethanesulfonate hydrochloride;
3-(2-Amino-1-methyl-5-oxo-4-phenyl-4,5-dihydro-1H-imidazol-4-yl)phenyl methanesulfonate hydrochloride;
2-Amino-5-(3-hydroxyphenyl)-3-methyl-5-phenyl-3,5-dihydro-4H-imidazol-4-one;
3-(2-Amino-1-methyl-5-oxo-4-phenyl-4,5-dihydro-1H-imidazol-4-yl)phenyl trifluoromethanesulfonate hydrochloride;
2-Amino-5-(3-bromo-4-hydroxyphenyl)-3-methyl-5-phenyl-3,5-dihydro-4H-imidazol-4-one;
2-Amino-5-(6-hydroxy-3′-methoxybiphenyl-3-yl)-3-methyl-5-phenyl-3,5-dihydro-4H-imidazol-4-one;
5-(2-Amino-1-methyl-5-oxo-4-phenyl-4,5-dihydro-1H-imidazol-4-yl)-3′-methoxybiphenyl-2-yl trifluoromethanesulfonate;
3-[2-Amino-4-(3′-methoxybiphenyl-3-yl)-1-methyl-5-oxo-4,5-dihydro-1H-imidazol-4-yl]phenyl trifluoromethanesulfonate hydrochloride;
3-[2-Amino-4-(3′-methoxybiphenyl-3-yl)-1-methyl-5-oxo-4,5-dihydro-1H-imidazol-4-yl]phenyl methanesulfonate hydrochloride;
2-Amino-5-[3-(2,3-dihydro-1-benzofuran-5-yl)phenyl]-3-methyl-5-phenyl-3,5-dihydro-4H-imidazol-4-one;
2-Amino-5-[3-(2,3-dihydro-1-benzofuran-5-yl)phenyl]-3-methyl-5-phenyl-3,5-dihydro-4H-imidazol-4-one hydrochloride;
2-Amino-5-(2,3-dihydro-1-benzofuran-5-yl)-3-methyl-5-phenyl-3,5-dihydro-4H-imidazol-4-one;
2-Amino-5-(2,3-dihydro-1-benzofuran-5-yl)-3-methyl-5-phenyl-3,5-dihydro-4H-imidazol-4-one hydrochloride;
(R)-4-[2-amino-4-(3′-methoxybiphenyl-3-yl)-1-methyl-5-oxo-4,5-dihydro-1H-imidazol-4-yl]phenyl methanesulfonate hydrochloride;
(S)-4-[2-amino-4-(3′-methoxybiphenyl-3-yl)-1-methyl-5-oxo-4,5-dihydro-1H-imidazol-4-yl]phenyl methanesulfonate hydrochloride;
2-Amino-3-methyl-5-[3-(4-methyl-3,4-dihydro-2H-1,4-benzoxazin-7-yl)phenyl]-5-phenyl-3,5-dihydro-4H-imidazol-4-one;
2-Amino-3-methyl-5-[3-(4-methyl-3,4-dihydro-2H-1,4-benzoxazin-7-yl)phenyl]-5-phenyl-3,5-dihydro-4H-imidazol-4-one hydrochloride;
5-[3-(2-Acetyl-1,2,3,4-tetrahydroisoquinolin-5-yl)phenyl]-2-amino-3-methyl-5-phenyl-3,5-dihydro-4H-imidazol-4-one;
5-[3-(2-Acetyl-1,2,3,4-tetrahydroisoquinolin-5-yl)phenyl]-2-amino-3-methyl-5-phenyl-3,5-dihydro-4H-imidazol-4-one hydrochloride;
(R)-4-[2-amino-4-(3′-methoxybiphenyl-3-yl)-1-methyl-5-oxo-4,5-dihydro-1H-imidazol-4-yl]phenyl trifluoromethanesulfonate hydrochloride;
(S)-4-[2-amino-4-(3′-methoxybiphenyl-3-yl)-1-methyl-5-oxo-4,5-dihydro-1H-imidazol-4-yl]phenyl trifluoromethanesulfonate hydrochloride;
2-Amino-3-methyl-5-(4-methyl-3,4-dihydro-2H-1,4-benzoxazin-7-yl)-5-phenyl-3,5-dihydro-4H-imidazol-4-one acetic acid salt;
2-Amino-3-methyl-5-phenyl-5-(5,6,7,8-tetrahydronaphthalen-2-yl)-3,5-dihydro-4H-imidazol-4-one hydrochloride;
5-(1-Acetyl-2,3-dihydro-1H-indol-5-yl)-2-amino-3-methyl-5-phenyl-3,5-dihydro-4H-imidazol-4-one hydrochloride;
5-[3-(1-Acetyl-2,3-dihydro-1H-indol-4-yl)phenyl]-2-amino-3-methyl-5-phenyl-3,5-dihydro-4H-imidazol-4-one hydrochloride;
2-Amino-5-[3-(3,4-dihydro-2H-chromen-8-yl)phenyl]-3-methyl-5-phenyl-3,5-dihydro-4H-imidazol-4-one acetic acid salt;
3′-(2-Amino-1-methyl-5-oxo-4-pyridin-4-yl-4,5-dihydro-1H-imidazol-4-yl)-5-methoxybiphenyl-3-yl methanesulfonate;
3′-(2-Amino-1-methyl-5-oxo-4-pyridin-2-yl-4,5-dihydro-1H-imidazol-4-yl)-5-methoxybiphenyl-3-yl methanesulfonate;
3′-[2-Amino-1-methyl-5-oxo-4-(1,3-thiazol-5-yl)-4,5-dihydro-1H-imidazol-4-yl]-5-methoxybiphenyl-3-yl methanesulfonate;
3′-[2-Amino-1-methyl-5-oxo-4-(1,3-thiazol-4-yl)-4,5-dihydro-1H-imidazol-4-yl]-5-methoxybiphenyl-3-yl methanesulfonate;
4-[2-Amino-4-(3-bromophenyl)-1-methyl-5-oxo-4,5-dihydro-1H-imidazol-4-yl]phenyl methanesulfonate;
4-[2-Amino-1-methyl-5-oxo-4-(3-pyridin-3-ylphenyl)-4,5-dihydro-1H-imidazol-4-yl]phenyl methanesulfonate 0.25 acetate;
4-[2-Amino-4-(3-bromophenyl)-1-methyl-5-oxo-4,5-dihydro-1H-imidazol-4-yl]phenyl 2-methoxyethanesulfonate;
4-[2-Amino-4-(3-bromophenyl)-1-methyl-5-oxo-4,5-dihydro-1H-imidazol-4-yl]phenyl propane-1-sulfonate;
4-[2-Amino-4-(5′-chloro-2′-methoxybiphenyl-3-yl)-1-methyl-5-oxo-4,5-dihydro-1H-imidazol-4-yl]phenyl methanesulfonate 0.25 acetate;
4-[2-Amino-4-(5′-fluoro-2′-methylbiphenyl-3-yl)-1-methyl-5-oxo-4,5-dihydro-1H-imidazol-4-yl]phenyl methanesulfonate;
Methyl 3′-(2-amino-1-methyl-4-{4-[(methylsulfonyl)oxy]phenyl}-5-oxo-4,5-dihydro-1H-imidazol-4-yl)biphenyl-3-carboxylate;
4-{2-Amino-4-[3-(1,3-benzodioxol-5-yl)phenyl]-1-methyl-5-oxo-4,5-dihydro-1H-imidazol-4-yl}phenyl methanesulfonate;
4-{2-Amino-4-[3-(1H-indol-5-yl)phenyl]-1-methyl-5-oxo-4,5-dihydro-1H-imidazol-4-yl}phenyl methanesulfonate 0.25 acetate;
4-[2-Amino-4-(3′-cyanobiphenyl-3-yl)-1-methyl-5-oxo-4,5-dihydro-1H-imidazol-4-yl]phenyl methanesulfonate;
4-{2-Amino-1-methyl-5-oxo-4-[3′-(trifluoromethoxy)biphenyl-3-yl]-4,5-dihydro-1H-imidazol-4-yl}phenyl methanesulfonate;
4-{2-Amino-4-[3-(2-formyl-3-thienyl)phenyl]-1-methyl-5-oxo-4,5-dihydro-1H-imidazol-4-yl}phenyl methanesulfonate;
4-{2-Amino-4-[3-(5-formyl-2-thienyl)phenyl]-1-methyl-5-oxo-4,5-dihydro-1H-imidazol-4-yl}phenyl methanesulfonate;
4-{2-Amino-4-[3-(2-chloropyridin-4-yl)phenyl]-1-methyl-5-oxo-4,5-dihydro-1H-imidazol-4-yl}phenyl methanesulfonate;
4-{4-[3′-(Acetylamino)biphenyl-3-yl]-2-amino-1-methyl-5-oxo-4,5-dihydro-1H-imidazol-4-yl}phenyl methanesulfonate 0.25 acetate;
4-[2-Amino-1-methyl-4-(3′-nitrobiphenyl-3-yl)-5-oxo-4,5-dihydro-1H-imidazol-4-yl]phenyl propane-1-sulfonate;
4-[2-Amino-4-(3′-cyanobiphenyl-3-yl)-1-methyl-5-oxo-4,5-dihydro-1H-imidazol-4-yl]phenyl propane-1-sulfonate;
4-[2-Amino-4-(2′,5′-dimethoxybiphenyl-3-yl)-1-methyl-5-oxo-4,5-dihydro-1H-imidazol-4-yl]phenyl propane-1-sulfonate;
4-[2-Amino-4-(3′-ethoxybiphenyl-3-yl)-1-methyl-5-oxo-4,5-dihydro-1H-imidazol-4-yl]phenyl propane-1-sulfonate;
4-[2-Amino-4-(2′-fluoro-3′-methoxybiphenyl-3-yl)-1-methyl-5-oxo-4,5-dihydro-1H-imidazol-4-yl]phenyl propane-1-sulfonate;
4-[2-Amino-4-(2′-fluoro-5′-methoxybiphenyl-3-yl)-1-methyl-5-oxo-4,5-dihydro-1H-imidazol-4-yl]phenyl propane-1-sulfonate;
4-[2-Amino-4-(2′,6′-difluoro-3′-methoxybiphenyl-3-yl)-1-methyl-5-oxo-4,5-dihydro-1H-imidazol-4-yl]phenyl propane-1-sulfonate;
4-[2-Amino-4-(3′-cyano-4′-fluorobiphenyl-3-yl)-1-methyl-5-oxo-4,5-dihydro-1H-imidazol-4-yl]phenyl propane-1-sulfonate;
4-[2-Amino-4-(5′-cyano-2′-fluorobiphenyl-3-yl)-1-methyl-5-oxo-4,5-dihydro-1H-imidazol-4-yl]phenyl propane-1-sulfonate;
4-[2-Amino-1-methyl-5-oxo-4-(3-pyridin-3-ylphenyl)-4,5-dihydro-1H-imidazol-4-yl]phenyl propane-1-sulfonate;
4-{2-Amino-4-[3-(2-fluoropyridin-3-yl)phenyl]-1-methyl-5-oxo-4,5-dihydro-1H-imidazol-4-yl}phenyl propane-1-sulfonate;
4-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
4-{2-Amino-4-[3-(6-fluoropyridin-3-yl)phenyl]-1-methyl-5-oxo-4,5-dihydro-1H-imidazol-4-yl}phenyl propane-1-sulfonate;
4-[2-Amino-1-methyl-5-oxo-4-(3-pyrimidin-5-ylphenyl)-4,5-dihydro-1H-imidazol-4-yl]phenyl propane-1-sulfonate;
4-{2-Amino-4-[3-(2-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-3-fluoropyridin-4-yl)phenyl]-1-methyl-5-oxo-4,5-dihydro-1H-imidazol-4-yl}phenyl propane-1-sulfonate;
4-{2-Amino-4-[3-(2-chloro-5-fluoropyridin-3-yl)phenyl]-1-methyl-5-oxo-4,5-dihydro-1H-imidazol-4-yl}phenyl propane-1-sulfonate;
4-{2-Amino-4-[3-(2,6-difluoropyridin-3-yl)phenyl]-1-methyl-5-oxo-4,5-dihydro-1H-imidazol-4-yl}phenyl propane-1-sulfonate;
4-[2-Amino-1-methyl-4-(3′-nitrobiphenyl-3-yl)-5-oxo-4,5-dihydro-1H-imidazol-4-yl]phenyl 2-methoxyethanesulfonate;
4-[2-Amino-4-(3′-cyanobiphenyl-3-yl)-1-methyl-5-oxo-4,5-dihydro-1H-imidazol-4-yl]phenyl 2-methoxyethanesulfonate;
4-[2-Amino-4-(2′,5′-dimethoxybiphenyl-3-yl)-1-methyl-5-oxo-4,5-dihydro-1H-imidazol-4-yl]phenyl 2-methoxyethanesulfonate;
4-[2-Amino-4-(3′-ethoxybiphenyl-3-yl)-1-methyl-5-oxo-4,5-dihydro-1H-imidazol-4-yl]phenyl 2-methoxyethanesulfonate;
4-[2-Amino-4-(2′-fluoro-3′-methoxybiphenyl-3-yl)-1-methyl-5-oxo-4,5-dihydro-1H-imidazol-4-yl]phenyl 2-methoxyethanesulfonate;
4-[2-Amino-4-(2′-fluoro-5′-methoxybiphenyl-3-yl)-1-methyl-5-oxo-4,5-dihydro-1H-imidazol-4-yl]phenyl 2-methoxyethanesulfonate;
4-[2-Amino-4-(2′,6′-difluoro-3′-methoxybiphenyl-3-yl)-1-methyl-5-oxo-4,5-dihydro-1H-imidazol-4-yl]phenyl 2-methoxyethanesulfonate;
4-[2-Amino-4-(3′-cyano-4′-fluorobiphenyl-3-yl)-1-methyl-5-oxo-4,5-dihydro-1H-imidazol-4-yl]phenyl 2-methoxyethanesulfonate;
4-[2-Amino-4-(5′-cyano-2′-fluorobiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyridin-3-ylphenyl)-4,5-dihydro-1H-imidazol-4-yl]phenyl 2-methoxyethanesulfonate;
4-{2-Amino-4-[3-(2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
4-{2-Amino-4-[3-(6-fluoropyridin-3-yl)phenyl]-1-methyl-5-oxo-4,5-dihydro-1H-imidazol-4-yl}phenyl 2-methoxyethanesulfonate;
4-[2-Amino-1-methyl-5-oxo-4-(3-pyrimidin-5-ylphenyl)-4,5-dihydro-1H-imidazol-4-yl]phenyl 2-methoxyethanesulfonate;
4-{2-Amino-4-[3-(2-chloro-3-fluoropyridin-4-yl)phenyl]-1-methyl-5-oxo-4,5-dihydro-1H-imidazol-4-yl}phenyl 2-methoxyethanesulfonate;
4-{2-Amino-4-[3-(2-chloro-5-fluoropyridin-3-yl)phenyl]-1-methyl-5-oxo-4,5-dihydro-1H-imidazol-4-yl}phenyl 2-methoxyethanesulfonate;
4-{2-Amino-4-[3-(2,6-difluoropyridin-3-yl)phenyl]-1-methyl-5-oxo-4,5-dihydro-1H-imidazol-4-yl}phenyl 2-methoxyethanesulfonate;
5-(4-Hydroxyphenyl)-3-methyl-5-(3-phenoxyphenyl)-2-thioxoimidazolidin-4-one;
4-(2-Amino-4-{3′-methoxy-5′-[(methylsulfonyl)oxy]biphenyl-3-yl}-1-methyl-5-oxo-4,5-dihydro-1H-imidazol-4-yl)phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl propane-1-sulfonate;
4-[2-Amino-4-(4′-fluoro-3′-methoxybiphenyl-3-yl)-1-methyl-5-oxo-4,5-dihydro-1H-imidazol-4-yl]phenyl propane-1-sulfonate;
4-{2-Amino-4-[3-(5-fluoropyridin-3-yl)phenyl]-1-methyl-5-oxo-4,5-dihydro-1H-imidazol-4-yl}phenyl 2-methoxyethanesulfonate;
4-[2-Amino-4-(4′-fluoro-3′-methoxybiphenyl-3-yl)-1-methyl-5-oxo-4,5-dihydro-1H-imidazol-4-yl]phenyl 2-methoxyethanesulfonate;
4-[2-Amino-1-methyl-5-oxo-4-(3-pyrazin-2-ylphenyl)-4,5-dihydro-1H-imidazol-4-yl]phenyl 2-methoxyethanesulfonate 0.25 acetate;
4-[2-Amino-1-methyl-5-oxo-4-(3-pyrazin-2-ylphenyl)-4,5-dihydro-1H-imidazol-4-yl]phenyl propane-1-sulfonate 0.25 acetate;
(R)-4-[2-Amino-1-methyl-5-oxo-4-(3-pyridin-3-ylphenyl)-4,5-dihydro-1H-imidazol-4-yl]phenyl methanesulfonate;
(S)-4-[2-Amino-1-methyl-5-oxo-4-(3-pyridin-3-ylphenyl)-4,5-dihydro-1H-imidazol-4-yl]phenyl methanesulfonate;
3′-(2-Amino-1-methyl-5-oxo-4-phenyl-4,5-dihydro-1H-imidazol-4-yl)-5-methoxybiphenyl-3-yl methanesulfonate hydrochloride;
3-[2-Amino-4-(3-bromophenyl)-1-methyl-5-oxo-4,5-dihydro-1H-imidazol-4-yl]phenyl methanesulfonate;
3-[2-Amino-4-(3-bromophenyl)-1-methyl-5-oxo-4,5-dihydro-1H-imidazol-4-yl]phenyl 2-methoxyethanesulfonate;
3-[4-(3-Bromophenyl)-1-methyl-5-oxo-2-thioxoimidazolidin-4-yl]phenyl propane-1-sulfonate;
3-[2-Amino-4-(3-bromophenyl)-1-methyl-5-oxo-4,5-dihydro-1H-imidazol-4-yl]phenyl propane-1-sulfonate;
3-[4-(3-Bromophenyl)-1-methyl-5-oxo-2-thioxoimidazolidin-4-yl]phenyl propane-2-sulfonate;
3-[2-Amino-4-(3-bromophenyl)-1-methyl-5-oxo-4,5-dihydro-1H-imidazol-4-yl]phenyl propane-2-sulfonate;
3-[4-(3-Bromophenyl)-1-methyl-5-oxo-2-thioxoimidazolidin-4-yl]phenyl cyclopropanesulfonate;
3-[2-Amino-4-(3-bromophenyl)-1-methyl-5-oxo-4,5-dihydro-1H-imidazol-4-yl]phenyl cyclopropanesulfonate;
3-[4-(3-Bromophenyl)-1-methyl-5-oxo-2-thioxoimidazolidin-4-yl]phenyl dimethylsulfamate;
3-[2-Amino-4-(3-bromophenyl)-1-methyl-5-oxo-4,5-dihydro-1H-imidazol-4-yl]phenyl dimethylsulfamate;
3-[4-(3-Bromophenyl)-1-methyl-5-oxo-2-thioxoimidazolidin-4-yl]phenyl morpholine-4-sulfonate;
3-[2-Amino-4-(3-bromophenyl)-1-methyl-5-oxo-4,5-dihydro-1H-imidazol-4-yl]phenyl morpholine-4-sulfonate;
3-[4-(3-Bromophenyl)-1-methyl-5-oxo-2-thioxoimidazolidin-4-yl]phenyl ethanesulfonate;
3-[2-Amino-4-(3-bromophenyl)-1-methyl-5-oxo-4,5-dihydro-1H-imidazol-4-yl]phenyl ethanesulfonate;
2-Amino-5-(3-bromophenyl)-5-(3-hydroxyphenyl)-3-methyl-3,5-dihydro-4H-imidazol-4-one;
3-{2-Amino-4-[3-(5-methoxypyridin-3-yl)phenyl]-1-methyl-5-oxo-4,5-dihydro-1H-imidazol-4-yl}phenyl ethanesulfonate 0.75 acetate;
4-{2-Amino-4-[3-(5-methoxypyridin-3-yl)phenyl]-1-methyl-5-oxo-4,5-dihydro-1H-imidazol-4-yl}phenyl methanesulfonate acetic acid;
2-Amino-5-[3-(2-fluoropyridin-3-yl)phenyl]-5-(3-hydroxyphenyl)-3-methyl-3,5-dihydro-4H-imidazol-4-one;
2-Amino-5-(3-hydroxyphenyl)-3-methyl-5-(3-pyrimidin-5-ylphenyl)-3,5-dihydro-4H-imidazol-4-one;
2-Amino-5-(3-hydroxyphenyl)-3-methyl-5-(3-pyridin-3-ylphenyl)-3,5-dihydro-4H-imidazol-4-one;
2-Amino-5-(3-hydroxyphenyl)-5-[3-(5-methoxypyridin-3-yl)phenyl]-3-methyl-3,5-dihydro-4H-imidazol-4-one;
3-{2-Amino-4-[3-(2-fluoropyridin-3-yl)phenyl]-1-methyl-5-oxo-4,5-dihydro-1H-imidazol-4-yl}phenyl trifluoromethanesulfonate hydrochloride;
3-[2-Amino-1-methyl-5-oxo-4-(3-pyrimidin-5-ylphenyl)-4,5-dihydro-1H-imidazol-4-yl]phenyl trifluoromethanesulfonate hydrochloride;
3-[2-Amino-1-methyl-5-oxo-4-(3-pyridin-3-ylphenyl)-4,5-dihydro-1H-imidazol-4-yl]phenyl trifluoromethanesulfonate hydrochloride;
3-{2-Amino-4-[3-(5-methoxypyridin-3-yl)phenyl]-1-methyl-5-oxo-4,5-dihydro-1H-imidazol-4-yl}phenyl trifluoromethanesulfonate hydrochloride;
(R)-3′-(2-amino-1-methyl-5-oxo-4-phenyl-4,5-dihydro-1H-imidazol-4-yl)-5-methoxybiphenyl-3-yl methanesulfonate hydrochloride;
(S)-3′-(2-amino-1-methyl-5-oxo-4-phenyl-4,5-dihydro-1H-imidazol-4-yl)-5-methoxybiphenyl-3-yl methanesulfonate hydrochloride;
4-[2-Amino-4-(3′,5′-dichlorobiphenyl-3-yl)-1-methyl-5-oxo-4,5-dihydro-1H-imidazol-4-yl]phenyl methanesulfonate hydrochloride;
3-[2-Amino-1-methyl-5-oxo-4-(3-pyridin-3-ylphenyl)-4,5-dihydro-1H-imidazol-4-yl]phenyl methanesulfonate;
3-{2-Amino-4-[3-(6-fluoropyridin-3-yl)phenyl]-1-methyl-5-oxo-4,5-dihydro-1H-imidazol-4-yl}phenyl methanesulfonate;
3-{2-Amino-4-[3-(2-fluoropyridin-3-yl)phenyl]-1-methyl-5-oxo-4,5-dihydro-1H-imidazol-4-yl}phenyl methanesulfonate;
3-{2-Amino-4-[3-(5-chloro-2-fluoropyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-5-ylphenyl)-4,5-dihydro-1H-imidazol-4-yl]phenyl methanesulfonate;
3-[2-Amino-1-methyl-5-oxo-4-(3-pyridin-3-ylphenyl)-4,5-dihydro-1H-imidazol-4-yl]phenyl propane-1-sulfonate;
3-{2-Amino-4-[3-(6-fluoropyridin-3-yl)phenyl]-1-methyl-5-oxo-4,5-dihydro-1H-imidazol-4-yl}phenyl propane-1-sulfonate;
3-{2-Amino-4-[3-(2-fluoropyridin-3-yl)phenyl]-1-methyl-5-oxo-4,5-dihydro-1H-imidazol-4-yl}phenyl propane-1-sulfonate;
3-{2-Amino-4-[3-(5-chloro-2-fluoropyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-5-ylphenyl)-4,5-dihydro-1H-imidazol-4-yl]phenyl propane-1-sulfonate;
3-[2-Amino-1-methyl-5-oxo-4-(3-pyridin-3-ylphenyl)-4,5-dihydro-1H-imidazol-4-yl]phenyl propane-2-sulfonate;
3-{2-Amino-4-[3-(6-fluoropyridin-3-yl)phenyl]-1-methyl-5-oxo-4,5-dihydro-1H-imidazol-4-yl}phenyl propane-2-sulfonate;
3-{2-Amino-4-[3-(2-fluoropyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-5-ylphenyl)-4,5-dihydro-1H-imidazol-4-yl]phenyl propane-2-sulfonate;
3-[2-Amino-1-methyl-5-oxo-4-(3-pyridin-3-ylphenyl)-4,5-dihydro-1H-imidazol-4-yl]phenyl 2-methoxyethanesulfonate;
3-{2-Amino-4-[3-(6-fluoropyridin-3-yl)phenyl]-1-methyl-5-oxo-4,5-dihydro-1H-imidazol-4-yl}phenyl 2-methoxyethanesulfonate;
3-{2-Amino-4-[3-(2-fluoropyridin-3-yl)phenyl]-1-methyl-5-oxo-4,5-dihydro-1H-imidazol-4-yl}phenyl 2-methoxyethanesulfonate acetate;
3-[2-Amino-1-methyl-5-oxo-4-(3-pyrimidin-5-ylphenyl)-4,5-dihydro-1H-imidazol-4-yl]phenyl 2-methoxyethanesulfonate;
3-{2-Amino-4-[3-(5-chloro-2-fluoropyridin-3-yl)phenyl]-1-methyl-5-oxo-4,5-dihydro-1H-imidazol-4-yl}phenyl 2-methoxyethanesulfonate;
3-[2-Amino-1-methyl-5-oxo-4-(3-pyridin-3-ylphenyl)-4,5-dihydro-1H-imidazol-4-yl]phenyl dimethylsulfamate;
3-{2-Amino-4-[3-(6-fluoropyridin-3-yl)phenyl]-1-methyl-5-oxo-4,5-dihydro-1H-imidazol-4-yl}phenyl dimethylsulfamate;
3-{2-Amino-4-[3-(5-chloro-2-fluoropyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-5-ylphenyl)-4,5-dihydro-1H-imidazol-4-yl]phenyl dimethylsulfamate;
3-[2-Amino-1-methyl-5-oxo-4-(3-pyridin-3-ylphenyl)-4,5-dihydro-1H-imidazol-4-yl]phenyl cyclopropanesulfonate;
3-{2-Amino-4-[3-(6-fluoropyridin-3-yl)phenyl]-1-methyl-5-oxo-4,5-dihydro-1H-imidazol-4-yl}phenyl cyclopropanesulfonate;
3-{2-Amino-4-[3-(2-fluoropyridin-3-yl)phenyl]-1-methyl-5-oxo-4,5-dihydro-1H-imidazol-4-yl}phenyl cyclopropanesulfonate;
3-{2-Amino-4-[3-(5-chloro-2-fluoropyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-5-ylphenyl)-4,5-dihydro-1H-imidazol-4-yl]phenyl cyclopropanesulfonate;
3-[2-Amino-1-methyl-5-oxo-4-(3-pyridin-3-ylphenyl)-4,5-dihydro-1H-imidazol-4-yl]phenyl morpholine-4-sulfonate;
3-{2-Amino-4-[3-(6-fluoropyridin-3-yl)phenyl]-1-methyl-5-oxo-4,5-dihydro-1H-imidazol-4-yl}phenyl morpholine-4-sulfonate;
3-{2-Amino-4-[3-(2-fluoropyridin-3-yl)phenyl]-1-methyl-5-oxo-4,5-dihydro-1H-imidazol-4-yl}phenyl morpholine-4-sulfonate;
3-[2-Amino-1-methyl-5-oxo-4-(3-pyrimidin-5-ylphenyl)-4,5-dihydro-1H-imidazol-4-yl]phenyl morpholine-4-sulfonate;
3-{2-Amino-4-[3-(5-methoxypyridin-3-yl)phenyl]-1-methyl-5-oxo-4,5-dihydro-1H-imidazol-4-yl}phenyl methanesulfonate;
3-[2-Amino-1-methyl-5-oxo-4-(3-pyrimidin-2-ylphenyl)-4,5-dihydro-1H-imidazol-4-yl]phenyl methanesulfonate;
3-{2-Amino-4-[3-(5-methoxypyridin-3-yl)phenyl]-1-methyl-5-oxo-4,5-dihydro-1H-imidazol-4-yl}phenyl propane-1-sulfonate;
3-[2-Amino-1-methyl-5-oxo-4-(3-pyrimidin-2-ylphenyl)-4,5-dihydro-1H-imidazol-4-yl]phenyl propane-1-sulfonate;
3-{2-Amino-4-[3-(5-methoxypyridin-3-yl)phenyl]-1-methyl-5-oxo-4,5-dihydro-1H-imidazol-4-yl}phenyl propane-2-sulfonate;
3-[2-Amino-1-methyl-5-oxo-4-(3-pyrimidin-2-ylphenyl)-4,5-dihydro-1H-imidazol-4-yl]phenyl propane-2-sulfonate;
3-{2-Amino-4-[3-(5-methoxypyridin-3-yl)phenyl]-1-methyl-5-oxo-4,5-dihydro-1H-imidazol-4-yl}phenyl 2-methoxyethanesulfonate;
3-{2-Amino-4-[3-(5-methoxypyridin-3-yl)phenyl]-1-methyl-5-oxo-4,5-dihydro-1H-imidazol-4-yl}phenyl dimethylsulfamate;
3-[2-Amino-1-methyl-5-oxo-4-(3-pyrimidin-2-ylphenyl)-4,5-dihydro-1H-imidazol-4-yl]phenyl dimethylsulfamate;
3-{2-Amino-4-[3-(5-methoxypyridin-3-yl)phenyl]-1-methyl-5-oxo-4,5-dihydro-1H-imidazol-4-yl}phenyl cyclopropanesulfonate;
3-[2-Amino-1-methyl-5-oxo-4-(3-pyrimidin-2-ylphenyl)-4,5-dihydro-1H-imidazol-4-yl]phenyl cyclopropanesulfonate;
3-[2-Amino-4-(4-methoxyphenyl)-1-methyl-5-oxo-4,5-dihydro-1H-imidazol-4-yl]phenyl 3-methoxypropane-1-sulfonate hydrochloride;
3-[2-Amino-4-(4-methoxyphenyl)-1-methyl-5-oxo-4,5-dihydro-1H-imidazol-4-yl]phenyl methanesulfonate hydrochloride;
3-[2-Amino-4-(4-methoxyphenyl)-1-methyl-5-oxo-4,5-dihydro-1H-imidazol-4-yl]phenyl 2-ethoxyethanesulfonate hydrochloride;
3-[2-Amino-4-(4-methoxyphenyl)-1-methyl-5-oxo-4,5-dihydro-1H-imidazol-4-yl]phenyl 2-methoxyethanesulfonate hydrochloride;
4-[2-Amino-1-methyl-5-oxo-4-(3-phenoxyphenyl)-4,5-dihydro-1H-imidazol-4-yl]phenyl methanesulfonate hydrochloride;
4-[2-Amino-1-methyl-5-oxo-4-(3-phenoxyphenyl)-4,5-dihydro-1H-imidazol-4-yl]phenyl propane-1-sulfonate hydrochloride;
4-{2-Amino-4-[3-(3-methoxyphenoxy)phenyl]-1-methyl-5-oxo-4,5-dihydro-1H-imidazol-4-yl}phenyl methanesulfonate hydrochloride;
3-[2-Amino-4-(3′-methoxybiphenyl-3-yl)-1-methyl-5-oxo-4,5-dihydro-1H-imidazol-4-yl]phenyl propane-2-sulfonate hydrochloride;
2-Amino-5-(3′-hydroxy-5′-methoxybiphenyl-3-yl)-3-methyl-5-phenyl-3,5-dihydro-4H-imidazol-4-one;
3′-(2-Amino-1-methyl-5-oxo-4-phenyl-4,5-dihydro-1H-imidazol-4-yl)-5-methoxybiphenyl-3-yl trifluoromethanesulfonate hydrochloride;
4-{2-Amino-4-[3-(5-methoxypyridin-3-yl)phenyl]-1-methyl-5-oxo-4,5-dihydro-1H-imidazol-4-yl}phenyl propane-1-sulfonate hydrochloride;
2-Amino-5-(3-bromophenyl)-5-(6-methoxy-5,6,7,8-tetrahydronaphthalen-2-yl)-3-methyl-3,5-dihydro-4H-imidazol-4-one;
5-(1-Acetyl-1,2,3,4-tetrahydroquinolin-6-yl)-2-amino-5-(3-bromophenyl)-3-methyl-3,5-dihydro-4H-imidazol-4-one;
2-Amino-5-(3-bromophenyl)-3-methyl-5-(5,6,7,8-tetrahydronaphthalen-2-yl)imidazolidin-4-one;
2-Amino-5-(3-bromophenyl)-5-(2,3-dihydro-1H-inden-5-yl)-3-methylimidazolidin-4-one;
2-Amino-5-(3′-methoxybiphenyl-3-yl)-5-(6-methoxy-5,6,7,8-tetrahydronaphthalen-2-yl)-3-methyl-3,5-dihydro-4H-imidazol-4-one 0.25 acetate;
2-Amino-5-(2′-fluoro-5′-methoxybiphenyl-3-yl)-5-(6-methoxy-5,6,7,8-tetrahydronaphthalen-2-yl)-3-methyl-3,5-dihydro-4H-imidazol-4-one;
5-(1-Acetyl-1,2,3,4-tetrahydroquinolin-6-yl)-2-amino-5-(3′-methoxybiphenyl-3-yl)-3-methyl-3,5-dihydro-4H-imidazol-4-one;
5-(1-Acetyl-1,2,3,4-tetrahydroquinolin-6-yl)-2-amino-5-(2′,5′-dimethoxybiphenyl-3-yl)-3-methyl-3,5-dihydro-4H-imidazol-4-one 0.25 acetate;
3′-[4-(1-Acetyl-1,2,3,4-tetrahydroquinolin-6-yl)-2-amino-1-methyl-5-oxo-4,5-dihydro-1H-imidazol-4-yl]biphenyl-3-carbonitrile;
3′-[4-(1-Acetyl-1,2,3,4-tetrahydroquinolin-6-yl)-2-amino-1-methyl-5-oxo-4,5-dihydro-1H-imidazol-4-yl]-4-fluorobiphenyl-3-carbonitrile;
5-(1-Acetyl-1,2,3,4-tetrahydroquinolin-6-yl)-2-amino-5-(2′-fluoro-5′-methoxybiphenyl-3-yl)-3-methyl-3,5-dihydro-4H-imidazol-4-one 0.25 acetate;
5-(1-Acetyl-1,2,3,4-tetrahydroquinolin-6-yl)-2-amino-3-methyl-5-(3′-nitrobiphenyl-3-yl)-3,5-dihydro-4H-imidazol-4-one 0.25 acetate;
5-(1-Acetyl-1,2,3,4-tetrahydroquinolin-6-yl)-2-amino-5-(2′-fluoro-3′-methoxybiphenyl-3-yl)-3-methyl-3,5-dihydro-4H-imidazol-4-one;
5-(1-Acetyl-1,2,3,4-tetrahydroquinolin-6-yl)-2-amino-5-[3-(3-furyl)phenyl]-3-methyl-3,5-dihydro-4H-imidazol-4-one;
2-Amino-5-(3′-methoxybiphenyl-3-yl)-3-methyl-5-(5,6,7,8-tetrahydronaphthalen-2-yl)-3,5-dihydro-4H-imidazol-4-one;
3′-[2-Amino-1-methyl-5-oxo-4-(5,6,7,8-tetrahydronaphthalen-2-yl)-4,5-dihydro-1H-imidazol-4-yl]-6-fluorobiphenyl-3-carbonitrile;
2-Amino-5-(2′,5′-dimethoxybiphenyl-3-yl)-3-methyl-5-(5,6,7,8-tetrahydronaphthalen-2-yl)-3,5-dihydro-4H-imidazol-4-one;
2-Amino-5-[3-(1,3-benzodioxol-5-yl)phenyl]-3-methyl-5-(5,6,7,8-tetrahydronaphthalen-2-yl)-3,5-dihydro-4H-imidazol-4-one 0.25 acetate;
2-Amino-5-(3′-ethoxybiphenyl-3-yl)-3-methyl-5-(5,6,7,8-tetrahydronaphthalen-2-yl)-3,5-dihydro-4H-imidazol-4-one;
3′-[2-Amino-1-methyl-5-oxo-4-(5,6,7,8-tetrahydronaphthalen-2-yl)-4,5-dihydro-1H-imidazol-4-yl]biphenyl-3-carbonitrile;
3′-[2-Amino-1-methyl-5-oxo-4-(5,6,7,8-tetrahydronaphthalen-2-yl)-4,5-dihydro-1H-imidazol-4-yl]-4-fluorobiphenyl-3-carbonitrile;
2-Amino-5-(2′-fluoro-5′-methoxybiphenyl-3-yl)-3-methyl-5-(5,6,7,8-tetrahydronaphthalen-2-yl)-3,5-dihydro-4H-imidazol-4-one;
2-Amino-3-methyl-5-(3′-nitrobiphenyl-3-yl)-5-(5,6,7,8-tetrahydronaphthalen-2-yl)-3,5-dihydro-4H-imidazol-4-one;
2-Amino-5-(2′-fluoro-3′-methoxybiphenyl-3-yl)-3-methyl-5-(5,6,7,8-tetrahydronaphthalen-2-yl)-3,5-dihydro-4H-imidazol-4-one;
2-Amino-5-[3-(3-furyl)phenyl]-3-methyl-5-(5,6,7,8-tetrahydronaphthalen-2-yl)-3,5-dihydro-4H-imidazol-4-one;
2-Amino-5-(2,3-dihydro-1H-inden-5-yl)-5-(3′-methoxybiphenyl-3-yl)-3-methyl-3,5-dihydro-4H-imidazol-4-one 0.25 acetate;
3′-[2-Amino-4-(6-methoxy-5,6,7,8-tetrahydronaphthalen-2-yl)-1-methyl-5-oxo-4,5-dihydro-1H-imidazol-4-yl]-5-methoxybiphenyl-3-yl methanesulfonate;
3′-[4-(1-Acetyl-1,2,3,4-tetrahydroquinolin-6-yl)-2-amino-1-methyl-5-oxo-4,5-dihydro-1H-imidazol-4-yl]-5-methoxybiphenyl-3-yl methanesulfonate;
3′-[2-Amino-1-methyl-5-oxo-4-(5,6,7,8-tetrahydronaphthalen-2-yl)-4,5-dihydro-1H-imidazol-4-yl]-5-methoxybiphenyl-3-yl methanesulfonate;
3′-[2-Amino-4-(2,3-dihydro-1H-inden-5-yl)-1-methyl-5-oxo-4,5-dihydro-1H-imidazol-4-yl]-5-methoxybiphenyl-3-yl methanesulfonate;
2-Amino-5-(3-bromophenyl)-5-(2,3-dihydro-1-benzofuran-5-yl)-3-methyl-3,5-dihydro-4H-imidazol-4-one;
2-Amino-5-(3-bromophenyl)-5-(3,4-dihydro-2H-chromen-6-yl)-3-methyl-3,5-dihydro-4H-imidazol-4-one;
2-Amino-5-(2,3-dihydro-1-benzofuran-5-yl)-5-(3′-methoxybiphenyl-3-yl)-3-methyl-3,5-dihydro-4H-imidazol-4-one hydrochloride;
5-[3-(2-Acetyl-2,3-dihydro-1H-isoindol-4-yl)phenyl]-2-amino-3-methyl-5-phenyl-3,5-dihydro-4H-imidazol-4-one acetate;
2-Amino-5-(3-bromophenyl)-5-(3,4-dihydro-1H-isochromen-7-yl)-3-methyl-3,5-dihydro-4H-imidazol-4-one;
2-Amino-5-(2,3-dihydro-1-benzofuran-5-yl)-5-(3′-ethoxybiphenyl-3-yl)-3-methyl-3,5-dihydro-4H-imidazol-4-one 0.25 acetate;
2-Amino-5-(2,3-dihydro-1-benzofuran-5-yl)-5-(2′,5′-dimethoxybiphenyl-3-yl)-3-methyl-3,5-dihydro-4H-imidazol-4-one 0.25 acetate;
2-Amino-5-[3-(1,3-benzodioxol-5-yl)phenyl]-5-(2,3-dihydro-1-benzofuran-5-yl)-3-methyl-3,5-dihydro-4H-imidazol-4-one 0.25 acetate;
2-Amino-5-(2,3-dihydro-1-benzofuran-5-yl)-5-(2′-fluoro-5′-methoxybiphenyl-3-yl)-3-methyl-3,5-dihydro-4H-imidazol-4-one 0.25 acetate;
2-Amino-5-(2,3-dihydro-1-benzofuran-5-yl)-5-(2′-fluoro-3′-methoxybiphenyl-3-yl)-3-methyl-3,5-dihydro-4H-imidazol-4-one 0.25 acetate;
3′-[2-Amino-4-(2,3-dihydro-1-benzofuran-5-yl)-1-methyl-5-oxo-4,5-dihydro-1H-imidazol-4-yl]-6-fluorobiphenyl-3-carbonitrile 0.25 acetate;
2-Amino-5-(2,3-dihydro-1-benzofuran-5-yl)-3-methyl-5-(3′-nitrobiphenyl-3-yl)-3,5-dihydro-4H-imidazol-4-one 0.25 acetate;
2-Amino-5-(2,3-dihydro-1-benzofuran-5-yl)-5-[3-(3-furyl)phenyl]-3-methyl-3,5-dihydro-4H-imidazol-4-one 0.25 acetate;
2-Amino-5-[3-(1-benzofuran-2-yl)phenyl]-5-(2,3-dihydro-1-benzofuran-5-yl)-3-methyl-3,5-dihydro-4H-imidazol-4-one 0.25 acetate;
2-Amino-5-(2,3-dihydro-1-benzofuran-5-yl)-3-methyl-5-[3′-(trifluoromethoxy)biphenyl-3-yl]-3,5-dihydro-4H-imidazol-4-one 0.25 acetate;
2-Amino-5-(3′-chlorobiphenyl-3-yl)-5-(2,3-dihydro-1-benzofuran-5-yl)-3-methyl-3,5-dihydro-4H-imidazol-4-one x 0.25 acetate;
2-Amino-5-(3,4-dihydro-2H-chromen-6-yl)-5-(3′-methoxybiphenyl-3-yl)-3-methyl-3,5-dihydro-4H-imidazol-4-one 0.25 acetate;
2-Amino-5-(3,4-dihydro-2H-chromen-6-yl)-5-(3′-ethoxybiphenyl-3-yl)-3-methyl-3,5-dihydro-4H-imidazol-4-one;
2-Amino-5-(3,4-dihydro-2H-chromen-6-yl)-5-(2′,5′-dimethoxybiphenyl-3-yl)-3-methyl-3,5-dihydro-4H-imidazol-4-one 0.25 acetate;
2-Amino-5-[3-(1,3-benzodioxol-5-yl)phenyl]-5-(3,4-dihydro-2H-chromen-6-yl)-3-methyl-3,5-dihydro-4H-imidazol-4-one 0.25 acetate;
2-Amino-5-(3,4-dihydro-2H-chromen-6-yl)-5-(2′-fluoro-5′-methoxybiphenyl-3-yl)-3-methyl-3,5-dihydro-4H-imidazol-4-one;
2-Amino-5-(3,4-dihydro-2H-chromen-6-yl)-5-(2′-fluoro-3′-methoxybiphenyl-3-yl)-3-methyl-3,5-dihydro-4H-imidazol-4-one;
3′-[2-Amino-4-(3,4-dihydro-2H-chromen-6-yl)-1-methyl-5-oxo-4,5-dihydro-1H-imidazol-4-yl]-4-fluorobiphenyl-3-carbonitrile;
3′-[2-Amino-4-(3,4-dihydro-2H-chromen-6-yl)-1-methyl-5-oxo-4,5-dihydro-1H-imidazol-4-yl]-6-fluorobiphenyl-3-carbonitrile 0.25 acetate;
2-Amino-5-(3,4-dihydro-2H-chromen-6-yl)-3-methyl-5-(3′-nitrobiphenyl-3-yl)-3,5-dihydro-4H-imidazol-4-one 0.25 acetate;
2-Amino-5-(3,4-dihydro-2H-chromen-6-yl)-5-[3-(3-furyl)phenyl]-3-methyl-3,5-dihydro-4H-imidazol-4-one 0.25 acetate;
2-Amino-5-[3-(1-benzofuran-2-yl)phenyl]-5-(3,4-dihydro-2H-chromen-6-yl)-3-methyl-3,5-dihydro-4H-imidazol-4-one;
2-Amino-5-(3,4-dihydro-2H-chromen-6-yl)-3-methyl-5-[3′-(trifluoromethoxy)biphenyl-3-yl]-3,5-dihydro-4H-imidazol-4-one 0.25 acetate;
2-Amino-5-(3′-chlorobiphenyl-3-yl)-5-(3,4-dihydro-2H-chromen-6-yl)-3-methyl-3,5-dihydro-4H-imidazol-4-one;
2-Amino-5-(3,4-dihydro-1H-isochromen-7-yl)-5-(2′-fluoro-5′-methoxybiphenyl-3-yl)-3-methyl-3,5-dihydro-4H-imidazol-4-one;
3′-[2-Amino-4-(3,4-dihydro-1H-isochromen-7-yl)-1-methyl-5-oxo-4,5-dihydro-1H-imidazol-4-yl]-6-fluorobiphenyl-3-carbonitrile 0.25 acetate;
2-Amino-5-(3,4-dihydro-1H-isochromen-7-yl)-5-[3-(3-furyl)phenyl]-3-methyl-3,5-dihydro-4H-imidazol-4-one 0.25 acetate;
3′-[2-Amino-4-(2,3-dihydro-1-benzofuran-5-yl)-1-methyl-5-oxo-4,5-dihydro-1H-imidazol-4-yl]-5-methoxybiphenyl-3-yl methanesulfonate 0.25 acetate;
3′-[2-Amino-4-(3,4-dihydro-2H-chromen-6-yl)-1-methyl-5-oxo-4,5-dihydro-1H-imidazol-4-yl]-5-methoxybiphenyl-3-yl methanesulfonate 0.25 acetate;
3′-[2-Amino-4-(3,4-dihydro-1H-isochromen-7-yl)-1-methyl-5-oxo-4,5-dihydro-1H-imidazol-4-yl]-5-methoxybiphenyl-3-yl methanesulfonate;
4-[2-Amino-4-(3-bromo-4-fluorophenyl)-1-methyl-5-oxo-4,5-dihydro-1H-imidazol-4-yl]phenyl methanesulfonate;
4-[2-Amino-4-(3′-cyano-6-fluorobiphenyl-3-yl)-1-methyl-5-oxo-4,5-dihydro-1H-imidazol-4-yl]phenyl methanesulfonate;
4-[2-Amino-4-(3′-cyano-6-fluorobiphenyl-3-yl)-5-oxo-4,5-dihydro-1H-imidazol-4-yl]phenyl methanesulfonate;
4-[2-Amino-4-(2′,6-difluoro-3′-methoxybiphenyl-3-yl)-5-oxo-4,5-dihydro-1H-imidazol-4-yl]phenyl methanesulfonate;
4-[2-Amino-4-(2′,6-difluoro-5′-methoxybiphenyl-3-yl)-5-oxo-4,5-dihydro-1H-imidazol-4-yl]phenyl methanesulfonate;
4-[2-Amino-4-(3′-cyano-4′,6-difluorobiphenyl-3-yl)-5-oxo-4,5-dihydro-1H-imidazol-4-yl]phenyl methanesulfonate;
4-[2-Amino-4-(5′-cyano-2′,6-difluorobiphenyl-3-yl)-5-oxo-4,5-dihydro-1H-imidazol-4-yl]phenyl methanesulfonate;
4-[2-Amino-4-(4-fluoro-3-pyridin-3-ylphenyl)-5-oxo-4,5-dihydro-1H-imidazol-4-yl]phenyl methanesulfonate;
4-{2-Amino-4-[4-fluoro-3-(2-fluoropyridin-3-yl)phenyl]-5-oxo-4,5-dihydro-1H-imidazol-4-yl}phenyl methanesulfonate;
4-{2-Amino-4-[3-(5-chloro-2-fluoropyridin-3-yl)-4-fluorophenyl]-5-oxo-4,5-dihydro-1H-imidazol-4-yl}phenyl methanesulfonate;
4-{2-Amino-4-[4-fluoro-3-(6-fluoropyridin-3-yl)phenyl]-5-oxo-4,5-dihydro-1H-imidazol-4-yl}phenyl methanesulfonate;
4-{2-Amino-4-[4-fluoro-3-(2-fluoropyridin-4-yl)phenyl]-5-oxo-4,5-dihydro-1H-imidazol-4-yl}phenyl methanesulfonate;
4-{2-Amino-4-[3-(2-chloro-3-fluoropyridin-4-yl)-4-fluorophenyl]-5-oxo-4,5-dihydro-1H-imidazol-4-yl}phenyl methanesulfonate;
4-{2-Amino-4-[3-(2-chloro-5-fluoropyridin-3-yl)-4-fluorophenyl]-5-oxo-4,5-dihydro-1H-imidazol-4-yl}phenyl methanesulfonate;
4-{2-Amino-4-[3-(2,6-difluoropyridin-3-yl)-4-fluorophenyl]-5-oxo-4,5-dihydro-1H-imidazol-4-yl}phenyl methanesulfonate;
4-{2-Amino-4-[6-fluoro-3′-(trifluoromethoxy)biphenyl-3-yl]-5-oxo-4,5-dihydro-1H-imidazol-4-yl}phenyl methanesulfonate;
4-[2-Amino-4-(3′-chloro-6-fluorobiphenyl-3-yl)-5-oxo-4,5-dihydro-1H-imidazol-4-yl]phenyl methanesulfonate;
4-{2-Amino-4-[3-(1,3-benzodioxol-5-yl)-4-fluorophenyl]-5-oxo-4,5-dihydro-1H-imidazol-4-yl}phenyl methanesulfonate;
4-[2-Amino-4-(4-fluoro-3-pyridin-4-ylphenyl)-5-oxo-4,5-dihydro-1H-imidazol-4-yl]phenyl methanesulfonate;
4-[2-Amino-4-(4-fluoro-3-pyrimidin-5-ylphenyl)-5-oxo-4,5-dihydro-1H-imidazol-4-yl]phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-{2-Amino-4-[3-(2-chloro-5-methoxypyridin-3-yl)-4-fluorophenyl]-5-oxo-4,5-dihydro-1H-imidazol-4-yl}phenyl methanesulfonate;
4-(2-Amino-4-{6-fluoro-3′-methoxy-5′-[(methylsulfonyl)oxy]biphenyl-3-yl}-5-oxo-4,5-dihydro-1H-imidazol-4-yl)phenyl methanesulfonate;
4-{2-Amino-4-[4-fluoro-3-(5-fluoropyridin-3-yl)phenyl]-5-oxo-4,5-dihydro-1H-imidazol-4-yl}phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
4-[2-Amino-4-(4-fluoro-3-pyrazin-2-ylphenyl)-5-oxo-4,5-dihydro-1H-imidazol-4-yl]phenyl methanesulfonate;
2-Amino-5-(4-hydroxyphenyl)-5-(3′-methoxybiphenyl-3-yl)-3-methyl-3,5-dihydro-4H-imidazol-4-one;
4-[2-Amino-4-(3′-methoxybiphenyl-3-yl)-1-methyl-5-oxo-4,5-dihydro-1H-imidazol-4-yl]phenyl propane-1-sulfonate hydrochloride;
4-[2-Amino-4-(3′-methoxybiphenyl-3-yl)-1-methyl-5-oxo-4,5-dihydro-1H-imidazol-4-yl]phenyl propane-2-sulfonate hydrochloride;
4-[2-Amino-4-(3′-methoxybiphenyl-3-yl)-1-methyl-5-oxo-4,5-dihydro-1H-imidazol-4-yl]phenyl dimethylsulfamate hydrochloride;
4-[2-Amino-4-(3′-methoxybiphenyl-3-yl)-1-methyl-5-oxo-4,5-dihydro-1H-imidazol-4-yl]phenyl morpholine-4-sulfonate; and
4-[2-Amino-4-(3′-methoxybiphenyl-3-yl)-1-methyl-5-oxo-4,5-dihydro-1H-imidazol-4-yl]phenyl 2-methoxyethanesulfonate hydrochloride;
as a free base or a pharmaceutically acceptable salt, solvate or solvate of a salt thereof.

30. A pharmaceutical formulation comprising as active ingredient a therapeutically effective amount of a compound according to claim 1 in association with a pharmaceutically acceptable excipient, carrier or diluent.

31-35. (canceled)

36. A method of inhibiting activity of BACE comprising contacting said BACE with a compound of claim 1.

37. A method of treating or preventing an Aβ-related pathology in a mammal, comprising administering to said patient a therapeutically effective amount of a compound of claim 1.

38. The method of claim 37, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

39. The method of claim 37, wherein said mammal is a human.

40. A method of treating or preventing an Aβ-related pathology in a mammal, comprising administering to said patient a therapeutically effective amount of a compound of claim 1 and at least one cognitive enhancing agent, memory enhancing agent, or choline esterase inhibitor.

41. The method of claim 40, wherein said Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

42. The method of claim 40, wherein said mammal is a human.

43. A compound, said compound being:
2-Acetyl-7-bromo-1,2,3,4-tetrahydroisoquinoline;
2-Acetyl-7-(4,4,5,5-tetramethyl-1,3,2-dioxaborolan-2-yl)-1,2,3,4-tetrahydroisoquinoline;
5-(3-Bromo-phenyl)-3-methyl-5-phenyl-2-thioxo-imidazolidin-4-one;
1-Acetyl-5-(4,4,5,5-tetramethyl-1,3,2-dioxaborolan-2-yl)indoline;
6-(4,4,5,5-Tetramethyl-1,3,2-dioxaborolan-2-yl)-2,3-dihydro-1-benzofuran;
(4-{[tert-Butyl(diphenyl)silyl]oxy}phenyl) [2-(3′-methoxybiphenyl-3-yl)-1,3-dithian-2-yl]methanol;
1-(4-{[tert-Butyl(diphenyl)silyl]oxy}phenyl)-2-(3′-methoxybiphenyl-3-yl)ethane-1,2-dione;
5-(4-Hydroxyphenyl)-5-(3′-methoxybiphenyl-3-yl)-3-methyl-2-thioxoimidazolidin-4-one;
4-[4-(3′-Methoxybiphenyl-3-yl)-1-methyl-5-oxo-2-thioxoimidazolidin-4-yl]phenyl methanesulfonate;
4-[4-(3′-Methoxybiphenyl-3-yl)-1-methyl-5-oxo-2-thioxoimidazolidin-4-yl]phenyl trifluoromethanesulfonate;
4-Methoxy-2-(4,4,5,5-tetramethyl-1,3,2-dioxaborolan-2-yl)phenol;
1-(3-Hydroxyphenyl)-2-phenylethane-1,2-dione;
5-(3-Hydroxyphenyl)-3-methyl-5-phenyl-2-thioxoimidazolidin-4-one;
Methyl-5-oxo-4-phenyl-2-thioxoimidazolidin-4-yl)phenyl methanesulfonate;
3-Bromo-4-{[tert-butyl(diphenyl)silyl]oxy}benzaldehyde;
3-Bromo-4-{[tert-butyl(diphenyl)silyl]oxy}phenyl)(2-phenyl-1,3-dithian-2-yl)methanol;
1-(3-Bromo-4-{[tert-butyl(diphenyl)silyl]oxy}phenyl)-2-phenylethane-1,2-dione;
5-(3-Bromo-4-hydroxyphenyl)-3-methyl-5-phenyl-2-thioxoimidazolidin-4-one;
2-(3′-Methoxybiphenyl-3-yl)-1,3-dithiane;
(3-{[tert-Butyl(diphenyl)silyl]oxy}phenyl)[2-(3′-methoxybiphenyl-3-yl)-1,3-dithian-2-yl]methanol;
1-(3-{[tert-Butyl(diphenyl)silyl]oxy}phenyl)-2-(3′-methoxybiphenyl-3-yl)ethane-1,2-dione;
5-(3-Hydroxyphenyl)-5-(3′-methoxybiphenyl-3-yl)-3-methyl-2-thioxoimidazolidin-4-one;
3-[4-(3′-Methoxybiphenyl-3-yl)-1-methyl-5-oxo-2-thioxoimidazolidin-4-yl]phenyl trifluoromethanesulfonate;
3-[4-(3′-Methoxybiphenyl-3-yl)-1-methyl-5-oxo-2-thioxoimidazolidin-4-yl]phenyl methanesulfonate;
5-(4,4,5,5-Tetramethyl-1,3,2-dioxaborolan-2-yl)-2,3-dihydro-1-benzofuran;
2,3-Dihydro-1-benzofuran-5-yl(2-phenyl-1,3-dithian-2-yl)methanol;
1-(2,3-Dihydro-1-benzofuran-5-yl)-2-phenylethane-1,2-dione;
5-(2,3-Dihydro-1-benzofuran-5-yl)-3-methyl-5-phenyl-2-thioxoimidazolidin-4-one;
2-Acetyl-5-chloro-1,2,3,4-tetrahydroisoquinoline;
2-Acetyl-5-(4,4,5,5-tetramethyl-1,3,2-dioxaborolan-2-yl)-1,2,3,4-tetrahydroisoquinoline;
(4-Methyl-3,4-dihydro-2H-1,4-benzoxazin-7-yl)(2-phenyl-1,3-dithian-2-yl)methanol
1-(4-Methyl-3,4-dihydro-2H-1,4-benzoxazin-7-yl)-2-phenylethane-1,2-dione;
3-Methyl-5-(4-methyl-3,4-dihydro-2H-1,4-benzoxazin-7-yl)-5-phenyl-2-thioxoimidazolidin-4-one;
6-Iodo-1,2,3,4-tetrahydronaphthalene;
6-(Phenylethynyl)-1,2,3,4-tetrahydronaphthalene;
1-Phenyl-2-(5,6,7,8-tetrahydronaphthalen-2-yl)ethane-1,2-dione;
3-Methyl-5-phenyl-5-(5,6,7,8-tetrahydronaphthalen-2-yl)-2-thioxoimidazolidin-4-one;
1-Acetyl-5-iodoindoline;
1-Acetyl-5-(phenylethynyl)indoline;
1-(1-Acetyl-2,3-dihydro-1H-indol-5-yl)-2-phenylethane-1,2-dione;
5-(1-Acetyl-2,3-dihydro-1H-indol-5-yl)-3-methyl-5-phenyl-2-thioxoimidazolidin-4-one;
1-Acetyl-4-(4,4,5,5-tetramethyl-1,3,2-dioxaborolan-2-yl)indoline;
8-(4,4,5,5-Tetramethyl-1,3,2-dioxaborolan-2-yl)chromane;
3-Methoxy-5-(4,4,5,5-tetramethyl-1,3,2-dioxaborolan-2-yl)phenol;
3′-Hydroxy-5′-methoxybiphenyl-3-carbaldehyde;
3′-{[tert-Butyl(diphenyl)silyl]oxy}-5′-methoxybiphenyl-3-carbaldehyde;
tert-Butyl {[3′-(1,3-dithian-2-yl)-5-methoxybiphenyl-3-yl]oxy}diphenylsilane;
[2-(3′-{[tert-Butyl(diphenyl)silyl]oxy}-5′-methoxybiphenyl-3-yl)-1,3-dithian-2-yl](pyridin-4-yl)methanol;
1-(3′-{[tert-Butyl(diphenyl)silyl]oxy}-5′-methoxybiphenyl-3-yl)-2-pyridin-4-ylethane-1,2-dione;
5-(3′-Hydroxy-5′-methoxybiphenyl-3-yl)-3-methyl-5-pyridin-4-yl-2-thioxoimidazolidin-4-one;
5-Methoxy-3′-(1-methyl-5-oxo-4-pyridin-4-yl-2-thioxoimidazolidin-4-yl)biphenyl-3-yl methanesulfonate;
[2-(3′-{[tert-Butyl(diphenyl)silyl]oxy}-5′-methoxybiphenyl-3-yl)-1,3-dithian-2-yl] (pyridin-2-yl)methanol;
1-(3′-{[tert-Butyl(diphenyl)silyl]oxy}-5′-methoxybiphenyl-3-yl)-2-pyridin-2-ylethane-1,2-dione;
5-(3′-Hydroxy-5′-methoxybiphenyl-3-yl)-3-methyl-5-pyridin-2-yl-2-thioxoimidazolidin-4-one;
5-Methoxy-3′-(1-methyl-5-oxo-4-pyridin-2-yl-2-thioxoimidazolidin-4-yl)biphenyl-3-yl methanesulfonate;
[2-(3′-{[tert-Butyl(diphenyl)silyl]oxy}-5′-methoxybiphenyl-3-yl)-1,3-dithian-2-yl](3-furyl)methanol;
1-(3′-{[tert-Butyl(diphenyl)silyl]oxy}-5′-methoxybiphenyl-3-yl)-2-(3-furyl)ethane-1,2-dione;
5-(3-Furyl)-5-(3′-hydroxy-5′-methoxybiphenyl-3-yl)-3-methyl-2-thioxoimidazolidin-4-one;
3′-[4-(3-Furyl)-1-methyl-5-oxo-2-thioxoimidazolidin-4-yl]-5-methoxybiphenyl-3-yl methanesulfonate;
3′-[2-Amino-4-(3-furyl)-1-methyl-5-oxo-4,5-dihydro-1H-imidazol-4-yl]-5-methoxybiphenyl-3-yl methanesulfonate;
[2-(3′-{[tert-Butyl(diphenyl)silyl]oxy}-5′-methoxybiphenyl-3-yl)-1,3-dithian-2-yl](1,3-thiazol-5-yl)methanol;
1-(3′-{[tert-Butyl(diphenyl)silyl]oxy}-5′-methoxybiphenyl-3-yl)-2-(1,3-thiazol-5-yl)ethane-1,2-dione;
5-(3′-Hydroxy-5′-methoxybiphenyl-3-yl)-3-methyl-5-(1,3-thiazol-5-yl)-2-thioxoimidazolidin-4-one;
5-Methoxy-3′-[1-methyl-5-oxo-4-(1,3-thiazol-5-yl)-2-thioxoimidazolidin-4-yl]biphenyl-3-yl methanesulfonate;
[2-(3′-{[tert-Butyl(diphenyl)silyl]oxy}-5′-methoxybiphenyl-3-yl)-1,3-dithian-2-yl](1,3-thiazol-4-yl)methanol;
1-(3′-{[tert-Butyl(diphenyl)silyl]oxy}-5′-methoxybiphenyl-3-yl)-2-(1,3-thiazol-4-yl)ethane-1,2-dione;
5-(3′-Hydroxy-5′-methoxybiphenyl-3-yl)-3-methyl-5-(1,3-thiazol-4-yl)-2-thioxoimidazolidin-4-one;
5-Methoxy-3′-[1-methyl-5-oxo-4-(1,3-thiazol-4-yl)-2-thioxoimidazolidin-4-yl]biphenyl-3-yl methanesulfonate;
4-Bromo-1-fluoro-2-methoxybenzene;
2-(4-Fluoro-3-methoxyphenyl)-4,4,5,5-tetramethyl-1,3,2-dioxaborolane 4-[(3-Bromophenyl)ethynyl]phenol;
1-(3-Bromophenyl)-2-(4-hydroxyphenyl)ethane-1,2-dione;
5-(3-Bromophenyl)-5-(4-hydroxyphenyl)-3-methyl-2-thioxoimidazolidin-4-one;
4-[4-(3-Bromophenyl)-1-methyl-5-oxo-2-thioxoimidazolidin-4-yl]phenyl methanesulfonate;
4-[4-(3-Bromophenyl)-1-methyl-5-oxo-2-thioxoimidazolidin-4-yl]phenyl 2-methoxyethanesulfonate;
4-[4-(3-Bromophenyl)-1-methyl-5-oxo-2-thioxoimidazolidin-4-yl]phenyl propane-1-sulfonate;
3-Methoxy-5-(4,4,5,5-tetramethyl-1,3,2-dioxaborolan-2-yl)phenyl methanesulfonate;
3-[(3-Bromophenyl)ethynyl]phenol;
1-(3-Bromophenyl)-2-(3-hydroxyphenyl)ethane-1,2-dione;
5-(3-Bromophenyl)-5-(3-hydroxyphenyl)-3-methyl-2-thioxoimidazolidin-4-one;
3-[4-(3-Bromophenyl)-1-methyl-5-oxo-2-thioxoimidazolidin-4-yl]phenyl methanesulfonate;
3-[4-(3-Bromophenyl)-1-methyl-5-oxo-2-thioxoimidazolidin-4-yl]phenyl 2-methoxyethanesulfonate;
2-(4,4,5,5-Tetramethyl-1,3,2-dioxaborolan-2-yl)pyrimidine;
1-Ethynyl-3-(3-methoxyphenoxy)benzene;
3-[(4-Methoxyphenyl)ethynyl]phenol;
4-{[3-(3-Methoxyphenoxy)phenyl]ethynyl}phenol;
1-(3-Hydroxyphenyl)-2-(4-methoxyphenyl)ethane-1,2-dione;
5-(3-Hydroxyphenyl)-5-(4-methoxyphenyl)-3-methyl-2-thioxoimidazolidin-4-one;
1-(4-Hydroxyphenyl)-2-[3-(3-methoxyphenoxy)phenyl]ethane-1,2-dione;
5-(4-Hydroxyphenyl)-5-[3-(3-methoxyphenoxy)phenyl]-3-methyl-2-thioxoimidazolidin-4-one;
1-(4-Methoxyphenyl)-2-(3-phenoxyphenyl)ethane-1,2-dione;
5-(4-Methoxyphenyl)-3-methyl-5-(3-phenoxyphenyl)-2-thioxoimidazolidin-4-one;
3-Methoxypropane-1-sulfonyl chloride;
3-[4-(3′-Methoxybiphenyl-3-yl)-1-methyl-5-oxo-2-thioxoimidazolidin-4-yl]phenyl propane-2-sulfonate;
3-[4-(3′-Methoxybiphenyl-3-yl)-1-methyl-5-oxo-2-thioxoimidazolidin-4-yl]phenyl morpholine-4-sulfonate;
3-[2-Amino-4-(3′-methoxybiphenyl-3-yl)-1-methyl-5-oxo-4,5-dihydro-1H-imidazol-4-yl]phenyl morpholine-4-sulfonate hydrochloride;
6-Iodo-1,2,3,4-tetrahydronaphthalene;
6-[(3-Bromophenyl)ethynyl]-1,2,3,4-tetrahydronaphthalene;
1-(3-Bromophenyl)-2-(5,6,7,8-tetrahydronaphthalen-2-yl)ethane-1,2-dione;
5-(3-Bromophenyl)-3-methyl-5-(5,6,7,8-tetrahydronaphthalen-2-yl)-2-thioxoimidazolidin-4-one;
6-Bromo-1,2,3,4-tetrahydronaphthalen-2-yl methyl ether;
[(6-Methoxy-5,6,7,8-tetrahydronaphthalen-2-yl)ethynyl](trimethyl)silane;
6-Ethynyl-1,2,3,4-tetrahydronaphthalen-2-yl methyl ether;
6-[(3-Bromophenyl)ethynyl]-1,2,3,4-tetrahydronaphthalen-2-yl methyl ether;
1-(3-Bromophenyl)-2-(6-methoxy-5,6,7,8-tetrahydronaphthalen-2-yl)ethane-1,2-dione;
5-(3-Bromophenyl)-5-(6-methoxy-5,6,7,8-tetrahydronaphthalen-2-yl)-3-methyl-2-thioxoimidazolidin-4-one;
1-Acetyl-6-iodo-1,2,3,4-tetrahydroquinoline;
1-Acetyl-6-[(3-bromophenyl)ethynyl]-1,2,3,4-tetrahydroquinoline;
1-(1-Acetyl-1,2,3,4-tetrahydroquinolin-6-yl)-2-(3-bromophenyl)ethane-1,2-dione;
5-(1-Acetyl-1,2,3,4-tetrahydroquinolin-6-yl)-5-(3-bromophenyl)-3-methyl-2-thioxoimidazolidin-4-one;
5-[(3-Bromophenyl)ethynyl]indane;
1-(3-Bromophenyl)-2-(2,3-dihydro-1H-inden-5-yl)ethane-1,2-dione;
5-(3-Bromophenyl)-5-(2,3-dihydro-1H-inden-5-yl)-3-methyl-2-thioxoimidazolidin-4-one;
2,3-Dihydro-1-benzofuran-5-yl[2-(3′-methoxybiphenyl-3-yl)-1,3-dithian-2-yl]methanol;
1-(2,3-Dihydro-1-benzofuran-5-yl)-2-(3′-methoxybiphenyl-3-yl)ethane-1,2-dione;
5-(2,3-Dihydro-1-benzofuran-5-yl)-5-(3′-methoxybiphenyl-3-yl)-3-methyl-2-thioxoimidazolidin-4-one;
2-Acetyl-4-chloroisoindoline;
5-[(3-Bromophenyl)ethynyl]-2,3-dihydro-1-benzofuran;
1-(3-Bromophenyl)-2-(2,3-dihydro-1-benzofuran-5-yl)ethane-1,2-dione;
5-(3-Bromophenyl)-5-(2,3-dihydro-1-benzofuran-5-yl)-3-methyl-2-thioxoimidazolidin-4-one;
6-[(3-Bromophenyl)ethynyl]chromane;
1-(3-Bromophenyl)-2-(3,4-dihydro-2H-chromen-6-yl)ethane-1,2-dione;
5-(3-Bromophenyl)-5-(3,4-dihydro-2H-chromen-6-yl)-3-methyl-2-thioxoimidazolidin-4-one;
(3,4-Dihydro-1H-isochromen-7-ylethynyl)(trimethyl)silane;
7-Ethynyl-3,4-dihydro-1H-isochromene;
7-[(3-Bromophenyl)ethynyl]-3,4-dihydro-1H-isochromene;
1-(3-Bromophenyl)-2-(3,4-dihydro-1H-isochromen-7-yl)ethane-1,2-dione;
5-(3-Bromophenyl)-5-(3,4-dihydro-1H-isochromen-7-yl)-3-methyl-2-thioxoimidazolidin-4-one;
4-{[tert-Butyl(diphenyl)silyl]oxy}benzaldehyde;
tert-Butyl[4-(1,3-dithian-2-yl)phenoxy]diphenylsilane;
(3-Bromo-4-fluorophenyl) [2-(4-{[tert-butyl(diphenyl)silyl]oxy}phenyl)-1,3-dithian-2-yl]methanol;
1-(3-Bromo-4-fluorophenyl)-2-(4-{[tert-butyl(diphenyl)silyl]oxy}phenyl)ethane-1,2-dione;
5-(3-Bromo-4-fluorophenyl)-5-(4-hydroxyphenyl)-3-methyl-2-thioxoimidazolidin-4-one; and
4-[4-(3-Bromo-4-fluorophenyl)-1-methyl-5-oxo-2-thioxoimidazolidin-4-yl]phenyl methanesulfonate.

44. (canceled)
